(19) United States
(12) Patent Application Publication
(10) Pub. No.: US 2004/0043021 A1
(43) Pub. Date: Mar. 4, 2004

(54) Nucleotide and amino acid sequences relating to respiratory diseases and obesity

(76) Inventors: Tim Keith, Bedford, MA (US); Randall D. Little, Newtonville, MA (US); Josee Dupuis, West Newton, MA (US); Paul Van Eerdewegh, Weston, MA (US); Richard G. Del Mastro, Norfolk, MA (US); Kristina Allen, Hopkinton, MA (US)

Correspondence Address: MORGAN & FINNEGAN, L.L.P., 345 PARK AVENUE, NEW YORK, NY, 10154, US

(21) Appl. No.: 10/271,416

(22) Filed: Oct. 11, 2002

Related U.S. Application Data
(60) Provisional application No. 60/328,424, filed on Oct. 11, 2001.

(51) Int. Cl. A61K 39/395; C07H 21/04; C12P 21/02; C12N 5/06; C07K 14/47; C07K 16/42

(52) U.S. Cl. 424/131.1; 435/69.1; 435/320.1; 435/325; 530/350; 530/387.2; 536/23.5

(57) ABSTRACT

This invention relates to ADAM and Interactor genes which are associated with various diseases, including asthma. The invention also relates to the nucleotide sequences of these genes, isolated nucleic acids comprising these nucleotide sequences, and isolated polypeptides or peptides encoded thereby. The invention further relates to vectors and host cells comprising the disclosed nucleotide sequences, or fragments thereof, as well as antibodies that bind to the encoded polypeptides or peptides. Also related are ligands that modulate the activity of the disclosed genes or gene products. In addition the invention relates to methods and compositions employing the disclosed nucleic acids, polypeptides or peptides, antibodies, or ligands for use in diagnostics and therapeutics for asthma and other diseases.

### RELATED APPLICATIONS

This application claims priority to U.S. Application Serial No. 60/328,424, filed Oct. 11, 2001, which is hereby incorporated by reference in its entirety.

### FIELD OF THE INVENTION

This invention relates to ADAM family genes and other Interactor genes identified to be associated with asthma, atopy, obesity, inflammatory bowel disease, and other human diseases. The invention also relates to the nucleotide sequences of these genes, including genomic DNA sequences, cDNA sequences, single nucleotide polymorphisms, alleles, haplotypes, and alternate splice variants. The invention further relates to isolated nucleic acids comprising these nucleotide sequences, and isolated polypeptides or peptides encoded thereby. Also related are expression vectors and host cells comprising the disclosed nucleic acids or fragments thereof, as well as antibodies that bind to the encoded polypeptides or peptides. The present invention further relates to ligands that modulate the activity of the disclosed genes or gene products. The invention further relates to diagnostics and therapeutics for various diseases, including asthma, utilizing ADAM genes and other Interactor genes, polypeptides, peptides, antibodies, or ligands.

### BACKGROUND

Asthma has been linked to specific markers on human chromosomes (Wilson et al., 1998, Genomics, 53: 251-259). Furthermore, asthma has been associated with other diseases, particularly, inflammatory lung disease phenotypes such as Chronic Obstructive Lung Disease (COPD), Adult Respiratory Distress Syndrome (ARDS), atopy, obesity, and inflammatory bowel disease.

Recently, an ADAM (A Disintegrin And Metalloprotease) family gene, ADAM33 (Gene 216), has been linked to asthma as described in U.S. patent application Ser. No. 09/834,597. The ADAM gene family, of which there are currently 33 members, is a sub-group of the zinc-dependent metalloprotease superfamily. ADAMs have a complex domain organization that includes a signal sequence, propeptide, metalloprotease, disintegrin, cysteine-rich, and epidermal growth factor-like domains, as well as a transmembrane region and cytoplasmic tail. ADAM proteins have been implicated in many processes such as proteolysis in the secretory pathway and extracellular matrix, extra- and intra-cellular signaling, processing of plasma membrane proteins and procytokine conversion.

Thus, there is a need in the art for the identification of other ADAM gene family members, substrates, and interactors that are involved in asthma and related disorders. Identification and characterization of such genes will allow the development of effective diagnostics and therapeutic means to diagnose, prevent, and treat lung related disorders, especially asthma, as well as the other diseases described herein.

### SUMMARY OF THE INVENTION

This invention relates to ADAM family genes and other Interactor genes associated with asthma, and related diseases thereof. In specific embodiments, the invention relates to the ADAM and Interactor genes shown in Table 2, as well as complementary sequences, sequence variants, or fragments thereof, as described herein. The present invention also encompasses nucleic acid probes and primers useful for assaying a biological sample for the presence or expression of ADAM and Interactor genes. In particular, this invention relates to the use of ADAM family and Interactor genes for the treatment and prevention of asthma, and related diseases thereof.

The invention further encompasses novel nucleic acid variants comprising alleles or haplotypes of single nucleotide polymorphisms (SNPs) identified in several of the ADAM and Interactor genes. Nucleic acid variants comprising SNP alleles or haplotypes can be used to diagnose diseases such as asthma, or to determine a genetic predisposition thereto. In addition, the present invention encompasses nucleic acids comprising alternate splicing variants.

This invention also relates to vectors and host cells comprising ADAM and Interactor genes and nucleic acid sequences disclosed herein. Such vectors can be used for nucleic acid preparations, including antisense nucleic acids, and for the expression of encoded polypeptides or peptides. Host cells can be prokaryotic or eukaryotic cells. In specific embodiments, an expression vector comprises a DNA sequence encoding a known ADAM or Interactor gene, sequence variants, or fragments thereof, as described herein.

The present invention further relates to isolated ADAM or Interactor gene polypeptides and peptides. In specific embodiments, the polypeptides or peptides comprise the amino acid sequences encoded by the ADAM or Interactor gene sequence variants, or portions thereof, as described herein. In addition, this invention encompasses isolated fusion proteins comprising ADAM and Interactor polypeptides or peptides.

The present invention also relates to isolated antibodies, including monoclonal and polyclonal antibodies, and antibody fragments, that are specifically reactive with the ADAM and Interactor polypeptides, fusion proteins, variants, or portions thereof, as disclosed herein. In specific embodiments, monoclonal antibodies are prepared to be specifically reactive with a ADAM or Interactor polypeptides, peptides, or sequence variants thereof.

In addition, the present invention relates to methods of obtaining ADAM and Interactor polynucleotides and polypeptides, variant sequences, or fragments thereof, as disclosed herein. Also related are methods of obtaining antibodies and antibody fragments that bind to ADAM and Interactor polypeptides, variant sequences, or fragments thereof. The present invention also encompasses methods of obtaining ADAM and Interactor ligands, e.g., agonists, antagonists, inhibitors, and binding factors. Such ligands can be used as therapeutics for asthma and related diseases.

The present invention also relates to diagnostic methods and kits utilizing ADAM and Interactor (wild-type, mutant, or variant) nucleic acids, polypeptides, antibodies, or functional fragments thereof. Such factors can be used, for example, in diagnostic methods and kits for measuring expression levels or obtaining ADAM or Interactor gene expression, and to screen for various diseases, especially asthma. In addition, the ADAM and Interactor nucleic acids described herein can be used to identify chromosomal abnormalities correlating with asthma and other related diseases.

The present invention further relates to methods and therapeutics for the treatment of various diseases, including asthma, atopy, obesity, and inflammatory bowel disease. In various embodiments, therapeutics comprising the disclosed ADAM and Interactor gene nucleic acids, polypeptides, antibodies, ligands, variants, derivatives, or portions thereof, are administered to a subject to treat, prevent, or ameliorate such diseases. Specifically related are therapeutics comprising ADAM and Interactor gene antisense nucleic acids, monoclonal antibodies, and gene therapy vectors. Such therapeutics can be administered alone, or in combination with one or more disease treatments.

In addition, this invention relates to non-human transgenic animals and cell lines comprising one or more of the disclosed ADAM or Interactor gene nucleic acids, which can be used for drug screening, protein production, and other purposes. Also related are non-human knock-out animals and cell lines, wherein one or more endogenous ADAM or Interactor genes (i.e., orthologs), or portions thereof, are deleted or replaced by marker genes.

This invention further relates to methods of identifying ADAM and Interactor proteins that are candidates for being involved in asthma and related diseases (i.e., a “candidate protein”). Such proteins are identified by a method comprising: 1) identifying a protein in a first individual having the asthma phenotype; 2) identifying an ADAM-related or Interactor protein in a second individual not having the asthma phenotype; and 3) comparing the protein of the first individual to the protein(s) of the second individual, wherein a) the protein that is present in the second individual but not the first individual is the candidate protein; or b) the protein that is present in a higher amount in the second individual than in the first individual is the candidate protein; or c) the protein that is present in a lower amount in the second individual than in the first individual is the candidate protein.

### BRIEF DESCRIPTION OF THE FIGURES

FIG. 1 shows the cDNA sequence for Gene 803 splice variant 1 (Accession No. NM_{—}003025) with the SNPs underlined.

FIG. 2 shows the cDNA sequence for Gene 803 splice variant 2 (Accession No. AK_{—}097616) with the SNPs underlined.

FIG. 3 shows the cDNA sequence of Gene 845 (Accession No. NM_{—}023038) with the SNPs underlined.

FIG. 4 shows the cDNA sequence for Gene 847 splice variant 1 (Accession No. NM_{—}004883) with the SNPs underlined.

FIG. 5 shows the cDNA sequence for Gene 847 splice variant 2 (Accession No. NM_{—}013981) with the SNPs underlined.

FIG. 6 shows the cDNA sequence for Gene 847 splice variant 3 (Accession No. NM_{—}013982) with the SNPs underlined.

FIG. 7 shows the cDNA sequence for Gene 847 splice variant 4 (Accession No. NM_{—}013983) with the SNPs underlined.

FIG. 8 shows the cDNA sequence for Gene 847 splice variant 5 (Accession No. NM_{—}013984) with the SNPs underlined.

FIG. 9 shows the cDNA sequence for Gene 847 splice variant 6 (Accession No. NM_{—}013985) with the SNPs underlined.

FIG. 10 shows the cDNA sequence for Gene 874 (Accession No. NM_{—}003026) with the SNPs underlined.

FIG. 11 shows the cDNA sequence for Gene 962 splice variant 1 (Accession No. NM_{—}014244) with the SNPs underlined.

FIG. 12 shows the cDNA sequence for Gene 962 splice variant 2 (Accession No. NM_{—}021599) with the SNPs underlined.

### DETAILED DESCRIPTION OF THE INVENTION

This invention is based on the discovery that ADAM genes and Interactor genes are associated with various diseases, including asthma, atopy, inflammatory bowel disease, and obesity.

To aid in the understanding of the specification and claims, the following definitions are provided.

### DEFINITIONS

“ADAM genes” or “ADAM family genes” or “ADAM-related genes” refers to the zinc-dependent metalloprotease gene superfamily comprised of multiple subgroups. Currently, there are 33 members of the ADAM family. The ADAM genes encode proteins of approximately 750 amino acids with 8 different domains. Domain I is a pre-domain and contains the signal sequence peptide that facilitates secretion through the plasma membrane. Domain II is a pro-domain that is cleaved before the protein is secreted resulting in activation of the catalytic domain. Domain III is a catalytic domain containing metalloprotease activity. Domain IV is a disintegrin-like domain and is believed to interact with integrins or other receptors. Domain V is a cysteine-rich domain and is speculated to be involved in protein-protein interactions or in the presentation of the disintegrin-like domain. Domain VI is an EGF-like domain that plays a role in stimulating membrane fusion. Domain VII is a transmembrane domain that anchors the ADAM protein to the membrane. Domain VIII is a cytoplasmic domain and contains binding sites for cytoskeletal-associated proteins and SH3 binding domains that may play a role in bi-directional signaling.

“Interactor genes” or “Interactors” refer to genes or proteins whose members interact with, are ligands or substrates for, or otherwise act in concert with ADAM family genes in the cellular processes or pathways associated with the diseases described herein. Examples of Interactor genes include those shown in Table 2, such as the Neuregulin and Endophilin family genes.

“Disorder region” refers to a portion of the human chromosome correlated with the disease type. A “disorder-associated” nucleic acid or “disorder-associated” polypeptide sequence refers to a nucleic acid sequence that maps to the disorder region and polypeptides encoded thereby. For nucleic acid sequences, this encompasses sequences that are homologous or complementary to the reference sequence, as well as “sequence-conservative variants” and “function-conservative variants.” For polypeptide sequences, this encompasses “function-conservative variants.” Also encompassed are naturally occurring mutations associated with respiratory diseases including, but not limited to, asthma and atopy, as well as other diseases arising from mutations in this region including those described in detail herein. These mutations are not limited to mutations that cause inappropriate expression (e.g., lack of expression, over-expression, and expression in an inappropriate tissue type).

The term “SNP” as used herein refers to a site in a nucleic acid sequence that contains a nucleotide polymorphism. In accordance with this invention, a SNP may comprise one of two possible “alleles”. For example SNP E+1 may comprise allele C or T (Table 5, below). Thus, a nucleic acid molecule comprising SNP E+1 may include a C or T at the polymorphic position. For a combination of SNPs, the term “haplotype” is used. As an example, the haplotype A/C is observed for SNP combination G1/V−1 (Table 24, below). Thus, A is present at the polymorphic position in SNP G1 and C is present in the polymorphic position in SNP V−1. It should be noted that haplotype representation “A/C” does not indicate “A or C”. Instead, the haplotype representation “A/C” indicates that both the A allele and the C allele are present in their respective SNPs. In addition, the SNP representation “G1/V−1” does not indicate “G1 or V−1”. Instead, “G1/V−1” indicates that both SNPs are present. In some instances, a specific allele or haplotype may be associated with susceptibility to a disease or condition of interest, e.g. asthma. In other instances, an allele or haplotype may be associated with a decrease in susceptibility to a disease or condition of interest, i.e., a protective sequence.

“Sequence-conservative” variants are those in which a change of one or more nucleotides in a given codon position results in no alteration in the amino acid encoded at that position (i.e., silent mutations). “Function-conservative” variants are those in which a change in one or more nucleotides in a given codon position results in a polypeptide sequence in which a given amino acid residue in a polypeptide has been changed without substantially altering the overall conformation and function of the native polypeptide, including, but not limited to, replacement of an amino acid with one having similar physico-chemical properties (such as, for example, acidic, basic, hydrophobic, and the like). “Function-conservative” variants also include analogs of a given polypeptide and any polypeptides that have the ability to elicit antibodies specific to a designated polypeptide.

“Nucleic acid or “polynucleotide” as used herein refers to purine- and pyrimidine-containing polymers of any length, either polyribonucleotides or polydeoxyribonucleotide or mixed polyribo-polydeoxyribonucleotides. This includes single-and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases.

A “coding sequence” or a “protein-coding sequence” is a polynucleotide sequence capable of being transcribed into mRNA and capable of being translated into a polypeptide. The boundaries of the coding sequence are typically determined by a translation start codon at the 5′-terminus and a translation stop codon at the 3′-terminus.

As used herein, the “reference sequence” refers to the sequence used to compare individuals in identifying single nucleotide polymorphisms and the like. Reference sequences may be referred to herein by their GenBank Accession number, GeneBank Protein Accession number, GeneBank Genomic Contig Accession number, Genebank Genomic Clone Accession number, or by specific markers. “Variant” sequences refer to nucleotide sequences (and in some cases, the encoded amino acid sequences) that differ from the reference sequence(s) at one or more positions. Non-limiting examples of variant sequences include the disclosed single nucleotide polymorphisms (SNPs), including SNP alleles and haplotypes, alternate splice variants, and the amino acid sequences encoded by these variants.

“Expressed Sequence Tag (EST)” is a nucleic acid that encodes for a portion of or a full-length protein sequence.

A “complement” of a nucleic acid sequence as used herein refers to the “antisense” sequence that participates in Watson-Crick base-pairing with the original sequence.

A “probe” refers to a nucleic acid or oligonucleotide that forms a hybrid structure with a sequence in a target region due to complementarily of at least one sequence in the probe with a sequence in the target region.

Nucleic acids are “hybridizable” to each other when at least one strand of nucleic acid can anneal to another nucleic acid strand under defined stringency conditions. As is well known in the art, stringency of hybridization is determined, e.g., by (a) the temperature at which hybridization and washing is performed, and (b) the ionic strength and polarity (e.g., formamide) of the hybridization and washing solutions, as well as other parameters. Hybridization requires that the two nucleic acids contain substantially complementary sequences; depending on the stringency of hybridization, however, mismatches may be tolerated. The appropriate stringency for hybridizing nucleic acids depends on the length of the nucleic acids and the degree of complementarily, variables well known in the art.

“Gene” refers to a DNA sequence that encodes through its template or messenger RNA a sequence of amino acids characteristic of a specific peptide, polypeptide, or protein. The term “gene” as used herein with reference to genomic DNA includes intervening, non-coding regions, as well as regulatory regions, and can include 5′ and 3′ ends.

“Gene sequence” refers to a DNA molecule, including a DNA molecule that contains a non-transcribed or non-translated sequence. The term is also intended to include any combination of gene(s), gene fragment(s), non-transcribed sequence(s), or non-translated sequence(s) that are present on the same DNA molecule.

A gene sequence is “wild-type” if such sequence is usually found in individuals unaffected by the disease or condition of interest. However, environmental factors and other genes can also play an important role in the ultimate determination of the disease. In the context of complex diseases involving multiple genes (“oligogenic disease”), the “wild type”, or normal sequence can also be associated with a measurable risk or susceptibility, receiving its reference status based on its frequency in the general population. As used herein, “wild-type” refers to the reference sequence. The wild-type sequences are used to identify the variants (single nucleotide polymorphisms) described in detail herein.

A gene sequence is a “mutant” sequence if it differs from the wild-type sequence. For example, an ADAM-related gene nucleic acid sequence containing a single nucleotide polymorphism is a mutant sequence. In some cases, the individual carrying such genes has increased susceptibility toward the disease or condition of interest. In other cases, the “mutant” sequence might also refer to a sequence that decreases the susceptibilty toward a disease or condition of interest, and thus acting in a protective manner. Also a gene is a “mutant” gene if too much (“overexpressed”) or too little (“underexpressed”) of such gene is expressed in the tissues in which such gene is normally expressed, thereby causing the disease or condition of interest.

“cDNA” refers to complementary or copy DNA produced from an RNA template by the action of RNA-dependent DNA polymerase (reverse transcriptase). Thus, a “cDNA clone” means a duplex DNA sequence complementary to an RNA molecule of interest, carried in a cloning vector or PCR amplified. This term includes genes from which the intervening sequences have been removed.

“Recombinant DNA” means a molecule that has been recombined by in vitro splicing/and includes cDNA or a genomic DNA sequence.

“Cloning” refers to the use of in vitro recombination techniques to insert a particular gene or other DNA sequence into a vector molecule. In order to successfully clone a desired gene, it is necessary to use methods for generating DNA fragments, for joining the fragments to vector molecules, for introducing the composite DNA molecule into a host cell in which it can replicate, and for selecting the clone having the target gene from amongst the recipient host cells.

“cDNA library” refers to a collection of recombinant DNA molecules containing cDNA inserts, which together comprise the entire genome of an organism. Such a cDNA library can be prepared by methods known to one skilled in the art and described by, for example, Cowell and Austin, 1997, “cDNA Library Protocols,” Methods in Molecular Biology. Generally, RNA is first isolated from the cells of an organism from whose genome it is desired to clone a particular gene.

The term “vector” as used herein refers to a nucleic acid molecule capable of replicating another nucleic acid to which it has been linked. A vector, for example, can be a plasmid.

“Cloning vector” refers to a plasmid or phage DNA or other DNA sequence that is able to replicate in a host cell. The cloning vector is characterized by one or more endonuclease recognition sites at which such DNA sequences may be cut in a determinable fashion without loss of an essential biological function of the DNA, which may contain a marker suitable for use in the identification of transformed cells.

“Expression vector” refers to a vehicle or vector similar to a cloning vector but which is capable of expressing a nucleic acid sequence that has been cloned into it, after transformation into a host. A nucleic acid sequence is “expressed” when it is transcribed to yield an mRNA sequence. In most cases, this transcript will be translated to yield amino acid sequence. The cloned gene is usually placed under the control of (i.e., operably linked to) an expression control sequence.

“Expression control sequence” or “regulatory sequence” refers to a nucleotide sequence that controls or regulates expression of structural genes when operably linked to those genes. These include, for example, the lac systems, the trp system, major operator and promoter regions of the phage lambda, the control region of fd coat protein and other sequences known to control the expression of genes in prokaryotic or eukaryotic cells. Expression control sequences will vary depending on whether the vector is designed to express the operably linked gene in a prokaryotic or eukaryotic host, and may contain transcriptional elements such as enhancer elements, termination sequences, tissue-specificity elements or translational initiation and termination sites.

“Operably linked” means that the promoter controls the initiation of expression of the gene. A promoter is operably linked to a sequence of proximal DNA if upon introduction into a host cell the promoter determines the transcription of the proximal DNA sequence(s) into one or more species of RNA. A promoter is operably linked to a DNA sequence if the promoter is capable of initiating transcription of that DNA sequence.

“Host” includes prokaryotes and eukaryotes. The term includes an organism or cell that is the recipient of a replicable expression vector.

The introduction of the nucleic acids into the host cell by any method known in the art, including those described herein, will be referred to herein as “transformation.” The cells into which have been introduced nucleic acids described above are meant to also include the progeny of such cells.

“Amplification of nucleic acids” refers to methods such as polymerase chain reaction (PCR), ligation amplification (or ligase chain reaction, LCR) and amplification methods based on the use of Q-beta replicase. These methods are well known in the art and described, for example, in U.S. Pat. Nos. 4,683,195 and 4,683,202. Reagents and hardware for conducting PCR are commercially available. Primers useful for amplifying sequences from the disorder region are preferably complementary to, and preferably hybridize specifically to, sequences in the disorder region_or in regions that flank a target region therein. Genes generated by amplification may be sequenced directly. Alternatively, the amplified sequence(s) may be cloned prior to sequence analysis.

A nucleic acid or fragment thereof is “substantially homologous” or “substantially similar” to another if, when optimally aligned (with appropriate nucleotide insertions or deletions) with the other nucleic acid (or its complementary strand), there is nucleotide sequence identity in at least 60% of the nucleotide bases, usually at least 70%, more usually at least 80%, preferably at least 90%, and more preferably at least 95-98% of the nucleotide bases.

Alternatively, substantial homology or similarity exists when a nucleic acid or fragment thereof will hybridize, under selective hybridization conditions, to another nucleic acid (or a complementary strand thereof). Selectivity of hybridization exists when hybridization which is substantially more selective than total lack of specificity occurs. Typically, selective hybridization will occur when there is at least 55% homology over a stretch of at least nine or more nucleotides, preferably at least 65%, more preferably at least 75%, and most preferably at least 90% (see, M. Kanehisa, 1984, Nucl. Acids Res. 11:203-213). The length of homology comparison, as described, may be over longer stretches, and in certain embodiments will often be over a stretch of at least 14 nucleotides, usually at least 20 nucleotides, more usually at least 24 nucleotides, typically at least 28 nucleotides, more typically at least 32 nucleotides, and preferably at least 36 or more nucleotides.

Nucleic acids referred to herein as “isolated” are nucleic acids separated away from the nucleic acids of the genomic DNA or cellular RNA of their source of origin (e.g., as it exists in cells or in a mixture of nucleic acids such as a library), and may have undergone further processing. “Isolated”, as used herein, refers to nucleic or amino acid sequences that are at least 60% free, preferably 75% free, and most preferably 90% free from other components with which they are naturally associated. “Isolated” nucleic acids (polynucleotides) include nucleic acids obtained by methods described herein, similar methods or other suitable methods, including essentially pure nucleic acids, nucleic acids produced by chemical synthesis, by combinations of biological and chemical methods, and recombinant nucleic acids which are isolated. Nucleic acids referred to herein as “recombinant” are nucleic acids which have been produced by recombinant DNA methodology, including those nucleic acids that are generated by procedures which rely upon a method of artificial replication, such as the polymerase chain reaction (PCR) or cloning into a vector using restriction enzymes. “Recombinant” nucleic acids are also those that result from recombination events that occur through the natural mechanisms of cells, but are selected for after the introduction to the cells of nucleic acids designed to allow or make probable a desired recombination event. Portions of the isolated nucleic acids which code for polypeptides having a certain function can be identified and isolated by, for example, the method of Jasin, M., et al., U.S. Pat. No. 4,952,501.

In the context of this invention, the term “oligonucleotide” refers to naturally occurring species or synthetic species formed from naturally occurring subunits or their close homologs. The term may also refer to moieties that function similarly to oligonucleotides, but have non-naturally-occurring portions. Thus, oligonucleotides may have altered sugar moieties or inter-sugar linkages. Exemplary among these are phosphorothioate and other sulfur containing species which are known in the art.

As used herein, the terms “protein” and “polypeptide” are synonymous. “Peptides” are defined as fragments or portions of polypeptides, preferably fragments or portions having at least one functional activity (e.g., proteolysis, adhesion, fusion, antigenic, or intracellular activity) as the complete polypeptide sequence.

As used herein, “isolated” proteins or polypeptides are proteins or polypeptides purified to a state beyond that in which they exist in cells. In a preferred embodiment, they are at least 10% pure; i.e., most preferably they are substantially purified to 80 or 90% purity. “Isolated” proteins or polypeptides include proteins or polypeptides obtained by methods described infra, similar methods or other suitable methods, and include essentially pure proteins or polypeptides, proteins or polypeptides produced by chemical synthesis or by combinations of biological and chemical methods, and recombinant proteins or polypeptides which are isolated. Proteins or polypeptides referred to herein as “recombinant” are proteins or polypeptides produced by the expression of recombinant nucleic acids.

A “portion” as used herein with regard to a protein or polypeptide, refers to fragments of that protein or polypeptide. The fragments can range in size from 5 amino acid residues to all but one residue of the entire protein sequence. Thus, a portion or fragment can be at least 5, 5-50, 50-100, 100-200, 200-400, 400-800, or more consecutive amino acid residues of a protein or polypeptide, or variants thereof.

The term “immunogenic”, refers to the ability of a molecule (e.g., a polypeptide or peptide) to elicit a humoral or cellular immune response in a host animal.

The term “antigenic” refers to the ability of a molecule (e.g., a polypeptide or peptide) to bind to its specific antibody with sufficiently high affinity to form a detectable antigen-antibody complex.

“Antibodies” refer to polyclonal and monoclonal antibodies and fragments thereof, and immunologic binding equivalents thereof, that can bind to asthma proteins and fragments thereof or to nucleic acid sequences of ADAM-related or Interactor genes, particularly from chromosomal regions associated with asthma or a portion thereof. The term antibody is used both to refer to a homogeneous molecular entity, or a mixture such as a serum product made up of a plurality of different molecular entities.

The term “monoclonal antibody” or “monoclonal antibody composition”, as used herein, refers to a population of antibody molecules that contain only one species of an antigen binding site capable of immunoreacting with a particular epitope of a ADAM or Interactor polypeptide or peptide. A monoclonal antibody composition thus typically displays a single binding affinity for a particular ADAM or Interactor polypeptide or peptide with which it immunoreacts.

The term “ligand” as used herein describes any molecule, protein, peptide, or compound with the capability of directly or indirectly altering the physiological function, stability, or levels of a polypeptide.

A “sample” as used herein refers to a biological sample, such as, for example, tissue or fluid isolated from an individual (including, without limitation, plasma, serum, cerebrospinal fluid, lymph, tears, saliva, milk, pus, and tissue exudates and secretions) or from in vitro cell culture constituents, as well as samples obtained from, for example, a laboratory procedure.

As used herein, the term “ortholog” denotes a gene or polypeptide obtained from one species that has homology to an analogous gene or polypeptide from a different species. This is in contrast to “paralog”, which denotes a gene or polypeptide obtained from a given species that has homology to a distinct gene or polypeptide from that same species.

Standard reference works setting forth the general principles of recombinant DNA technology include J. Sambrook et al., 1989, Molecular Cloning: A Laboratory Manual, 2d Ed., Cold Spring Harbor Laboratory Press, Cold Spring Harbor, N.Y.; P. B. Kaufman et al., (eds), 1995, Handbook of Molecular and Cellular Methods in Biology and Medicine, CRC Press, Boca Raton; M. J. McPherson (ed), 1991, Directed Mutagenesis: A Practical Approach, IRL Press, Oxford; J. Jones, 1992, Amino Acid and Peptide Synthesis, Oxford Science Publications, Oxford; B. M. Austen and O. M. R. Westwood, 1991, Protein Targeting and Secretion, IRL Press, Oxford; D. N Glover (ed), 1985, DNA Cloning, Volumes I and II; M. J. Gait (ed), 1984, Oligonucleotide Synthesis; B. D. Hames and S. J. Higgins (eds), 1984, Nucleic Acid Hybridization; Wu and Grossman (eds), Methods in Enzymoloqy (Academic Press, Inc.), Vol. 154 and Vol. 155; Quirke and Taylor (eds), 1991, PCR-A Practical Approach; Hames and Higgins (eds), 1984, Transcription and Translation; R. I. Freshney (ed), 1986, Animal Cell Culture; Immobilized Cells and Enzymes, 1986, IRL Press; Perbal, 1984, A Practical Guide to Molecular Cloning; J. H. Miller and M. P. Calos (eds), 1987, Gene Transfer Vectors for Mammalian Cells, Cold Spring Harbor Laboratory Press; M. J. Bishop (ed), 1998, Guide to Human Genome Computing, 2d Ed., Academic Press, San Diego, Calif.; L. F. Peruski and A. H. Peruski, 1997, The Internet and the New Biology: Tools for Genomic and Molecular Research, American Society for Microbiology, Washington, D.C.

Standard reference works setting forth the general principles of immunology include S. Sell, 1996, Immunology, Immunopathology & Immunity, 5th Ed., Appleton & Lange, Publ., Stamford, Conn.; D. Male et al., 1996, Advanced Immunology, 3d Ed., Times Mirror Int'l Publishers Ltd., Publ., London; D. P. Stites and A. I. Terr, 1991, Basic and Clinical Immunology, 7th Ed., Appleton & Lange, Publ., Norwalk, Conn.; and A. K. Abbas et al., 1991, Cellular and Molecular Immunology, W. B. Saunders Co., Publ., Philadelphia, Pa. Any suitable materials and methods known to those of skill can be utilized in carrying out the present invention; however, preferred materials and methods are described. Materials, reagents, and the like to which reference is made in the following description and examples are generally obtainable from commercial sources, and specific vendors are cited herein.

### NUCLEIC ACIDS

The present invention relates to nucleic acids from ADAM and Interactor genes. In a specific embodiment, the invention relates to ADAM and Interactor nucleic acid sequences shown in column 4 of Table 2. RNA, fragments of the genomic, cDNA, or RNA nucleic acids comprising 20, 40, 60, 100, 200, 500 or more contiguous nucleotides, and the complements thereof. Closely related variants are also included as part of this invention, as well as recombinant nucleic acids comprising at least 50, 60, 70, 80, or 90% of the nucleic acids described above which would be identical to nucleic acids from ADAM and Interactor genes except for one or a few substitutions, deletions, or additions.

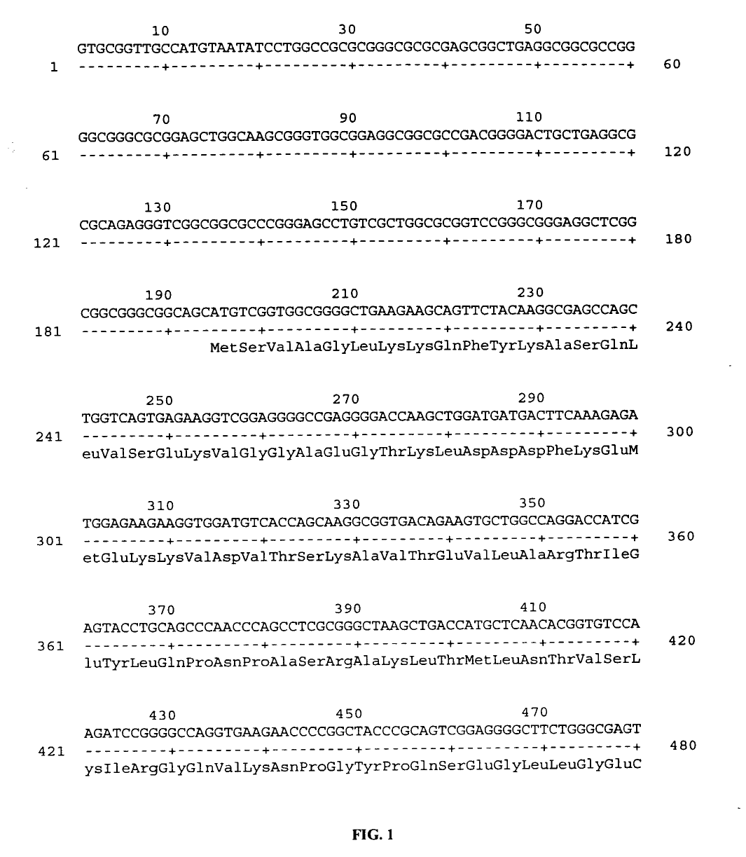

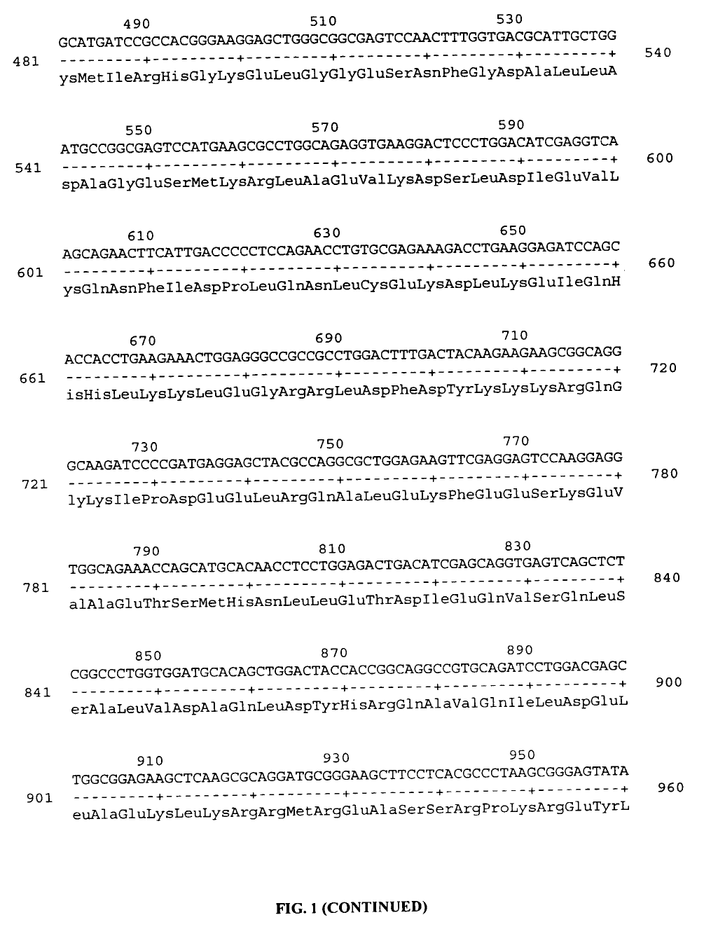

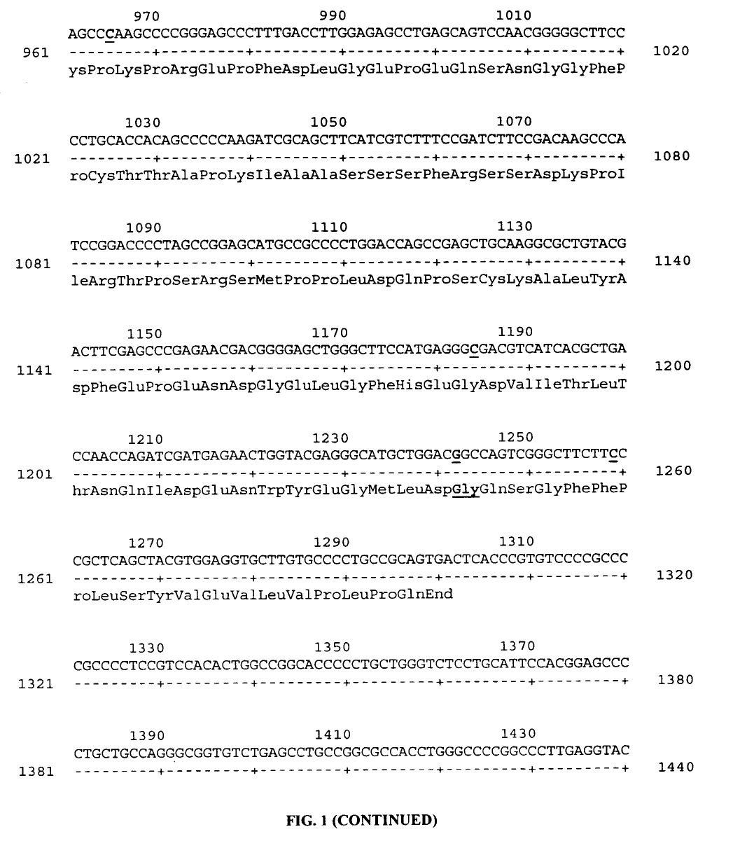

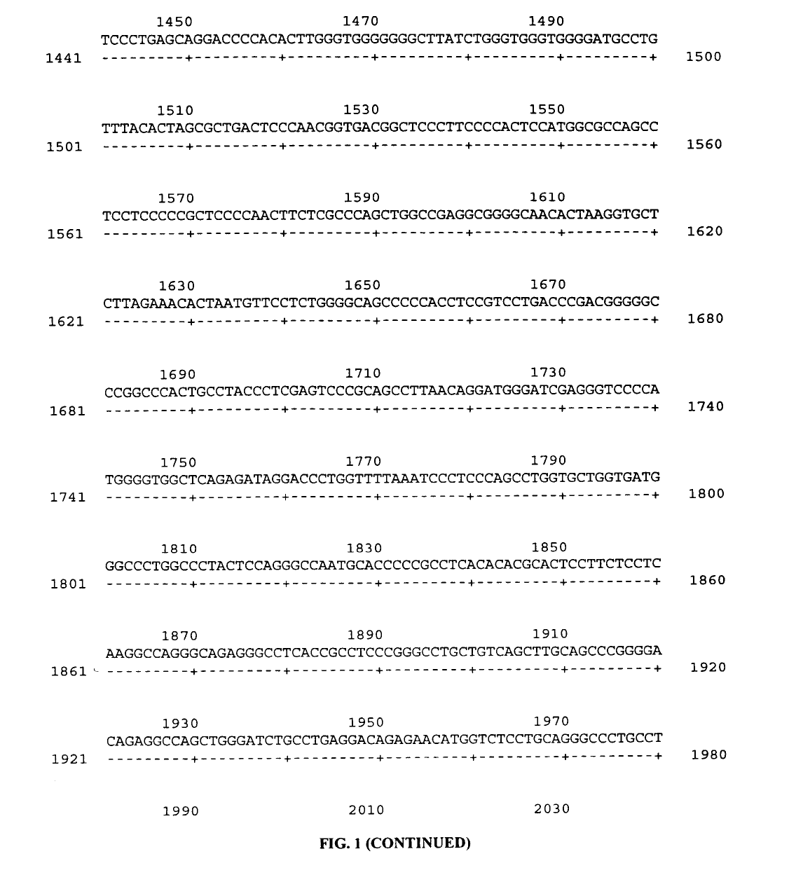

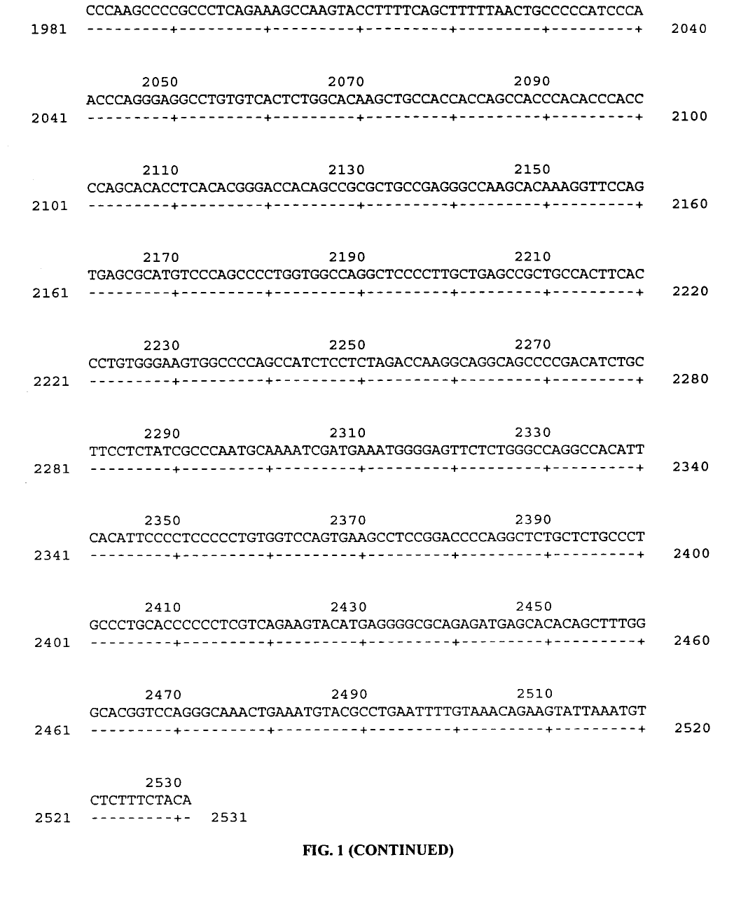

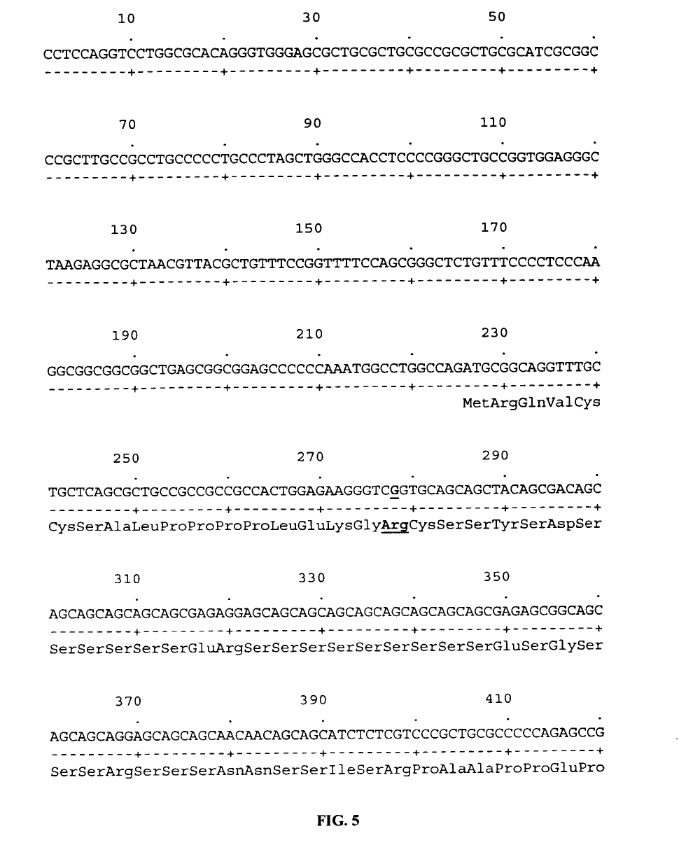

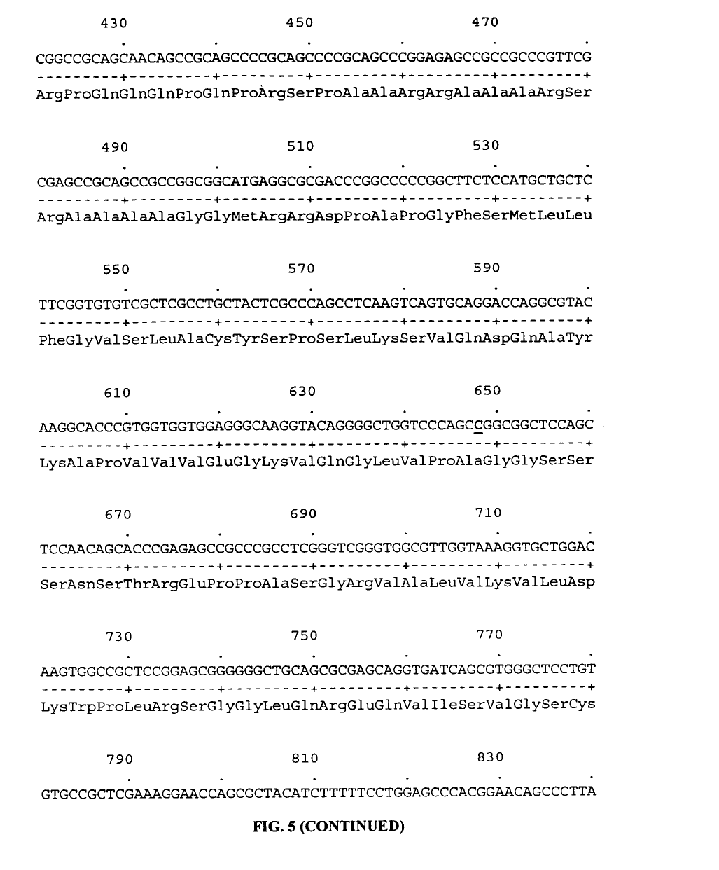

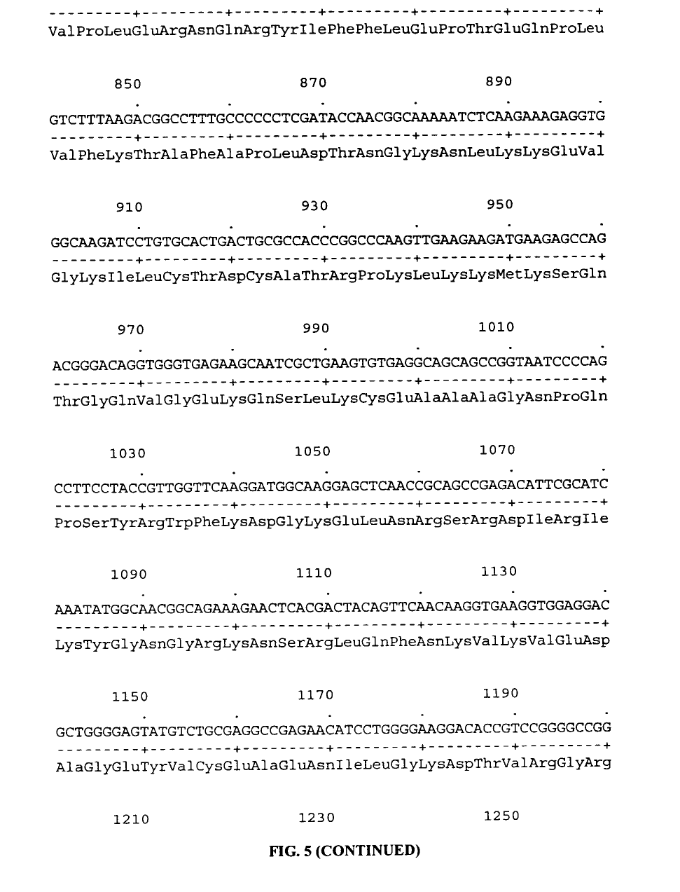

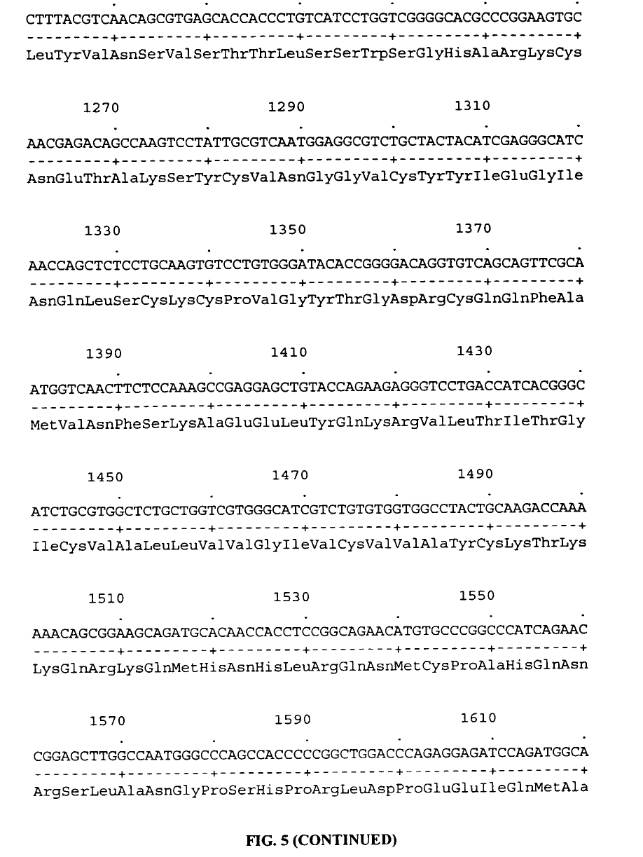

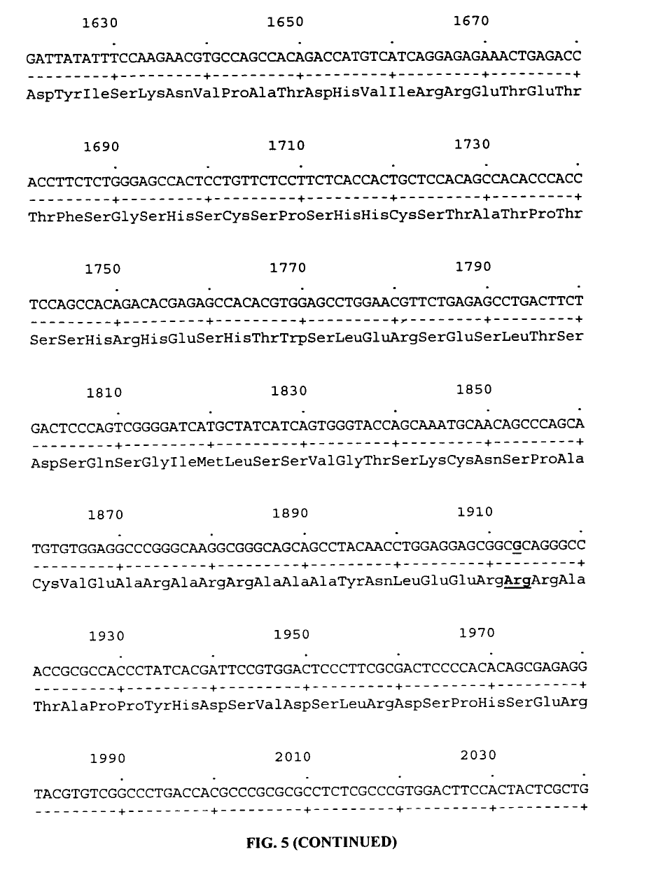

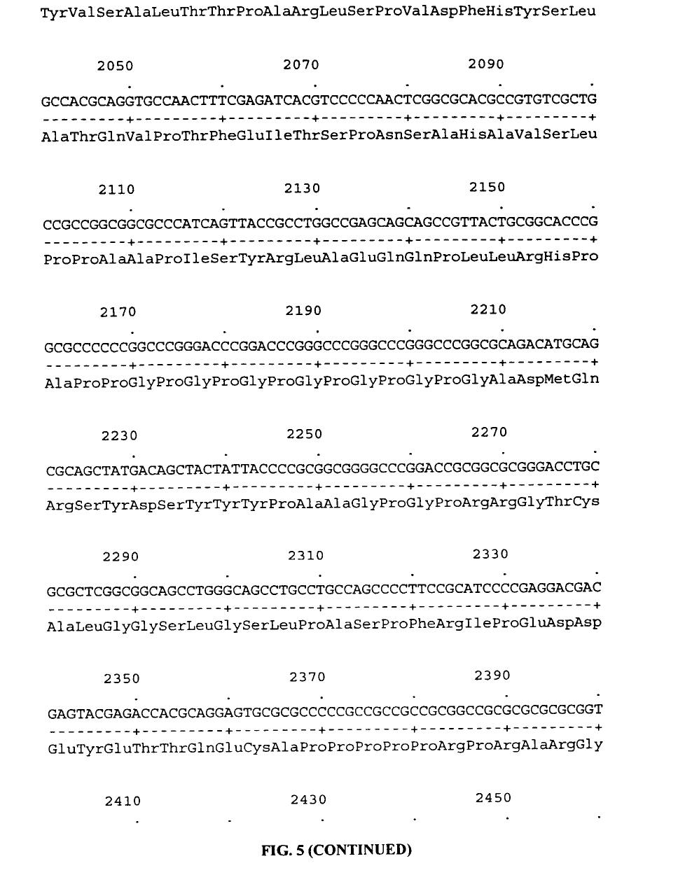

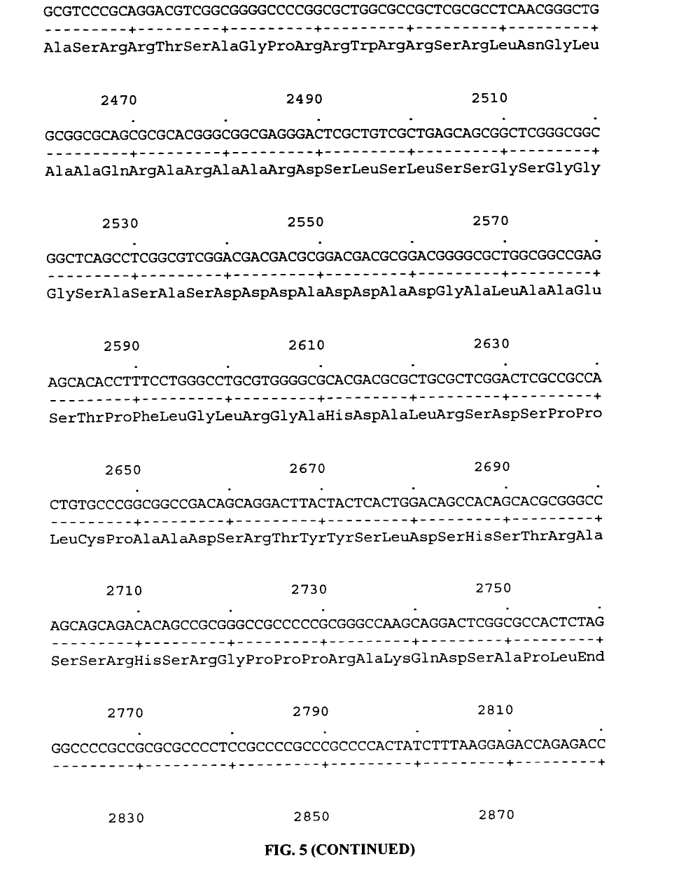

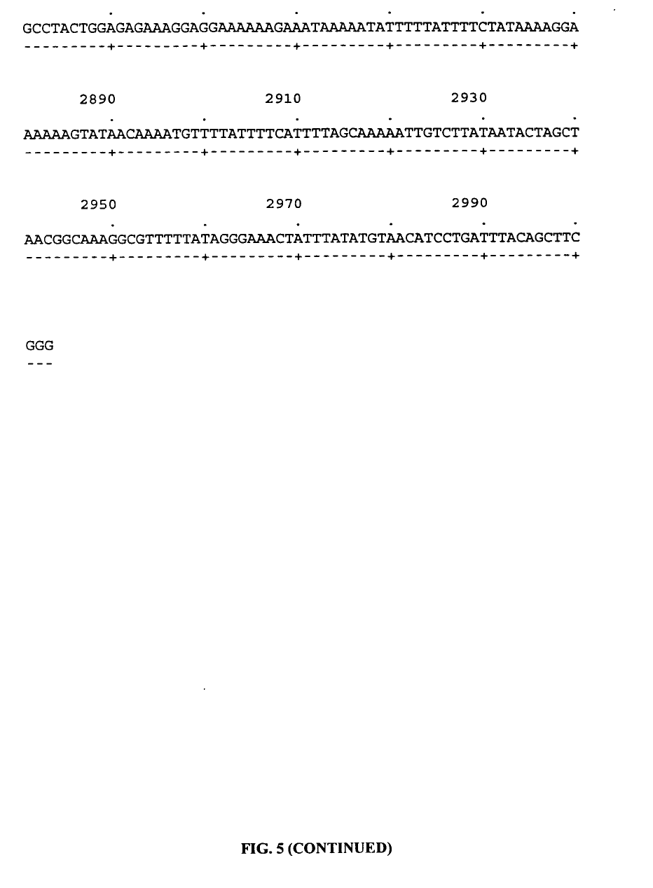

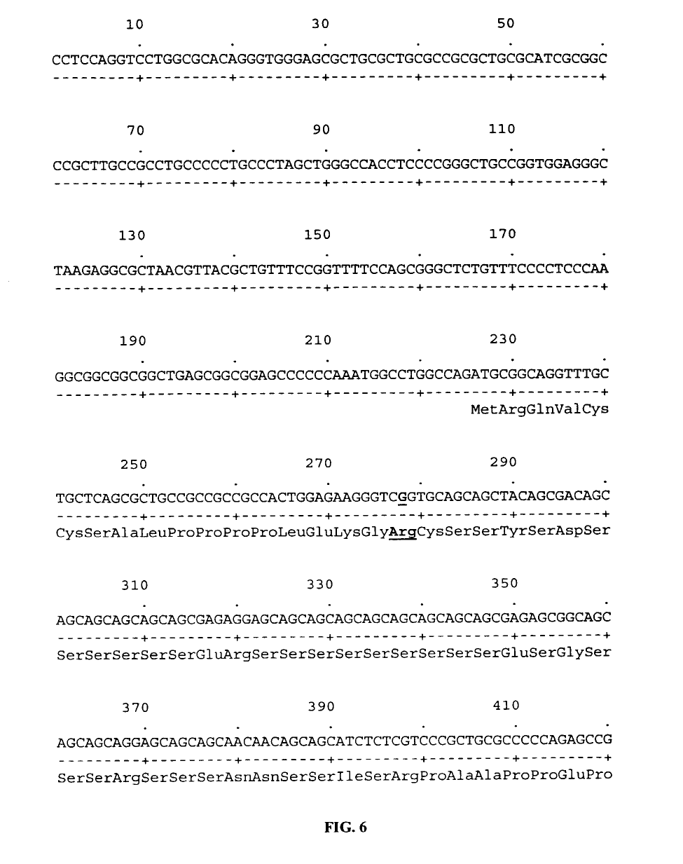

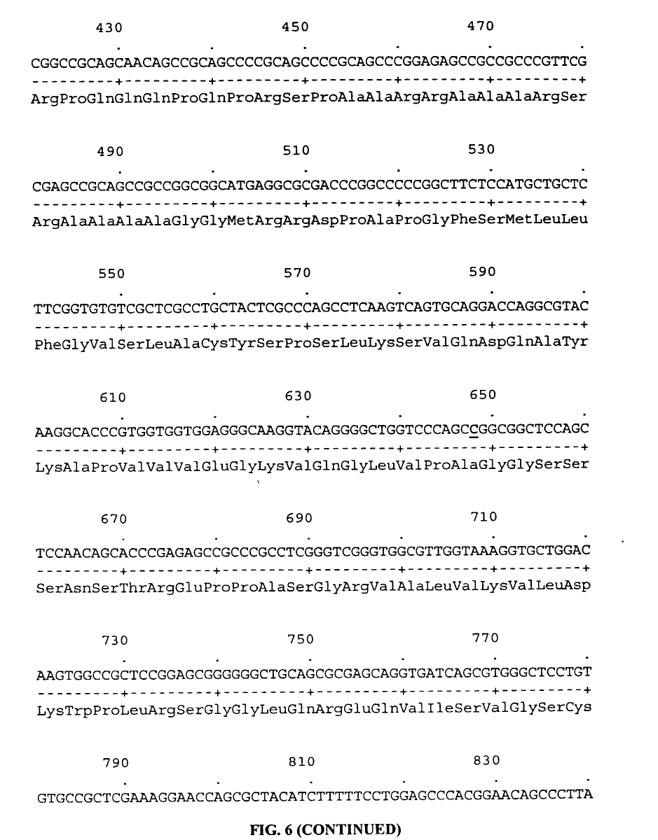

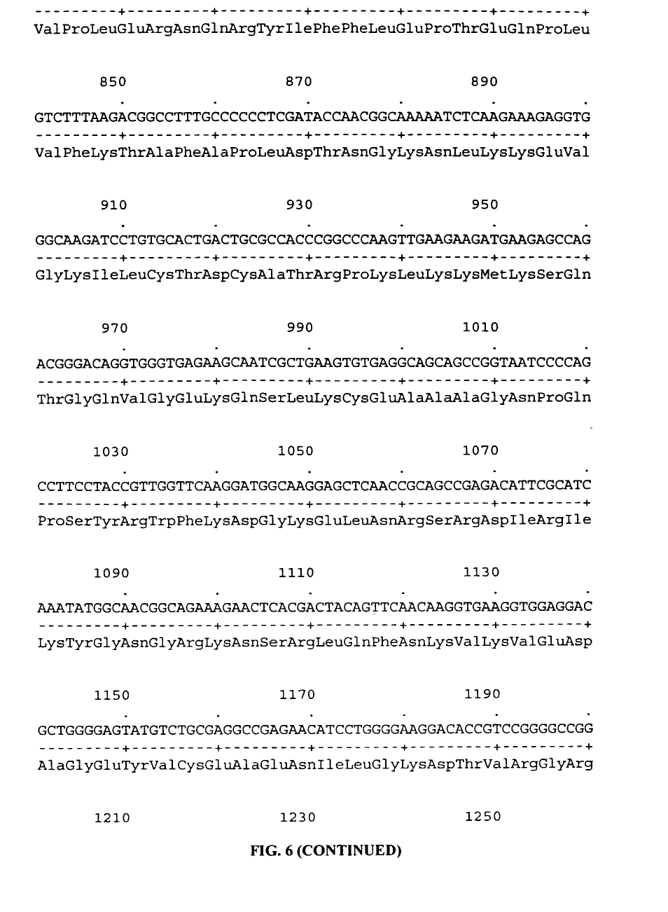

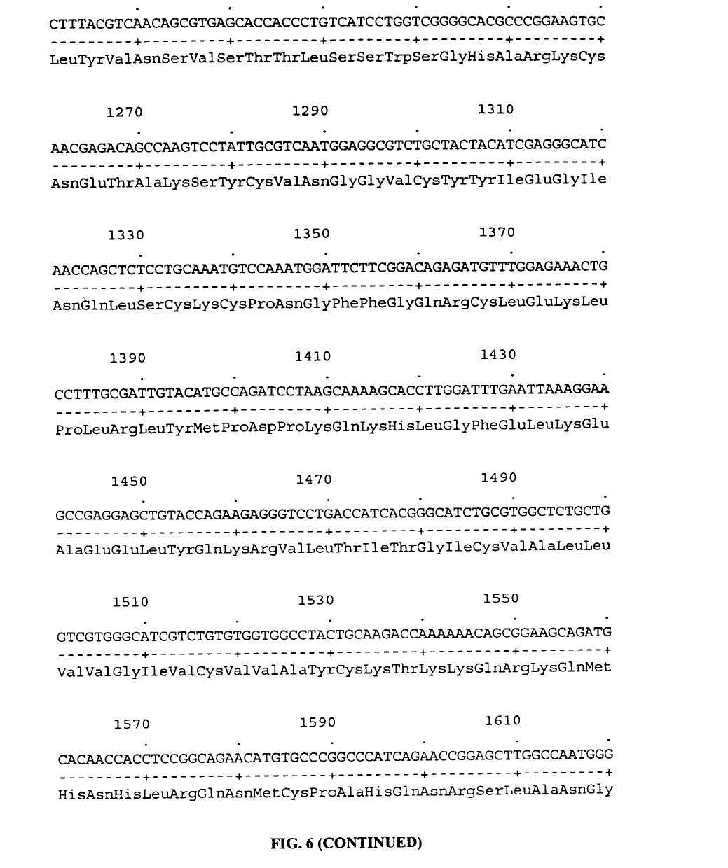

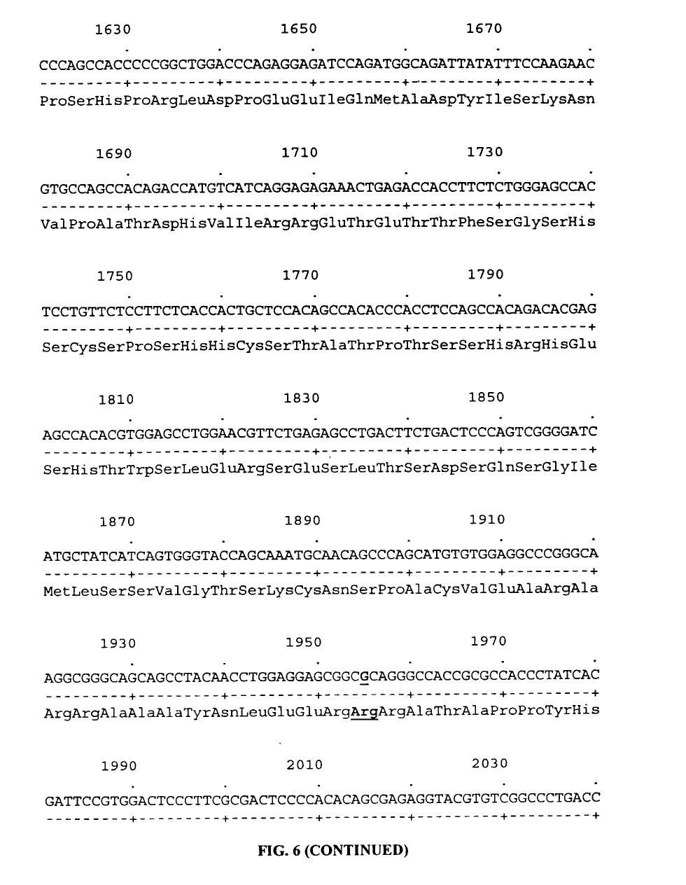

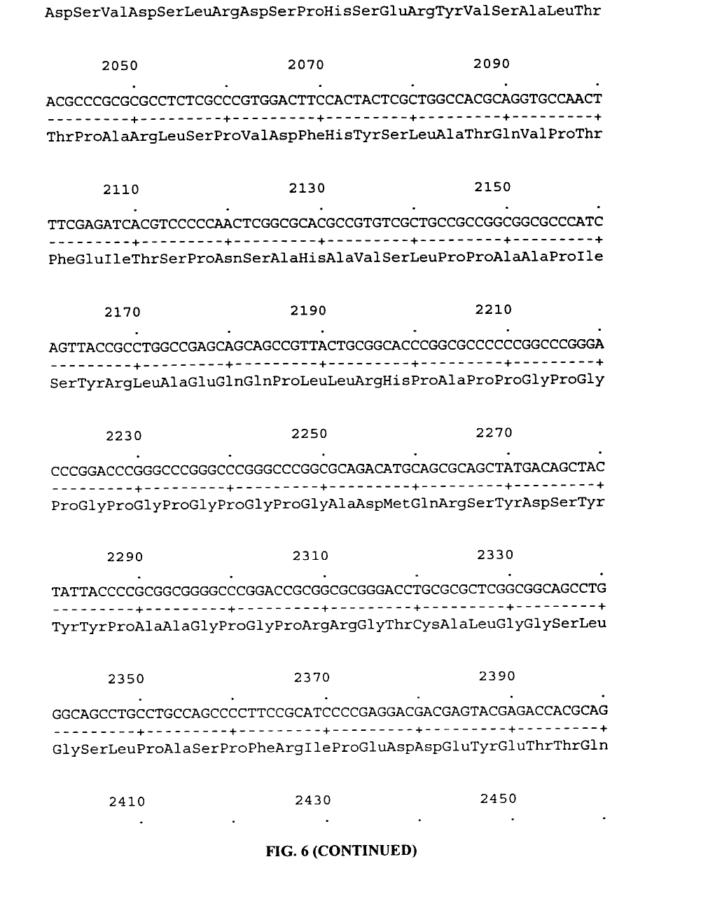

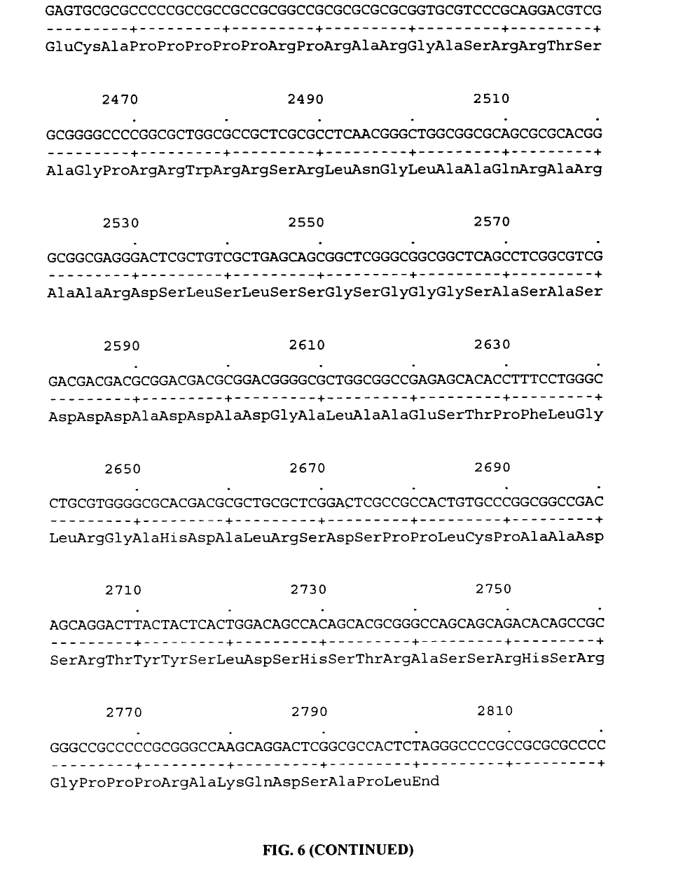

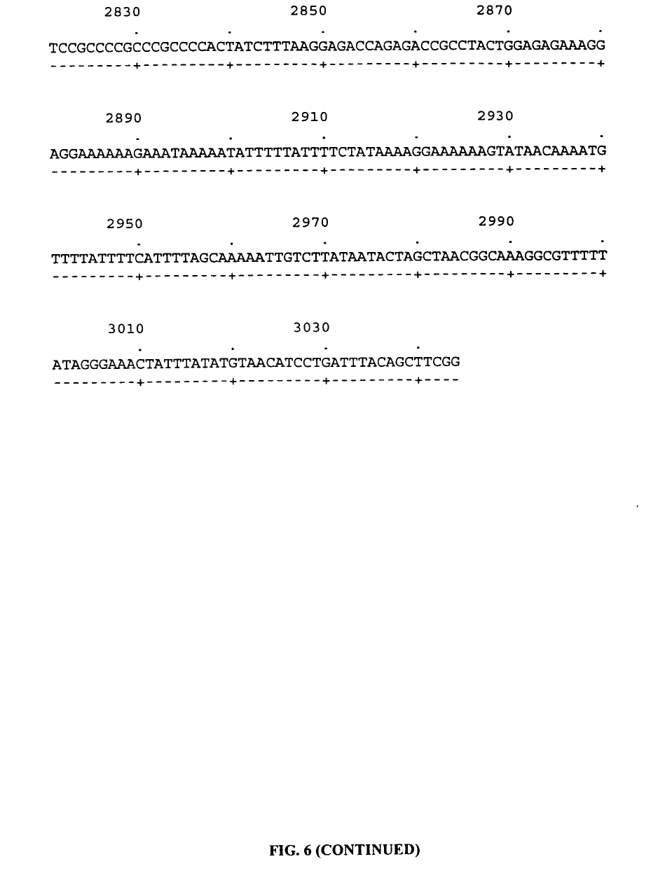

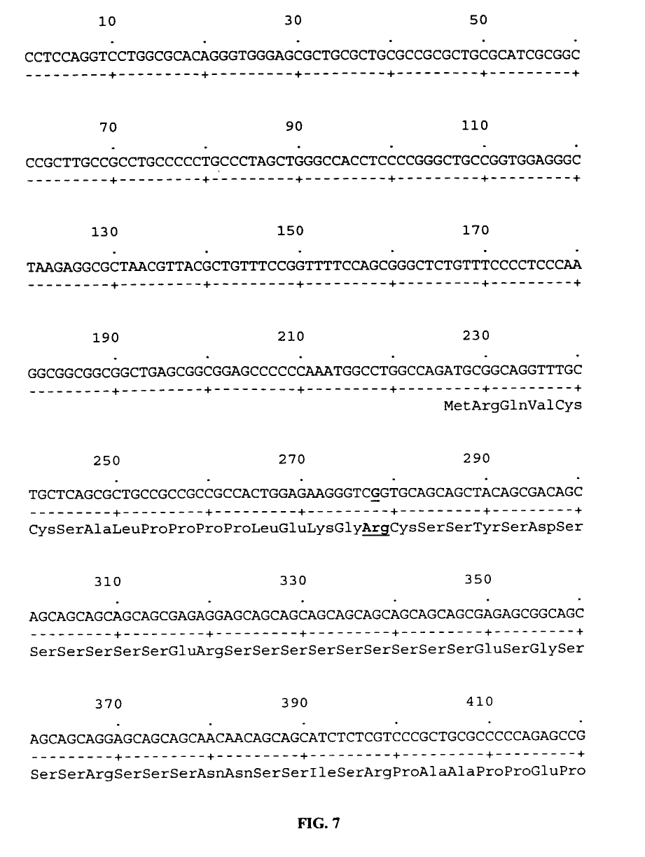

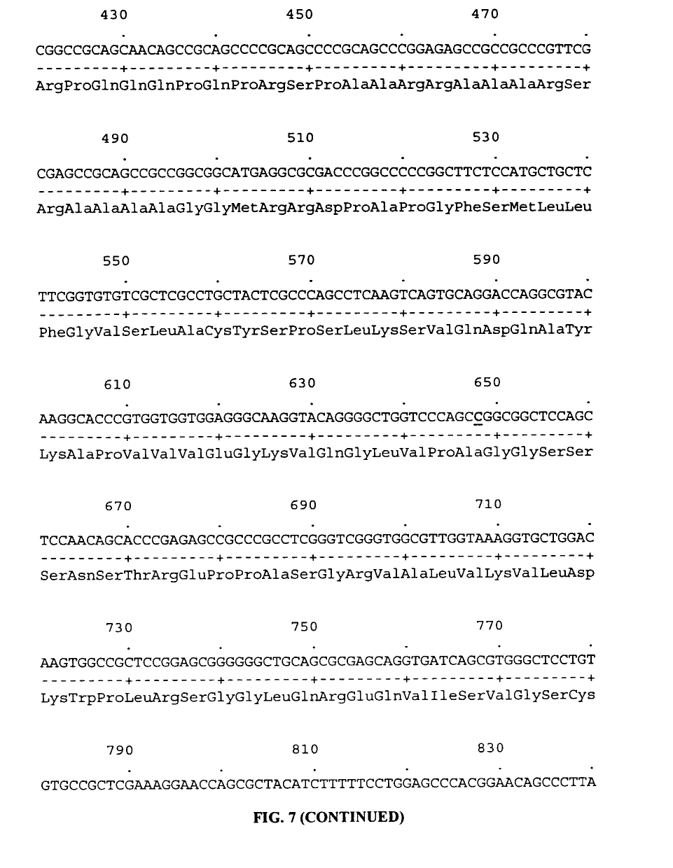

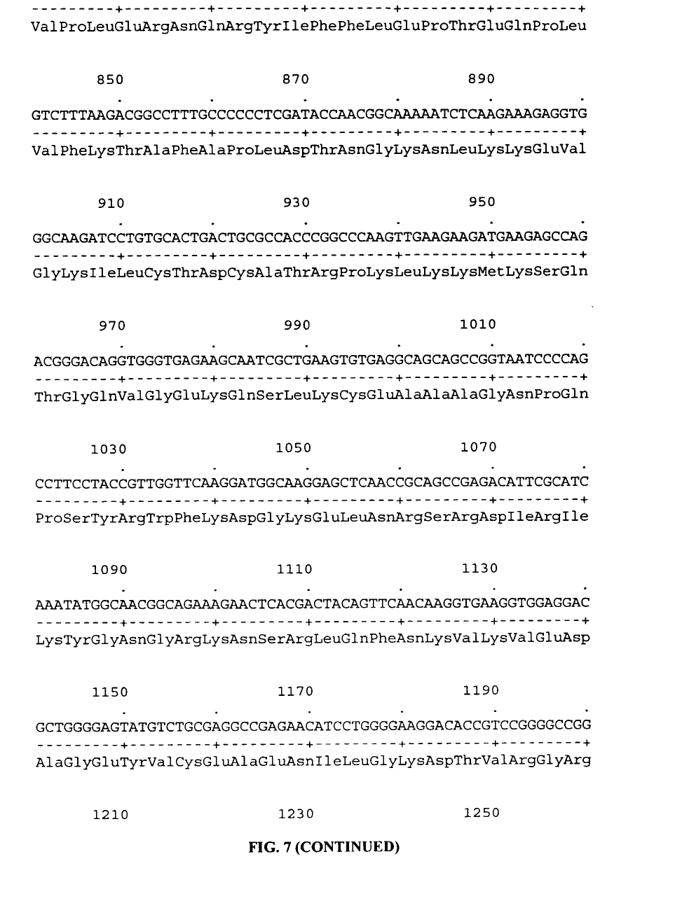

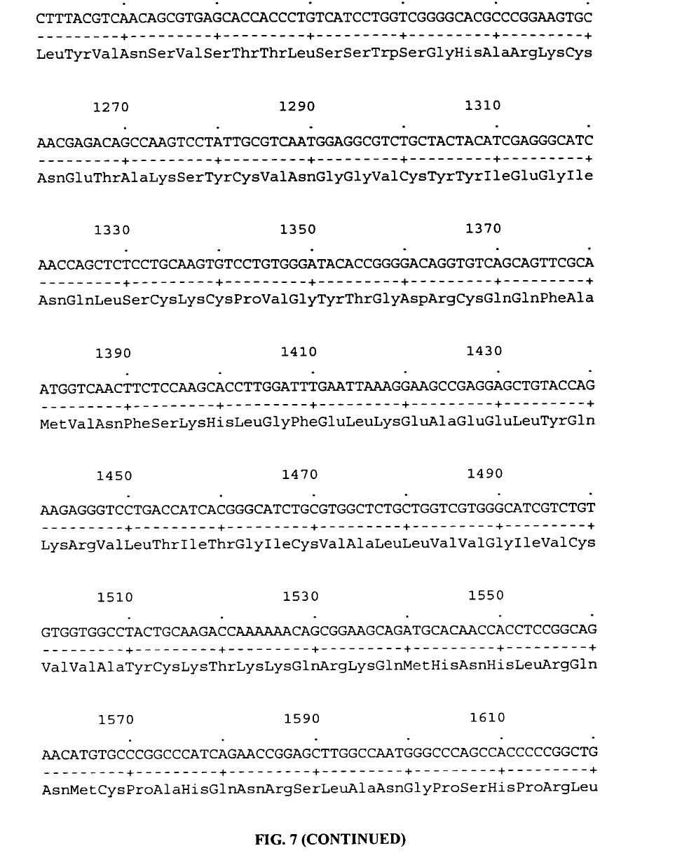

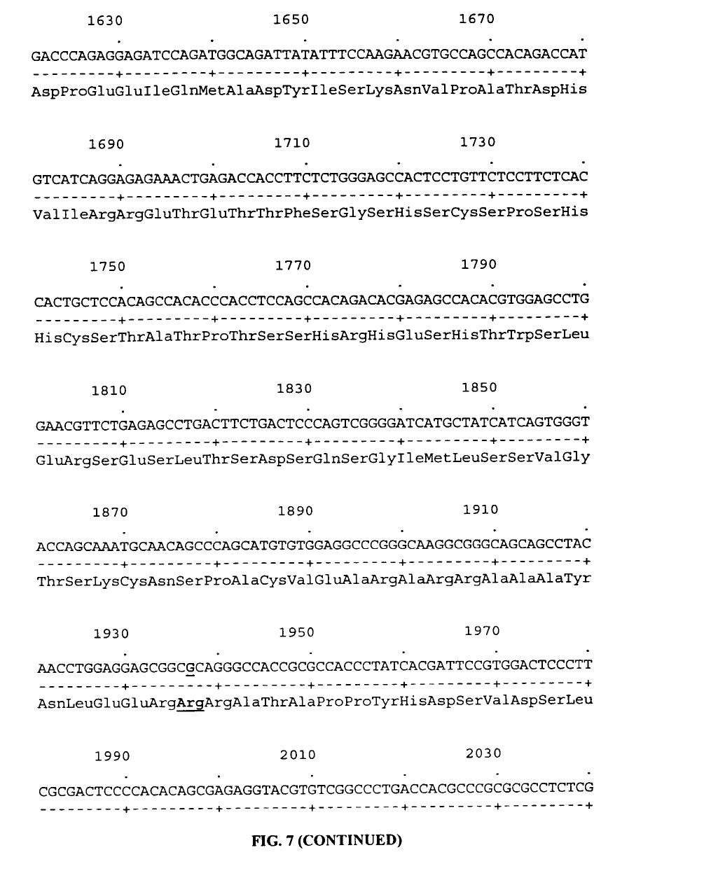

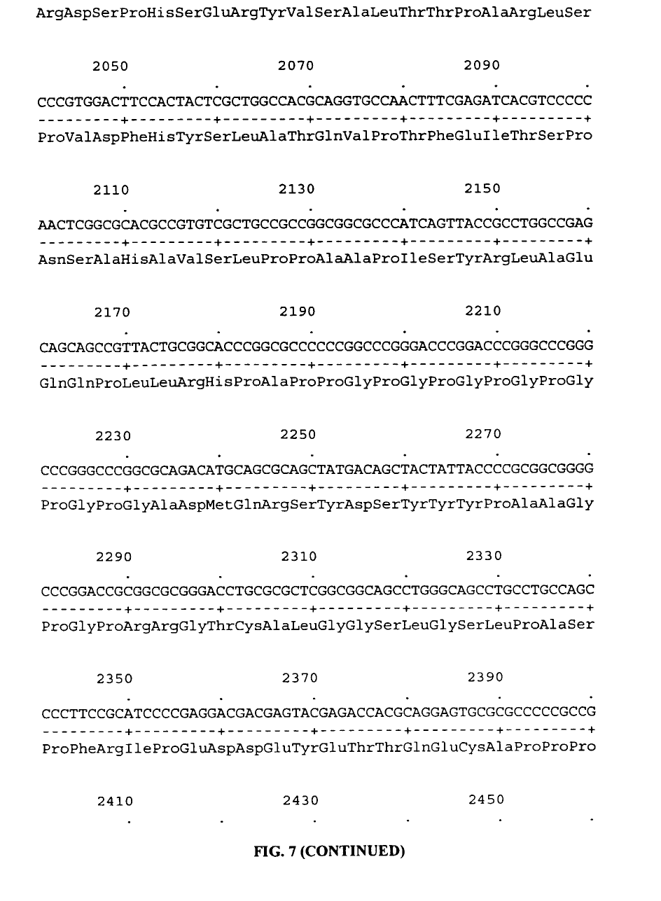

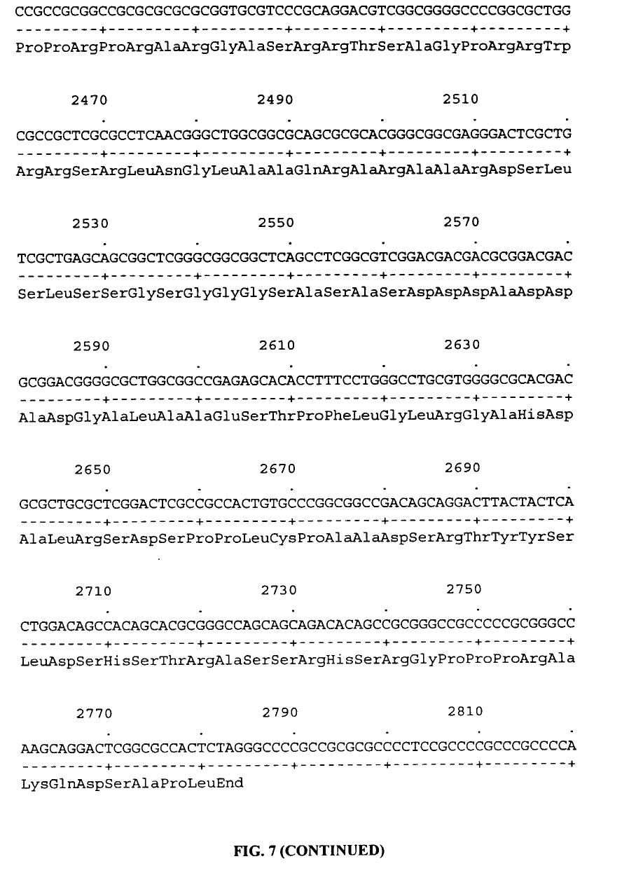

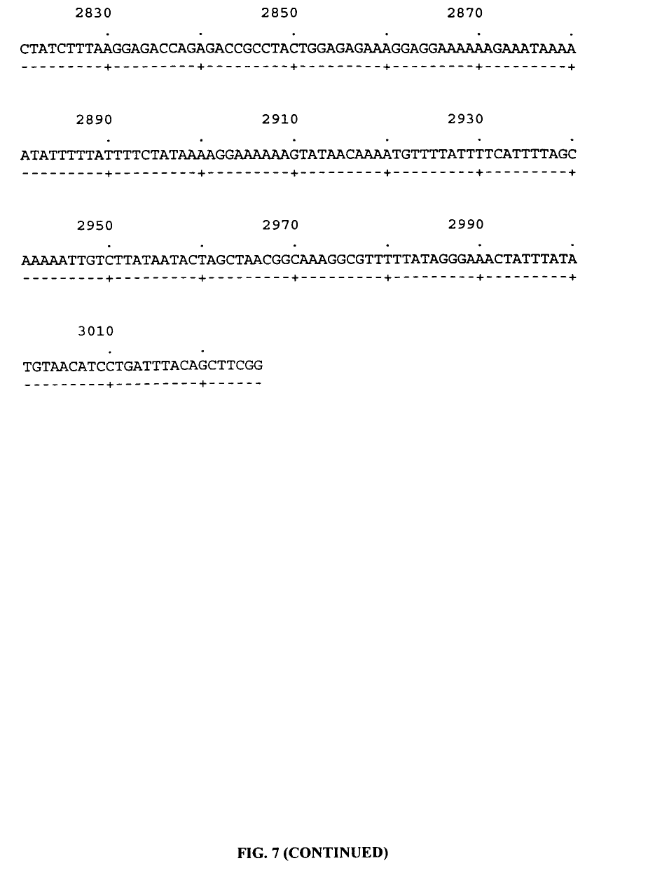

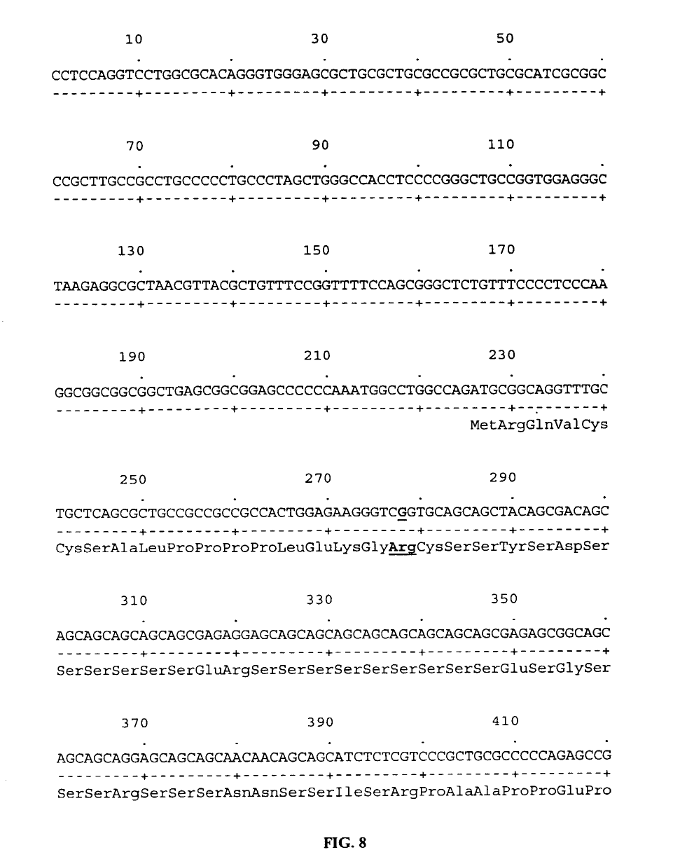

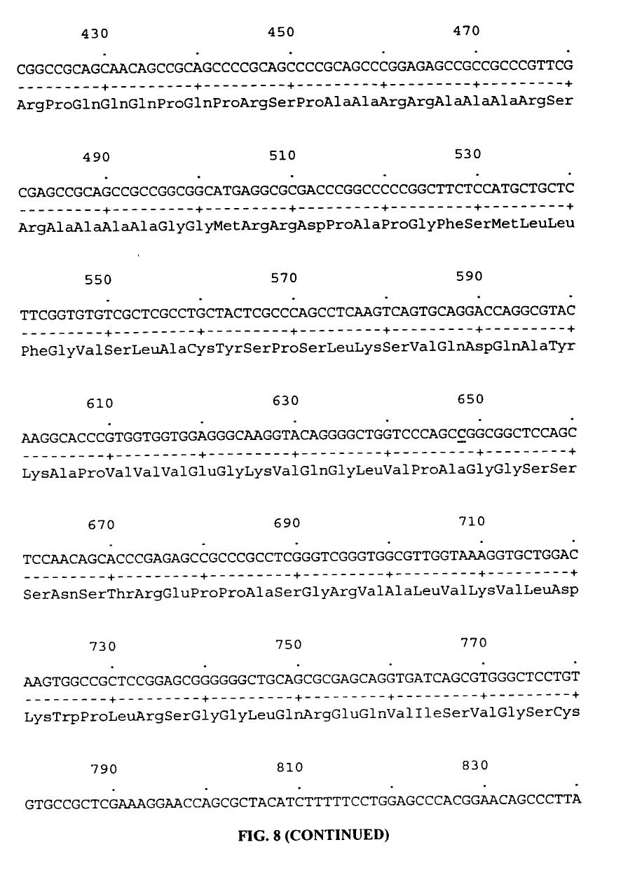

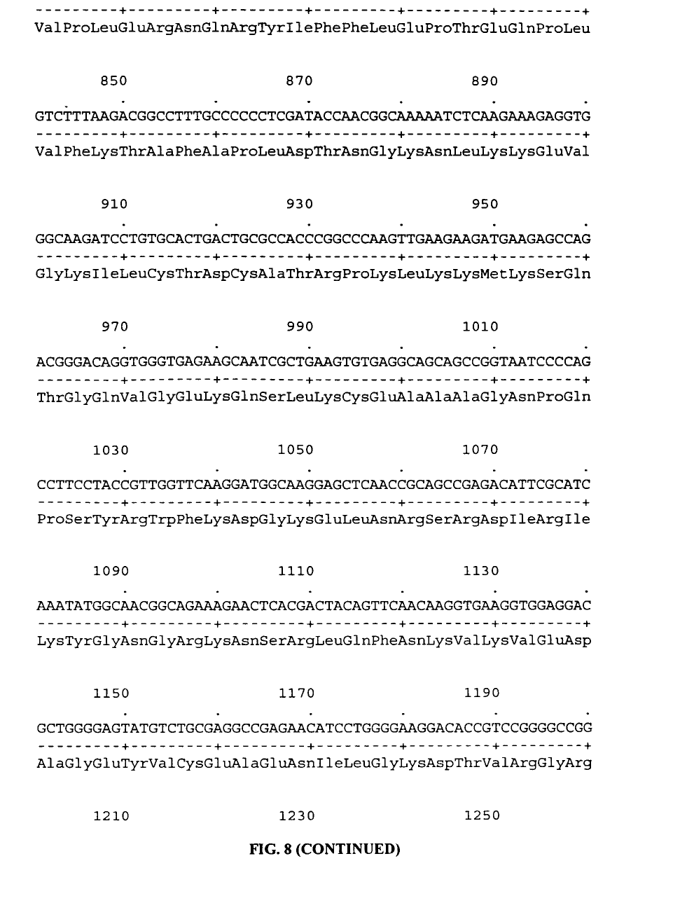

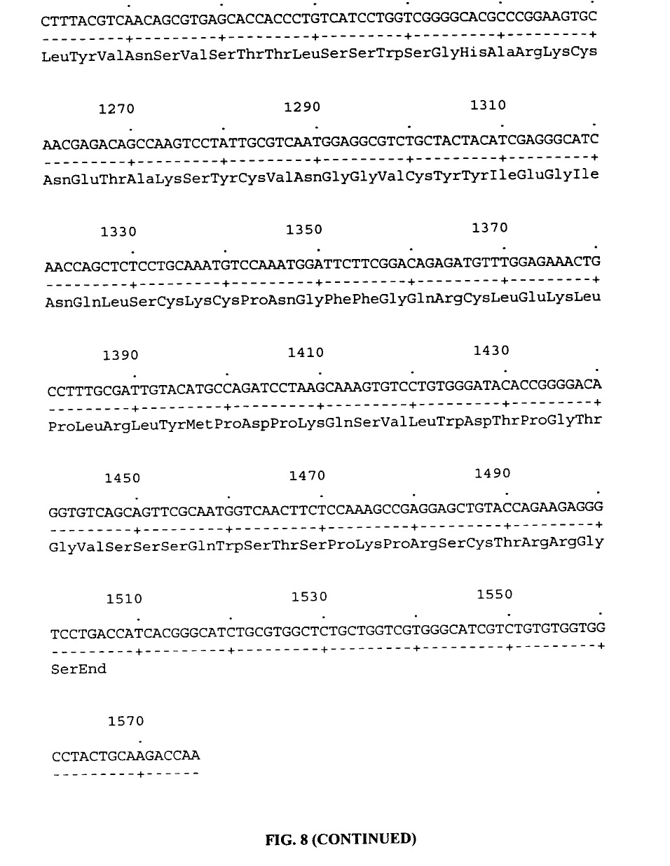

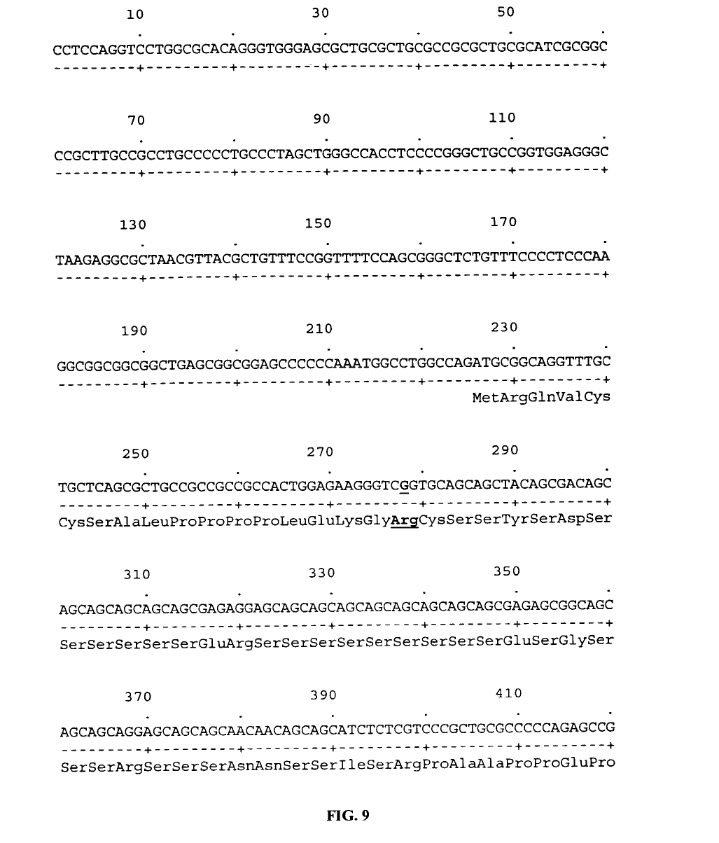

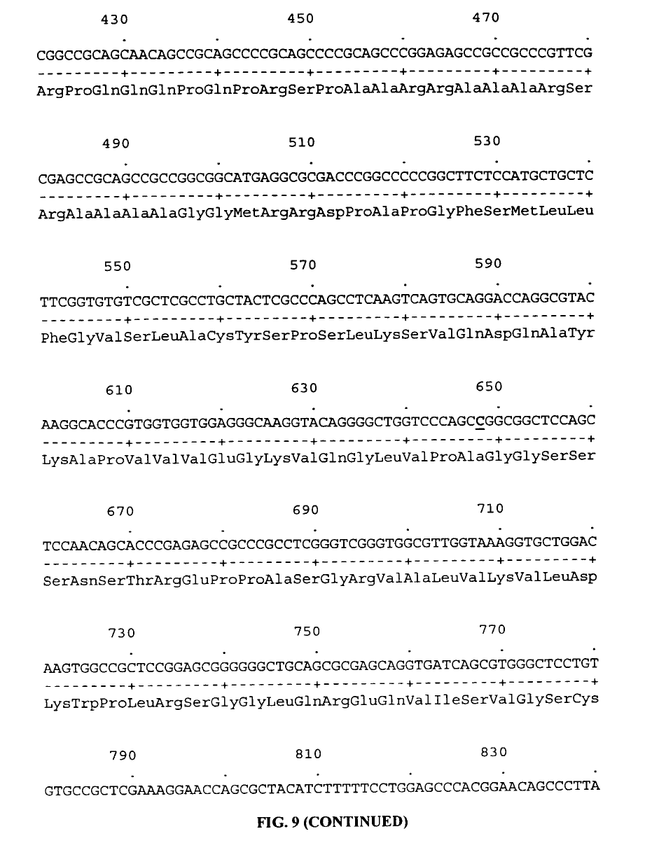

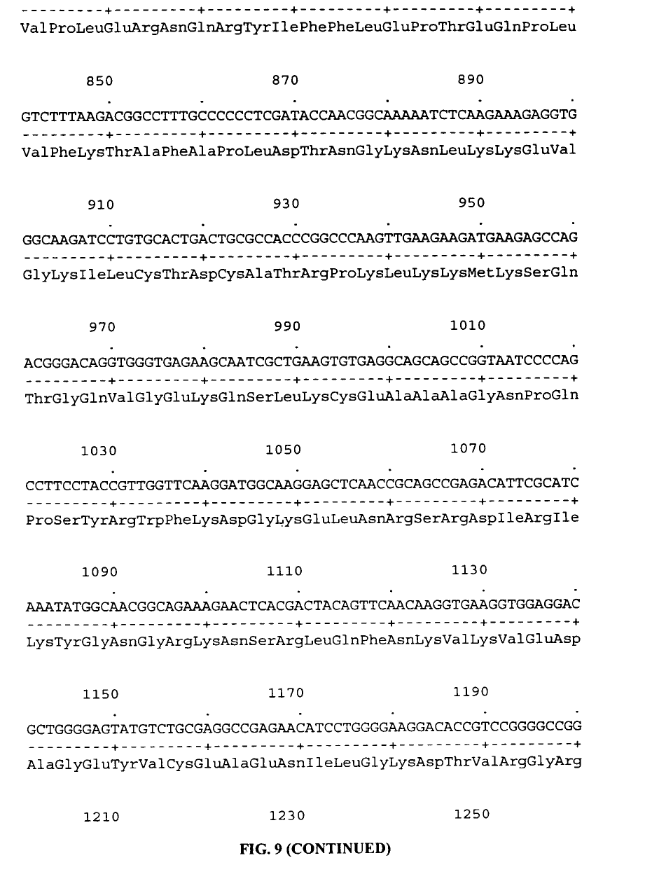

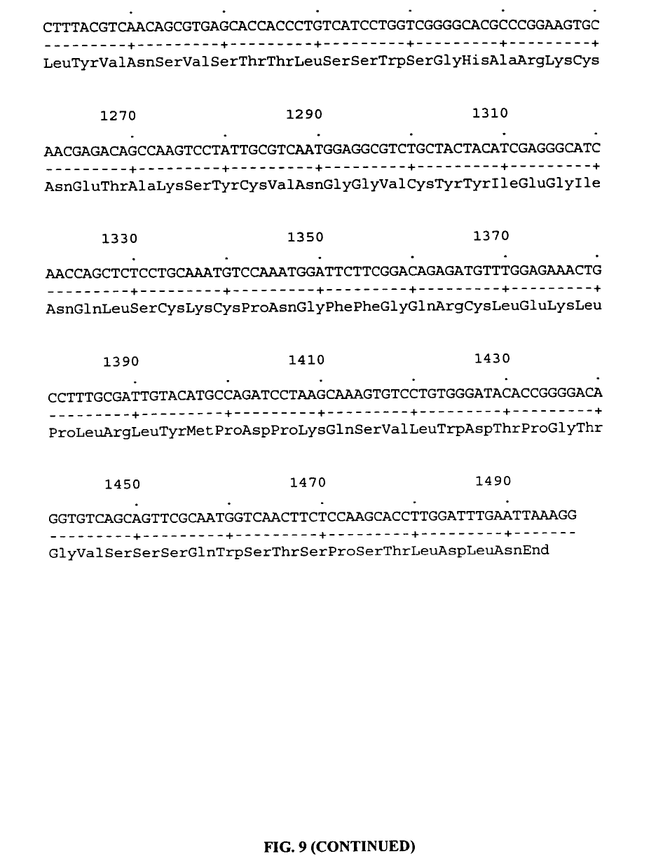

Further, the nucleic acids of this invention include the adjacent chromosomal regions of ADAM or Interactor genes required for accurate expression of the respective gene. In one embodiment, the present invention is directed to at least 15 contiguous nucleotides of the nucleic acid sequence of any of the sequences shown in column 4 of Table 2, SEQ ID NOs. 1-9, and FIGS. 1-12. More particularly, embodiments of this invention include BAC clones of the nucleic acid sequences of the invention.

The invention also relates to direct selected clones and EST's from ADAM and Interactor genes. In a preferred embodiment, the invention relates to clusters of nucleic acids combining the direct selected clones with EST's homologous to BAC sequences and BAC end sequences.

The invention also concerns the use of the nucleotide sequence of the nucleic acids of this invention to identify DNA probes for ADAM and Interactor genes, BAC end sequences, BACs, direct selected clones, and sequence clusters, PCR primers to amplify the ADAM and Interactor genes, nucleotide polymorphisms, and regulatory elements of the ADAM family and interactor genes.

This invention further relates to methods of using isolated or recombinant ADAM and Interactor gene sequences (DNA or RNA) that are characterized by their ability to hybridize to (a) a nucleic acid encoding a protein or polypeptide, such as a nucleic acid having any of the sequences shown in column 4 of Table 2, or (b) a fragment of the foregoing. For example, a fragment can comprise the minimum nucleotides of an ADAM or Interactor protein required to encode a functional ADAM or Interactor protein, or the minimum nucleotides to encode a polypeptide, or to encode a functional equivalent thereof. A functional equivalent can include a polypeptide, which, when incorporated into a cell, has all or part of the activity of an ADAM or Interactor protein. A functional equivalent of an ADAM or Interactor protein, therefore, would have a similar amino acid sequence (at least 65% sequence identity) and similar characteristics to, or perform in substantially the same way as an ADAM or Interactor protein. A nucleic acid which hybridizes to a nucleic acid encoding an ADAM or Interactor protein or polypeptide can be double- or single-stranded. Hybridization to DNA, such as DNA having a sequence set forth in Tables 2-5 and 7, includes hybridization to the strand shown, or to the complementary strand.

The sequences of the present invention may be derived from a variety of sources including DNA, cDNA, synthetic DNA, synthetic RNA, or combinations thereof. Such sequences may comprise genomic DNA, which may or may not include naturally occurring introns. Moreover, such genomic DNA may be obtained in association with promoter regions or poly (A) sequences. The sequences, genomic DNA, or cDNA may be obtained in any of several ways. Genomic DNA can be extracted and purified from suitable cells by means well known in the art. Alternatively, mRNA can be isolated from a cell and used to produce cDNA by reverse transcription or other means.

The present invention also relates to nucleic acids that encode a polypeptide having the amino acid sequence shown in column 5 of Table 2, or functional equivalents thereof. A functional equivalent of an ADAM or Interactor protein includes fragments or variants that perform at least one characteristic function of the ADAM or Interactor protein (e.g., antigenic or intracellular activity). Preferably, a functional equivalent will share at least 65% sequence identity with the ADAM or Interactor polypeptide.

Sequence identity calculations can be performed using computer programs, hybridization methods, or calculations. Preferred computer program methods to determine identity and similarity between two sequences include, but are not limited to, the GCG program package, BLASTN, BLASTX, TBLASTX, and FASTA (J. Devereux et al., 1984, Nucleic Acids Research 12(1):387; S. F. Altschul et al., 1990, J. Molec. Biol. 215:403-410; W. Gish and D. J. States, 1994, Nature Genet. 3:266-272; W. R. Pearson and D. J. Lipman, 1988, Proc Natl. Acad. Sci. USA 85(8):2444-8). The BLAST programs are publicly available from NCBI and other sources. The well-known Smith Waterman algorithm may also be used to determine identity.

For example, nucleotide sequence identity can be determined by comparing a query sequences to sequences in publicly available sequence databases (NCBI) using the BLASTN2 algorithm (S. F. Altschul et al., 1997, Nucl. Acids Res., 25:3389-3402). The parameters for a typical search are: E=0.05, v=50, B=50, wherein E is the expected probability score cutoff, V is the number of database entries returned in the reporting of the results, and B is the number of sequence alignments returned in the reporting of the results (S. F Altschul et al., 1990, J. Mol. Biol., 215:403-410).

In another approach, nucleotide sequence identity can be calculated using the following equation: % identity=(number of identical nucleotides)/(alignment length in nucleotides)*100. For this calculation, alignment length includes internal gaps but not includes terminal gaps. Alternatively, nucleotide sequence identity can be determined experimentally using the specific hybridization conditions described below.

In accordance with the present invention, polynucleotide alterations are selected from the group consisting of at least one nucleotide deletion, substitution, including transition and transversion, insertion, or modification (e.g., via RNA or DNA analogs). Alterations may occur at the 5′ or 3′ terminal positions of the reference nucleotide sequence or anywhere between those terminal positions, interspersed either individually among the nucleotides in the reference sequence or in one or more contiguous groups within the reference sequence. Alterations of a polynucleotide sequence of any one of the sequences shown in Table 2 may create nonsense, missense, or frameshift mutations in this coding sequence, and thereby alter the polypeptide encoded by the polynucleotide following such alterations.

Such altered nucleic acids, including DNA or RNA, can be detected and isolated by hybridization under high stringency conditions or moderate stringency conditions, for example, which are chosen to prevent hybridization of nucleic acids having non-complementary sequences. “Stringency conditions” for hybridizations is a term of art that refers to the conditions of temperature and buffer concentration that permit hybridization of a particular nucleic acid to another nucleic acid in which the first nucleic acid may be perfectly complementary to the second, or the first and second may share some degree of complementarity that is less than perfect.

For example, certain high stringency conditions can be used which distinguish perfectly complementary nucleic acids from those of less complementarity. “High stringency conditions” and “moderate stringency conditions” for nucleic acid hybridizations are explained in F. M. Ausubel et al. (eds), 1995, Current Protocols in Molecular Biology, John Wiley and Sons, Inc., New York, N.Y., the teachings of which are hereby incorporated by reference. In particular, see pages 2.10.1-2.10.16 (especially pages 2.10.8-2.10.11) and pages 6.3.1-6.3.6. The exact conditions which determine the stringency of hybridization depend not only on ionic strength, temperature and the concentration of destabilizing agents such as formamide, but also on factors such as the length of the nucleic acid sequence, base composition, percent mismatch between hybridizing sequences and the frequency of occurrence of subsets of that sequence within other non-identical sequences. Thus, high or moderate stringency conditions can be determined empirically.

By varying hybridization conditions from a level of stringency at which no hybridization occurs to a level at which hybridization is first observed, conditions which will allow a given sequence to hybridize with the most similar sequences in the sample can be determined. Preferably the hybridizing sequences will have 60-70% sequence identity, more preferably 70-85% sequence identity, and even more preferably 90-100% sequence identity.

Typically, the hybridization reaction is initially performed under conditions of low stringency, followed by washes of varying, but higher stringency. Reference to hybridization stringency, e.g., high, moderate, or low stringency, typically relates to such washing conditions. Hybridization conditions are based on the melting temperature (Tₘ) of the nucleic acid probe or primer and are typically classified by degree of stringency of the conditions under which hybridization is measured (Ausubel et al., 1995). For example, high stringency hybridization typically occurs at about 5-10% C. below the Tₘ; moderate stringency hybridization occurs at about 10-20% below the Tₘ; and low stringency hybridization occurs at about 20-25% below the Tₘ. The melting temperature can be approximated by the formulas as known in the art, depending on a number of parameters, such as the length of the hybrid or probe in number of nucleotides, or hybridization buffer ingredients and conditions. As a general guide, Tₘ decreases approximately 1° C. with every 1% decrease in sequence identity at any given SSC concentration. Generally, doubling the concentration of SSC results in an increase in Tₘ of ˜17° C. Using these guidelines, the washing temperature can be determined empirically for moderate or low stringency, depending on the level of mismatch sought.

High stringency hybridization conditions are typically carried out at 65 to 68° C. in 0.1×SSC and 0.1% SDS. Highly stringent conditions allow hybridization of nucleic acid molecules having about 95 to 100% sequence identity. Moderate stringency hybridization conditions are typically carried out at 50 to 65° C. in 1×SSC and 0.1% SDS. Moderate stringency conditions allow hybridization of sequences having at least 80 to 95% nucleotide sequence identity. Low stringency hybridization conditions are typically carried out at 40 to 50° C. in 6×SSC and 0.1% SDS. Low stringency hybridization conditions allow detection of specific hybridization of nucleic acid molecules having at least 50 to 80% nucleotide sequence identity.

For example, high stringency conditions can be attained by hybridization in 50% formamide, 5× Denhardt's solution, 5×SSPE or SSC (1×SSPE buffer comprises 0.15 M NaCl, 10 mM Na₂HPO₄, 1 mM EDTA; 1×SSC buffer comprises 150 mM NaCl, 15 mM sodium citrate, pH 7.0), 0.2% SDS at about 42° C., followed by washing in 1×SSPE or SSC and 0.1% SDS at a temperature of at least 42° C., preferably about 55° C., more preferably about 65° C. Moderate stringency conditions can be attained, for example, by hybridization in 50% formamide, 5× Denhardt's solution, 5×SSPE or SSC, and 0.2% SDS at 42° C. to about 50° C., followed by washing in 0.2×SSPE or SSC and 0.2% SDS at a temperature of at least 42° C., preferably about 55° C., more preferably about 65° C. Low stringency conditions can be attained, for example, by hybridization in 10% formamide, 5× Denhardt's solution, 6×SSPE or SSC, and 0.2% SDS at 42° C., followed by washing in 1×SSPE or SSC, and 0.2% SDS at a temperature of about 45° C., preferably about 50° C. in 4×SSC at 60° C. for 30 min.

High stringency hybridization procedures typically (1) employ low ionic strength and high temperature for washing, such as 0.015 M NaCl/0.0015 M sodium citrate, pH 7.0 (0.1×SSC) with 0.1% sodium dodecyl sulfate (SDS) at 50° C.; (2) employ during hybridization 50% (vol/vol) formamide with 5× Denhardt's solution (0.1% weight/volume highly purified bovine serum albumin/0.1% wt/vol Ficoll/0.1% wt/vol polyvinylpyrrolidone), 50 mM sodium phosphate buffer at pH 6.5 and 5×SSC at 42° C.; or (3) employ hybridization with 50% formamide, 5×SSC, 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC and 0.1% SDS.

In one particular embodiment, high stringency hybridization conditions may be attained by:

Prehybridization treatment of the support (e.g., nitrocellulose filter or nylon membrane), to which is bound the nucleic acid capable of hybridizing with any of the sequences of the invention, is carried out at 65° C. for 6 hr with a solution having the following composition: 4×SSC, 10× Denhardt's (1× Denhardt's comprises 1% Ficoll, 1% polyvinylpyrrolidone, 1% BSA (bovine serum albumin); 1×SSC comprises of 0.15 M of NaCl and 0.015 M of sodium citrate, pH 7);

Replacement of the pre-hybridization solution in contact with the support by a buffer solution having the following composition: 4×SSC, 1× Denhardt's, 25 mM NaPO₄, pH 7, 2 mM EDTA, 0.5% SDS, 100 μg/ml of sonicated salmon sperm DNA containing a nucleic acid derived from the sequences of the invention as probe, in particular a radioactive probe, and previously denatured by a treatment at 100° C. for 3 min;

Incubation for 12 hr at 65° C.;

Successive washings with the following solutions: 1) four washings with 2×SSC, 1× Denhardt's, 0.5% SDS for 45 min at 65° C.; 2) two washings with 0.2×SSC, 0.1×SSC for 45 min at 65° C.; and 3) 0.1×SSC, 0.1% SDS for 45 min at 65° C.

Additional examples of high, medium, and low stringency conditions can be found in Sambrook et al., 1989. Exemplary conditions are also described in M. H. Krause and S. A. Aaronson, 1991, Methods in Enzymology, 200:546-556; Ausubel et al., 1995. It is to be understood that the low, moderate and high stringency hybridization/washing conditions may be varied using a variety of ingredients, buffers, and temperatures well known to and practiced by the skilled practitioner.

Isolated or recombinant nucleic acids that are characterized by their ability to hybridize to a) a nucleic acid encoding an ADAM or Interactor polypeptide, such as the nucleic acids depicted in column 4 of Table 2, SEQ ID NOs. 1-9, and FIGS. 1-12; b) the complement of (a); c) or a portion of (a) or (b) (e.g., under high or moderate stringency conditions), may further encode a protein or polypeptide having at least one function characteristic of an ADAM or Interactor polypeptide, or binding of antibodies that also bind to non-recombinant ADAM or Interactor proteins or polypeptides. The catalytic or binding function of a protein or polypeptide encoded by the hybridizing nucleic acid may be detected by standard enzymatic assays for activity or binding (e.g., assays that measure the binding of a transit peptide or a precursor, or other components of the translocation machinery). Enzymatic assays, complementation tests, or other suitable methods can also be used in procedures for the identification and isolation of nucleic acids which encode a polypeptide such as a polypeptide of the amino acid sequences shown in column 5 of Table 2, or a functional equivalent of these polypeptides. The antigenic properties of proteins or polypeptides encoded by hybridizing nucleic acids can be determined by immunological methods employing antibodies that bind to an ADAM or Interactor polypeptide such as immunoblot, immunoprecipitation and radioimmunoassay. PCR methodology, including RAGE (Rapid Amplification of Genomic DNA Ends), can also be used to screen for and detect the presence of nucleic acids which encode ADAM or Interactor-like proteins and polypeptides, and to assist in cloning such nucleic acids from genomic DNA. PCR methods for these purposes can be found in Innis, M. A., et al., 1990, PCR Protocols: A Guide to Methods and Applications, Academic Press, Inc., San Diego, Calif., incorporated herein by reference.

It is understood that, as a result of the degeneracy of the genetic code, many nucleic acid sequences are possible which encode ADAM or Interactor gene-like proteins or polypeptides. Some of these will have little homology to the nucleotide sequences of any known or naturally-occurring ADAM or Interactor genes but can be used to produce the proteins and polypeptides of this invention by selection of combinations of nucleotide triplets based on codon choices. Such variants, while not hybridizable to a naturally occurring ADAM or Interactor gene, are contemplated within this invention.

Also encompassed by the present invention are alternate splice variants produced by differential processing of the primary transcript(s) of ADAM or Interactor genomic DNA. An alternate splice variant may comprise, for example, the sequences shown in Table 2 or FIGS. 1-12. Alternate splice variants can also comprise other combinations of introns/exons of ADAM or Interactor genes, which can be determined by those of skill in the art. Alternate splice variants can be determined experimentally, for example, by isolating and analyzing cellular RNAs (e.g., Southern blotting or PCR), or by screening cDNA libraries using the 12q23-qter nucleic acid probes or primers described herein. In another approach, alternate splice variants can be predicted using various methods, computer programs, or computer systems available to practitioners in the field.

General methods for splice site prediction can be found in Nakata, 1985, Nucleic Acids Res. 13:5327-5340. In addition, splice sites can be predicted using, for example, the GRAIL™ (E. C. Uberbacher and R. J. Mural, 1991, Proc. Natl. Acad. Sci. USA, 88:11261-11265; E. C. Uberbacher, 1995, Trends Biotech., 13:497-500; http://grail.lsd.ornl.gov/grailexp); GenView (L. Milanesi et al., 1993, Proceedings of the Second International Conference on Bioinformatics, Supercomputing, and Complex Genome Analysis, H. A. Lim et al. (eds), World Scientific Publishing, Singapore, pp. 573-588; http://l25.itba.mi.cnr.it/˜webgene/wwwgene_help.html); SpliceView (http://www.itba.mi.cnr.it/webgene); and HSPL (V. V. Solovyev et al., 1994, Nucleic Acids Res. 22:5156-5163; V. V. Solovyev et al., 1994, “The Prediction of Human Exons by Oligonucleotide Composition and Discriminant Analysis of Spliceable Open Reading Frames,” R. Altman et al. (eds), The Second International conference on Intelligent systems for Molecular Biology, AAAI Press, Menlo Park, Calif., pp. 354-362; V. V. Solovyev et al., 1993, “Identification Of Human Gene Functional Regions Based On Oligonucleotide Composition,” L. Hunter et al. (eds), In Proceedings of First International conference on Intelligent System for Molecular Biology, Bethesda, pp. 371-379) computer systems.

Additionally, computer programs such as GeneParser (E. E. Snyder and G. D. Stormo, 1995, J. Mol. Biol. 248: 1-18; E. E. Snyder and G. D. Stormo, 1993, Nucl. Acids Res. 21(3): 607-613; http://mcdb.colorado.edu/˜eesnyder/GeneParser.html); MZEF (M. Q. Zhang, 1997, Proc. Natl. Acad. Sci. USA, 94:565-568; http://argon.cshl.org/genefinder); MORGAN (S. Salzberg et al., 1998, J. Comp. Biol. 5:667-680; S. Salzberg et al. (eds), 1998, Computational Methods in Molecular Biology, Elsevier Science, New York, N.Y., pp. 187-203); VEIL (J. Henderson et al., 1997, J. Comp. Biol. 4:127-141); GeneScan (S. Tiwari et al., 1997, CABIOS (BioInformatics) 13: 263-270); GeneBuilder (L. Milanesi et al., 1999, Bioinformatics 15:612-621); Eukaryotic GeneMark (J. Besemer et al., 1999, Nucl. Acids Res. 27:3911-3920); and FEXH (V. V. Solovyev et al., 1994, Nucleic Acids Res. 22:5156-5163). In addition, splice sites (i.e., former or potential splice sites) in cDNA sequences can be predicted using, for example, the RNASPL (V. V. Solovyev et al., 1994, Nucleic Acids Res. 22:5156-5163); or INTRON (A. Globek et al., 1991, INTRON version 1.1 manual, Laboratory of Biochemical Genetics, NIMH, Washington, D.C.) programs.

The present invention also encompasses naturally-occurring polymorphisms of ADAM or Interactor genes. As will be understood by those in the art, the genomes of all organisms undergo spontaneous mutation in the course of their continuing evolution generating variant forms of gene sequences (Gusella, 1986, Ann. Rev. Biochem. 55:831-854). Restriction fragment length polymorphisms (RFLPs) include variations in DNA sequences that alter the length of a restriction fragment in the sequence (Botstein et al., 1980, Am. J. Hum. Genet. 32, 314-331). RFLPs have been widely used in human and animal genetic analyses (see WO 90/13668; WO90/11369; Donis-Keller, 1987, Cell 51:319-337; Lander et al., 1989, Genetics 121: 85-99). Short tandem repeats (STRs) include tandem di-, tri- and tetranucleotide repeated motifs, also termed variable number tandem repeat (VNTR) polymorphisms. VNTRs have been used in identity and paternity analysis (U.S. Pat. No. 5,075,217; Armour et al., 1992, FEBS Lett. 307:113-115; Horn et al., WO 91/14003; Jeffreys, EP 370,719), and in a large number of genetic mapping studies.

Single nucleotide polymorphisms (SNPs) are far more frequent than RFLPS, STRs, and VNTRs. SNPs may occur in protein coding (e.g., exon), or non-coding (e.g., intron, 5′UTR, 3′UTR) sequences. SNPs in protein coding regions may comprise silent mutations that do not alter the amino acid sequence of a protein. Alternatively, SNPs in protein coding regions may produce conservative or non-conservative amino acid changes, described in detail below. In some cases, SNPs, including SNP alleles and haplotypes, may give rise to the expression of a defective or other variant protein and, potentially, a genetic disease. SNPs within protein-coding sequences can give rise to genetic diseases, for example, in the β-globin (sickle cell anemia) and CFTR (cystic fibrosis) genes. In non-coding sequences, SNPs may also result in defective protein expression (e.g., as a result of defective splicing). Other single nucleotide polymorphisms have no phenotypic effects.

Single nucleotide polymorphisms can be used in the same manner as RFLPs and VNTRs, but offer several advantages. Single nucleotide polymorphisms tend to occur with greater frequency and are typically spaced more uniformly throughout the genome than other polymorphisms. Also, different SNPs are often easier to distinguish than other types of polymorphisms (e.g., by use of assays employing allele-specific hybridization probes or primers). In one embodiment of the present invention, an ADAM or Interactor nucleic acid contains at least one SNP as set forth in Tables 2-5 and 7, SEQ ID NOs. 1-9, and FIGS. 1-12, described herein. Various combinations, alleles and haplotypes of these SNPs are also encompassed by the invention. In a preferred aspect, an ADAM or Interactor SNP allele or haplotype is associated with a lung-related disorder, such as asthma. Nucleic acids comprising such SNP alleles and haplotypes can be used as diagnostic or therapeutic reagents.

The nucleic acid sequences of the present invention may be derived from a variety of sources including DNA, cDNA, synthetic DNA, synthetic RNA, or combinations thereof. Such sequences may comprise genomic DNA, which may or may not include naturally occurring introns. Moreover, such genomic DNA may be obtained in association with promoter regions or poly(A)+ sequences. The sequences, genomic DNA, or cDNA may be obtained in any of several ways. Genomic DNA can be extracted and purified from suitable cells by means well known in the art. Alternatively, mRNA can be isolated from a cell and used to produce cDNA by reverse transcription or other means.

The nucleic acids described herein are used in the methods of the present invention for production of proteins or polypeptides, through incorporation into cells, tissues, or organisms. In one embodiment, DNA containing all or part of the coding sequence for an ADAM or Interactor polypeptide, or DNA which hybridizes to DNA having the sequence of any one of the sequences shown in Tables 2-5 and 7, SEQ ID NOs. 1-9, and FIGS. 1-12, or a fragment thereof, is incorporated into a vector for expression of the encoded polypeptide in suitable host cells. The encoded amino acid sequence consisting of an ADAM or Interactor polypeptide, or its functional equivalent is capable of normal activity, such as antigenic or intracellular activity.

The invention also concerns the use of the nucleotide sequence of the nucleic acids of this invention to identify DNA probes for ADAM or Interactor genes, PCR primers to amplify ADAM or Interactor genes, nucleotide polymorphisms in ADAM or Interactor genes, and regulatory elements of ADAM or Interactor genes.

The nucleic acids of the present invention find use as primers and templates for the recombinant production of disorder-associated peptides or polypeptides, for chromosome and gene mapping, to provide antisense sequences, for tissue distribution studies, to locate and obtain full length genes, to identify and obtain homologous sequences (wild-type and mutants), and in diagnostic applications. The primers of this invention may comprise all or a portion of the nucleotide sequence of any one shown in Tables 2-5 and 7, SEQ ID NOs. 1-9, and FIGS. 1-12, or a complementary sequence thereof.

Probes may also be used for the detection of ADAM or Interactor-related sequences, and should preferably contain at least 50%, preferably at least 80%, identity to an ADAM or Interactor polynucleotide, or a complementary sequence, or fragments thereof. The probes of this invention may be DNA or RNA, the probes may comprise all or a portion of the nucleotide sequences shown in Tables 2-5 and 7, SEQ ID NOs. 1-9, and FIGS. 1-12, or a complementary sequence thereof, and may include promoter, enhancer elements, and introns of the naturally occurring ADAM or Interactor polynucleotide.

The probes and primers based on the ADAM and Interactor gene sequences disclosed herein are used to identify homologous ADAM and Interactor gene sequences and proteins in other species. These ADAM and Interactor gene sequences and proteins are used in the diagnostic/prognostic, therapeutic and drug-screening methods described herein for the species from which they have been isolated.

### VECTORS AND HOST CELLS

The nucleic acids described herein are used in the methods of the present invention for production of proteins or polypeptides, through incorporation into cells, tissues, or organisms. In one embodiment, DNA containing all or part of the coding sequence for an ADAM or Interactor polypeptide, or DNA which hybridizes to DNA having the sequences shown in Tables 2-5 and 7, SEQ ID NOs. 1-9, and FIGS. 1-12, is incorporated into a vector for expression of the encoded polypeptide in suitable host cells. The encoded polypeptides consisting of ADAM or Interactor genes, or their functional equivalents and are capable of normal activity. A large number of vectors, including bacterial, yeast, and mammalian vectors, have been described for replication and expression in various host cells or cell-free systems, and may be used for gene therapy as well as for simple cloning or protein expression.

In one aspect, an expression vectors comprises a nucleic acid encoding an ADAM or Interactor polypeptide or peptide, as described herein, operably linked to at least one regulatory sequence. Regulatory sequences are known in the art and are selected to direct expression of the desired protein in an appropriate host cell. Accordingly, the term regulatory sequence includes promoters, enhancers and other expression control elements (see D. V. Goeddel, 1990, Methods Enzymol. 185:3-7). Enhancer and other expression control sequences are described in Enhancers and Eukaryotic Gene Expression, 1983, Cold Spring Harbor Press, Cold Spring Harbor, N.Y. It should be understood that the design of the expression vector may depend on such factors as the choice of the host cell to be transfected or the type of polypeptide to be expressed.

Several regulatory elements (e.g., promoters) have been isolated and shown to be effective in the transcription and translation of heterologous proteins in the various hosts. Such regulatory regions, methods of isolation, manner of manipulation, etc. are known in the art. Non-limiting examples of bacterial promoters include the β-lactamase (penicillinase) promoter; lactose promoter; tryptophan (trp) promoter; araBAD (arabinose) operon promoter; lambda-derived P₁ promoter and N gene ribosome binding site; and the hybrid tac promoter derived from sequences of the trp and lac UV5 promoters. Non-limiting examples of yeast promoters include the 3-phosphoglycerate kinase promoter, glyceraldehyde-3-phosphate dehydrogenase (GAPDH) promoter, galactokinase (GAL1) promoter, galactoepimerase promoter, and alcohol dehydrogenase (ADH1) promoter. Suitable promoters for mammalian cells include, without limitation, viral promoters, such as those from Simian Virus 40 (SV40), Rous sarcoma virus (RSV), adenovirus (ADV), and bovine papilloma virus (BPV). Preferred replication and inheritance systems include M13, ColE1, SV40, baculovirus, lambda, adenovirus, CEN ARS, 2 μm ARS and the like. While expression vectors may replicate autonomously, they may also replicate by being inserted into the genome of the host cell, by methods well known in the art.

To obtain expression in eukaryotic cells, terminator sequences, polyadenylation sequences, and enhancer sequences that modulate gene expression may be required. Sequences that cause amplification of the gene may also be desirable. These sequences are well known in the art. Furthermore, sequences that facilitate secretion of the recombinant product from cells, including, but not limited to, bacteria, yeast, and animal cells, such as secretory signal sequences or preprotein or proprotein sequences, may also be included. Such sequences are well described in the art.

Expression and cloning vectors will likely contain a selectable marker, a gene encoding a protein necessary for survival or growth of a host cell transformed with the vector. The presence of this gene ensures growth of only those host cells that express the inserts. Typical selection genes encode proteins that 1) confer resistance to antibiotics or other toxic substances, e.g., ampicillin, neomycin, methotrexate, etc.; 2) complement auxotrophic deficiencies, or 3) supply critical nutrients not available from complex media, e.g., the gene encoding D-alanine racemase for Bacilli. Markers may be an inducible or non-inducible gene and will generally allow for positive selection. Non-limiting examples of markers include the ampicillin resistance marker (i.e., beta-lactamase), tetracycline resistance marker, neomycin/kanamycin resistance marker (i.e., neomycin phosphotransferase), dihydrofolate reductase, glutamine synthetase, and the like. The choice of the proper selectable marker will depend on the host cell, and appropriate markers for different hosts as understood by those of skill in the art.

Suitable expression vectors for use with the present invention include, but are not limited to, pUC, pBluescript (Stratagene), pET (Novagen, Inc., Madison, Wis.), and pREP (Invitrogen) plasmids. Vectors can contain one or more replication and inheritance systems for cloning or expression, one or more markers for selection in the host, e.g., antibiotic resistance, and one or more expression cassettes. The inserted coding sequences can be synthesized by standard methods, isolated from natural sources, or prepared as hybrids. Ligation of the coding sequences to transcriptional regulatory elements (e.g., promoters, enhancers, and insulators) or to other amino acid encoding sequences can be carried out using established methods.

Suitable cell-free expression systems for use with the present invention include, without limitation, rabbit reticulocyte lysate, wheat germ extract, canine pancreatic microsomal membranes, E. coli S30 extract, and coupled transcription/translation systems (Promega Corp., Madison, Wis.). These systems allow the expression of recombinant polypeptides or peptides upon the addition of cloning vectors, DNA fragments, or RNA sequences containing protein-coding regions and appropriate promoter elements.

Non-limiting examples of suitable host cells include bacteria, archea, insect, fungi (e.g., yeast), plant, and animal cells (e.g., mammalian, especially human). Of particular interest are Escherichia coli, Bacillus subtilis, Saccharomyces cerevisiae, SF9 cells, C129 cells, 293 cells, Neurospora, and immortalized mammalian myeloid and lymphoid cell lines. Techniques for the propagation of mammalian cells in culture are well-known (see, Jakoby and Pastan (eds), 1979, Cell Culture. Methods in Enzymology, volume 58, Academic Press, Inc., Harcourt Brace Jovanovich, N.Y.). Examples of commonly used mammalian host cell lines are VERO and HeLa cells, CHO cells, and WI38, BHK, and COS cell lines, although it will be appreciated by the skilled practitioner that other cell lines may be used, e.g., to provide higher expression desirable glycosylation patterns, or other features.

Host cells can be transformed, transfected, or infected as appropriate by any suitable method including electroporation, calcium chloride-, lithium chloride-, lithium acetate/polyethylene glycol-, calcium phosphate-, DEAE-dextran-, liposome-mediated DNA uptake, spheroplasting, injection, microinjection, microprojectile bombardment, phage infection, viral infection, or other established methods. Alternatively, vectors containing the nucleic acids of interest can be transcribed in vitro, and the resulting RNA introduced into the host cell by well-known methods, e.g., by injection (see, Kubo et al., 1988, FEBS Letts. 241:119). The cells into which have been introduced nucleic acids described above are meant to also include the progeny of such cells.

The nucleic acids of the invention may be isolated directly from cells. Alternatively, the polymerase chain reaction (PCR) method can be used to produce the nucleic acids of the invention, using either RNA (e.g., mRNA) or DNA (e.g., genomic DNA) as templates. Primers used for PCR can be synthesized using the sequence information provided herein and can further be designed to introduce appropriate new restriction sites, if desirable, to facilitate incorporation into a given vector for recombinant expression.

Using the information provided in Tables 2-5 and 7, SEQ ID NOs. 1-9, and FIGS. 1-12, one skilled in the art will be able to clone and sequence all representative nucleic acids of interest, including nucleic acids encoding complete protein-coding sequences. It is to be understood that non-protein-coding sequences contained within the sequences shown in Tables 2-5 and 7, SEQ ID NOs. 1-9, and FIGS. 1-12 are also within the scope of the invention. Such sequences include, without limitation, sequences important for replication, recombination, transcription, and translation. Non-limiting examples include promoters and regulatory binding sites involved in regulation of gene expression, and 5′- and 3′-untranslated sequences (e.g., ribosome-binding sites) that form part of mRNA molecules.

The nucleic acids of this invention can be produced in large quantities by replication in a suitable host cell. Natural or synthetic nucleic acid fragments, comprising at least ten contiguous bases coding for a desired peptide or polypeptide can be incorporated into recombinant nucleic acid constructs, usually DNA constructs, capable of introduction into and replication in a prokaryotic or eukaryotic cell. Usually the nucleic acid constructs will be suitable for replication in a unicellular host, such as yeast or bacteria, but may also be intended for introduction to (with and without integration within the genome) cultured mammalian or plant or other eukaryotic cells, cell lines, tissues, or organisms. The purification of nucleic acids produced by the methods of the present invention is described, for example, in Sambrook et al., 1989; F. M. Ausubel et al., 1992, Current Protocols in Molecular Biology, J. Wiley and Sons, New York, N.Y.

The nucleic acids of the present invention can also be produced by chemical synthesis, e.g., by the phosphoramidite method described by Beaucage et al., 1981, Tetra. Letts. 22:1859-1862, or the triester method according to Matteucci et al., 1981, J. Am. Chem. Soc., 103:3185, and can performed on commercial, automated oligonucleotide synthesizers. A double-stranded fragment may be obtained from the single-stranded product of chemical synthesis either by synthesizing the complementary strand and annealing the strands together under appropriate conditions or by adding the complementary strand using DNA polymerase with an appropriate primer sequence.

These nucleic acids can encode full-length variant forms of proteins as well as the wild-type protein. The variant proteins (which could be especially useful for detection and treatment of disorders) will have the variant amino acid sequences encoded by the polymorphisms described in Tables 2-5 and 7, SEQ ID NOs. 1-9, and FIGS. 1-12 when said polymorphisms are read so as to be in-frame with the full-length coding sequence of which it is a component.

Large quantities of the nucleic acids and proteins of the present invention may be prepared by expressing the ADAM or Interactor gene nucleic acids or portions thereof in vectors or other expression vectors in compatible prokaryotic or eukaryotic host cells. The most commonly used prokaryotic hosts are strains of Escherichia coli, although other prokaryotes, such as Bacillus subtilis or Pseudomonas may also be used. Mammalian or other eukaryotic host cells, such as those of yeast, filamentous fungi, plant, insect, or amphibian or avian species, may also be useful for production of the proteins of the present invention. For example, insect cell systems (i.e., lepidopteran host cells and baculovirus expression vectors) are particularly suited for large-scale protein production.

Host cells carrying an expression vector (i.e., transformants or clones) are selected using markers depending on the mode of the vector construction. The marker may be on the same or a different DNA molecule, preferably the same DNA molecule. In prokaryotic hosts, the transformant may be selected, e.g., by resistance to ampicillin, tetracycline or other antibiotics. Production of a particular product based on temperature sensitivity may also serve as an appropriate marker.

Prokaryotic or eukaryotic cells comprising the nucleic acids of the present invention will be useful not only for the production of the nucleic acids and proteins of the present invention, but also, for example, in studying the characteristics of ADAM or Interactor proteins and protein variants. Cells and animals that carry an ADAM or Interactor gene can be used as model systems to study and test for substances that have potential as therapeutic agents. The cells are typically cultured mesenchymal stem cells. These may be isolated from individuals with a somatic or germine ADAM or Interactor gene. Alternatively, the cell line can be engineered to carry an ADAM or Interactor gene, as described above. After a test substance is applied to the cells, the transformed phenotype of the cell is determined. Any trait of transformed cells can be assessed, including respiratory diseases including asthma, atopy, and response to application of putative therapeutic agents.

### ANTISENSE NUCLEIC ACIDS

A further embodiment of the invention is antisense nucleic acids or oligonucleotides which are complementary, in whole or in part, to a target molecule comprising a sense strand, and can hybridize with the target molecule. The target can be DNA, or its RNA counterpart (i.e., wherein T residues of the DNA are U residues in the RNA counterpart). When introduced into a cell, antisense nucleic acids or oligonucleotides can inhibit the expression of the gene encoded by the sense strand or the mRNA transcribed from the sense strand. Antisense nucleic acids can be produced by standard techniques. See, for example, Shewmaker, et al., U.S. Pat. No. 5,107,065.

In a particular embodiment, an antisense nucleic acid or oligonucleotide is wholly or partially complementary to and can hybridize with a target nucleic acid (either DNA or RNA), wherein the target nucleic acid can hybridize to a nucleic acid having the sequence of the complement of the strands shown in Tables 2-5 and 7, SEQ ID NOs. 1-9, and FIGS. 1-12. For example, an antisense nucleic acid or oligonucleotide can be complementary to a target nucleic acid having the sequence shown as the strand of the open reading frames in column 4 of Table 2, or nucleic acids encoding functional equivalents of ADAM or Interactor genes, or to a portion of these nucleic acids sufficient to allow hybridization. A portion, for example a sequence of 16 nucleotides, could be sufficient to inhibit expression of the protein. Or, an antisense nucleic acid or oligonucleotide, complementary to 5′ or 3′ untranslated regions, or overlapping the translation initiation codons (5′ untranslated and translated regions), of ADAM or Interactor genes, or genes encoding a functional equivalent can also be effective. In another embodiment, the antisense nucleic acid is wholly or partially complementary to and can hybridize with a target nucleic acid that encodes an ADAM or Interactor polypeptide.

In addition to the antisense nucleic acids of the invention, oligonucleotides can be constructed which will bind to duplex nucleic acids either in the genes or the DNA:RNA complexes of transcription, to form stable triple helix-containing or triplex nucleic acids to inhibit transcription and expression of a gene encoding an ADAM or Interactor gene, or their functional equivalents (Frank-Kamenetskii, M. D. and Mirkin, S. M., 1995, Ann. Rev. Biochem. 64:65-95). Such oligonucleotides of the invention are constructed using the base-pairing rules of triple helix formation and the nucleotide sequences of the genes or mRNAs for ADAM or Interactor genes.

In preferred embodiments, at least one of the phosphodiester bonds of an antisense oligonucleotide has been substituted with a structure that functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention.

Oligonucleotides may also include species that include at least some modified base forms. Thus, purines and pyrimidines other than those normally found in nature may be so employed. Similarly, modifications on the furanosyl portions of the nucleotide subunits may also be effected, as long as the essential tenets of this invention are adhered to. Examples of such modifications are 2′-O-alkyl- and 2′-halogen-substituted nucleotides. Some non-limiting examples of modifications at the 2′ position of sugar moieties which are useful in the present invention include OH, SH, SCH₃, F, OCH₃, OCN, O(CH₂)ₙNH₂ and O(CH₂)ₙ CH₃, where n is from 1 to about 10. Such oligonucleotides are functionally interchangeable with natural oligonucleotides or synthesized oligonucleotides, which have one or more differences from the natural structure. All such analogs are comprehended by this invention so long as they function effectively to hybridize with an ADAM or Interactor nucleic acid to inhibit the function thereof.

The oligonucleotides in accordance with this invention preferably comprise from about 3 to about 50 subunits. It is more preferred that such oligonucleotides and analogs comprise from about 8 to about 25 subunits and still more preferred to have from about 12 to about 20 subunits. As defined herein, a “subunit” is a base and sugar combination suitably bound to adjacent subunits through phosphodiester or other bonds.

Antisense nucleic acids or oligonulcleotides can be produced by standard techniques (see, e.g., Shewmaker et al., U.S. Pat. No. 5,107,065. The oligonucleotides used in accordance with this invention may be conveniently and routinely made through the well-known technique of solid phase synthesis. Equipment for such synthesis is available from several vendors, including PE Applied Biosystems (Foster City, Calif.). Any other means for such synthesis may also be employed, however, the actual synthesis of the oligonucleotides is well within the abilities of the practitioner. It is also well known to prepare other oligonucleotide such as phosphorothioates and alkylated derivatives.

The oligonucleotides of this invention are designed to be hybridizable with ADAM or Interactor RNA (e.g., mRNA) or DNA. For example, an oligonucleotide (e.g., DNA oligonucleotide) that hybridizes to ADAM or Interactor mRNA can be used to target the mRNA for RnaseH digestion. Alternatively, an oligonucleotide that hybridizes to the translation initiation site of ADAM or Interactor mRNA can be used to prevent translation of the mRNA. In another approach, oligonucleotides that bind to the double-stranded DNA of an ADAM or Interactor gene can be administered. Such oligonucleotides can form a triplex construct and inhibit the transcription of the DNA encoding ADAM or Interactor polypeptides. Triple helix pairing prevents the double helix from opening sufficiently to allow the binding of polymerases, transcription factors, or regulatory molecules. Recent therapeutic advances using triplex DNA have been described (see, e.g., J. E. Gee et al., 1994, Molecular and Immunologic Approaches, Futura Publishing Co., Mt. Kisco, N.Y.).

As non-limiting examples, antisense oligonucleotides may be targeted to hybridize to the following regions: mRNA cap region; translation initiation site; translational termination site; transcription initiation site; transcription termination site; polyadenylation signal; 3′ untranslated region; 5′ untranslated region; 5′ coding region; mid coding region; and 3′ coding region. Preferably, the complementary oligonucleotide is designed to hybridize to the most unique 5′ sequence of an ADAM or Interactor gene, including any of about 15-35 nucleotides spanning the 5′ coding sequence. Appropriate oligonucleotides can be designed using OLIGO software (Molecular Biology Insights, Inc., Cascade, Colo.; http://www.oligo.net).

In accordance with the present invention, an antisense oligonucleotide can be synthesized, formulated as a pharmaceutical composition, and administered to a subject. The synthesis and utilization of antisense and triplex oligonucleotides have been previously described (e.g., H. Simon et al., 1999, Antisense Nucleic Acid Drug Dev. 9:527-31; F. X. Barre et al., 2000, Proc. Natl. Acad. Sci. USA 97:3084-3088; R. Elez et al., 2000, Biochem. Biophys. Res. Commun. 269:352-6; E. R. Sauter et al., 2000, Clin. Cancer Res. 6:654-60). Alternatively, expression vectors derived from retroviruses, adenovirus, herpes or vaccinia viruses, or from various bacterial plasmids may be used for delivery of nucleotide sequences to the targeted organ, tissue or cell population. Methods that are well known to those skilled in the art can be used to construct recombinant vectors that will express nucleic acid sequence that is complementary to the nucleic acid sequence encoding an ADAM or Interactor polypeptide. These techniques are described both in Sambrook et al., 1989 and in Ausubel et al., 1992. For example, ADAM or Interactor gene expression can be inhibited by transforming a cell or tissue with an expression vector that expresses high levels of untranslatable ADAM or Interactor sense or antisense sequences. Even in the absence of integration into the DNA, such vectors may continue to transcribe RNA molecules until they are disabled by endogenous nucleases. Transient expression may last for a month or more with a non-replicating vector, and even longer if appropriate replication elements included in the vector system.

Various assays may be used to test the ability of antisense oligonucleotides to inhibit ADAM or Interactor gene expression. For example, ADAM or Interactor mRNA levels can be assessed Northern blot analysis (Sambrook et al., 1989; Ausubel et al., 1992; J. C. Alwine et al. 1977, Proc. Natl. Acad. Sci. USA 74:5350-5354; I. M. Bird, 1998, Methods Mol. Biol. 105:325-36), quantitative or semi-quantitative RT-PCR analysis (see, e.g., W. M. Freeman et al., 1999, Biotechniques 26:112-122; Ren et al., 1998, Mol. Brain Res. 59:256-63; J. M. Cale et al., 1998, Methods Mol. Biol. 105:351-71), or in situ hybridization (reviewed by A. K. Raap, 1998, Mutat. Res. 400:287-298). Alternatively, ADAM or Interactor polypeptide levels can be measured, e.g., by western blot analysis, indirect immunofluorescence, immunoprecipitation techniques (see, e.g., J. M. Walker, 1998, Protein Protocols on CD-ROM, Humana Press, Totowa, N.J.).

### POLYPEPTIDES

The invention also relates to ADAM or Interactor proteins or polypeptides encoded by the nucleic acids described herein, see Table 2, or portions or variants thereof. The proteins and polypeptides of this invention can be isolated or recombinant. In a preferred embodiment, the proteins or portions thereof have at least one function characteristic of an ADAM or Interactor protein or polypeptide. These proteins are referred to as analogs, and the genes encoding them include, for example, naturally occurring ADAM or Interactor genes, variants (e.g., mutants) encoding those proteins or portions thereof. Such protein or polypeptide variants include mutants differing by the addition, deletion or substitution of one or more amino acid residues, or modified polypeptides in which one or more residues are modified (e.g., by phosphorylation, sulfation, acylation, etc.), and mutants comprising one or more modified residues. The variant can have “conservative” changes, wherein a substituted amino acid has similar structural or chemical properties, e.g., replacement of leucine with isoleucine. More infrequently, a variant can have “nonconservative” changes, e.g., replacement of a glycine with a tryptophan. Guidance in determining which amino acid residues can be substituted, inserted, or deleted without abolishing biological or immunological activity can be determined using computer programs well known in the art, for example, DNASTAR software (DNASTAR, Inc., Madison, Wis.).

As non-limiting examples, conservative substitutions in an ADAM or Interactor amino acid sequence can be made in accordance with the following table:

| Original | Conservative | Original | Conservative |
|---|---|---|---|
| Residue | Substitution(s) | Residue | Substitution(s) |
| Ala | Ser | Leu | Ile, Val |
| Arg | Lys | Lys | Arg, Gln, Glu |
| Asn | Gln, His | Met | Leu, Ile |
| Asp | Glu | Phe | Met, Leu, Tyr |
| Cys | Ser | Ser | Thr |
| Gln | Asn | Thr | Ser |
| Glu | Asp | Trp | Tyr |
| Gly | Pro | Tyr | Trp, Phe |
| His | Asn, Gln | Val | Ile, Leu |
| Ile | Leu, Val | | |

Substantial changes in function or immunogenicity can be made by selecting substitutions that are less conservative than those shown in the table, above. For example, non-conservative substitutions can be made which more significantly affect the structure of the polypeptide in the area of the alteration, for example, the alpha-helical, or beta-sheet structure; the charge or hydrophobicity of the molecule at the target site; or the bulk of the side chain. The substitutions which generally are expected to produce the greatest changes in the polypeptide's properties are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine.

In one embodiment, the percent amino acid sequence identity between an ADAM or Interactor polypeptide such as those shown in Table 2, and functional equivalents thereof is at least 50%. In a preferred embodiment, the percent amino acid sequence identity between such an ADAM or Interactor polypeptide and its functional equivalents is at least 65%. More preferably, the percent amino acid sequence identity of an ADAM or Interactor polypeptide and its functional equivalents is at least 75%, still more preferably, at least 80%, and even more preferably, at least 90%.

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The well-known Smith Waterman algorithm may also be used to determine identity.

Exemplary parameters for amino acid sequence comparison include the following: 1) algorithm from Needleman and Wunsch, 1970, J Mol. Biol. 48:443-453; 2) BLOSSUM62 comparison matrix from Hentikoff and Hentikoff, 1992, Proc. Natl. Acad. Sci. USA 89:10915-10919; 3) gap penalty=12; and 4) gap length penalty=4. A program useful with these parameters is publicly available as the “gap” program (Genetics Computer Group, Madison, Wis.). The aforementioned parameters are the default parameters for polypeptide comparisons (with no penalty for end gaps).

Alternatively, polypeptide sequence identity can be calculated using the following equation: % identity=(the number of identical residues)/(alignment length in amino acid residues)*100. For this calculation, alignment length includes internal gaps but does not include terminal gaps.

In accordance with the present invention, polypeptide sequences may be identical to the sequence of any one of the sequences shown in Table 2, or may include up to a certain integer number of amino acid alterations. Polypeptide alterations are selected from the group consisting of at least one amino acid deletion, substitution, including conservative and non-conservative substitution, or insertion. Alterations may occur at the amino- or carboxy-terminal positions of the reference polypeptide sequence or anywhere between those terminal positions, interspersed either individually among the amino acids in the reference sequence or in one or more contiguous groups within the reference sequence.

In specific embodiments, a polypeptide variant may be encoded by an ADAM or Interactor nucleic acid comprising a SNP, allele, haplotype, or an alternate splice variant. For example, a polypeptide variant may be encoded by an ADAM or Interactor gene variant comprising a nucleotide sequence of any one of sequences shown in Tables 2-5 and 7, SEQ ID NOs. 1-9, and FIGS. 1-12.

The invention also relates to isolated, synthesized or recombinant portions or fragments of ADAM or Interactor protein or polypeptide as described herein. Polypeptide fragments (i.e., peptides) can be made which have full or partial function on their own, or which when mixed together (though fully, partially, or nonfunctional alone), spontaneously assemble with one or more other polypeptides to reconstitute a functional protein having at least one functional characteristic of an ADAM or Interactor protein of this invention. In addition, ADAM or Interactor polypeptide fragments may comprise, for example, one or more domains of the ADAM or Interactor polypeptide, disclosed herein.

Polypeptides according to the invention can comprise at least 5 contiguous amino acid residues; preferably the polypeptides comprise at least 12 contiguous residues; more preferably the polypeptides comprise at least 20 contiguous residues; and yet more preferably the polypeptides comprise at least 30 contiguous residues. Nucleic acids comprising protein-coding sequences can be used to direct the expression of asthma-associated polypeptides in intact cells or in cell-free translation systems. The coding sequence can be tailored, if desired, for more efficient expression in a given host organism, and can be used to synthesize oligonucleotides encoding the desired amino acid sequences. The resulting oligonucleotides can be inserted into an appropriate vector and expressed in a compatible host organism or translation system.

The polypeptides of the present invention, including function-conservative variants, may be isolated from wild-type or mutant cells (e.g., human cells or cell lines), from heterologous organisms or cells (e.g., bacteria, yeast, insect, plant, and mammalian cells), or from cell-free translation systems (e.g., wheat germ, microsomal membrane, or bacterial extracts) in which a protein-coding sequence has been introduced and expressed. Furthermore, the polypeptides may be part of recombinant fusion proteins. The polypeptides can also, advantageously, be made by synthetic chemistry. Polypeptides may be chemically synthesized by commercially available automated procedures, including, without limitation, exclusive solid phase synthesis, partial solid phase methods, fragment condensation or classical solution synthesis.

Methods for polypeptide purification are well-known in the art, including, without limitation, preparative disc-gel electrophoresis, isoelectric focusing, HPLC, reversed-phase HPLC, gel filtration, ion exchange and partition chromatography, and countercurrent distribution. For some purposes, it is preferable to produce the polypeptide in a recombinant system in which the protein contains an additional sequence (e.g., epitope or protein) tag that facilitates purification. Non-limiting examples of epitope tags include c-myc, haemagglutinin (HA), polyhistidine (6X-HIS) (SEQ ID NO:), GLU-GLU, and DYKDDDDK (SEQ ID NO:) (FLAG®) epitope tags. Non-limiting examples of protein tags include glutathione-S-transferase (GST), green fluorescent protein (GFP), and maltose binding protein (MBP).

In one approach, the coding sequence of a polypeptide or peptide can be cloned into a vector that creates a fusion with a sequence tag of interest. Suitable vectors include, without limitation, pRSET (Invitrogen Corp., San Diego, Calif.), PGEX (Amersham-Pharmacia Biotech, Inc., Piscataway, N.J.), pEGFP (CLONTECH Laboratories, Inc., Palo Alto, Calif.), and PMAL™ (New England BioLabs (NEB), Inc., Beverly, Mass.) plasmids. Following expression, the epitope, or protein tagged polypeptide or peptide can be purified from a crude lysate of the translation system or host cell by chromatography on an appropriate solid-phase matrix. In some cases, it may be preferable to remove the epitope or protein tag (i.e., via protease cleavage) following purification. As an alternative approach, antibodies produced against a disorder-associated protein or against peptides derived therefrom can be used as purification reagents. Other purification methods are also possible.

The present invention also encompasses modifications of an ADAM or Interactor polypeptides. The isolated polypeptides may be modified by, for example, phosphorylation, sulfation, acylation, or other protein modifications. They may also be modified with a label capable of providing a detectable signal, either directly or indirectly, including, but not limited to, radioisotopes and fluorescent compounds, as described in detail herein.

Both the naturally occurring and recombinant forms of the polypeptides of the invention can advantageously be used to screen compounds for binding activity. Many methods of screening for binding activity are known by those skilled in the art and may be used to practice the invention. Several methods of automated assays have been developed in recent years so as to permit screening of tens of thousands of compounds in a short period of time. Such high-throughput screening methods are particularly preferred. The use of high-throughput screening assays to test for inhibitors is greatly facilitated by the availability of large amounts of purified polypeptides, as provided by the invention. The polypeptides of the invention also find use as therapeutic agents as well as antigenic components to prepare antibodies.

The polypeptides of this invention find use as immunogenic components useful as antigens for preparing antibodies by standard methods. It is well known in the art that immunogenic epitopes generally contain at least 5 contiguous amino acid residues (Ohno et al., 1985, Proc. Natl. Acad. Sci. USA 82:2945). Therefore, the immunogenic components of this invention will typically comprise at least 5 contiguous amino acid residues of the sequence of the complete polypeptide chains. Preferably, they will contain at least 7, and most preferably at least 10 contiguous amino acid residues or more to ensure that they will be immunogenic. Whether a given component is immunogenic can readily be determined by routine experimentation. Such immunogenic components can be produced by proteolytic cleavage of larger polypeptides or by chemical synthesis or recombinant technology and are thus not limited by proteolytic cleavage sites. The present invention thus encompasses antibodies that specifically recognize asthma-associated immunogenic components.

### STRUCTURAL STUDIES

A purified ADAM or Interactor polypeptide, or portions or complexes thereof, can be analyzed by well-established methods (e.g., X-ray crystallography, NMR, CD, etc.) to determine the three-dimensional structure of the molecule. The three-dimensional structure, in turn, can be used to model intermolecular interactions. Exemplary methods for crystallization and X-ray crystallography are found in P. G. Jones, 1981, Chemistry in Britain, 17:222-225; C. Jones et al. (eds), Crystallographic Methods and Protocols, Humana Press, Totowa, N.J.; A. McPherson, 1982, Preparation and Analysis of Protein Crystals, John Wiley & Sons, New York, N.Y.; T. L. Blundell and L. N. Johnson, 1976, Protein Crystallography, Academic Press, Inc., New York, N.Y.; A. Holden and P. Singer, 1960, Crystals and Crystal Growing, Anchor Books-Doubleday, New York, N.Y.; R. A. Laudise, 1970, The Growth of Single Crystals, Solid State Physical Electronics Series, N. Holonyak, Jr., (ed), Prentice-Hall, Inc.; G. H. Stout and L. H. Jensen, 1989, X-ray Structure Determination: A Practical Guide, 2nd edition, John Wiliey & Sons, New York, N.Y.; Fundamentals of Analytical Chemistry, 3rd. edition, Saunders Golden Sunburst Series, Holt, Rinehart and Winston, Philadelphia, Pa., 1976; P. D. Boyle of the Department of Chemistry of North Carolina State University at http://laue.chem.ncsu.edu/web/GrowXtal.html; M. B. Berry, 1995, Protein Crystalization: Theory and Practice, Structure and Dynamics of E. coli Adenylate Kinase, Doctoral Thesis, Rice University, Houston Tex.; www.bioc.rice.edu/˜berry/papers/crystalization/crystalization.html.

For X-ray diffraction studies, single crystals can be grown to suitable size. Preferably, a crystal has a size of 0.2 to 0.4 mm in at least two of the three dimensions. Crystals can be formed in a solution comprising an ADAM or Interactor polypeptide (e.g., 1.5-200 mg/ml) and reagents that reduce the solubility to conditions close to spontaneous precipitation. Factors that affect the formation of polypeptide crystals include: 1) purity; 2) substrates or co-factors; 3) pH; 4) temperature; 5) polypeptide concentration; and 6) characteristics of the precipitant. Preferably, the ADAM or Interactor polypeptides are pure, i.e., free from contaminating components (at least 95% pure), and free from denatured ADAM or Interactor polypeptides. In particular, polypeptides can be purified by FPLC and HPLC techniques to assure homogeneity (see, Lin et al., 1992, J. Crystal. Growth. 122:242-245). Optionally, ADAM or Interactor polypeptide substrates or co-factors can be added to stabilize the quaternary structure of the protein and promote lattice packing.

Suitable precipitants for crystallization include, but are not limited to, salts (e.g., ammonium sulphate, potassium phosphate); polymers (e.g., polyethylene glycol (PEG) 6000); alcohols (e.g., ethanol); polyalcohols (e.g., 1-methyl-2,4 pentane diol (MPD)); organic solvents; sulfonic dyes; and deionized water. The ability of a salt to precipitate polypeptides can be generally described by the Hofmeister series: PO₄³⁻>HPO₄²⁻=SO₄²⁻>citrate>CH₃CO₂⁻>Cl⁻>Br⁻>NO₃⁻>ClO₄⁻>SCN⁻; and NH₄⁺>K⁺>Na⁺>Li⁺. Non-limiting examples of salt precipitants are shown below (see Berry, 1995).

| Precipitant | Maximum concentration |
|---|---|
| (NH₄⁺/Na⁺/Li⁺)₂ or | 4.0/1.5/2.1/2.5 M |
| Mg₂ + SO₄²⁻ | |
| NH₄⁺/Na⁺/K⁺ PO₄³⁻ | 3.0/4.0/4.0 M |
| NH₄⁺/K⁺/Na⁺ Li⁺ citrate | ˜1.8 M |
| NH₄⁺/K⁺/Na⁺/Li⁺ | ˜3.0 M |
| acetate | |
| NH₄⁺/K⁺/Na⁺/Li⁺ Cl⁻ | 5.2/9.8/4.2/5.4 M |
| NH₄⁺NO₃⁻ | ˜8.0 M |

High molecular weight polymers useful as precipitating agents include polyethylene glycol (PEG), dextran, polyvinyl alcohol, and polyvinyl pyrrolidone (A. Polson et al., 1964, Biochem. Biophys. Acta. 82:463-475). In general, polyethylene glycol (PEG) is the most effective for forming crystals. PEG compounds with molecular weights less than 1000 can be used at concentrations above 40% v/v. PEGs with molecular weights above 1000 can be used at concentration 5-50% w/v. Typically, PEG solutions are mixed with ˜0.1% sodium azide to prevent bacterial growth.

Typically, crystallization requires the addition of buffers and a specific salt content to maintain the proper pH and ionic strength for a protein's stability. Suitable additives include, but are not limited to sodium chloride (e.g., 50-500 mM as additive to PEG and MPD; 0.15-2 M as additive to PEG); potassium chloride (e.g., 0.05-2 M); lithium chloride (e.g., 0.05-2 M); sodium fluoride (e.g., 20-300 mM); ammonium sulfate (e.g., 20-300 mM); lithium sulfate (e.g., 0.05-2 M); sodium or ammonium thiocyanate (e.g., 50-500 mM); MPD (e.g., 0.5-50%); 1,6 hexane diol (e.g., 0.5-10%); 1,2,3 heptane triol (e.g., 0.5-15%); and benzamidine (e.g., 0.5-15%).

Detergents may be used to maintain protein solubility and prevent aggregation. Suitable detergents include, but are not limited to non-ionic detergents such as sugar derivatives, oligoethyleneglycol derivatives, dimethylamine-N-oxides, cholate derivatives, N-octyl hydroxyalkylsulphoxides, sulphobetains, and lipid-like detergents. Sugar-derived detergents include alkyl glucopyranosides (e.g., C8-GP, C9-GP), alkyl thio-glucopyranosides (e.g., C8-tGP), alkyl maltopyranosides (e.g., C10-M, C12-M; CYMAL-3, CYMAL-5, CYMAL-6), alkyl thio-maltopyranosides, alkyl galactopyranosides, alkyl sucroses (e.g., N-octanoylsucrose), and glucamides (e.g., HECAMEG, C-HEGA-10; MEGA-8). Oligoethyleneglycol-derived detergents include alkyl polyoxyethylenes (e.g., C8-E5, C8-En; C12-E8; C12-E9) and phenyl polyoxyethylenes (e.g., Triton X-100). Dimethylamine-N-oxide detergents include, e.g., C10-DAO; DDAO; LDAO. Cholate-derived detergents include, e.g., Deoxy-Big CHAP, digitonin. Lipid-like detergents include phosphocholine compounds. Suitable detergents further include zwitter-ionic detergents (e.g., ZWITTERGENT 3-10; ZWITTERGENT 3-12); and ionic detergents (e.g., SDS).

Crystallization of macromolecules has been performed at temperatures ranging from 60° C. to less than 0° C. However, most molecules can be crystallized at 4° C. or 22° C. Lower temperatures promote stabilization of polypeptides and inhibit bacterial growth. In general, polypeptides are more soluble in salt solutions at lower temperatures (e.g., 4° C.), but less soluble in PEG and MPD solutions at lower temperatures. To allow crystallization at 4° C. or 22° C., the precipitant or protein concentration can be increased or decreased as required. Heating, melting, and cooling of crystals or aggregates can be used to enlarge crystals. In addition, crystallization at both 4° C. and 22° C. can be assessed (A. McPherson, 1992, J. Cryst. Growth. 122:161-167; C. W. Carter, Jr. and C. W. Carter, 1979, J. Biol Chem. 254:12219-12223; T. Bergfors, 1993, Crystalization Lab Manual).

A crystallization protocol can be adapted to a particular polypeptide or peptide. In particular, the physical and chemical properties of the polypeptide can be considered (e.g., aggregation, stability, adherence to membranes or tubing, internal disulfide linkages, surface cysteines, chelating ions, etc.). For initial experiments, the standard set of crystalization reagents can be used (Hampton Research, Laguna Niguel, Calif.). In addition, the CRYSTOOL program can provide guidance in determining optimal crystallization conditions (Brent Segelke, 1995, Efficiency analysis of sampling protocols used in protein crystallization screening and crystal structure from two novel crystal forms of PLA2, Ph.D. Thesis, University of California, San Diego; http://www.ccp14.ac.uk/ccp/web-mirrors/llnlrupp/crystool/crystool.htm). Exemplary crystallization conditions are shown below (see Berry, 1995).

| | | Concentration | |
|---|---|---|---|
| | | of Major | Concentration |
| Major Precipitant | Additive | Precipitant | of Additive |
| (NH₄)₂SO₄ | PEG 400-2000, | 2.0-4.0 M | 6%-0.5% |
| | MPD, ethanol, | | |
| | or methanol | | |
| Na citrate | PEG 400-2000, | 1.4-1.8 M | 6%-0.5% |
| | MPD, ethanol, | | |
| | or methanol | | |
| PEG 1000-20000 | (NH 4)₂SO₄, NaCl, | 40-50% | 0.2-0.6 M |
| | or Na formate | | |

Robots can be used for automatic screening and optimization of crystallization conditions. For example, the IMPAX and Oryx systems can be used (Douglas Instruments, Ltd., East Garston, United Kingdom). The CRYSTOOL program (Segelke, supra) can be integrated with the robotics programming. In addition, the Xact program can be used to construct, maintain, and record the results of various crystallization experiments (see, e.g., D. E. Brodersen et al., 1999, J. Appl. Cryst. 32: 1012-1016; G. R. Andersen and J. Nyborg, 1996, J. Appl. Cryst. 29:236-240). The Xact program supports multiple users and organizes the results of crystallization experiments into hierarchies. Advantageously, Xact is compatible with both CRYSTOOL and Microsoft® Excel programs.

Four methods are commonly employed to crystallize macromolecules: vapor diffusion, free interface diffusion, batch, and dialysis. The vapor diffusion technique is typically performed by formulating a 1:1 mixture of a solution comprising the polypeptide of interest and a solution containing the precipitant at the final concentration that is to be achieved after vapor equilibration. The drop containing the 1:1 mixture of protein and precipitant is then suspended and sealed over the well solution, which contains the precipitant at the target concentration, as either a hanging or sitting drop. Vapor diffusion can be used to screen a large number of crystallization conditions or when small amounts of polypeptide are available. For screening, drop sizes of 1 to 2 μl can be used. Once preliminary crystallization conditions have been determined, drop sizes such as 10 μl can be used. Notably, results from hanging drops may be improved with agarose gels (see K. Provost and M.-C. Robert, 1991, J. Cryst. Growth. 110:258-264). Free interface diffusion is performed by layering of a low-density solution onto one of higher density, usually in the form of concentrated protein onto concentrated salt. Since the solute to be crystallized must be concentrated, this method typically requires relatively large amounts of protein. However, the method can be adapted to work with small amounts of protein. In a representative experiment, 2 to 5 μl of sample is pipetted into one end of a 20 μl microcapillary pipet. Next, 2 to 5 μl of precipitant is pipetted into the capillary without introducing an air bubble, and the ends of the pipet are sealed. With sufficient amounts of protein, this method can be used to obtain relatively large crystals (see, e.g., S. M. Althoff et al., 1988, J. Mol. Biol. 199:665-666).

The batch technique is performed by mixing concentrated polypeptide with concentrated precipitant to produce a final concentration that is supersaturated for the solute macromolecule. Notably, this method can employ relatively large amounts of solution (e.g., milliliter quantities), and can produce large crystals. For that reason, the batch technique is not recommended for screening initial crystallization conditions.

The dialysis technique is performed by diffusing precipitant molecules through a semipermeable membrane to slowly increase the concentration of the solute inside the membrane. Dialysis tubing can be used to dialyze milliliter quantities of sample, whereas dialysis buttons can be used to dialyze microliter quantities (e.g., 7-200 μl). Dialysis buttons may be constructed out of glass, perspex, or Teflon™ (see, e.g., Cambridge Repetition Engineers Ltd., Greens Road, Cambridge CB4 3EQ, UK; Hampton Research). Using this method, the precipitating solution can be varied by moving the entire dialysis button or sack into a different solution. In this way, polypeptides can be “reused” until the correct conditions for crystallization are found (see, e.g., C. W. Carter, Jr. et al., 1988, J. Cryst. Growth. 90:60-73). However, this method is not recommended for precipitants comprising concentrated PEG solutions.

Various strategies have been designed to screen crystallization conditions, including 1) pl screening; 2) grid screening; 3) factorials; 4) solubility assays; 5) perturbation; and 6) sparse matrices. In accordance with the pl screening method, the pl of a polypeptide is presumed to be its crystallization point. Screening at the pl can be performed by dialysis against low concentrations of buffer (less than 20 mM) at the appropriate pH, or by use of conventional precipitants.

The grid screening method can be performed on two-dimensional matrices. Typically, the precipitant concentration is plotted against pH. The optimal conditions can be determined for each axis, and then combined. At that point, additional factors can be tested (e.g., temperature, additives). This method works best with fast-forming crystals, and can be readily automated (see M. J. Cox and P. C. Weber, 1988, J. Cryst. Growth. 90:318-324). Grid screens are commercially available for popular precipitants such as ammonium sulphate, PEG 6000, MPD, PEG/LiCl, and NaCl (see, e.g., Hamilton Research).

The incomplete factorial method can be performed by 1) selecting a set of ˜20 conditions; 2) randomly assigning combinations of these conditions; 3) grading the success of the results of each experiment using an objective scale; and 4) statistically evaluating the effects of each of the conditions on crystal formation (see, e.g., C. W. Carter, Jr. et al., 1988, J. Cryst. Growth. 90:60-73). In particular, conditions such as pH, temperature, precipitating agent, and cations can be tested. Dialysis buttons are preferably used with this method. Typically, optimal conditions/combinations can be determined within 35 tests. Similar approaches, such as “footprinting” conditions, may also be employed (see, e.g., E. A. Stura et al., 1991, J. Cryst. Growth. 110:1-2).

The perturbation approach can be performed by altering crystallization conditions by introducing a series of additives designed to test the effects of altering the structure of bulk solvent and the solvent dielectric on crystal formation (see, e.g., Whitaker et al., 1995, Biochem. 34:8221-8226). Additives for increasing the solvent dialectric include, but are not limited to, NaCl, KCl, or LiCl (e.g., 200 mM); Na formate (e.g., 200 mM); Na₂HPO₄ or K₂HPO₄ (e.g., 200 mM); urea, triachloroacetate, guanidium HCl, or KSCN (e.g., 20-50 mM). A non-limiting list of additives for decreasing the solvent dialectric include methanol, ethanol, isopropanol, or tert-butanol (e.g., 1-5%); MPD (e.g., 1%); PEG 400, PEG 600, or PEG 1000 (e.g., 1-4%); PEG MME (monomethylether) 550, PEG MME 750, PEG MME 2000 (e.g., 1-4%).

As an alternative to the above-screening methods, the sparse matrix approach can be used (see, e.g., J. Jancarik and S.-H. J. Kim, 1991, Appl. Cryst. 24:409-411; A. McPherson, 1992, J. Cryst. Growth. 122:161-167; B. Cudney et al., 1994, Acta. Cryst. D50:414-423). Sparse matrix screens are commercially available (see, e.g., Hampton Research; Molecular Dimensions, Inc., Apopka, Fla.; Emerald Biostructures, Inc., Lemont, Ill.). Notably, data from Hampton Research sparse matrix screens can be stored and analyzed using ASPRUN software (Douglas Instruments).

Exemplary conditions for an initial screen are shown below (see Berry, 1995).

### CRYSTALIZATION CONDITIONS

Tray 1:

| | | | | | | Ammonium sulfate (wells 7-12) | | | | | |
|---|---|---|---|---|---|---|---|---|---|---|---|
| PEG 8000 (wells 1-6) | | | | | | | | | | | 1 |
| 20% | 20% | 20% | 35% | 35% | 35% | 2.0 M | 2.0 M | 2.0 M | 2 5 M | 2.5 M | 2.5 M |
| pH 5.0 | pH 7 0 | pH 8 6 | pH 5.0 | pH 7.0 | pH 8.6 | pH 5.0 | pH 7.0 | pH 8.8 | pH 5.0 | pH 7.0 | pH 8.8 |
| MPD (wells 13-16) | +TC,17/32 Na Citrate (wells 17-20) | | | | | | | Na/K Phosphate (wells 21-24) | | | |
| 13 | 14 | 15 | 16 | 17 | 18 | 19 | 20 | 21 | 22 | 23 | 24 |
| 30% | 30% | 50% | 50% | 1.3 M | 1.3 M | 1.5 M | 1.5 M | 2.0 M | 2.0 M | 2.5 M | 2.5 M |
| pH 5.8 | pH 7.6 | pH 5.8 | pH 7.6 | pH 5.8 | pH 7.5 | pH 5.8 | pH 7.5 | pH 6.0 | pH 7.4 | pH 6.0 | pH 7.4 |

Tray 2:

PEG 2000 MME/0.2 M Ammon. sulfate (wells 25-30)

| 25 | 26 | 27 | 28 | 29 | 30 |
|---|---|---|---|---|---|
| 25% | 25% | 25% | 40% | 40% | 40% |
| pH 5.5 | pH 7.0 | pH 8.5 | pH 5.5 | pH 7.0 | pH 8.5 |

Random for wells 31 to 48

The initial screen can be used with hanging or sitting drops. To conserve the sample, tray 2 can be set up several weeks following tray 1. Wells 31-48 of tray 2 can comprise a random set of solutions. Alternatively, solutions can be formulated using sparse methods. Preferably, test solutions cover a broad range of precipitants, additives, and pH (especially pH 5.0-9.0).

Seeding can be used to trigger nucleation and crystal growth (Stura and Wilson, 1990, J. Cryst. Growth. 110:270-282; C. Thaller et al., 1981, J. Mol. Biol. 147:465-469; A. McPherson and P. Schlichta, 1988, J. Cryst. Growth. 90:47-50). In general, seeding can performed by transferring crystal seeds into a polypeptide solution to allow polypeptide molecules to deposit on the surface of the seeds and produce crystals. Two seeding methods can be used: microseeding and macroseeding. For microseeding, a crystal can be ground into tiny pieces and transferred into the protein solution. Alternatively, seeds can be transferred by adding 1-2 μl of the seed solution directly to the equilibrated protein solution. In another approach, seeds can be transferred by dipping a hair in the seed solution and then streaking the hair across the surface of the drop (streak seeding; see Stura and Wilson, supra). For macroseeding, an intact crystal can be transferred into the protein solution (see, e.g., C. Thaller et al., 1981, J. Mol. Biol. 147:465-469). Preferably, the surface of the crystal seed is washed to regenerate the growing surface prior to being transferred. Optimally, the protein solution for crystallization is close to saturation and the crystal seed is not completely dissolved upon transfer.

### ANTIBODIES

Another aspect of the invention pertains to antibodies directed to ADAM or Interactor polypeptides, or portions or variants thereof. The invention provides polyclonal and monoclonal antibodies that bind ADAM or Interactor polypeptides or peptides. The antibodies may be elicited in an animal host (e.g., rabbit, goat, mouse, or other non-human mammal) by immunization with disorder-associated immunogenic components. Antibodies may also be elicited by in vitro immunization (sensitization) of immune cells. The immunogenic components used to elicit the production of antibodies may be isolated from cells or chemically synthesized. The antibodies may also be produced in recombinant systems programmed with appropriate antibody-encoding DNA. Alternatively, the antibodies may be constructed by biochemical reconstitution of purified heavy and light chains. The antibodies include hybrid antibodies, chimeric antibodies, and univalent antibodies. Also included are Fab fragments, including Fab¹ and Fab(ab)² fragments of antibodies.

In accordance with the present invention, antibodies are directed to ADAM or Interactor genes (e.g., such as the sequences shown in Tables 2-5 and 7, SEQ ID NOs. 1-9, and FIGS. 1-12), or variants, or portions thereof. For example, antibodies can be produced to bind to an ADAM or Interactor gene polypeptide encoded by an alternate splice variant comprising the nucleotide sequences shown in FIGS. 1-12. As another example, antibodies can be produced to bind to an ADAM or Interactor polypeptide variant encoded by a nucleic acid containing one or more ADAM or Interactor gene SNPs as set forth in SEQ ID. NOs.: 1-9. Such antibodies can be used as diagnostic or therapeutic reagents.

An isolated ADAM or Interactor gene polypeptide, or variant, or portion thereof, can be used as an immunogen to generate antibodies using standard techniques for polyclonal and monoclonal antibody preparation. A full-length ADAM or Interactor polypeptide can be used or, alternatively, the invention provides antigenic peptide portions of ADAM or Interactor polypeptides for use as immunogens. The antigenic peptide of an ADAM or Interactor comprises at least 5 contiguous amino acid residues of the amino acid sequence shown in any one of column 5 of Table 2, or a variant thereof, and encompasses an epitope of an ADAM or Interactor polypeptide such that an antibody raised against the peptide forms a specific immune complex with an ADAM or Interactor amino acid sequence.

An appropriate immunogenic preparation can contain, for example, recombinantly produced ADAM or Interactor polypeptide or a chemically synthesized ADAM or Interactor polypeptide, or portions thereof. The preparation can further include an adjuvant, such as Freund's complete or incomplete adjuvant, or similar immunostimulatory agent. A number of adjuvants are known and used by those skilled in the art. Non-limiting examples of suitable adjuvants include incomplete Freund's adjuvant, mineral gels such as alum, aluminum phosphate, aluminum hydroxide, aluminum silica, and surface-active substances such as lysolecithin, pluronic polyols, polyanions, peptides, oil emulsions, keyhole limpet hemocyanin, and dinitrophenol. Further examples of adjuvants include N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-Lalanyl-D-isoglutaminyl-L-alanine-2-(1′-2′-dipalmitoyl-sn-glycero-3 hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. A particularly useful adjuvant comprises 5% (wt/vol) squalene, 2.5% Pluronic L121 polymer and 0.2% polysorbate in phosphate buffered saline (Kwak et al., 1992, New Eng. J. Med. 327:1209-1215). Preferred adjuvants include complete BCG, Detox, (RIBI, Immunochem Research Inc.), ISCOMS, and aluminum hydroxide adjuvant (Superphos, Biosector). The effectiveness of an adjuvant may be determined by measuring the amount of antibodies directed against the immunogenic peptide.

Polyclonal antibodies to ADAM or Interactor polypeptides can be prepared as described above by immunizing a suitable subject with an ADAM or Interactor gene immunogen. The antibody titer in the immunized subject can be monitored over time by standard techniques, such as with an enzyme linked immunosorbent assay (ELISA) using immobilized ADAM or Interactor polypeptide or peptide. If desired, the antibody molecules can be isolated from the mammal (e.g., from the blood) and further purified by well-known techniques, such as protein A chromatography to obtain the IgG fraction.

At an appropriate time after immunization, e.g., when the antibody titers are highest, antibody-producing cells can be obtained from the subject and used to prepare monoclonal antibodies by standard techniques, such as the hybridoma technique (see Kohler and Milstein, 1975, Nature 256:495-497; Brown et al., 1981, J. Immunol. 127:539-46; Brown et al., 1980, J. Biol. Chem. 255:4980-83; Yeh et al., 1976, PNAS 76:2927-31; and Yeh et al., 1982, Int J. Cancer 29:269-75), the human B cell hybridoma technique (Kozbor et al., 1983, Immunol. Today 4:72), the EBV-hybridoma technique (Cole et al., 1985, Monoclonal Antibodies and Cancer Therapy, Alan R. Liss, Inc., pp. 77-96) or trioma techniques.

The technology for producing hybridomas is well-known (see generally R. H. Kenneth, 1980, Monoclonal Antibodies: A New Dimension In Biological Analyses, Plenum Publishing Corp., New York, N.Y.; E. A. Lerner, 1981, Yale J. Biol. Med., 54:387-402; M. L. Gefter et al., 1977, Somatic Cell Genet. 3:231-36). In general, an immortal cell line (typically a myeloma) is fused to lymphocytes (typically splenocytes) from a mammal immunized with an ADAM or Interactor immunogen as described above, and the culture supernatants of the resulting hybridoma cells are screened to identify a hybridoma producing a monoclonal antibody that binds ADAM or Interactor polypeptides or peptides.

Any of the many well known protocols used for fusing lymphocytes and immortalized cell lines can be applied for the purpose of generating a monoclonal antibody to an ADAM or Interactor polypeptide (see, e.g., G. Galfre et al., 1977, Nature 266:55052; Gefter et al., 1977; Lerner, 1981; Kenneth, 1980). Moreover, the ordinarily skilled worker will appreciate that there are many variations of such methods. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin, and thymidine (HAT medium). Any of a number of myeloma cell lines can be used as a fusion partner according to standard techniques, e.g., the P3-NS1/1-Ag4-1, P3-x63-Ag8.653, or Sp2/O-Ag14 myeloma lines. These myeloma lines are available from ATCC (American Type Culture Collection, Manassas, Va.). Typically, HAT-sensitive mouse myeloma cells are fused to mouse splenocytes using polyethylene glycol (PEG). Hybridoma cells resulting from the fusion arc then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind ADAM or Interactor polypeptides or peptides, e.g., using a standard ELISA assay.

Alternative to preparing monoclonal antibody-secreting hybridomas, a monoclonal antibody can be identified and isolated by screening a recombinant combinatorial immunoglobulin library (e.g., an antibody phage display library) with the corresponding ADAM or Interactor polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612).

Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J 12:725-734; Hawkins et al., 1992, J. Mol. Biol. 226:889-896; Clarkson et al., 1991, Nature 352:624-628; Gram et al., 1992, PNAS 89:3576-3580; Garrad et al., 1991, Bio/Technology 9:1373-1377; Hoogenboom et al., 1991, Nuc. Acid Res. 19:4133-4137; Barbas et al., 1991, PNAS 88:7978-7982; and McCafferty et al., 1990, Nature 348:552-55.

Additionally, recombinant antibodies to an ADAM or Interactor polypeptide, such as chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al., 1988, Science 240:1041-1043; Liu et al., 1987, PNAS 84:3439-3443; Liu et al., 1987, J. Immunol. 139:3521-3526; Sun et al., 1987, PNAS 84:214-218; Nishimura et al., 1987, Canc. Res. 47:999-1005; Wood et al., 1985, Nature 314:446-449; and Shaw et al., 1988, J. Natl. Cancer Inst. 80:1553-1559; S. L. Morrison, 1985, Science 229:1202-1207; Oi et al., 1986, BioTechniques 4:214; Winter U.S. Pat. No. 5,225,539; Jones et al., 1986, Nature 321:552-525; Verhoeyan et al., 1988, Science 239:1534; and Bcidler et al., 1988, J. Immunol. 141:4053-4060.

An antibody against an ADAM or Interactor polypeptide (e.g., monoclonal antibody) can be used to isolate the corresponding polypeptide by standard techniques, such as affinity chromatography or immunoprecipitation. For example, antibodies can facilitate the purification of a natural ADAM or Interactor gene polypeptide from cells and of a recombinantly produced ADAM or Interactor polypeptide or peptide expressed in host cells. In addition, an antibody that binds to an ADAM or Interactor polypeptide can be used to detect the corresponding protein (e.g., in a cellular lysate or cell supernatant) in order to evaluate the abundance and pattern of expression of the protein. Such antibodies can also be used diagnostically to monitor ADAM or Interactor protein levels in tissue as part of a clinical testing procedure, e.g., to, for example, determine the efficacy of a given treatment regimen as described in detail herein. In addition, antibodies to an ADAM or Interactor polypeptide can be used as therapeutics for the treatment of diseases related to asthma, atopy, inflammatory bowel disease and obesity.

### LIGANDS

The ADAM or Interactor polypeptides, polynucleotides, variants, or fragments or portions thereof (e.g. Tables 2-5 and 7, SEQ ID NOs. 1-9, and FIGS. 1-12), can be used to screen for ligands (e.g., agonists, antagonists, or inhibitors) that modulate the levels or activity of the ADAM or Interactor polypeptide. In addition, these ADAM or Interactor molecules can be used to identify endogenous ligands that bind to ADAM or Interactor polypeptides or polynucleotides in the cell. In one aspect of the present invention, the full-length ADAM or Interactor polypeptide is used to identify ligands. Alternatively, variants or portions of an ADAM or Interactor polypeptide are used. Such portions may comprise, for example, one or more domains of the ADAM or Interactor polypeptide (e.g., intracellular, extracellular, SH3, fibronectin III repeat, cysteine-rich, and Ser/Thr-XXX-Val domains) disclosed herein. Of particular interest are screening assays that identify agents that have relatively low levels of toxicity in human cells. A wide variety of assays may be used for this purpose, including in vitro protein-protein binding assays, electrophoretic mobility shift assays, immunoassays, and the like.

Ligands that bind to the ADAM or Interactor polypeptides or polynucleotides of the invention are potentially useful in diagnostic applications and pharmaceutical compositions, as described in detail herein. Ligands may encompass numerous chemical classes, though typically they are organic molecules, e.g., small molecules. Preferably, small molecules have a molecular weight of less than 5000 daltons, more preferably, small molecules have a molecular weight of more than 50 and less than 2,500 daltons. Such molecules can comprise functional groups necessary for structural interaction with proteins, particularly hydrogen bonding, and typically include at least an amine, carbonyl, hydroxyl or carboxyl group, preferably at least two of the functional chemical groups. Useful molecules often comprise cyclical carbon or heterocyclic structures or aromatic or polyaromatic structures substituted with one or more of the above functional groups. Such molecules can also comprise biomolecules including peptides, saccharides, fatty acids, steroids, purines, pyrimidines, derivatives, structural analogs, or combinations thereof.

Ligands may include, for example, 1) peptides such as soluble peptides, including Ig-tailed fusion peptides and members of random peptide libraries (see, e.g., Lam et al., 1991, Nature 354:82-84; Houghten et al., 1991, Nature 354:84-86) and combinatorial chemistry-derived molecular libraries made of D- or L-configuration amino acids; 2) phosphopeptides (e.g., members of random and partially degenerate, directed phosphopeptide libraries, see, e.g., Songyang et al, 1993, Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules.

Test agents useful for identifying ADAM or Interactor ligands can be obtained from a wide variety of sources including libraries of synthetic or natural compounds. Synthetic compound libraries are commercially available from, for example, Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich Chemical Company, Inc. (Milwaukee, Wis.). Natural compound libraries comprising bacterial, fungal, plant or animal extracts are available from, for example, Pan Laboratories (Bothell, WA). In addition, numerous means are available for random and directed synthesis of a wide variety of organic compounds and biomolecules, including expression of randomized oligonucleotides.

Alternatively, libraries of natural compounds in the form of bacterial, fungal, plant and animal extracts can be readily produced. Methods for the synthesis of molecular libraries are readily available (see, e.g., DeWiff et al., 1993, Proc. Nat. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carell et al., 1994, Angew. Chem. Int. Ed. Engl. 33:2059; Carell et al., 1994, Angew. Chem. Int. Ed. Engl. 33:2061; and in Gallop et al., 1994, J. Med. Chem. 37:1233). In addition, natural or synthetic compound libraries and compounds can be readily modified through conventional chemical, physical and biochemical means (see, e.g., Blondelle et al., 1996, Trends in Biotech. 14:60), and may be used to produce combinatorial libraries. In another approach, previously identified pharmacological agents can be subjected to directed or random chemical modifications, such as acylation, alkylation, esterification, amidification, and the analogs can be screened for ADAM or Interactor gene-modulating activity.

Numerous methods for producing combinatorial libraries are known in the art, including those involving biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer, or small molecule libraries of compounds (K. S. Lam, 1997, Anticancer Drug Des. 12:145).

Non-limiting examples of small molecules, small molecule libraries, combinatorial libraries, and screening methods are described in B. Seligmann, 1995, “Synthesis, Screening, Identification of Positive Compounds and Optimization of Leads from Combinatorial Libraries: Validation of Success” p. 69-70. Symposium: Exploiting Molecular Diversity: Small Molecule Libraries for Drug Discovery, La Jolla, Calif., Jan. 23-25, 1995 (conference summary available from Wendy Warr & Associates, 6 Berwick Court, Cheshire, UK CW4 7HZ); E. Martin et al., 1995, J. Med. Chem. 38:1431-1436; E. Martin et al., 1995, “Measuring diversity: Experimental design of combinatorial libraries for drug discovery” Abstract, ACS Meeting, Anaheim, Calif., COMP 32; and E. Martin, 1995, “Measuring Chemical Diversity: Random Screening or Rationale Library Design” p. 27-30, Symposium: Exploiting Molecular Diversity: Small Molecule Libraries for Drug Discovery, La Jolla, Calif. Jan. 23-25, 1995 (conference summary available from Wendy Warr & Associates, 6 Berwick Court, Cheshire, UK CW4 7HZ).

Libraries may be screened in solution (e.g., Houghten, 1992, Biotechniques 13:412-421), or on beads (Lam, 1991, Nature 354:82-84), chips (Fodor, 1993, Nature 364:555-556), bacteria or spores (Ladner U.S. Pat. No. 5,223,409), plasmids (Cull et al., 1992, Proc. Natl. Acad. Sci. USA 89:1865-1869), or on phage (Scott and Smith, 1990, Science 249:386-390; Devlin, 1990, Science 249:404-406; Cwirla et al., 1990, Proc. Natl. Acad. Sci. USA 97:6378-6382; Felici, 1991, J. Mol. Biol. 222:301-310; Ladner, supra).

Where the screening assay is a binding assay, an ADAM or Interactor polypeptide, polynucleotide, analog, or fragment thereof, may be joined to a label, where the label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g., albumin, detergents, etc., that are used to facilitate optimal protein-protein binding or reduce non-specific or background interactions. Reagents that improve the efficiency of the assay, such as protease inhibitors, nuclease inhibitors, anti-microbial agents, etc., may be used. The components are added in any order that produces the requisite binding. Incubations are performed at any temperature that facilitates optimal activity, typically between 40 and 40° C. Incubation periods are selected for optimum activity, but may also be optimized to facilitate rapid high-throughput screening. Normally, between 0.1 and 1 hr will be sufficient. In general, a plurality of assay mixtures is run in parallel with different agent concentrations to obtain a differential response to these concentrations. Typically, one of these concentrations serves as a negative control, i.e., at zero concentration or below the level of detection.

To perform cell-free ligand screening assays, it may be desirable to immobilize either an ADAM or Interactor polypeptide, polynucleotide, or fragment to a surface to facilitate identification of ligands that bind to these molecules, as well as to accommodate automation of the assay. For example, a fusion protein comprising an ADAM or Interactor polypeptide and an affinity tag can be produced. In one embodiment, a glutathione-S-transferase/phosphodiesterase fusion protein comprising an ADAM or Interactor polypeptide is adsorbed onto glutathione sepharose beads (Sigma Chemical, St. Louis, Mo.) or glutathione-derivatized microtiter plates. Cell lysates (e.g., containing ³⁵S-labeled polypeptides) are added to the coated beads under conditions to allow complex formation (e.g., at physiological conditions for salt and pH). Following incubation, the coated beads are washed to remove any unbound polypeptides, and the amount of immobilized radiolabel is determined. Alternatively, the complex is dissociated and the radiolabel present in the supernatant is determined. In another approach, the beads are analyzed by SDS-PAGE to identify the bound polypeptides.

Ligand-binding assays can be used to identify agonist or antagonists that alter the function or levels of an ADAM or Interactor polypeptide. Such assays are designed to detect the interaction of test agents (e.g., small molecules) with ADAM or Interactor polypeptides, polynucleotides, analogs, or fragments or portions thereof. Interactions may be detected by direct measurement of binding. Alternatively, interactions may be detected by indirect indicators of binding, such as stabilization/destabilization of protein structure, or activation/inhibition of biological function. Non-limiting examples of useful ligand-binding assays are detailed below.

Ligands that bind to ADAM or Interactor polypeptides, polynucleotides, analogs, or fragments or portions thereof, can be identified using real-time Bimolecular Interaction Analysis (BIA; Sjolander et al., 1991, Anal. Chem. 63:2338-2345; Szabo et al., 1995, Curr. Opin. Struct. Biol. 5:699-705). BIA-based technology (e.g., BIAcore™; LKB Pharmacia, Sweden) allows study of biospecific interactions in real time, without labeling. In BIA, changes in the optical phenomenon surface plasmon resonance (SPR) is used determine real-time interactions of biological molecules.

Ligands can also be identified by scintillation proximity assays (SPA, described in U.S. Pat. No. 4,568,649). In a modification of this assay that is currently undergoing development, chaperoning are used to distinguish folded and unfolded proteins. A tagged protein is attached to SPA beads, and test agents are added. The bead is then subjected to mild denaturing conditions (such as, e.g., heat, exposure to SDS, etc.) and a purified labeled chaperonin is added. If a test agent binds to a target, the labeled chaperonin will not bind; conversely, if no test agent binds, the protein will undergo some degree of denaturation and the chaperonin will bind.

Ligands can also be identified using a binding assay based on mitochondrial targeting signals (Hurt et al., 1985, EMBO J. 4:2061-2068; Eilers and Schatz, 1986, Nature 322:228-231). In a mitochondrial import assay, expression vectors are constructed in which nucleic acids encoding particular target proteins are inserted downstream of sequences encoding mitochondrial import signals. The chimeric proteins are synthesized and tested for their ability to be imported into isolated mitochondria in the absence and presence of test compounds. A test compound that binds to the target protein should inhibit its uptake into isolated mitochondria in vitro.

The ligand-binding assay described in Fodor et al., 1991, Science 251:767-773, which involves testing the binding affinity of test compounds for a plurality of defined polymers synthesized on a solid substrate, can also be used.

Ligands that bind to ADAM or Interactor polypeptides or peptides can be identified using two-hybrid assays (see, e.g., U.S. Pat. No. 5,283,317; Zervos et al., 1993, Cell 72:223-232; Madura et al., 1993, J. Biol. Chem. 268:12046-12054; Bartel et al., 1993, Biotechniques 14:920-924; Iwabuchi et al., 1993, Oncogene 8:1693-1696; and Brent WO 94/10300). The two-hybrid system relies on the reconstitution of transcription activation activity by association of the DNA-binding and transcription activation domains of a transcriptional activator through protein-protein interaction. The yeast GAL4 transcriptional activator may be used in this way, although other transcription factors have been used and are well known in the art. To carryout the two-hybrid assay, the GAL4 DNA-binding domain, and the GAL4 transcription activation domain are expressed, separately, as fusions to potential interacting polypeptides.

In one embodiment, the “bait” protein comprises an ADAM or Interactor polypeptide fused to the GAL4 DNA-binding domain. The “fish” protein comprises, for example, a human cDNA library encoded polypeptide fused to the GAL4 transcription activation domain. If the two, coexpressed fusion proteins interact in the nucleus of a host cell, a reporter gene (e.g., LacZ) is activated to produce a detectable phenotype. The host cells that show two-hybrid interactions can be used to isolate the containing plasmids containing the cDNA library sequences. These plasmids can be analyzed to determine the nucleic acid sequence and predicted polypeptide sequence of the candidate ligand. Alternatively, methods such as the three-hybrid (Licitra et al., 1996, Proc. Nat. Acad. Sci. USA 93:12817-12821), and reverse two-hybrid (Vidal et al., 1996, Proc. Natl. Acad. Sci. USA 93:10315-10320) systems may be used. Commercially available two-hybrid systems such as the CLONTECH Matchmaker™ systems and protocols (CLONTECH Laboratories, Inc., Palo Alto, Calif.) may be also be used (see also, A. R. Mendelsohn et al., 1994, Curr. Op. Biotech. 5:482; E. M. Phizicky et al., 1995, Microbiological Rev. 59:94; M. Yang et al., 1995, Nucleic Acids Res. 23:1152; S. Fields et al., 1994, Trends Genet. 10:286; and U.S. Pat. No. 6,283,173 and 5,468,614).

Several methods of automated assays have been developed in recent years so as to permit screening of tens of thousands of test agents in a short period of time. High-throughput screening methods are particularly preferred for use with the present invention. The ligand-binding assays described herein can be adapted for high-throughput screens, or alternative screens may be employed. For example, continuous format high throughput screens (CF-HTS) using at least one porous matrix allows the researcher to test large numbers of test agents for a wide range of biological or biochemical activity (see U.S. Pat. No. 5,976,813 to Beutel et al.). Moreover, CF-HTS can be used to perform multi-step assays.

### DIAGNOSTICS

As discussed herein, ADAM or Interactor genes are associated with various diseases and disorders, including but not limited to, asthma, atopy, obesity, and inflammatory bowel disease. The present invention therefore provides nucleic acids and antibodies that can be useful in diagnosing individuals with disorders associated with aberrant ADAM or Interactor gene expression or mutated ADAM or Interactor genes. In particular, nucleic acids comprising ADAM or Interactor SNP alleles and haplotypes can be used to identify chromosomal abnormalities linked to these diseases. Additionally, antibodies directed against the amino acid variants encoded by the ADAM or Interactor SNPs can be used to identify disease-associated polypeptides. Examples 5 and 6 herein further illustrate the use of ADAM and Interactor genes for identifying polymorphisms.

Antibody-based diagnostic methods: In a further embodiment of the present invention, antibodies which specifically bind to an ADAM or Interactor polypeptide encoded by the nucleic acids shown in Tables 2-5 and 7, SEQ ID NOs. 1-9, and FIGS. 1-12, may be used for the diagnosis of conditions or diseases characterized by underexpression or overexpression of the ADAM or Interactor polynucleotide or polypeptide, or in assays to monitor patients being treated with an ADAM or Interactor polypeptide, polynucleotide, or antibody, or an ADAM or Interactor agonist, antagonist, or inhibitor.

The antibodies useful for diagnostic purposes may be prepared in the same manner as those for use in therapeutic methods, described herein. Antibodies may be raised to a full-length ADAM or Interactor polypeptide sequence. Alternatively, the antibodies may be raised to portions or variants of the ADAM or Interactor polypeptide. Such variants include polypeptides encoded by the disclosed ADAM or Interactor SNPs or alternate splice variants. In one aspect of the invention, antibodies are prepared to bind to an ADAM or Interactor polypeptide fragment comprising one or more domains of the ADAM or Interactor polypeptide (e.g., transmembrane, intracellular, extracellular, SH3, fibronectin III repeat, cysteine-rich, and Ser/Thr-XXX-Val domains), as described in detail herein.

Diagnostic assays for an ADAM or Interactor polypeptide include methods that utilize the antibody and a label to detect the protein in biological samples (e.g., human body fluids, cells, tissues, or extracts of cells or tissues). The antibodies may be used with or without modification, and may be labeled by joining them, either covalently or non-covalently, with a reporter molecule. A wide variety of reporter molecules that are known in the art may be used, several of which are described herein.

The invention provides methods for detecting disease-associated antigenic components in a biological sample, which methods comprise the steps of: 1) contacting a sample suspected to contain a disease-associated antigenic component with an antibody specific for an disease-associated antigen, extracellular or intracellular, under conditions in which an antigen-antibody complex can form between the antibody and disease-associated antigenic components in the sample; and 2) detecting any antigen-antibody complex formed in step (1) using any suitable means known in the art, wherein the detection of a complex indicates the presence of disease-associated antigenic components in the sample. It will be understood that assays that utilize antibodies directed against altered ADAM or Interactor amino acid sequences (i.e., epitopes encoded by SNPs, modifications, mutations, or variants) are within the scope of the invention.

Many immunoassay formats are known in the art, and the particular format used is determined by the desired application. An immunoassay can use, for example, a monoclonal antibody directed against a single disease-associated epitope, a combination of monoclonal antibodies directed against different epitopes of a single disease-associated antigenic component, monoclonal antibodies directed towards epitopes of different disease-associated antigens, polyclonal antibodies directed towards the same disease-associated antigen, or polyclonal antibodies directed towards different disease-associated antigens. Protocols can also, for example, use solid supports, or may involve immunoprecipitation.

In accordance with the present invention, “competitive” (U.S. Pat. Nos. 3,654,090 and 3,850,752), “sandwich” (U.S. Pat. No. 4,016,043), and “double antibody,” or “DASP” assays may be used. Several procedures for measuring the amount of an ADAM or Interactor polypeptide in a sample (e.g., ELISA, RIA, and FACS) are known in the art and provide a basis for diagnosing altered or abnormal levels of ADAM or Interactor polypeptide expression. Normal or standard values for an ADAM or Interactor polypeptide expression are established by incubating biological samples taken from normal subjects, preferably human, with antibody to an ADAM or Interactor polypeptide under conditions suitable for complex formation. The amount of standard complex formation may be quantified by various methods; photometric means are preferred. Levels of the ADAM or Interactor polypeptide expressed in the subject sample, negative control (normal) sample, and positive control (disease) sample are compared with the standard values. Deviation between standard and subject values establishes the parameters for diagnosing disease.

Typically, immunoassays use either a labeled antibody or a labeled antigenic component (i.e., to compete with the antigen in the sample for binding to the antibody). A number of fluorescent materials are known and can be utilized as labels for antibodies or polypeptides. These include, for example, Cy3, Cy5, GFP (e.g., EGFP, DsRed, dEFP, etc. (CLONTECH, Palo Alto, Calif.)), Alexa, BODIPY, fluorescein (e.g., FluorX, DTAF, and FITC), rhodamine (e.g., TRITC), auramine, Texas Red, AMCA blue, and Lucifer Yellow. Antibodies or polypeptides can also be labeled with a radioactive element or with an enzyme. Preferred isotopes include ³H, ¹⁴C, 32 P, ³⁵S, ³⁶Cl, ⁵¹Cr, ⁵⁷CO, ⁵⁸CO, ⁵⁹Fe, ⁹⁰Y, ¹²⁵I, ¹³¹I, and ¹⁸⁶Re.

Preferred enzymes include peroxidase, β-glucuronidase, β-D-glucosidase, β-D-galactosidase, urease, glucose oxidase plus peroxidase, and alkaline phosphatase (see, e.g., U.S. Pat. Nos. 3,654,090; 3,850,752 and 4,016,043). Enzymes can be conjugated by reaction with bridging molecules such as carbodiimides, diisocyanates, glutaraldehyde, and the like. Enzyme labels can be detected visually, or measured by calorimetric, spectrophotometric, fluorospectrophotometric, amperometric, or gasometric techniques. Other labeling systems, such as avidin/biotin, Tyramide Signal Amplification (TSA™), are known in the art, and are commercially available (see, e.g., ABC kit, Vector Laboratories, Inc., Burlingame, Calif.; NEN® Life Science Products, Inc., Boston, Mass.).

Kits suitable for antibody-based diagnostic applications typically include one or more of the following components:

(1) Antibodies: The antibodies may be pre-labeled; alternatively, the antibody may be unlabeled and the ingredients for labeling may be included in the kit in separate containers, or a secondary, labeled antibody is provided; and

(2) Reaction components: The kit may also contain other suitably packaged reagents and materials needed for the particular immunoassay protocol, including solid-phase matrices, if applicable, and standards.

The kits referred to above may include instructions for conducting the test. Furthermore, in preferred embodiments, the diagnostic kits are adaptable to high-throughput or automated operation.

Nucleic-acid-based diagnostic methods: The invention provides methods for detecting altered levels or sequences of ADAM or Interactor nucleic acids (e.g., such as the sequences shown in Tables 2-5 and 7, SEQ ID NOs. 1-9, and FIGS. 1-12) in a sample, such as in a biological sample, comprising the steps of: 1) contacting a sample suspected to contain a disease-associated nucleic acid with one or more disease-associated nucleic acid probes under conditions in which hybrids can form between any of the probes and disease-associated nucleic acid in the sample; and 2) detecting any hybrids formed in step (1) using any suitable means known in the art, wherein the detection of hybrids indicates the presence of the disease-associated nucleic acid in the sample. Exemplary methods are described in the Examples, herein below. To detect disease-associated nucleic acids present in low levels in biological samples, it may be necessary to amplify the disease-associated sequences or the hybridization signal as part of the diagnostic assay. Techniques for amplification are known to those of skill in the art.

The presence of an ADAM or Interactor polynucleotide sequences can be detected by DNA-DNA or DNA-RNA hybridization, or by amplification using probes or primers comprising at least a portion of an ADAM or Interactor polynucleotide, or a sequence complementary thereto. In particular, nucleic acid amplification-based assays can use ADAM or Interactor oligonucleotides or oligomers to detect transformants containing ADAM or Interactor DNA or RNA. Preferably, ADAM or Interactor nucleic acids useful as probes in diagnostic methods include oligonucleotides at least 15 contiguous nucleotides in length, more preferably at least 20 contiguous nucleotides in length, and most preferably at least 25-55 contiguous nucleotides in length, that hybridize specifically with ADAM or Interactor nucleic acids. As non-limiting examples, probes or primers useful for diagnostics may comprise any of the ADAM or Interactor DNA nucleotide sequences shown in Tables 3 and 4.

Several methods can be used to produce specific probes for ADAM or Interactor polynucleotides. For example, labeled probes can be produced by oligo-labeling, nick translation, end-labeling, or PCR amplification using a labeled nucleotide. Alternatively, ADAM or Interactor polynucleotide sequences, or any portions or fragments thereof, may be cloned into a vector for the production of an mRNA probe. Such vectors are known in the art, are commercially available, and may be used to synthesize RNA probes in vitro by addition of an appropriate RNA polymerase, such as T7, T3, or SP(6) end labeled nucleotides. These procedures may be conducted using a variety of commercially available kits (e.g., from Amersham-Pharmacia; Promega Corp.; and U.S. Biochemical Corp., Cleveland, Ohio). Suitable reporter molecules or labels which may be used include radionucleotides, enzymes, fluorescent, chemiluminescent, or chromogenic agents, as well as substrates, cofactors, inhibitors, magnetic particles, and the like.

A sample to be analyzed, such as, for example, a tissue sample (e.g., hair or buccal cavity) or body fluid sample (e.g., blood or saliva), may be contacted directly with the nucleic acid probes. Alternatively, the sample may be treated to extract the nucleic acids contained therein. It will be understood that the particular method used to extract DNA will depend on the nature of the biological sample. The resulting nucleic acid from the sample may be subjected to gel electrophoresis or other size separation techniques, or, the nucleic acid sample may be immobilized on an appropriate solid matrix without size separation.

Kits suitable for nucleic acid-based diagnostic applications typically include the following components:

(1)Probe DNA: The probe DNA may be prelabeled; alternatively, the probe DNA may be unlabeled and the ingredients for labeling may be included in the kit in separate containers; and

(2)Hybridization reagents: The kit may also contain other suitably packaged reagents and materials needed for the particular hybridization protocol, including solid-phase matrices, if applicable, and standards.

In cases where a disease condition is suspected to involve an alteration of an ADAM or Interactor nucleotide sequence, specific oligonucleotides may be constructed and used to assess the level of disease mRNA in cells affected or other tissue affected by the disease. For example, PCR can be used to test whether a person has a disease-related polymorphism (i.e., mutation). Specific methods of polymorphism identification are described herein, but are not intended to limit the present invention. The detection of polymorphisms in DNA sequences can be accomplished by a variety of methods including, but not limited to, RFLP detection based on allele-specific restriction-endonuclease cleavage (Kan and Dozy, 1978, Lancet ii:910-912), hybridization with allele-specific oligonucleotide probes (Wallace et al., 1978, Nucl Acids Res. 6:3543-3557), including immobilized oligonucleotides (Saiki et al., 1969, Proc. Natl. Acad. Sci. USA 86:6230-6234) or oligonucleotide arrays (Maskos and Southern, 1993, Nucl. Acids Res. 21:2269-2270), allele-specific PCR (Newton et al., 1989, Nucl. Acids Res. 17:2503-2516), mismatch-repair detection (MRD) (Faham and Cox, 1995, Genome Res. 5:474-482), binding of MutS protein (Wagner et al., 1995, Nucl. Acids Res. 23:3944-3948), denaturing-gradient gel electrophoresis (DGGE) (Fisher and Lerman et al., 1983, Proc. Natl. Acad. Sci. USA. 80:1579-1583), single-strand-conformation-polymorphism detection (Orita et al., 1983, Genomics 5:874-879), RNAase cleavage at mismatched base-pairs (Myers et al., 1985, Science 230:1242), chemical (Cotton et al., 1988, Proc. Natl. Acad. Sci. USA 8:4397-4401) or enzymatic (Youil et al., 1995, Proc. Natl. Acad. Sci. USA 92:87-91) cleavage of heteroduplex DNA, methods based on allele specific primer extension (Syvanen et al., 1990, Genomics 8:684-692), genetic bit analysis (GBA) Nikiforov et al., 1994, Nucl. Acids 22:4167-4175), the oligonucleotide-ligation assay (OLA) (Landegren et al., 1988, Science 241:1077), the allele-specific ligation chain reaction (LCR) (Barrany, 1991, Proc. Natl. Acad. Sci. USA 88:189-193), gap-LCR (Abravaya et al., 1995, Nucl. Acids Res. 23:675-682), radioactive or fluorescent DNA sequencing using standard procedures well known in the art, and peptide nucleic acid (PNA) assays (Orum et al., 1993, Nucl. Acids Res. 21:5332-5356).

For PCR analysis, ADAM or Interactor oligonucleotides may be chemically synthesized, generated enzymatically, or produced from a recombinant source. Oligomers will preferably comprise two nucleotide sequences, one with a sense orientation (5′→3′) and another with an antisense orientation (3′→5′), employed under optimized conditions for identification of a specific gene or condition. The same two oligomers, nested sets of oligomers, or even a degenerate pool of oligomers may be employed under less stringent conditions for detection and quantification of closely related DNA or RNA sequences.

In accordance with PCR analysis, two oligonucleotides are synthesized by standard methods or are obtained from a commercial supplier of custom-made oligonucleotides. The length and base composition are determined by standard criteria using the Oligo 4.0 primer Picking program (W. Rychlik, 1992; available from Molecular Biology Insights, Inc., Cascade, Colo.). One of the oligonucleotides is designed so that it will hybridize only to the disease gene DNA under the PCR conditions used. The other oligonucleotide is designed to hybridize a segment of genomic DNA such that amplification of DNA using these oligonucleotide primers produces a conveniently identified DNA fragment. Samples may be obtained from hair follicles, whole blood, or the buccal cavity. The DNA fragment generated by this procedure is sequenced by standard techniques.

In one particular aspect, ADAM or Interactor oligonucleotides can be used to perform Genetic Bit Analysis (GBA) of ADAM or Interactor genes in accordance with published methods (T. T. Nikiforov et al., 1994, Nucleic Acids Res. 22(20):4167-75; T. T. Nikiforov T T et al., 1994, PCR Methods Appl. 3(5):285-91). In PCR-based GBA, specific fragments of genomic DNA containing the polymorphic site(s) are first amplified by PCR using one unmodified and one phosphorothioate-modified primer. The double-stranded PCR product is rendered single-stranded and then hybridized to immobilized oligonucleotide primer in wells of a multi-well plate. The primer is designed to anneal immediately adjacent to the polymorphic site of interest. The 3′ end of the primer is extended using a mixture of individually labeled dideoxynucleoside triphosphates. The label on the extended base is then determined. Preferably, GBA is performed using semi-automated ELISA or biochip formats (see, e.g., S. R. Head et al., 1997, Nucleic Acids Res. 25(24):5065-71; T. T. Nikiforov et al., 1994, Nucleic Acids Res. 22(20):4167-75).

Other amplification techniques besides PCR may be used as alternatives, such as ligation-mediated PCR or techniques involving Q-beta replicase (Cahill et al., 1991, Clin. Chem., 37(9):1482-5). Products of amplification can be detected by agarose gel electrophoresis, quantitative hybridization, or equivalent techniques for nucleic acid detection known to one skilled in the art of molecular biology (Sambrook et al., 1989). Other alterations in the disease gene may be diagnosed by the same type of amplification-detection procedures, by using oligonucleotides designed to contain and specifically identify those alterations.

In accordance with the present invention, ADAM or Interactor polynucleotides may also be used to detect and quantify levels of ADAM or Interactor mRNA in biological samples in which altered expression of ADAM or Interactor polynucleotide may be correlated with disease. These diagnostic assays may be used to distinguish between the absence, presence, increase, and decrease of ADAM or Interactor mRNA levels, and to monitor regulation of ADAM or Interactor polynucleotide levels during therapeutic treatment or intervention. For example, ADAM or Interactor polynucleotide sequences, or fragments, or complementary sequences thereof, can be used in Southern or Northern analysis, dot blot, or other membrane-based technologies; in PCR technologies; or in dip stick, pin, ELISA or biochip assays utilizing fluids or tissues from patient biopsies to detect the status of, e.g., levels or overexpression of ADAM or Interactor genes, or to detect altered ADAM or Interactor gene expression. Such qualitative or quantitative methods are well known in the art (G. H. Keller and M. M. Manak, 1993, DNA Probes, 2^{nd} Ed, Macmillan Publishers Ltd., England; D. W. Dieffenbach and G. S. Dveksler, 1995, PCR Primer: A Laboratory Manual, Cold Spring Harbor Press, Plainview, N.Y.; B. D. Hames and S. J. Higgins, 1985, Gene Probes 1, 2, IRL Press at Oxford University Press, Oxford, England).

Methods suitable for quantifying the expression of ADAM or Interactor genes include radiolabeling or biotinylating nucleotides, co-amplification of a control nucleic acid, and standard curves onto which the experimental results are interpolated (P. C. Melby et al., 1993, J. Immunol. Methods 159:235-244; and C. Duplaa et al., 1993, Anal. Biochem. 212(1):229-36.). The speed of quantifying multiple samples may be accelerated by running the assay in an ELISA format where the oligomer of interest is presented in various dilutions and a spectrophotometric or colorimetric response gives rapid quantification.

In accordance with these methods, the specificity of the probe, i.e., whether it is made from a highly specific region (e.g., at least 8 to 10 or 12 or 15 contiguous nucleotides in the 5′ regulatory region), or a less specific region (e.g., especially in the 3′ coding region), and the stringency of the hybridization or amplification (e.g., high, moderate, or low) will determine whether the probe identifies naturally occurring sequences encoding the ADAM or Interactor polypeptide, or alleles, SNPs, SNP alleles and haplotypes, mutants, or related sequences.

In a particular aspect, an ADAM or Interactor nucleic acid sequence (e.g., such as shown in Tables 2-5 and 7, SEQ ID NOs. 1-9, and FIGS. 1-12), or a sequence complementary thereto, or fragment thereof, may be useful in assays that detect ADAM or Interactor-related diseases such as asthma. An ADAM or Interactor polynucleotide can be labeled by standard methods, and added to a biological sample from a subject under conditions suitable for the formation of hybridization complexes. After a suitable incubation period, the sample can be washed and the signal is quantified and compared with a standard value. If the amount of signal in the test sample is significantly altered from that of a comparable negative control (normal) sample, the altered levels of an ADAM or Interactor nucleotide sequence can be correlated with the presence of the associated disease. Such assays may also be used to evaluate the efficacy of a particular prophylactic or therapeutic regimen in animal studies, in clinical trials, or for an individual patient.

To provide a basis for the diagnosis of a disease associated with altered expression of a ADAM or Interactor gene, a normal or standard profile for expression is established. This may be accomplished by incubating biological samples taken from normal subjects, either animal or human, with a sequence complementary to the ADAM or Interactor polynucleotide, or a fragment thereof, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for the disease. Deviation between standard and subject (patient) values is used to establish the presence of the condition.

Once the disease is diagnosed and a treatment protocol is initiated, hybridization assays may be repeated on a regular basis to evaluate whether the level of expression in the patient begins to approximate that which is observed in a normal individual. The results obtained from successive assays may be used to show the efficacy of treatment over a period ranging from several days to months.

With respect to diseases such as asthma, the presence of an abnormal amount of an ADAM or Interactor transcript in a biological sample (e.g., body fluid, cells, tissues, or cell or tissue extracts) from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the disease.

Microarrays: In another embodiment of the present invention, oligonucleotides, or longer fragments derived from an ADAM or Interactor polynucleotide sequence described herein may be used as targets in a microarray (e.g., biochip) system. The microarray can be used to monitor the expression level of large numbers of genes simultaneously (to produce a transcript image), and to identify genetic variants, mutations, and polymorphisms. This information may be used to determine gene function, to understand the genetic basis of a disease, to diagnose disease, and to develop and monitor the activities of therapeutic or prophylactic agents. Preparation and use of microarrays have been described in WO 95/11995 to Chee et al.; D. J. Lockhart et al., 1996, Nature Biotechnology 14:1675-1680; M. Schena et al., 1996, Proc. Natl. Acad. Sci. USA 93:10614-10619; U.S. Pat. No. 6,015,702 to P. Lal et al; J. Worley et al., 2000, Microarray Biochip Technology, M. Schena, ed., Biotechniques Book, Natick, Mass., pp. 65-86; Y. H. Rogers et al., 1999, Anal. Biochem. 266(1):23-30; S.R. Head et al., 1999, Mol. Cell. Probes. 13(2):81-7; S. J. Watson et al., 2000, Biol. Psychiatry 48(12): 1147-56.

In one application of the present invention, microarrays containing arrays of ADAM or Interactor polynucleotide sequences can be used to measure the expression levels of ADAM or Interactor nucleic acids in an individual. In particular, to diagnose an individual with an ADAM or Interactor -related condition or disease, a sample from a human or animal (containing nucleic acids, e.g., mRNA) can be used as a probe on a biochip containing an array of ADAM or Interactor polynucleotides (e.g., DNA) in decreasing concentrations (e.g., 1 ng, 0.1 ng, 0.01 ng, etc.). The test sample can be compared to samples from diseased and normal samples. Biochips can also be used to identify ADAM or Interactor mutations or polymorphisms in a population, including but not limited to, deletions, insertions, and mismatches. For example, mutations can be identified by: 1) placing ADAM or Interactor polynucleotides of this invention onto a biochip; 2) taking a test sample (containing, e.g., mRNA) and adding the sample to the biochip; 3) determining if the test samples hybridize to the 12q23-qter polynucleotides attached to the chip under various hybridization conditions (see, e.g., V. R. Chechetkin et al., 2000, J. Biomol. Struct. Dyn. 18(1):83-101). Alternatively microarray sequencing can be performed (see, e.g., E. P. Diamandis, 2000, Clin. Chem. 46(10):1523-5).

Chromosome mapping: In another application of this invention, ADAM or Interactor nucleic acid sequences (e.g. such as those shown in Tables 2-5 and 7, SEQ ID NOs. 1-9, and FIGS. 1-12), or complementary sequences, or fragments thereof, can be used as probes to map genomic sequences. The sequences may be mapped to a particular chromosome, to a specific region of a chromosome, or to human artificial chromosome constructions (HACs), yeast artificial chromosomes (YACs), bacterial artificial chromosomes (BACs), bacterial PI constructions, or single chromosome cDNA libraries (see, e.g., C. M. Price, 1993, Blood Rev., 7:127-134; B. J. Trask, 1991, Trends Genet 7:149-154).

In another of its aspects, the invention relates to a diagnostic kit for detecting an ADAM or Interactor polynucleotide or polypeptide as it relates to a disease or susceptibility to a disease, particularly asthma. Also related is a diagnostic kit that can be used to detect or assess asthma conditions. Such kits comprise one or more of the following:

(a) an ADAM or Interactor polynucleotide, preferably the nucleotide sequence of any of the sequences shown in Tables 2-5 and 7, SEQ ID NOs. 1-9, and FIGS. 1-12, or a fragment thereof; or

(b) a nucleotide sequence complementary to that of (a); or

(c) an ADAM or Interactor polypeptide, preferably the polypeptide of any of the sequences shown in Tables 2-5 and 7, SEQ ID NOs. 1-9, and FIGS. 1-12, or a fragment thereof; or

(d) an antibody to an ADAM or Interactor polypeptide, preferably to the polypeptide of any one of the sequences shown in Tables 2-7, SEQ ID NOs: 1-9, and FIGS. 1-12, or an antibody bindable fragment thereof. It will be appreciated that in any such kits, (a), (b), (c), or (d) may comprise a substantial component and that instructions for use can be included. The kits may also contain peripheral reagents such as buffers, stabilizers, etc.

The present invention also includes a test kit for genetic screening that can be utilized to identify mutations in ADAM or Interactor genes. By identifying patients with mutated ADAM or Interactor DNA and comparing the mutation to a database that contains known mutations in ADAM or Interactor and a particular condition or disease, identification and confirmation of, a particular condition or disease can be made. Accordingly, such a kit would comprise a PCR-based test that would involve transcribing the patients mRNA with a specific primer, and amplifying the resulting cDNA using another set of primers. The amplified product would be detectable by gel electrophoresis and could be compared with known standards for ADAM or Interactor genes. Preferably, this kit would utilize a patient's blood, serum, or saliva sample, and the DNA would be extracted using standard techniques. Primers flanking a known mutation would then be used to amplify a fragment of an ADAM or Interactor gene. The amplified piece would then be sequenced to determine the presence of a mutation.

Genomic Screening: Polymorphic genetic markers linked to a ADAM or Interactor genes can be used to predict susceptibility to the diseases genetically linked to that chromosomal region. Similarly, the identification of polymorphic genetic markers within ADAM or Interactor genes will allow the identification of specific allelic variants that are in linkage disequilibrium with other genetic lesions that affect one of the disease states discussed herein including respiratory disorders, obesity, and inflammatory bowel disease. SSCP (see below) allows the identification of polymorphisms within the genomic and coding region of the disclosed genes.

The present invention provides sequences for primers that can be used identify exons that contain SNPs, as well as sequences for primers that can be used to identify the sequence changes of the SNPs. In particular, Tables 3 and 4 show polymorphic primers, probes, or genetic markers within the ADAM or Interactor genes, which can be used to identify specific allelic variants that are in linkage disequilibrium with other genetic lesions that affect one of the disease states discussed herein, including asthma, atopy, obesity, and inflammatory bowel disease. Such markers can be used in conjunction with SSCP to identify polymorphisms within the genomic and coding region of the disclosed gene. In particular, Table 7 describes the specific methods used to identify the SNPs described herein.

This information can be used to identify additional SNPs and SNP alleles and haplotypes in accordance with the methods disclosed herein. Suitable methods for genomic screening have also been described by, e.g., Sheffield et al., 1995, Genet. 4:1837-1844; LeBlanc-Straceski et al., 1994, Genomics 19:341-9; Chen et al., 1995, Genomics 25:1-8. In employing these methods, the disclosed reagents can be used to predict the risk for disease (e.g., respiratory disorders, obesity, and inflammatory bowel disease) in a population or individual.

### THERAPEUTICS

As discussed herein, ADAM or Interactor genes are associated with various diseases and disorders, including but not limited to, asthma, atopy, obesity, and inflammatory bowel disease (B. Wallaert et al., 1995, J. Exp. Med. 182:1897-1904). The present invention therefore provides compositions (e.g., pharmaceutical compositions) comprising ADAM and Interactor nucleic acids, polypeptides, antibodies, ligands, or variants, portions, or fragments thereof that can be useful in treating individuals with these disorders. Also provided are methods employing ADAM or Interactor nucleic acids, polypeptides, antibodies, ligands, or variants, portions, or fragments thereof to identify drug candidates that can be used to prevent, treat, or ameliorate such disorders.

Drug screening and design: The present invention provides methods of screening for drugs using an ADAM or Interactor polypeptide, or portion thereof, in competitive binding assays, according to methods well-known in the art. For example, competitive drug screening assays can be employed using neutralizing antibodies capable of specifically binding an ADAM or Interactor polypeptide compete with a test compound for binding to the ADAM or Interactor polypeptide or fragments thereof.

The present invention further provides methods of rational drug design employing an ADAM or Interactor polypeptide, antibody, or portion or functional equivalent thereof. The goal of rational drug design is to produce structural analogs of biologically active polypeptides of interest or of small molecules with which they interact (e.g., agonists, antagonists, or inhibitors). In turn, these analogs can be used to fashion drugs which are, for example, more active or stable forms of the polypeptide, or which, e.g., enhance or interfere with the function of the polypeptide in vivo (see, e.g., Hodgson, 1991, Bio/Technology, 9:19-21). An example of rational drug design is the development of HIV protease inhibitors (Erickson et al., 1990, Science, 249:527-533).

In one approach, one first determines the three-dimensional structure of a protein of interest or, for example, of an ADAM or Interactor polypeptide or ligand complex, by x-ray crystallography, computer modeling, or a combination thereof. Useful information regarding the structure of a polypeptide can also be gained by computer modeling based on the structure of homologous proteins. In addition, ADAM or Interactor polypeptides, or portions thereof, can be analyzed by an alanine scan (Wells, 1991, Methods in Enzymol., 202:390-411). In this technique, each amino acid residue in an ADAM or Interactor polypeptide is replaced by alanine, and its effect on the activity of the polypeptide is determined.

In another approach, an antibody specific to an ADAM or Interactor polypeptide can be isolated, selected by a functional assay, and then analyzed to solve its crystal structure. In principle, this approach can yield a pharmacore upon which subsequent drug design can be based. Alternatively, it is possible to bypass protein crystallography altogether by generating anti-idiotypic antibodies (anti-ids) to a functional, pharmacologically active antibody. As a mirror image of a mirror image, the binding site of the anti-ids is predicted to be an analog of the corresponding ADAM or Interactor polypeptide. The anti-id can then be used to identify and isolate peptides from banks of chemically or biologically produced banks of peptides. Selected peptides can subsequently be used as pharmacores.

Non-limiting examples of methods and computer tools for drug design are described in R. Cramer et al., 1974, J. Med. Chem. 17:533; H. Kubinyi (ed) 1993, 3D QSAR in Drug Design, Theory, Methods, and Applications, ESCOM, Leiden, Holland; P. Dean (ed) 1995, Molecular Similarity in Drug Design, K. Kim “Comparative molecular field analysis (ComFA)” p. 291-324, Chapman & Hill, London, UK; Y. et al., 1993, J. Comp.—Aid. Mol. Des. 7:83-102; G. Lauri and P. A. Bartlett, 1994, J. Comp.—Aid. Mol. Des. 8:51-66; P. J. Gane and P. M. Dean, 2000, Curr. Opin. Struct. Biol. 10(4):401-4; H. O. Kim and M. Kahn, 2000, Comb. Chem. High Throughput Screen. 3(3):167-83; G. K. Farber, 1999, Pharmacol Ther. 84(3):327-32; and H. van de Waterbeemd (ed) 1996, Structure-Property Correlations in Drug Research, Academic Press, San Diego, Calif.

In another aspect of the present invention, cells and animals that carry an ADAM or Interactor gene or an analog thereof can be used as model systems to study and test for substances that have potential as therapeutic agents. After a test agent is administered to animals or applied to the cells, the phenotype of the animals/cells can be determined.

In accordance with these methods, one may design drugs that result in, for example, altered ADAM or Interactor polypeptide activity or stability. Such drugs may act as inhibitors, agonists, or antagonists of an ADAM or Interactor polypeptide. By virtue of the availability of cloned ADAM or Interactor gene sequences, sufficient amounts of the ADAM or Interactor polypeptide may be produced to perform such analytical studies as x-ray crystallography. In addition, the knowledge of the ADAM or Interactor polypeptide sequence will guide those employing computer-modeling techniques in place of, or in addition to x-ray crystallography.

Pharmaceutical compositions: The present invention contemplates compositions comprising a ADAM or Interactor polynucleotides, polypeptide, antibody, ligand (e.g., agonist, antagonist, or inhibitor), or fragments, variants, or analogs thereof, and a physiologically acceptable carrier, excipient, or diluent as described in detail herein. The present invention further contemplates pharmaceutical compositions useful in practicing the therapeutic methods of this invention. Preferably, a pharmaceutical composition includes, in admixture, a pharmaceutically acceptable excipient (carrier) and one or more of an ADAM or Interactor polypeptide, polynucleotide, ligand, antibody, or fragment, portion, or variant thereof, as described herein, as an active ingredient. The preparation of pharmaceutical compositions that contain ADAM or Interactor molecules as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH-buffering agents, which enhance the effectiveness of the active ingredient.

An ADAM or Interactor polypeptide, polynucleotide, ligand, antibody, or fragment, portion, or variant thereof can be formulated into the pharmaceutical composition as neutralized physiologically acceptable salt forms. Suitable salts include the acid addition salts (i.e., formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The pharmaceutical compositions can be administered systemically by oral or parenteral routes. Non-limiting parenteral routes of administration include subcutaneous, intramuscular, intraperitoneal, intravenous, transdermal, inhalation, intranasal, intra-arterial, intrathecal, enteral, sublingual, or rectal. Intravenous administration, for example, can be performed by injection of a unit dose. The term “unit dose” when used in reference to a pharmaceutical composition of the present invention refers to physically discrete units suitable as unitary dosage for humans, each unit containing a predetermined quantity of active material calculated to produce the desired therapeutic effect in association with the required diluent; i.e., carrier, or vehicle.

In one particular embodiment of the present invention, the disclosed pharmaceutical compositions are administered via mucoactive aerosol therapy (see, e.g., M. Fuloria and B. K. Rubin, 2000, Respir. Care 45:868-873; I. Gonda, 2000, J. Pharm. Sci. 89:940-945; R. Dhand, 2000, Curr. Opin. Pulm. Med. 6(1):59-70; B. K. Rubin, 2000, Respir. Care 45(6):684-94; S. Suarez and A. J. Hickey, 2000, Respir. Care. 45(6):652-66).

Pharmaceutical compositions are administered in a manner compatible with the dosage formulation, and in a therapeutically effective amount. The quantity to be administered depends on the subject to be treated, capacity of the subject's immune system to utilize the active ingredient, and degree of modulation of ADAM or Interactor gene activity desired. Precise amounts of active ingredient required to be administered depend on the judgment of the practitioner and are specific for each individual. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusions sufficient to maintain concentrations of 10 nM to 10 μM in the blood are contemplated. An exemplary pharmaceutical formulation comprises: ADAM or Interactor antagonist or inhibitor (5.0 mg/ml); sodium bisulfite USP (3.2 mg/ml); disodium edetate USP (0.1 mg/ml); and water for injection q.s.a.d. (1.0 ml). As used herein, “pg” means picogram, “ng” means nanogram, “μg” means microgram, “mg” means milligram, “μl” means microliter, “ml” means milliliter, and “l” means L.

For further guidance in preparing pharmaceutical formulations, see, e.g., Gilman et al. (eds), 1990, Goodman and Gilman's: The Pharmacological Basis of Therapeutics, 8th ed., Pergamon Press; and Remington's Pharmaceutical Sciences, 17th ed., 1990, Mack Publishing Co., Easton, Pa.; Avis et al. (eds), 1993, Pharmaceutical Dosage Forms: Parenteral Medications, Dekker, New York; Lieberman et al. (eds), 1990, Pharmaceutical Dosage Forms: Disperse Systems, Dekker, New York.

In yet another aspect of this invention, antibodies that specifically react with an ADAM or Interactor polypeptide or peptides derived therefrom can be used as therapeutics. In particular, such antibodies can be used to block the activity of an ADAM or Interactor polypeptide. Antibodies or fragments thereof can be formulated as pharmaceutical compositions and administered to a subject. It is noted that antibody-based therapeutics produced from non-human sources can cause an undesired immune response in human subjects. To minimize this problem, chimeric antibody derivatives can be produced. Chimeric antibodies combine a non-human animal variable region with a human constant region. Chimeric antibodies can be constructed according to methods known in the art (see Morrison et al., 1985, Proc. Natl. Acad. Sci. USA 81:6851; Takeda et al., 1985, Nature 314:452; U.S. Pat. No. 4,816,567 of Cabilly et al.; U.S. Pat. No. 4,816,397 of Boss et al.; European Patent Publication EP 171496; EP 0173494; United Kingdom Patent GB 2177096B).

In addition, antibodies can be further “humanized” by any of the techniques known in the art, (e.g., Teng et al., 1983, Proc. Natl. Acad. Sci. USA 80:7308-7312; Kozbor et al., 1983, Immunology Today 4: 7279; Olsson et al., 1982, Meth. Enzymol. 92:3-16; International Patent Application WO92/06193; EP 0239400). Humanized antibodies can also be obtained from commercial sources (e.g., Scotgen Limited, Middlesex, England). Immunotherapy with a humanized antibody may result in increased long-term effectiveness for the treatment of chronic disease situations or situations requiring repeated antibody treatments.

### PHARMACOGENOMICS

Pharmacogenetics: The ADAM or Interactor polynucleotides and polypeptides (e.g., shown in Tables 2-5 and 7, SEQ ID NOs. 1-9, and FIGS. 1-12) of the invention are also useful in pharmacogenetic analysis (i.e., the study of the relationship between an individual's genotype and that individual's response to a therapeutic composition or drug). See, e.g., M. Eichelbaum, 1996, Clin. Exp. Pharmacol. Physiol. 23(10-11):983-985, and M. W. Linder, 1997, Clin. Chem. 43(2):254-266. The genotype of the individual can determine the way a therapeutic acts on the body or the way the body metabolizes the therapeutic. Further, the activity of drug metabolizing enzymes affects both the intensity and duration of therapeutic activity. Differences in the activity or metabolism of therapeutics can lead to severe toxicity or therapeutic failure. Accordingly, a physician or clinician may consider applying knowledge obtained in relevant pharmacogenetic studies in determining whether to administer an ADAM or Interactor polypeptide, polynucleotide, analog, antagonist, inhibitor, or modulator, as well as tailoring the dosage and therapeutic or prophylactic treatment regimen.

In general, two types of pharmacogenetic conditions can be differentiated. Genetic conditions can be due to a single factor that alters the way the drug act on the body (altered drug action), or a factor that alters the way the body metabolizes the drug (altered drug metabolism). These conditions can occur either as rare genetic defects or as naturally-occurring polymorphisms. For example, glucose-6-phosphate dehydrogenase deficiency (G6PD) is a common inherited enzymopathy which results in haemolysis after ingestion of oxidant drugs (anti-malarials, sulfonamides, analgesics, nitrofurans) and consumption of fava beans.

The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. The gene coding for CYP2D6 is highly polymorphic and several mutations have been identified in PM, which all lead to the absence of functional CYP2D6. Poor metabolizers quite frequently experience exaggerated drug response and side effects when they receive standard doses. If a metabolite is the active therapeutic moiety, PM show no therapeutic response. This has been demonstrated for the analgesic effect of codeine mediated by its CYP2D6-formed metabolite morphine. At the other extreme, ultra-rapid metabolizers fail to respond to standard doses. Recent studies have determined that ultra-rapid metabolism is attributable to CYP2D6 gene amplification.

By analogy, genetic polymorphism or mutation may lead to allelic variants of ADAM or Interactor genes in the population which have different levels of activity. The ADAM or Interactor polypeptides or polynucleotides thereby allow a clinician to ascertain a genetic predisposition that can affect treatment modality. In addition, genetic mutation or variants at other genes may potentiate or diminish the activity of ADAM or Interactor-targeted drugs. Thus, in an ADAM or Interactor gene-based treatment, a polymorphism or mutation may give rise to individuals that are more or less responsive to treatment. Accordingly, dosage would necessarily be modified to maximize the therapeutic effect within a given population containing the polymorphism. As an alternative to genotyping, specific polymorphic polypeptides or polynucleotides can be identified.

To identify genes that modify ADAM or Interactor-targeted drug response, several pharmacogenetic methods can be used. One pharmacogenomics approach, “genome-wide association”, relies primarily on a high-resolution map of the human genome. This high-resolution map shows previously identified gene-related markers (e.g., a “bi-allelic” gene marker map which consists of 60,000-100,000 polymorphic or variable sites on the human genome, each of which has two variants). A high-resolution genetic map can then be compared to a map of the genome of each of a statistically significant number of patients taking part in a Phase II/III drug trial to identify markers associated with a particular observed drug response or side effect. Alternatively, a high-resolution map can be generated from a combination of some ten million known single nucleotide polymorphisms (SNPs) in the human genome. Given a genetic map based on the occurrence of such SNPs, individuals can be grouped into genetic categories depending on a particular pattern of SNPs in their individual genome. In this way, treatment regimens can be tailored to groups of genetically similar individuals, taking into account traits that may be common among such genetically similar individuals (see, e.g., D. R. Pfost et al., 2000, Trends Biotechnol. 18(8):334-8).

As another example, the “candidate gene approach”, can be used. According to this method, if a gene that encodes a drug target is known, all common variants of that gene can be fairly easily identified in the population and it can be determined if having one version of the gene versus another is associated with a particular drug response.

As yet another example, a “gene expression profiling approach”, can be used. This method involves testing the gene expression of an animal treated with a drug (e.g., an ADAM or Interactor polypeptide, polynucleotide, analog, or modulator) to determine whether gene pathways related to toxicity have been turned on.

Information obtained from one of the approaches described herein can be used to establish a pharmacogenetic profile, which can be used to determine appropriate dosage and treatment regimens for prophylactic or therapeutic treatment an individual. A pharmacogenetic profile, when applied to dosing or drug selection, can be used to avoid adverse reactions or therapeutic failure and thus enhance therapeutic or prophylactic efficiency when treating a subject with an ADAM or Interactor polypeptide, polynucleotide, analog, antagonist, inhibitor, or modulator.

The ADAM or Interactor polypeptides or polynucleotides of the invention are also useful for monitoring therapeutic effects during clinical trials and other treatment. Thus, the therapeutic effectiveness of an agent that is designed to increase or decrease gene expression, polypeptide levels, or activity can be monitored over the course of treatment using the ADAM or Interactor compositions or modulators. For example, monitoring can be performed by: 1) obtaining a pre-administration sample from a subject prior to administration of the agent; 2) detecting the level of expression or activity of the protein in the pre-administration sample; 3) obtaining one or more post-administration samples from the subject; 4) detecting the level of expression or activity of the polypeptide in the post-administration samples; 5) comparing the level of expression or activity of the polypeptide in the pre-administration sample with the polypeptide in the post-administration sample or samples; and 6) increasing or decreasing the administration of the agent to the subject accordingly.

Gene Therapy: The ADAM or Interactor polynucleotides and polypeptides (e.g., shown in Tables 2-5 and 7, SEQ ID NOs. 1-9, and FIGS. 1-12) of the invention also find use as gene therapy reagents. In recent years, significant technological advances have been made in the area of gene therapy for both genetic and acquired diseases (Kay et al., 1997, Proc. Natl. Acad. Sci. USA, 94:12744-12746). Gene therapy can be defined as the transfer of DNA for therapeutic purposes. Improvement in gene transfer methods has allowed for development of gene therapy protocols for the treatment of diverse types of diseases. Gene therapy has also taken advantage of recent advances in the identification of new therapeutic genes, improvement in both viral and non-viral gene delivery systems, better understanding of gene regulation, and improvement in cell isolation and transplantation. Gene therapy would be carried out according to generally accepted methods as described by, for example, Friedman, 1991, Therapy for Genetic Diseases, Friedman, Ed., Oxford University Press, pages 105-121.

Vectors for introduction of genes both for recombination and for extrachromosomal maintenance are known in the art, and any suitable vector may be used. Methods for introducing DNA into cells such as electroporation, calcium phosphate co-precipitation, and viral transduction are known in the art, and the choice of method is within the competence of one skilled in the art (Robbins (ed), 1997, Gene Therapy Protocols, Human Press, NJ). Cells transformed with an ADAM or Interactor gene can be used as model systems to study asthma and other related disorders and to identify drug treatments for the treatment of such disorders.

Gene transfer systems known in the art may be useful in the practice of the gene therapy methods of the present invention. These include viral and non-viral transfer methods. A number of viruses have been used as gene transfer vectors, including polyoma, i.e., SV40 (Madzak et al., 1992, J. Gen. Virol., 73:1533-1536), adenovirus (Berkner, 1992, Curr. Top. Microbiol. Immunol., 158:39-6; Berkner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; Ohi et al., 1990, Gene, 89:279-282), herpes viruses including HSV and EBV (Margolskee, 1992, Curr. Top. Microbiol. Immunol., 158:67-90; Johnson et al., 1992, J. Virol., 66:2952-2965; Fink et al., 1992, Hum. Gene Ther., 3:11-19; Breakfield et al., 1987, Mol. Neurobiol., 1:337-371; Fresse et al., 1990, Biochem. Pharmacol., 40:2189-2199), and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Most human gene therapy protocols have been based on disabled murine retroviruses.

Non-viral gene transfer methods known in the art include chemical techniques such as calcium phosphate coprecipitation (Graham et al., 1973, Virology, 52:456-467; Pellicer et al., 1980, Science, 209:1414-1422), mechanical techniques, for example microinjection (Anderson et al., 1980, Proc. Natl. Acad. Sci. USA, 77:5399-5403; Gordon et al., 1980, Proc. Natl. Acad. Sci. USA, 77:7380-7384; Brinster et al., 1981, Cell, 27:223-231; Constantini et al., 1981, Nature, 294:92-94), membrane fusion-mediated transfer via liposomes (Felgner et al., 1987, Proc. Natl. Acad. Sci. USA, 84:7413-7417; Wang et al., 1989, Biochemistry, 28:9508-9514; Kaneda et al., 1989, J. Biol. Chem., 264:12126-12129; Stewart et al., 1992, Hum. Gene Ther., 3:267-275; Nabel et al., 1990, Science, 249:1285-1288; Lim et al., 1992, Circulation, 83:2007-2011), and direct DNA uptake and receptor-mediated DNA transfer (Wolff et al., 1990, Science, 247:1465-1468; Wu et al., 1991, BioTechniques, 11:474-485; Zenke et al., 1990, Proc. Natl. Acad. Sci. USA, 87:3655-3659; Wu et al., 1989, J. Biol. Chem., 264:16985-16987; Wolff et al., 1991, BioTechniques, 11:474-485; Wagner et al., 1991, Proc. Natl. Acad. Sci. USA, 88:4255-4259; Cotten et al., 1990, Proc. Natl. Acad. Sci. USA, 87:4033-4037; Curiel et al., 1991, Proc. Natl. Acad. Sci. USA, 88:8850-8854; Curiel et al., 1991, Hum. Gene Ther., 3:147-154).

In one approach, plasmid DNA is complexed with a polylysine-conjugated antibody specific to the adenovirus hexon protein, and the resulting complex is bound to an adenovirus vector. The trimolecular complex is then used to infect cells. The adenovirus vector permits efficient binding, internalization, and degradation of the endosome before the coupled DNA is damaged. In another approach, liposome/DNA is used to mediate direct in vivo gene transfer. While in standard liposome preparations the gene transfer process is non-specific, localized in vivo uptake and expression have been reported in tumor deposits, for example, following direct in situ administration (Nabel, 1992, Hum. Gene Ther., 3:399-410).

Suitable gene transfer vectors possess a promoter sequence, preferably a promoter that is cell-specific and placed upstream of the sequence to be expressed. The vectors may also contain, optionally, one or more expressible marker genes for expression as an indication of successful transfection and expression of the nucleic acid sequences contained in the vector. In addition, vectors can be optimized to minimize undesired immunogenicity and maximize long-term expression of the desired gene product(s) (see Nabe, 1999, Proc. Natl. Acad. Sci. USA 96:324-326). Moreover, vectors can be chosen based on cell-type that is targeted for treatment. Notably, gene transfer therapies have been initiated for the treatment of various pulmonary diseases (see, e.g., M. J. Welsh, 1999, J. Clin. Invest. 104(9):1165-6; D. L. Ennist, 1999, Trends Pharmacol. Sci. 20:260-266; S. M. Albelda et al., 2000, Ann. Intern. Med. 132:649-660; E. Alton and C. Kitson C., 2000, Expert Opin. Investig. Drugs. 9(7):1523-35).

Illustrative examples of vehicles or vector constructs for transfection or infection of the host cells include replication-defective viral vectors, DNA virus or RNA virus (retrovirus) vectors, such as adenovirus, herpes simplex virus and adeno-associated viral vectors. Adeno-associated virus vectors are single stranded and allow the efficient delivery of multiple copies of nucleic acid to the cell's nucleus. Preferred are adenovirus vectors. The vectors will normally be substantially free of any prokaryotic DNA and may comprise a number of different functional nucleic acid sequences. An example of such functional sequences may be a DNA region comprising transcriptional and translational initiation and termination regulatory sequences, including promoters (e.g., strong promoters, inducible promoters, and the like) and enhancers which are active in the host cells. Also included as part of the functional sequences is an open reading frame (polynucleotide sequence) encoding a protein of interest. Flanking sequences may also be included for site-directed integration. In some situations, the 5′-flanking sequence will allow homologous recombination, thus changing the nature of the transcriptional initiation region, so as to provide for inducible or non-inducible transcription to increase or decrease the level of transcription, as an example.

In general, the encoded and expressed ADAM or Interactor polypeptide may be intracellular, i.e., retained in the cytoplasm, nucleus, or in an organelle, or may be secreted by the cell. For secretion, the natural signal sequence present in an ADAM or Interactor polypeptide may be retained. When the polypeptide or peptide is a fragment of an ADAM or Interactor protein, a signal sequence may be provided so that, upon secretion and processing at the processing site, the desired protein will have the natural sequence. Specific examples of coding sequences of interest for use in accordance with the present invention include the ADAM or Interactor polypeptide-coding sequences disclosed herein.

As previously mentioned, a marker may be present for selection of cells containing the vector construct. The marker may be an inducible or non-inducible gene and will generally allow for positive selection under induction, or without induction, respectively. Examples of marker genes include neomycin, dihydrofolate reductase, glutamine synthetase, and the like. The vector employed will generally also include an origin of replication and other genes that are necessary for replication in the host cells, as routinely employed by those having skill in the art. As an example, the replication system comprising the origin of replication and any proteins associated with replication encoded by a particular virus may be included as part of the construct. The replication system must be selected so that the genes encoding products necessary for replication do not ultimately transform the cells. Such replication systems are represented by replication-defective adenovirus (see G. Acsadi et al., 1994, Hum. Mol. Genet. 3:579-584) and by Epstein-Barr virus. Examples of replication defective vectors, particularly, retroviral vectors that are replication defective, are BAG, (see Price et al., 1987, Proc. Natl. Acad. Sci. USA, 84:156; Sanes et al., 1986, EMBO J., 5:3133). It will be understood that the final gene construct may contain one or more genes of interest, for example, a gene encoding a bioactive metabolic molecule. In addition, cDNA, synthetically produced DNA or chromosomal DNA may be employed utilizing methods and protocols known and practiced by those having skill in the art.

According to one approach for gene therapy, a vector encoding an ADAM or Interactor polypeptide is directly injected into the recipient cells (in vivo gene therapy). Alternatively, cells from the intended recipients are explanted, genetically modified to encode an ADAM or Interactor polypeptide, and reimplanted into the donor (ex vivo gene therapy). An ex vivo approach provides the advantage of efficient viral gene transfer, which is superior to in vivo gene transfer approaches. In accordance with ex vivo gene therapy, the host cells are first transfected with engineered vectors containing at least one gene encoding an ADAM or Interactor polypeptide, suspended in a physiologically acceptable carrier or excipient such as saline or phosphate buffered saline, and the like, and then administered to the host. The desired gene product is expressed by the injected cells, which thus introduce the gene product into the host. The introduced gene products can thereby be utilized to treat or ameliorate a disorder (e.g., asthma, obesity, or inflammatory bowel disease) that is related to altered levels of the ADAM or Interactor polypeptide.

### ANIMAL MODELS

In accordance with the present invention, ADAM or Interactor polynucleotides (e.g., shown in Tables 2-5 and 7, SEQ ID NOs. 1-9, and FIGS. 1-12) can be used to generate genetically altered non-human animals or human cell lines. Any non-human animal can be used; however typical animals are rodents, such as mice, rats, or guinea pigs. Genetically engineered animals or cell lines can carry a gene that has been altered to contain deletions, substitutions, insertions, or modifications of the polynucleotide sequence (e.g., exon sequence). Such alterations may render the gene nonfunctional, (i.e., a null mutation) producing a “knockout” animal or cell line. In addition, genetically engineered animals can carry one or more exogenous or non-naturally occurring genes, i.e., “transgenes”, that are derived from different organisms (e.g., humans), or produced by synthetic or recombinant methods. Genetically altered animals or cell lines can be used to study ADAM or Interactor gene function, regulation, and treatments for ADAM or Interactor-related diseases. In particular, knockout animals and cell lines can be used to establish animal models and in vitro models for ADAM or Interactor-qter-related illnesses, respectively. In addition, transgenic animals expressing human ADAM or Interactor-qter can be used in drug discovery efforts.

A “transgenic animal” is any animal containing one or more cells bearing genetic information altered or received, directly or indirectly, by deliberate genetic manipulation at a subcellular level, such as by targeted recombination or microinjection or infection with recombinant virus. The term “transgenic animal” is not intended to encompass classical cross-breeding or in vitro fertilization, but rather is meant to encompass animals in which one or more cells are altered by, or receive, a recombinant DNA molecule. This recombinant DNA molecule may be specifically targeted to a defined genetic locus, may be randomly integrated within a chromosome, or it may be extrachromosomally replicating DNA.

Transgenic animals can be selected after treatment of germline cells or zygotes. For example, expression of an exogenous ADAM or Interactor gene or a variant can be achieved by operably linking the gene to a promoter and optionally an enhancer, and then microinjecting the construct into a zygote (see, e.g., Hogan et al., Manipulating the Mouse Embryo, A Laboratory Manual, Cold Spring Harbor Laboratory, Cold Spring Harbor, N.Y.). Such treatments include insertion of the exogenous gene and disrupted homologous genes. Alternatively, the gene(s) of the animals may be disrupted by insertion or deletion mutation of other genetic alterations using conventional techniques (see, e.g., Capecchi, 1989, Science, 244:1288; Valancuis et al., 1991, Mol. Cell Biol., 11:1402; Hasty et al., 1991, Nature, 350:243; Shinkai et al., 1992, Cell, 68:855; Mombaerts et al., 1992, Cell, 68:869; Philpott et al., 1992, Science, 256:1448; Snouwaert et al., 1992, Science, 257:1083; Donehower et al., 1992, Nature, 356:215).

In one aspect of the invention, ADAM or Interactor gene knockout mice can be produced in accordance with well-known methods (see, e.g., M. R. Capecchi, 1989, Science, 244:1288-1292; P. Li et al., 1995, Cell 80:401-411; L. A. Galli-Taliadoros et al., 1995, J. Immunol. Methods 181(1):1-15; C. H. Westphal et al., 1997, Curr. Biol. 7(7):530-3; S. S. Cheah et al., 2000, Methods Mol. Biol. 136:455-63). The disclosed murine ADAM or Interactor genomic clone can be used to prepare an ADAM or Interactor targeting construct that can disrupt ADAM or Interactor in the mouse by homologous recombination at the ADAM or Interactor chromosomal locus. The targeting construct can comprise a disrupted or deleted ADAM or Interactor gene sequence that inserts in place of the functioning portion of the native mouse gene. For example, the construct can contain an insertion in the ADAM or Interactor protein-coding region.

Preferably, the targeting construct contains markers for both positive and negative selection. The positive selection marker allows the selective elimination of cells that lack the marker, while the negative selection marker allows the elimination of cells that carry the marker. In particular, the positive selectable marker can be an antibiotic resistance gene, such as the neomycin resistance gene, which can be placed within the coding sequence of an ADAM or Interactor gene to render it non-functional, while at the same time rendering the construct selectable. The herpes simplex virus thymidine kinase (HSV tk) gene is an example of a negative selectable marker that can be used as a second marker to eliminate cells that carry it. Cells with the HSV tk gene are selectively killed in the presence of gangcyclovir. As an example, a positive selection marker can be positioned on a targeting construct within the region of the construct that integrates at the locus of the ADAM or Interactor gene. The negative selection marker can be positioned on the targeting construct outside the region that integrates at the locus of the ADAM or Interactor gene. Thus, if the entire construct is present in the cell, both positive and negative selection markers will be present. If the construct has integrated into the genome, the positive selection marker will be present, but the negative selection marker will be lost.

The targeting construct can be employed, for example, in embryonal stem cell (ES). ES cells may be obtained from pre-implantation embryos cultured in vitro (M. J. Evans et al., 1981, Nature 292:154-156; M. O. Bradley et al., 1984, Nature 309:255-258; Gossler et al., 1986, Proc. Natl. Acad. Sci. USA 83:9065-9069; Robertson et al., 1986, Nature 322:445-448; S. A. Wood et al., 1993, Proc. Natl. Acad. Sci. USA 90:4582-4584). Targeting constructs can be efficiently introduced into the ES cells by standard techniques such as DNA transfection or by retrovirus-mediated transduction. Following this, the transformed ES cells can be combined with blastocysts from a non-human animal. The introduced ES cells colonize the embryo and contribute to the germ line of the resulting chimeric animal (R. Jaenisch, 1988, Science 240:1468-1474). The use of gene-targeted ES cells in the generation of gene-targeted transgenic mice has been previously described (Thomas et al., 1987, Cell 51:503-512) and is reviewed elsewhere (Frohman et al., 1989, Cell 56:145-147; Capecchi, 1989, Trends in Genet. 5:70-76; Baribault et al., 1989, Mol. Biol. Med. 6:481-492; Wagner, 1990, EMBO J. 9:3025-3032; Bradley et al., 1992, Bio/Technology 10: 534-539).

Several methods can be used to select homologously recombined murine ES cells. One method employs PCR to screen pools of transformant cells for homologous insertion, followed by screening individual clones (Kim et al., 1988, Nucleic Acids Res. 16:8887-8903; Kim et al., 1991, Gene 103:227-233). Another method employs a marker gene is constructed which will only be active if homologous insertion occurs, allowing these recombinants to be selected directly (Sedivy et al., 1989, Proc. Natl. Acad. Sci. USA 86:227-231). For example, the positive-negative selection (PNS) method can be used as described above (see, e.g., Mansour et al., 1988, Nature 336:348-352; Capecchi, 1989, Science 244:1288-1292; Capecchi, 1989, Trends in Genet. 5:70-76). In particular, the PNS method is useful for targeting genes that are expressed at low levels.

The absence of functional ADAM or Interactor genes in the knockout mice can be confirmed, for example, by RNA analysis, protein expression analysis, and functional studies. For RNA analysis, RNA samples are prepared from different organs of the knockout mice and the ADAM or Interactor transcript is detected in Northern blots using oligonucleotide probes specific for the transcript. For protein expression detection, antibodies that are specific for the ADAM or Interactor polypeptide are used, for example, in flow cytometric analysis, immunohistochemical staining, and activity assays. Alternatively, functional assays are performed using preparations of different cell types collected from the knockout mice.

Several approaches can be used to produce transgenic mice. In one approach, a targeting vector is integrated into ES cell by homologous recombination, an intrachromosomal recombination event is used to eliminate the selectable markers, and only the transgene is left behind (A. L. Joyner et al., 1989, Nature 338(6211):153-6; P. Hasty et al., 1991, Nature 350(6315):243-6; V. Valancius and O. Smithies, 1991, Mol. Cell Biol. 11(3):1402-8; S. Fiering et al., 1993, Proc. Natl. Acad. Sci. USA 90(18):8469-73). In an alternative approach, two or more strains are created; one strain contains the gene knocked-out by homologous recombination, while one or more strains contain transgenes. The knockout strain is crossed with the transgenic strain to produce new line of animals in which the original wild-type allele has been replaced (although not at the same site) with a transgene. Notably, knockout and transgenic animals can be produced by commercial facilities (e.g., The Lerner Research Institute, Cleveland, OH; B&K Universal, Inc., Fremont, Calif.; DNX Transgenic Sciences, Cranbury, N.J.; Incyte Genomics, Inc., St. Louis, Mo.).

Transgenic animals (e.g., mice) containing a nucleic acid molecule which encodes a human ADAM or Interactor polypeptide, may be used as in vivo models to study the overexpression of an ADAM or Interactor gene. Such animals can also be used in drug evaluation and discovery efforts to find compounds effective to inhibit or modulate the activity of a 12q23-qter gene, such as for example compounds for treating respiratory disorders, diseases, or conditions. One having ordinary skill in the art can use standard techniques to produce transgenic animals which produce a human ADAM or Interactor polypeptide, and use the animals in drug evaluation and discovery projects (see, e.g., U.S. Pat. No. 4,873,191 to Wagner; U.S. Pat. No. 4,736,866 to Leder).

In another embodiment of the present invention, the transgenic animal can comprise a recombinant expression vector in which the nucleotide sequence that encodes a human ADAM or Interactor polypeptide is operably linked to a tissue specific promoter whereby the coding sequence is only expressed in that specific tissue. For example, the tissue specific promoter can be a mammary cell specific promoter and the recombinant protein so expressed is recovered from the animal's milk.

In yet another embodiment of the present invention, an ADAM or Interactor gene “knockout” can be produced by administering to the animal antibodies (e.g., neutralizing antibodies) that specifically recognize an endogenous ADAM or Interactor polypeptides. The antibodies can act to disrupt function of the endogenous ADAM or Interactor polypeptide, and thereby produce a null phenotype. In one specific example, an orthologous mouse ADAM or Interactor polypeptide or peptide can be used to generate antibodies. These antibodies can be given to a mouse to knockout the function of the mouse ADAM or Interactor ortholog.

In another embodiment of the present invention, non-mammalian organisms may be used to study ADAM or Interactor genes and ADAM or Interactor-related diseases. In particular, model organisms such as C. elegans, D. melanogaster, and S. cerevisiae may be used. Orthologs of ADAM or Interactor genes can be identified in these model organisms, and mutated or deleted to produce strains deficient for ADAM or Interactor genes. Human ADAM or Interactor genes can then be tested for the ability to “complement” the deficient strains. Such strains can also be used for drug screening. The ADAM or Interactor orthologs can be used to facilitate the understanding of the biological function of the human ADAM or Interactor genes, and assist in the identification of binding factors (e.g., agonists, antagonists, and inhibitors).

### EXAMPLES

The examples as set forth herein are meant to exemplify the various aspects of the present invention and are not intended to limit the invention in any way.

### Example 1

Family Collection

Asthma is a complex disorder that is influenced by a variety of factors, including both genetic and environmental effects. Complex disorders are typically caused by multiple interacting genes, some contributing to disease development and some conferring a protective effect. The success of linkage analyses in identifying chromosomes with significant LOD scores is achieved in part as a result of an experimental design tailored to the detection of susceptibility genes in complex diseases, even in the presence of epistasis and genetic heterogeneity. Also important are rigorous efforts in ascertaining asthmatic families that meet strict guidelines, and collecting accurate clinical information.

Given the complex nature of the asthma phenotype, non-parametric affected sib pair analyses were used to analyze the genetic data. This approach does not require parameter specifications such as mode of inheritance, disease allele frequency, penetrance of the disorder, or phenocopy rates. Instead, it determines whether the inheritance pattern of a chromosomal region is consistent with random segregation. If it is not, affected siblings inherit identical copies of alleles more often than expected by chance. Because no models for inheritance are assumed, allele-sharing methods tend to be more robust than parametric methods when analyzing complex disorders. They do, however, require larger sample sizes to reach statistically significant results.

At the outset of the program, the goal was to collect 400 affected sib-pair families for the linkage analyses. Based on a genome scan with markers spaced ˜10 cM apart, this number of families was predicted to provide >95% power to detect an asthma susceptibility gene that caused an increased risk to first-degree relatives of 3-fold or greater. The assumed relative risk of 3-fold was consistent with epidemiological studies in the literature that suggest an increased risk ranging from 3- to 7-fold. The relative risk was based on gender, different classifications of the asthma phenotype (i.e., bronchial hyper-responsiveness versus physician's diagnosis) and, in the case of offspring, whether one or both parents were asthmatic.

The family collection efforts exceeded the initial goal of 400, and resulted in a total of 444 affected sibling pair (ASP) families, with 342 families from the UK and 102 families from the US. The ASP families in the US collection were Caucasian with a minimum of two affected siblings that were identified through both private practice and community physicians as well as through advertising. A total of 102 families were collected in Kansas, Nebraska, and Southern California. In the UK collection, Caucasian families with a minimum of two affected siblings were identified through physicians' registers in a region surrounding Southampton and including the Isle of Wight. In both the US and UK collections, additional affected and unaffected sibs were collected whenever possible.

An additional 63 families from the United Kingdom were utilized from an earlier collection effort with different ascertainment criteria. These families were recruited either: 1) without reference to asthma and atopy; or 2) by having at least one family member or at least two family members affected with asthma. The randomly ascertained samples were identified from general practitioner registers in the Southampton area. For families with affected members, the probands were recruited from hospital based clinics in Southampton. Seven pedigrees extended beyond a single nuclear family. The phenotypic and genotypic data information for 17 markers for 21 of these 63 families was obtained from the website http://cedar.genetics.soton.ac.uk/pub/PROGRAMS/BETA/data/bet12.ped.

Families were included in the study if they met all of the following criteria: 1) the biological mother and biological father were Caucasian and agreed to participate in the study; 2) at least two biological siblings were alive, each with a current physician diagnosis of asthma, and were 5 to 21 years of age; and 3) the two siblings were currently taking asthma medications on a regular basis. This included regular, intermittent use of inhaled or oral bronchodilators and regular use of cromolyn, theophylline, or steroids.

Families were excluded from the study if they met any one of the following criteria: 1) both parents were affected (i.e., with a current diagnosis of asthma, having asthma symptoms, or on asthma medications at the time of the study); 2) any of the siblings to be included in the study was less than 5 years of age; 3) any asthmatic family member to be included in the study was taking beta-blockers at the time of the study, 4) any family member to be included in the study had congenital or acquired pulmonary disease at birth (e.g., cystic fibrosis), a history of serious cardiac disease (myocardial infarction), or any history of serious pulmonary disease (e.g., emphysema); or 5) any family member to be included in the study was pregnant.

An extensive clinical instrument was designed and data from all participating family members were collected. The case report form (CRF) included questions on demographics, medical history including medications, a health survey on the incidence and frequency of asthma, wheeze, eczema, hay fever, nasal problems, smoking, and questions on home environment. Data from a video questionnaire designed to show various examples of wheeze and asthmatic attacks were also included in the CRF. Clinical data, including skin prick tests to 8 common allergens, total and specific IgE levels, and bronchial hyper-responsiveness following a methacholine challenge, were also collected from all participating family members. All data were entered into a SAS dataset by IMTCI, a CRO; either by double data entry or scanning followed by on-screen visual validation. An extensive automated review of the data was performed on a routine basis and a full audit at the conclusion of the data entry was completed to verify the accuracy of the dataset.

### Example 2

Genome Scan

In order to identify chromosomal regions linked to asthma, the inheritance pattern of alleles from genetic markers spanning the genome was assessed on the collected family resources. As described above, combining these results with the segregation of the asthma phenotype in these families allows the identification of genetic markers that are tightly linked to asthma. In turn, this provides an indication of the location of genes predisposing affected individuals to asthma. The genotyping strategy was twofold: 1) to conduct a genome wide scan using markers spaced at approximately 10 cM intervals; and 2) to target ten chromosomal regions for high density genetic mapping. The initial candidate regions for high-density mapping were chosen based on suggestions of linkage to these regions by other investigators.

Genotypes of PCR amplified simple sequence microsatellite genetic linkage markers were determined using ABI model 377 Automated Sequencers (PE Applied Biosystems). Microsatellite markers were obtained from Research Genetics Inc. (Huntsville, Ala.) in the fluorescent dye-conjugated form (see Dubovsky et al., 1995, Hum. Mol. Genet. 4(3):449-452). The markers comprised a variation of a human linkage mapping panel as released from the Cooperative Human Linkage Center (CHLC), also known as the Weber lab screening set version 8. The variation of the Weber 8 screening set consisted of 529 markers with an average spacing of 6.9 cM (autosomes only) and 7.0 cM (all chromosomes). Eighty-nine percent of the markers consisted of either tri- or tetra-nucleotide microsatellites. There were no gaps present in chromosomal coverage greater than 17.5 cM.

Study subject genomic DNA (5 μl; 4.5 ng/μl) was amplified in a 10 μl PCR reaction using AmpliTaq Gold DNA polymerase (0.225 U); 1× PCR buffer (80 mM (NH₄)₂SO₄; 30 mM Tris-HCl (pH 8.8); 0.5% Tween-20); 200 μM each dATP, dCTP, dGTP and dTTP; 1.5-3.5 μM MgCl₂; and 250 μM forward and reverse PCR primers. PCR reactions were set up in 192 well plates (Costar) using a Tecan Genesis 150 robotic workstation equipped with a refrigerated deck. PCR reactions were overlaid with 20 μl mineral oil, and thermocycled on an MJ Research Tetrad DNA Engine equipped with four 192 well heads using the following conditions: 92° C. for 3 min; 6 cycles of 92° C. for 30 sec, 56° C. for 1 min, 72° C. for 45 sec; followed by 20 cycles of 92° C. for 30 sec, 55° C. for 1 min, 72° C. for 45 sec; and a 6 min incubation at 72° C.

PCR products of 8-12 microsatellite markers were subsequently pooled into two 96-well microtitre plates (2.0 μl PCR product from TET and FAM labeled markers, 3.0 μl HEX labeled markers) using a Tecan Genesis 200 robotic workstation and brought to a final volume of 25 μl with H₂O. Following this, 1.9 μl of pooled PCR product was transferred to a loading plate and combined with 3.0 μl loading buffer (2.5 μl formamide/blue dextran (9.0 mg/ml), 0.5 μl GS-500 TAMRA labeled size standard, ABI). Samples were denatured in the loading plate for 4 min at 95° C., placed on ice for 2 min, and electrophoresed on a 5% denaturing polyacrylamide gel (FMC on the ABI 377XL). Samples (0.8 μl) were loaded onto the gel using an 8 channel Hamilton Syringe pipettor.

Each gel consisted of 62 study subjects and 2 control subjects (CEPH parents ID #1331-01 and 1331-02, Coriell Cell Repository, Camden, N.J.). Genotyping gels were scored in duplicate by investigators blind to patient identity and affection status using GENOTYPER analysis software V 1.1.12 (ABI; PE Applied Biosystems). Nuclear families were loaded onto the gel with the parents flanking the siblings to facilitate error detection. The final tables obtained from the GENOTYPER output for each gel analysed were imported into a SYBASE Database.

Allele calling (binning) was performed using the SYBASE version of the ABAS software (Ghosh et al., 1997, Genome Research 7:165-178). Offsize bins were checked manually and incorrect calls were corrected or blanked. The binned alleles were then imported into the program MENDEL (Lange et al., 1988, Genetic Epidemiology, 5:471) for inheritance checking using the USERM13 subroutine (Boehnke et al., 1991, Am. J. Hum. Genet. 48:22-25). Non-inheritance was investigated by examining the genotyping traces and, once all discrepancies were resolved, the subroutine USERM13 was used to estimate allele frequencies.

### Example 3

Linkage Analysis

Chromosomal regions harboring asthma susceptibility genes were identified by linkage analysis of genotyping data and three separate phenotypes, asthma, bronchial hyper-responsiveness, and atopic status.

1. Asthma Phenotype: For the initial linkage analysis, the phenotype and asthma affection status were defined by a patient who answered the following questions in the affirmative: i) Have you ever had asthma? ii) Do you have a current physician's diagnosis of asthma? and iii) Are you currently taking asthma medications? Medications included inhaled or oral bronchodilators, cromolyn, theophylline, or steroids. Multipoint linkage analyses of allele sharing in affected individuals were performed using the MAPMAKER/SIBS analysis program (L. Kruglyak and E. S. Lander, 1995, Am. J. Hum. Genet. 57:439-454).

2. Phenotypic Subgroups: Nuclear families were ascertained by the presence of at least two affected siblings with a current physician's diagnosis of asthma, as well as the use of asthma medication. In the initial analysis (see above), the evidence was examined for linkage based on that dichotomous phenotype (asthma—yes/no). To further characterize the linkage signals, additional quantitative traits were measured in the clinical protocol. Since quantitative trait loci (QTL) analysis tools with correction for ascertainment were not available, the following approach was taken to refine the linkage and association analyses:

i. Phenotypic subgroups that could be indicative of an underlying genotypic heterogeneity were identified. Asthma subgroups were defined according to 1) bronchial hyper-responsiveness (BHR) to methacholine challenge; or 2) atopic status using quantitative measures like total serum IgE and specific IgE to common allergens.

ii. Non-parametric linkage analyses were performed on subgroups to test for the presence of a more homogeneous sub-sample. If genetic heterogeneity was present in the sample, the amount of allele sharing among phenotypically similar siblings was expected to increase in the appropriate subgroup in comparison to the full sample. A narrower region of significant increased allele sharing was also expected to result unless the overall LOD score decreased as a consequence of having a smaller sample size and of using an approximate partitioning of the data.

3. Results for BHR and IqE: PC₂₀, the concentration of methacholine resulting in a 20% drop in FEV₁ (forced expiratory volume), was polychotomized into four groups and analyses were performed on the subsets of asthmatic children with borderline to severe BHR (PC₂₀≦16 mg/ml) or PC₂₀(16). Total IgE was dichotomized using an age specific cutoff for elevated levels (one standard deviation above the mean: 52 kU/L for age 5-9; 63 kU/L for age 10-14; 75 kU/L for age 15-18; and 81 kU/L for adults). Similarly, a dichotomous variable was created using specific IgE to common allergens. An individual was assigned a high specific IgE value if his/her level was positive (grass or tree) or elevated (>0.35 KU/L for cat, dog, mite A, mite B, alternaria, or ragweed) for at least one such measure.

Based on the identification of an ADAM33 (Gene 216) located within chromosome 20 as described in U.S. patent application Ser. No. 09/834,597 and PCT/US01/12245 other family members, substrates and interactors were investigated as additional candidate genes (“disorder associated genes”). A pattern of evidence by linkage analysis pointed to the existence of several asthma susceptibility loci within the chromosomal regions identified in Table 1. This was supported by the initial analysis of the asthma (yes/no) phenotype with further localization by analyses of BHR, total IgE, and specific IgE in asthmatic individuals. Table 1 describes multipoint analysis results across the four phenotypes described above. The first column contains the gene name and the second column contains the chromosome number. The location of the gene is denoted in column three in centemorgans. The corresponding phenotypes and respective LOD scores are contained within the fourth, fifth, sixth and seventh columns. The results thus indicate that the genes located within these regions having a significant LOD score are involved in asthma and related diseases thereof.

TABLE 1

| | | | | Asthma & | Asthma & | Asthma & |
|---|---|---|---|---|---|---|
| | Chr. | Loc. (cM) | Asthma | BHR | Total IgE | Specific IgE |
| ADAMI9 | 5 | 159.8 | 1.11 | 1.33 | 0.95 | 1.67 |
| NRG2 | 5 | 142.9 | 0.78 | 1.49 | 0.70 | 0.69 |
| NRG1 | 8 | 61.6 | 1.04 | 1.09 | 1.08 | 0.64 |
| SH3GL2.EN | 9 | 18.0 | 0.94 | 2.99 | 1.00 | 1.22 |
| DOPHILIN1 | | | | | | |
| SH3GL1.EN | 19 | 15.6 | 2.86 | 2.30 | 2.22 | 2.66 |
| DOPHILIN2 | | | | | | |
| ADAM3A | 8 | 34.8-64.6 | 0.85 | 0.59 | 1.21 | 0.47 |
| ADAM7 | 8 | 48.1 | 0.94 | 0.47 | 1.30 | 0.40 |
| ADAM28 | 8 | 47.8 | 0.94 | 0.48 | 1.30 | 0.41 |
| ADAM9 | 8 | 60.0-65.8 | 1.10 | 1.14 | 1.00 | 0.70 |
| ADAM2 | 8 | 64.6 | 1.19 | 1.22 | 0.89 | 0.79 |
| ADAM18 | 8 | 64.6 | 1.19 | 1.22 | 0.89 | 0.79 |
| ADAMTS2 | 5 | 192.8 | 1.13 | 0.71 | 1.68 | 2.51 |
| ADAMTS3 | 4 | 80.9 | 0.77 | 0.71 | 1.48 | 0.31 |
| ADAMTS9 | 3 | 89.9 | 0.26 | 0.29 | 1.23 | 0.23 |
| ADAMDEC1 | 8 | 47.9 | 0.94 | 0.48 | 1.30 | 0.40 |

### Example 4

Gene Identification

Based on the linkage results above, genes were identified at the chromosomal locations described of Table 1 using the National Council for Biotechnology website (www.ncbi.gov). The genes and their related information are contained within Table 2. In addition, the alternate splice variants are also provided in Table 2. Column one, two and three of Table 2 contain the gene identifier, gene symbol and gene name, respectively. The accession numbers for the corresponding cDNA sequence are contained in the fourth column. The amino acid sequence accession number is listed in the fifth column. The genomic sequences accession numbers are provided in the sixth and seventh columns. In particular, the genomic contig for the region containing the gene is provided in the sixth column. The individual bacterial artificial chromosomes spanning the region containing the gene are listed with their respective accession numbers in seventh column. One skilled in the art could obtain the above described nucleic and amino acid sequences using the accession numbers provided herein. The eighth column provides the genetic marker of the location on the chromosome. And the gene description is provided in the ninth column.

Based on the linkage analysis, the genes described in Table 2 are involved in asthma and related diseases thereof.

TABLE 2

| | | | | | GenBank | | | |
|---|---|---|---|---|---|---|---|---|
| | | | | GenBank | Genomic | | | |
| | | | GenBank NT | Protein | Contig | GenBank | | |
| Gene Name | Gene Symbol | Gene Name | Accession # | Accession # | Accession # | Genomic Clone Accession # | Marker | Description |
| Gene845 | Adam19 | a disintegrin and metalloproteinase | NM_023038 | NP_075525 | NT_006788 | AC008676, AC008694 | stSG53531 | This variant (isoform-1) encodes a |
| | | domain 19 (meltrin beta) | | | | | | longer isoform which is divergent from |
| | | | | | | | | isoform 2 in the C-terminus. |
| Gene845 | Adam19 | a disintegrin and metalloproteinase | NM_033274 | NP_50377 | NT_006788 | AC008676, AC008694 | s1SG53531 | This variant (isoform-2) encodes a |
| | | domain 19 (mellna beta) | | | | | | shorter sotorm which is divergent from |
| | | | | | | | | isoform 1 in the C-terminus |
| Gene847 | NRG2 | neuregulin 2 | NM_004883 | NP_004874 | NT_007018 | AC008667, AC008523, AC011589, | | Splice vanant 1 lacks enons 6 and 7 |
| | | | | | | AC010292, AC026272, AC011379 | | |
| Gene847 | NRG2 | neuregulin 2 | NM_013981 | NP_053584 | NT_007018 | AC008667, AC008523, AC011589, | | Splice variant 2 lacks exons 5 and 7 |
| | | | | | | AC010292, AC028272, AC011379 | | |
| Gene847 | NRG2 | neuregulin 2 | NM_013982 | NP_053585 | NT_007018 | AC008667, AC008523, ACO11589, | | Splice variant 3 encludes enon 6 |
| | | | | | | AC010292, AC026272, AC011379 | | |
| Gene847 | NRG2 | neuregulin 2 | NM_013983 | NP_053586 | NT_007018 | AC008667, AC008523, AC011589, | | Splice variant 4 excludes exon 5 |
| | | | | | | AC010292, AC026272, AC011379 | | |
| Gene847 | NRG2 | neuregulin 2 | NM_013984 | NP_053587 | NT_007018 | AC008667, AC008523, AC011589, | | Splice variant 5 excludes enons 7, 9-12 |
| | | | | | | AC010292, AC026272, AC011379 | | and its enon 8 is missing 70 bps at the 3 |
| | | | | | | | | end. The protein product does not have |
| | | | | | | | | transmembrane and cytoplasmic tail |
| | | | | | | | | regions |
| Gene847 | NRG2 | neuregulin 2 | NM_013985 | NP_053588 | NT_007018 | AC008667, AC008523, AC011589, | | Splice variant 6 excludes exons 8-12 |
| | | | | | | AC010292, AC026272, AC011379 | | and its exon 7 is missing 3 bps at the 3 |
| | | | | | | | | end. The protein product does not have |
| | | | | | | | | tranumembrane and cytoplasmic tail |
| | | | | | | | | regions |
| Gene891 | NRG1 | neuregulin 1 | NM_004495 | NP_004486 | NT_007995 | AC083977, AC013561, AC128834, | stSG4083, | This variant (HRG-gamma) has a longer |
| | | | | | | AF181895 | SHGC-12780, | 5' UTR and longer 3' UTR than vanant |
| | | | | | | | WI-18803 | HRG-alpha The CDS is truncated on |
| | | | | | | | | the 3' end by 1289 bps resulhng in a |
| | | | | | | | | protein product equivalent to the N- |
| | | | | | | | | terminal 211 amino acids of the HRG- |
| | | | | | | | | alpha isoform. |
| Gene891 | NRG1 | neuregulin 1 | NM_013956 | NP_039250 | NT_007995 | AC083977, AC013561, AF128834, | stSG4083, | This vanant(HRG-betal), along with |
| | | | | | | AF181895 | SHGC-12780, | HRG-alpha, beta2, and beta3 vananits, |
| | | | | | | | WI-18803 | was idenrified in nartous normal tissues |
| | | | | | | | | and cancer cell lines The protein |
| | | | | | | | | product encoded by this vadant is |
| | | | | | | | | distinct from that of HRG-alpha in the |
| | | | | | | | | region of amino acids 213-239 |
| Gene891 | NRG1 | neuregulin 1 | NM_013957 | NP_039251 | NT_007995 | AC083977, AC013561, AF128834, | stSG4083, | This vadant (HRG-beta2), along with |
| | | | | | | AF181895 | SHGC-12780, | HRG-alpha, beta1, and beta3 variants, |
| | | | | | | | WI-18803 | was identified in various normal issues |
| | | | | | | | | and cancer cell lines. The protein |
| | | | | | | | | product encoded by this variant is |
| | | | | | | | | distinct from that of HRG-alpha at the |
| | | | | | | | | region of amino acids 213-231. |
| Gene891 | NRG1 | neuregulin 1 | NM_013958 | NP_039252 | NT_007995 | AC083977, AC013561, AF128834, | stSG4083, | This variant (HRG-beta3), along with |
| | | | | | | AF181895 | SHGC-12780, | HRG-alpha, beta1 and beta2 variants, |
| | | | | | | | WI-18803 | was identified in various normal tissues |
| | | | | | | | | and cancer cell lines. The protein |
| | | | | | | | | product encoded by this variant is 399 |
| | | | | | | | | amino acid shorter than, but the first N- |
| | | | | | | | | terminal 212 amino acids is the same |
| | | | | | | | | as, that of HRG-alpha |
| Gene891 | NRG1 | neuregulin 1 | NM_013959 | NP_039253 | NT_007995 | AC083977, AC013561, AF128834, | stSG4083, | This vanant (SMDF) is expressed mainly |
| | | | | | | AF181895 | SHGC-12780, | in the nervous system. It contains a C- |
| | | | | | | | WI-18803 | terminal EGF-like domain and a unique |
| | | | | | | | | N-terminal sequence which lacks an Ig- |
| | | | | | | | | like domain and is distinct from all |
| | | | | | | | | known HRG-variants |
| Gene891 | NRG1 | neuregulin 1 | NM_013980 | NP_039254 | NT_007995 | AC083977, AC013561, AF128834, | stSG4083, | This variant (ndf43) in one of the HRG- |
| | | | | | | AF181895 | SHGC-12780, | variants It has shorter 5 UTR, shorter |
| | | | | | | | WI-18803 | CDS, and longer 3' UTR than the variant |
| | | | | | | | | HRG-alpha Its amino acid sequence is |
| | | | | | | | | 178 amino acid shorter than, and the |
| | | | | | | | | last C-terminal 38 amino acids differs |
| | | | | | | | | from, that of the variant HRG-alpha. |
| Gene891 | NRG1 | neuregulin 1 | NM_013961 | NP_039255 | NT_007995 | AC083977, AC013561, AF128834, | s1SG4083, | The GGF (also called GGFHFB1) |
| | | | | | | AF161895 | ShIGC-12780, | variant is identical to HRG-beta3 variant, |
| | | | | | | | WI-18603 | except for its shorter 5' and 3' UTRs. |
| | | | | | | | | The GGF and GGF2 vanants are |
| | | | | | | | | expressed in the nervous system and |
| | | | | | | | | function as a neuronal signal that |
| | | | | | | | | promotes the proliferation and survival |
| | | | | | | | | of the oligodendrocyte and the |
| | | | | | | | | myelinating cells. |
| Gene891 | NRG1 | neuregulin 1 | NM_013962 | NP_039256 | NT_007995 | AC083977, AC013561, AF126834, | s1SG4083, | The GGF2 (also called GGFHBS5) |
| | | | | | | AF181895 | SHGC-12760, | variant differs from GGF variant at N- |
| | | | | | | | WI-18803 | terminal coding segments designated 1 |
| | | | | | | | | or 2, and their 5' UTR ate unrelated |
| | | | | | | | | Botrivadants are expressed in the |
| | | | | | | | | nervous system and function as a |
| | | | | | | | | neuronal signal that promotes the |
| | | | | | | | | proliferation and survival of the |
| | | | | | | | | oligodendrocyte and the myalinating |
| | | | | | | | | cell |
| Gene891 | NRG1 | neuregulin 1 | NM_013964 | NP_039258 | NT_007995 | AC083977, AC013561, AF128634, | stSG4083, | This variant (HRG-alpha), along with |
| | | | | | | AF181895 | SHGC-12780, | HRG-betal, beta2, and beta3 variants, |
| | | | | | | | WI-18603 | was identified in vanous normal tissues |
| | | | | | | | | and cancer cell lines. The protein |
| | | | | | | | | product encoded by this variant is |
| | | | | | | | | distinct from those of HRG-beta vanants |
| | | | | | | | | at the region of amino acids 213-234. |
| Gene874 | SH3GL2 | SH3-domain GRB2-like 2 | NM_003026 | NL_003017 | NT_029370 | AL139115 | | SH3-domain GRB2-like 2 |
| | (Endophilin1) | | | | | | | |
| Gene803 | SH3GL1 | SH3-domain GRB2-like 1 | NM_003025 | NP_003016 | NT_011245 | AF190465, AC007292 | | SH3-domain GRB2-like 1 This vanant |
| | (Endophilin2) | | | | | | | (1) has longer exons E and F as |
| | | | | | | | | compared to vanant 2. There are 369 |
| | | | | | | | | amino acids. |
| Gene803 | SH3GL1 | SH3-domain GRB2-like 1 | AK097616 | N/A | NT_011245 | AF190465, AC007292 | | SH3-domain GRB2-like 1 This variant |
| | (Enxophilin2) | | | | | | | (2) has shorter exons E and F as |
| | | | | | | | | compared to vanant 1. This variant is 64 |
| | | | | | | | | amino acids shorter than vanant 1 |
| Gene894 | ADAM3A | a disintegrin and metalloproteinase | X89657 | | no alignment | | | a disintegrin and metalloproteinase |
| | | domain 3a (cyntestin 1) | | | in Draft | | | domain 3a (cyntestin 1) pseudogene |
| | | | | | sequence | | | |
| Gene895 | ADAM28 | a disintegrin and metalloproteinase | NM_014265 | NP_055080 | NT_008130 | AC023202, AC044891 | stSG42867 | This variant (1) encodes isoform 1, |
| | | domain 28 | | | | | | which has the same amino acid length |
| | | | | | | | | as isoform 2 encoded by variant 2 |
| | | | | | | | | These two isoforms differ from each |
| | | | | | | | | other in the next to last amino acid. In |
| | | | | | | | | addition, variant 1 contains a 272 bps |
| | | | | | | | | longer 3' UTR than variant 2. |
| Gene895 | ADAM28 | a disintegrin and metalloproteinase | NM_021777 | NP_068547 | NT_008130 | AC023202, AC044891 | s1tG42867 | This variant (3) contains a shorter 3' |
| | | domain 28 | | | | | | coding region and a different 3' UTR |
| | | | | | | | | from variant 2. The isoform 3 encoded |
| | | | | | | | | by this variant lacks transmembrane and |
| | | | | | | | | cytoplasmic domains, as compared to |
| | | | | | | | | isoform 2 encoded by variant 2 |
| Gene895 | ADAM28 | a disintegrin and metatoproleinase | NM_021778 | NP_068548 | NT_008130 | AC023202, AC044891 | stSG42867 | This variant (2) contains a longer 3' |
| | | domain 28 | | | | | | coding region and different 3' UTR from |
| | | | | | | | | vacant 3 The isoform 2 encoded by this |
| | | | | | | | | variant contains transmembrane and |
| | | | | | | | | cytoplasmic domains, as compared to |
| | | | | | | | | isoform 3 encoded by variant 3. |
| Gene895 | ADAM7 | a disintegrin and metalloproteinase | AF215824 | AAG43987 | NT_023666 | AC024958, AC018422 | | a disintegrin and metalloprotenase |
| | | domain 7 | | | | | | domain 7 |
| Gene897 | ADAM9 | a disintegrin and metalloproteinase | NM_003816 | NP_003807 | no alignment | | D14665, | a disintegris and metalloproteinase |
| | | domain 9 (meltrin gamma) | | | is Draft | | | domain 9 preproprotein |
| | | | | | sequence | | RH25259 | |
| Gene898 | ADAM2 | a disintegrin and metalloproteinase | NM_001464 | NP_001455 | NT_008045 | AF178650, AC018807 | U52370 | a disintegrin and metalloproteinase |
| | | domain 2 (fertilin beta) | | | | | | domain 2 proprotein |
| Gene899 | ADAM18 | a disintegrin and melalloproteinase | NM_014237 | NP_055052 | NT_008045 | AF178650, AC018807 | | a disintegrin and metalloproteinase |
| | | domain 18 | | | | | | domain 18 preproprotein |
| Gene962 | ADAMTS2 | a disinlegrin-like and | NM_014244 | NP_055059 | NT_006802 | AC010216, AC008470, AC008544, | | This variant (1) and variant 2 share |
| | | metalloprolease (reprolysin type) | | | | AC034202, AC023587, AC016557 | | identical 5'-region of 1629 bps, which |
| | | with thrombospondin | | | | | | encodes N-terminal 543 amino acids, |
| | | type 1 motif, 2 | | | | | | but they have diverse 3-region Vanant |
| | | | | | | | | 1 is 1935 bps longer than variant 2 in |
| | | | | | | | | the 3-region, and the isoform 1 encoded |
| | | | | | | | | by variant 1 is 645 amino acids longer |
| | | | | | | | | than isoform 2 encoded by variant 2. |
| | | | | | | | | Isoform 1 contains 4 C-terminal TS |
| | | | | | | | | motifs, whereas isoform 2 lacks C- |
| | | | | | | | | terminal TS mofits. |
| Gene962 | ADAMTS2 | a disintegrin-like and | NM_021599 | NP_067610 | NT_006802 | AC010216, | | This variant (2) and variant 1 share |
| | | melalloprotease (reprolysin type) | | | | AC008470, | | identical 5'-region of 1629 bps, which |
| | | with thrombospondin | | | | AC008544, | | encodes N-terminal 543 amino acids, |
| | | type 1 motif, 2 | | | | AC034202, | | but they have diverse 3-region Vanant |
| | | | | | | AC023587, | | is 1935 bps shorter than variant 1 in |
| | | | | | | AC0165572 | | the 3-region, and the isoform 2 encoded |
| | | | | | | | | by variant 2 is 645 amino acids shorter |
| | | | | | | | | than isoform 1 encoded by variant 1 |
| | | | | | | | | Isoform 2 lacks C-terminal TS motifs, |
| | | | | | | | | whereas isoform 1 contains 4 C-terminal |
| | | | | | | | | TS motifs Variant 2 is resulted from |
| | | | | | | | | retention of a portion of an intron and |
| | | | | | | | | the use of a polyA signal which is |
| | | | | | | | | located within the intron sequence. |
| Gene901 | ADAMTS3 | a disintegrin-like and | XM_036683 | XP_036683 | NT_022833 | AC011819, | A009A40, | a disintegrin-like and melalloprotease |
| | | metalloprotease (reprolysin type) | | | | AC055844, | stSG39575, | (reprolysin type) with thrombospondin |
| | | with thrombospondin | | | | AC022843, | SHGC-50515 | type 1 motif, 3 |
| | | type 1 motif, 3 | | | | AC088203 | | |
| Gene902 | ADAMTS9 | a disintegrin-like and | NM_020249 | NP_084834 | no alignment | | | a disintegrin and metalloproteinase with |
| | | metalloprolease (reprolysin type) | | | in Draft | | | thrombospondin motifs-9 preproprotein |
| | | with thrombospondin | | | sequence | | | |
| | | type 1 motif, 9 | | | | | | |
| Gene903 | ADAMDEC1 | ADAM-like, decysin 1 | NM_014479 | NP_055294 | NT_023666 | AC024958 | | ADAM-like, decysin 1 a disintegrin |
| | | | | | | | | protease |

### Example 5

Mutation Analysis

Gene 803, Gene 845, Gene 847, Gene 874 and Gene 962 disorder-associated candidate genes were identified using the above procedures, and exons from these genes were subjected to mutation detection analysis. A combination of fluorescent single stranded confirmation (SSCP) analysis (ABI), DNA sequencing, and other sequence analysis methods described herein were utilized to precisely identify and determine nucleotide sequence variants. SSCP analysis was used to screen individual DNA sequences for variants. Briefly, PCR was used to generate templates from unrelated asthmatic individuals. Non-asthmatic individuals were used as controls. Enzymatic amplification of the disorder-associated genes was accomplished using primers flanking each exon and the putative 5′ regulatory elements of each gene. The primers were designed to amplify each exon, as well as 15 or more base pairs of each intron on either side of the splice site. The forward and the reverse primers had two different dye colors to allow analysis of each strand, and independent confirmation of variants. PCR reactions were optimized for each exon primer pair. Buffer and cycling conditions were specific to each primer set. PCR products were denatured using a formamide dye, and electrophoresed on non-denaturing acrylamide gels with varying concentrations of glycerol (at least two different glycerol concentrations).

Primers utilized in fluorescent SSCP experiments to screen coding and non-coding regions of Gene 803, Gene 845, Gene 847, Gene 874 and Gene 962 for polymorphisms are provided in Table 3. The first column list the genes targeted for mutation analysis. The second column list the specific exons analyzed. The assigned primer names are described in the third column. The fourth and fifth columns list the forward primer sequences and the reverse primer sequences, respectively. The genes listed in the first column of Table 3 correspond to the gene identifiers in the first column of Table 2.

TABLE 3
SSCP PRIMERS

| Gene | Exon | SSCP Assay | Forward Sequence | Reverse Sequence |
|---|---|---|---|---|
| 845 | B | 1792_845_B_F_1793_845_B_R | GGAGCGTCTCGACAGAGG | AAGGCTACTCCCAGGTCTCC |
| 845 | C | 1794_845_C_F_1795_845_C_R | CTGGGATTCCAATGGTTTTG | AGAGTGGGCCAGAAACAGAA |
| 845 | D | 1796_845_D_F_1797_845_D_R | TCCTGTGGATCAATGTTGGA | CTTCCTTCCAGAACAGCGAC |
| 845 | E | 1798_845_E_F_1799_845_E_R | GTTGTTGCTAGGAGGGTGGA | TCCCTTCAGAGGAGAGACCA |
| 845 | F | 1800_845_F_F_1801_845_F_R | CATCCATGACCATCCTGAGA | TTGAAACTTCCATTCCTGGG |
| 845 | G | 1802_845_G_F_1803_845_G_R | CTAAGACCTACACAAGGGACTTTT | CAGGTTGATTTCTGCACTGAG |
| 845 | H | 1804_845_H_F_1805_845_H_R | CGGAGGTAGCTGCTGCTTAT | AACATTTCCCCAAAGCAGTCT |
| 845 | I | 1806_845_I_F_1807_845_I_R | CAAGACAAGTAATGGGGCG | GGCACCTTTCCCACAAGTA |
| 845 | J | 1808_845_J_F_1809_845_J_R | ATCCCAGGTGGTATTGACAGA | CAGCCAAGGACAACACAACA |
| 845 | K | 1810_845_K_F_1811_845_K_R | CCACTTACTCCCAGGCACAT | CTGGTCTTGAAAGGCAGCTC |
| 845 | L | 1842_845_L_F_1813_845_L_R | GTCCCCTTGACCTTGACCTC | AACCCCTGGGTCACACTGTC |
| 845 | M | 1814_845_M_F_1815_845_M_R | AGGAGTGACAGCACGAGTGA | CAAATTCTTCCCCTCCCACT |
| 845 | N | 1816_845_N_F_1817_845_N_R | TTGCTAGAGAGCTGGGGTTC | AGTCCTGTGGTCCCACTGTC |
| 845 | O | 1818_845_O_F_1819_845_O_R | TGGGGAGGAGATTGACTGTG | CAAAACTACCCTGAGGGCCA |
| 845 | P | 1820_845_P_F_1821_845_P_R | GGGGCTTCTGACAGATGAGT | AGTTGGGCAACAGTGAGGAC |
| 845 | Q | 1822_845_Q_F_1823_845_Q_R | GACTGAAGCTCTCTGGTGCC | CTCCTCAGGACCTCGGTAGA |
| 845 | Q | 1824_845_Q_F_1825_845_Q_R | TGCCCATTGACACCACTATC | CATCCTTCCCTCAGACCTCA |
| 845 | R | 1826_845_R_F_1827_845_R_R | TCTTGCCTCCTAACTCCCAA | GACCTGGAGCAAAGAAAGGG |
| 845 | S | 1828_845_S_F_1829_845_S_R | CTGGGTTCTGGCTTCTCTGT | CTCACAAAAGCGGGCAGT |
| 845 | T | 1830_845_T_F_1831_845_T_R | AAATCTGCTGTAGCCGAGACA | TGGAGACCTTTGTGACCCAT |
| 845 | U | 1832_845_U_F_1833_845_U_R | ACTGTCCCCTGCTGAACATC | AAATACAGCATGGCCCTGAG |
| 845 | V | 1834_845_V_F_1835_845_V_R | CCCTTTGGGCTCTGGTTTAT | GGACGACTCCGTCCTCTCTA |
| 845 | V | 1836_845_V_F_1837_845_V_R | GATTATCTGCGTGGTGGGTC | AGTTTCACCTTCCCCACCTT |
| 845 | W | 1838_845_W_F_1839_845_W_R | TCTTTCAAACAGGCCTCTGG | ACCAGCTTTCACCTTGAGGG |
| 845 | X | 1840_845_X_F_1841_845_X_R | TGATCCCATTGATCAGCATC | TTTGGAGATGTGGAGGTTCC |
| 847 | A | 1925_847_A_F_1926_847_A_R | CTGTTTCCGGTTTTCCAGC | GACGAGAGATGCTGCTGTTG |
| 847 | A | 2009_847_A_F_2010_847_A_R | CAGGAGCAGCAGCAACAA | TGGTCCTGCACTGACTTGAG |
| 847 | A | 1929_847_A_F_1930_847_A_R | GGCTTCTCCATGCTGCTCT | CCACGCTGATCACCTGCT |
| 847 | A | 1931_847_A_F_1932_847_A_R | TAAAGGTGCTGGACAAGTGG | CCTTTCTCCAGCAAAGGGA |
| 847 | B | 1903_847_B_F_1904_847_B_R | ACCACCGTGCTCACCTACCT | AGCTGCTTGGATGGAGGAC |
| 847 | C | 1905_847_C_F_1906_847_C_R | CTCTGTGGAGAGAGGCMCC | CAACCCCTCTAGGACCCTT |
| 847 | D | 1907_847_D_F_1908_847_O_R | GGGGCCTAGGGATAGTCTCA | CTACCCTGTTCTTGCTCCCA |
| 847 | E | 1909_847_E_F_1910_847_E_R | CCAAGTGCCTGACTTGGTTT | GGAGCAGGGACTTGTGTTTG |
| 847 | F | 1911_847_F_F_1912_847_F_R | TCCTGGCTCTCTCUTCTGG | CTCTAAGGAGCGCAGGACAC |
| 847 | G | 1913_847_G_F_1914_847_G_R | CTAACCTGCTTTCACCTCGC | CATTCAGCACACATGGCATC |
| 847 | H | 1915_847_H_F_1916_847_H_R | AAGGGGTCTCTGCACCACTA | ACATTCTTGGAGGCCCATC |
| 847 | I | 1917_847_I_F_1918_847_I_R | TAGGGAAGTTCATCGTTGGC | AGAAGGCTGGCTGTCCACT |
| 847 | J | 1919_847_J_F_1920_847_J_R | CCTGTCCCCAACAAGAAAGA | TTTGCGCCAGATGAAGTATG |
| 847 | K | 1921_847_K_F_1922_847_K_R | GAGCTCGAGGTGGAAGAAGA | CTCCTCCAGGTTGTAGGCTG |
| 847 | K | 1923_847_K_F_1924_847_K_R | TCATCAGTGGGTACCAGCPA | TTTGGAGTGTTTCTGAGGGG |
| 847 | L | 2030_847_L_F_2031_847_L_R | CCACCCTATCACGATTCCGT | GCAGTAACGGCTGCTGCTC |
| 847 | L | 1933_847_L_F_1934_847_L_R | GTACGTGTCGGCCCTGA | ATAGCTGCGCTGCATGTCT |
| 847 | L | 1935_847_L_F_1936_847_L_R | CCCATCAGTTACCGCCTG | CTCCTGCGTGGTCTCGTACT |
| 847 | L | 1937_847_L_F_1938_847_L_R | GTACGAGACCACGCAGGAGT | TCAGCGACAGCGAGTCC |
| 847 | L | 1939_847_L_F_1940_847_L_R | GTAGACCACGCAGGAGT | CCCAGGAAAGGTGTGCTCT |
| 847 | L | 1941_847_L_F_1942_847_L_R | GGACTCGCTGTCGCTGAG | GCTGTGGCTGTCCAGTGAGTA |
| 847 | L | 1943_847_L_F_1944_847_L_R | AGAGCACACCTTTCCTGGG | CTCCTTAAAGATAGTGGGGCG |
| 803 | B | 2052_803_B_F_2053_803_B_R | GTGGCGGGGCTGAAGAAG | CCCTTGGTCTTCCCACCTG |
| 803 | C | 2054_803_C_F_2055_803_C_R | GTGTCCTGATCACTTGGCCT | AGTGCCACCACCACACAGA |
| 803 | D | 2056_803_D_F_2057_803_D_R | CCTGGTATGGGCTCTTAGGG | GTTCTGTCATCCCCTGCCT |
| 803 | E | 2058_803_E_F_2059_803_E_R | AAGGGTGGGGAGGAGATGT | CAGAGAGCACCACTCACCAA |
| 803 | E | 2060_803_E_F_2061_803_E_R | TCTGGGCGAGTGCATGAT | TTGGTCTGTGCAGTCTCCTG |
| 803 | F | 2062_803_F_F_2063_803_F_R | AGGCCCGGTATGATGGCTT | GAGGTGAGGGTGGCAGGAAT |
| 803 | G | 2064_803_G_F_2065_803_G_R | CGCTACTGGTGTGACCCAT | CCCAAGGGCATAGGTCTTCT |
| 803 | H | 2066_803_H_F_2067_803_H_R | AGGGAAGGCACAGGACAGT | CATGTGCTCACCCCACAG |
| 803 | I | 2068_803_I_F_2069_803_I_R | AAATACAAGAGTGGGGCTGC | CATTTGCCTCCGCAAGAG |
| 803 | J | 2070_803_J_F_2071_803_J_R | ACCGTTTTGAGCCCACAG | GCTTGAGATGGGCAGAGAAC |
| 803 | K | 2072_803_K_F_2073_803_K_R | CAGGTCACAGCAGGTCTGAG | GGACACGGGTGAGTCACTG |
| 803 | K | 2074_803_K_F_2075_803_K_R | AGCTACGTGGAGGTGCTTGT | TGGGAGTCAGCGCTAGTGTA |

Comparative DNA sequencing was used to determine the sequence changes in Gene 803, Gene 845, Gene 847, Gene 874 and Gene 962. Variants detected by SSCP analysis in the initial set of asthmatic and normal individuals were analyzed by fluorescent sequencing on an ABI 377 automated sequencer (Perkin-Elmer Applied Biosystems Division). Sequencing was performed using Amersham Energy Transfer Dye Primer chemistry (Amersham-Pharmacia Biotech) following the standard protocol described by the manufacturer. Primers used for dye primer sequencing are shown in Table 4. The first column lists the genes targeted for sequencing. The second column list the specific exons sequenced. The third and fourth list the forward primer names and the forward primer sequences, respectively. The fifth and sixth columns list the reverse primer names and reverse primer sequences, respectively.

TABLE 4
SEQUENCING PRIMERS

| Gene | Exon | Forward Primer | Forward Sequence | Reverse Primer | Reverse Sequence |
|---|---|---|---|---|---|
| 803 | B | MDSeq_523_803_B_F | TCCCGCGGTAGACAATG | MDSeq_523_803_B_R | GAGAAGGAGGGGAGAGGTC |
| 803 | B | MDSeq_540_803_B_B_F | AGGGTCACCTCCACGACTC | MDSeq_540_803_B_R | CCCTTGGTCTTCCCACCT |
| 803 | B | MDSeq_575_803_B_F | AACTCACTGACAGAGGCGG | MDSeq_575_803_B_R | GTCCCTTGGTCTTCGCACC |
| 803 | B | MDSeq_659_803_B_F | TGTGCACGTGCGCTCTTC | MDSeq_659_803_B_R | GTCCCFrGGTCTTCCCACCT |
| 803 | B | MDSeq_660_803_B_F | GCTGAACACTTAGACGAAOTGGATT | MDSeq_660_803_B_R | GGCACACACGTATCTTAGGAAAG |
| 803 | E | MDSeq_510_803_E_F | CTGAGGAGCTTGGTCACCTC | MDSeq_510_803_E_R | GTTTGCCCTTAACAGGTGGA |
| 803 | F | MDSeq_511_803_F_F | GGAAAGGATCCAGGTGTG | MDSeq_511_803_F_R | CTCCAGTTTCTTCAGGTGG |
| 803 | G | MDSeq_512_803_G_F | CTGAAGGAGATCCAGGTGCT | MDSeq_512_803_G_R | CTGTCCTGTGCCTTCCCT |
| 803 | H | MDSeq_513_803_H_F | ATGCACAACCTCCTGGAGAC | MDSeq_513_803_H_R | GGCTGGGTGTTGACTGAGA |
| 803 | I | MDSeq_514_803_I_F | CCCAGTCTAGCTGTGTCCC | MDSeq_514_803_I_R | TGTCGGAAGATCGGAAGAC |
| 803 | K | MDSeq_515_803_K_F | CCAAGCTTCTCCCATCCT | MDSeq_515_803_K_R | CTCAGGGAGTACCTGAAGGG |
| 845 | D | MDSeq_426_845_D_F | CCAGTGTTTCCCTTCACC | MDSeq_426_845_D_R | CCAGCAATCTCACATCGAG |
| 845 | E | MDSeq_433_845_E_F | GGAGTGATGTTCCCATAGTG | MDSeq_433_845_E_R | ATGTGGGTAATTACATAAAGCAA |
| 845 | F | MDSeq_442_845_F_F | AATGACATCTTCCCTGCCC | MDSeq_442_845_F_R | ATGGCAGTCATCTCCTGA |
| 845 | H | MDSeq_427_845_H_F | CTCCAGCAATAACCAAATG | MDSeq_427_845_H_R | TGCTACTGCCACAGCCT |
| 845 | H | MDSeq_504845_H_F | TAGCATGGGTAAGGCGTG | MDSeq_504_845_H_R | GCCTTCTCTGCTCACTCCAC |
| 845 | I | MDSeq_428_845_I_F | CAAGGGTTAGAGGAAGGCA | MDSeq_428_845_I_R | CGTAGTTCAGGGCTCTGTCA |
| 845 | J | MDSeq_436_845_J_F | GTTGCCTCCTTCTGTTGGA | MDSeq_436_845_J_R | TGGGTACAGAGCGCATGTT |
| 845 | K | MDSeq_429_845_K_F | CCAAGGAATCAGCTATGGG | MDSeq_429_845_K_R | CTTCAGGGTTCCTGAGCTTG |
| 845 | O | MDSeq_443_845_O_F | TGAGAAGGCTGAAGGTG | MDSeq_443_845_O_R | CTTAGGGCCATTTGCATT |
| 845 | P | MDSeq_430_845_P_F | GTTGAGAATATGGGGATGGA | MDSeq_430_845_P_R | GAAATGACCCAAAAGGGCT |
| 845 | R | MDSeq_432_845_R_F | ACAGACACAGGCCACCAG | MDSeq_432_845_R_R | TGTGGATGCTCTGCAACA |
| 845 | U | MDSeq_444_845_U_F | GGCCAACTCTGTTTCCTTGA | MDSeq_444_845_U_R | ATGGTGGTGGGCACCTG |
| 845 | V | MDSeq_437_845_V_F | CTAAGAGCCTCTGTGGGC | MDSeq_437_845_V_R | GTTGCTCTAACCTGCTGTG |
| 845 | W | MDSeq_445_845_W_F | CAGCTTGCTCTCCTGACTT | MDSeq_445_845_W_R | GGGCACCPAGAAACATGAAT |
| 845 | X | MDSeq_446_845_X_F | GGCTGACCATGCTGTATTC | MDSeq_446_845_X_R | GAGGAGAAGCTGCCAGTCAC |
| 847 | A | MDSeq_438_847_A_F | GCATCCTCCTCCAGGTCC | MDSeq_438_847_A_R | GTACCTTGCCCTCCACCAC |
| 847 | A | MDSeq_457_847_A_F | AACAGCAGCATCTCTCGTC | MDSeq_457_847_A_R | CCATTCTCCACCACCTCG |
| 847 | A | MDSeq_475_847_A_F | GCATCCTCCTCCAGGTCC | MDSeq_475_847_A_R | GTACCTTGCCCTCCACCAC |
| 847 | B | MDSeq_439_847_B_F | GGCATGAAGGAAACTCTCCA | MDSeq_439_847_B_R | GGGTCTTCCACTGATCAAGC |
| 847 | C | MDSeq_465_847_C_F | GGACATGTGAGCAGCCACTA | MDSeq_465_847_C_R | TTCCAGGCCCAGATAACAA |
| 847 | D | MDSeq_466_847_D_F | GGTTGCACTGGGTAAACG | MDSeq_466_847_D_R | CCTAAAGGGTGTTGGTGAA |
| 847 | E | MDSeq_447_847_E_F | TTTCCCATCTTCCCTCACC | MDSeq_447_847_E_R | ACCTGCAGCCCTGAACTTT |
| 847 | F | MDSeq_467_847_F_F | GTTCAGGGCTGCAGGTAA | MDSeq_467_847_F_R | AGACCCTTTGGTACCCTCA |
| 847 | G | MDSeq_478_847_G_F | TCTCTAAAGAGCCTGCCCTG | MDSeq_478_847_G_R | GCCCTTCTGTTCATGAGCTT |
| 847 | H | MDSeq_468_847_H_F | CCACTCATGTGCTCTGG | MDSeq_468_847_H_R | CCATTCTTCGTCAGTGCC |
| 847 | I | MDSeq_479_847_I_F | GCTTGAGTACAGGGACGAGC | MDSeq_479_847_I_R | CATGGAAGTGAGCAAACCA |
| 847 | J | MDSeq_454_847_J_F | GGTTTGCTCACTTCCATGA | MDSeq_454_847_J_R | GTCTGAGCCTTTGTGCTG |
| 847 | K | MDSeq_448_847_K_F | CAGCCCTCCTTCTTCCAATA | MDSeq_448_847_K_R | ACTGGCCCAACTCTAGTCC |
| 847 | L | MDSeq_450_847_L_F | TCGAAGATCCTGAGCGAGT | MDSeq_450_847_L_R | GGTCACTCCTCCCTCTGC |
| 847 | L | MDSeq_451_847_L_F | CCCATTTATCAGTGGTTGC | MDSeq_451_847_L_R | CTACCCCTTCCCGGCTC |
| 847 | L | MDSeq_461_847_L_F | GACTTCCACTACTCGOTGGC | MDSeq_461_847_L_R | CCAGGAAAGGTGTGCTCT |
| 847 | L | MDSeq_469_847_L_F | GACAGCTACTATTACCCCGC | MDSeq_469_847_L_R | CGCCTTTGCCGTTAG |
| 847 | L | MDSeq_501_847_L_F | GCCTGACTTCTGACTCCCA | MDSeq_501_847_L_R | TCCTGCGTGGTCTCGTACT |
| 847 | L | MDSeq_502_847_L_F | GCGCAGCTATGACAGCTACTA | MDSeq_502_847_L_R | GGTCTCCTTAAAGATAGTGGG |
| 847 | L | MDSeq_503_847_L_F | GGACTCGCTGTCGCTGAG | MDSeq_503_847_L_R | CCGAAGGTGTAAATCAGGA |
| 847 | L | MDSeq_516_847_L_F | CCACAGACCATGTCATCAGG | MDSeq_516_847_L_R | CCGAAGCTGTAAATCAGGA |
| 874 | R | MDSeq_592_874_R_F | GGCCCGCTGACTAGGGAT | MDSeq_592_874_R_R | GCGCTACCAGGCAGGAC |
| 874 | S | MDSeq_615_874_S_F | CAGACCCTCAGAGCCACA | MDSeq_615_874_S_R | CATCGTGACCTTTCACCTTCA |
| 874 | T | MDSeq_616_874_T_F | GGATAAACGGGCTTTCCACA | MDSeq_616_874_T_R | TGTGTCACCTGAACTGTTTGC |
| 874 | T | MDSeq_681_874_T_F | GGATAAACGGGCTTTCCACA | MDSeq_681_874_T_R | TGTGTCACCTGAACTGTTTGC |
| 874 | T | MDSeq_701_874_T_F | CCTCACCTACTGCGGGACTT | MDSeq_701_874_T_R | GTGGACCGAGGAAGCAA |
| 874 | U | MDSeq_617_874_U_F | ACAGTGAAGGGAGGATGGG | MDSeq_617_874_U_R | GGCCCATGTGTGTAGGA |
| 874 | V | MDSeq_618_874_V_F | ACAAGAGAGGGCAGGGAGC | MDSeq_618_874_V_R | AACGTGCCCAGAGCTGAC |
| 874 | W | MDSeq_619_874_W_F | AGCCTTTGMAGCACTGGC | MDSeq_619_874_W_R | CCCATCATAAGTTAAGGAGCATCTG |
| 874 | X | MDSeq_620_874_X_F | CACAGTCATCAGCACCACCA | MDSeq_620_874_X_R | CGCCTCTGTAGGATMGCGG |
| 874 | Y | MDSeq_621_874_Y_F | GATTTCCCACACTTCATCATGG | MDSeq_621_874_Y_R | GCAGACCAAACTGACAAGG |
| 874 | Z | MDSeq_622_874_Z_F | GCAAGTCACGGTCAGACTGG | MDSeq_622_874_Z_R | GCCCACCAGGCTAAGAGGAT |
| 874 | Z | MDSeq_682_874_Z_F | AACAGGAAGAGGPATGAGGG | MDSeq_682_874_Z_R | CCATGACTCCTGTGGGAGC |
| 962 | D | MDSeq_858_962_D_F | TCGCTCCTTCCCTCTCCC | MDSeq_858_962_D_R | GACTCAGGAGCCGCCAGT |
| 962 | E | MDSeq_859_962_E_F | GAGGTGCAGGCTGGCTTCT | MDSeq_859_962_E_R | CGACGTAGAGACAGCTCCC |
| 962 | E | MDSeq_860_962_E_F | CGTGGTGTCGGCAGCTA | MDSeq_860_962_E_R | GGAGCACGAGGCTCACTAA |
| 962 | F | MDSeq_861_962_F_F | CTCTGGGACCACATGTTCA | MDSeq_861_962_F_R | CTGAGCCACTGAGACCG |
| 962 | G | MDSeq_862_962_G_F | TTCCTCTGCACCTCGCTTT | MDSeq_862_962_G_R | CACTCACOTCCGGCTAACA |
| 962 | H | MDSeq_863_962_H_F | AGTTGATGGTGACGCCTGG | MDSeq_863_962_H_R | CCGTGGCCTGGTATGTCTCT |
| 962 | I | MDSeq_864_962_I_F | CCTGTCAAAGACTGGAGCCC | MDSeq_864_962_I_R | CCACACCCTCACCCAGCTA |
| 962 | J | MDSeq_865_962_J_F | CTGCTGTCAGCCAGATGTC | MDSeq_865_962_J_R | GGGAAGACAGGAGACCACA |
| 962 | K | MDSeq_866_962_K_F | AGCAGGTAGATGGTCGGTG | MDSeq_866_962_K_R | CAGGTGTGCCCTOTCTCCTC |
| 962 | L | MDSeq_867_962_L_F | TGCAGCAGGGAAACTGAGG | MDSeq_867_962_L_R | CCTGCCTAGGACCACGTCT |
| 962 | M | MDSeq_868_962_M_F | CACCGTATGGGCMGGTCT | MDSeq_868_962_M_R | GGTGTGCTCCAGCATCAGA |
| 962 | N | MDSeq_869_962_N_F | GGCGGAGCTTCTGAAAGAAA | MDSeq_869_962_N_R | CCGAATGGTGCAC1TCACTT |
| 962 | O | MDSeq_870_962_O_F | GCCAATGAGGCCTGTCTTCT | MDSeq_870_962_O_R | CCTGCACCTCTCCAGAACT |
| 962 | O | MDSeq_907_962_O_F | GCATCTAGGGGCGAGGAG | MDSeq_907_962_O_R | TCCCAGGTAGGGTGAGGC |
| 962 | P | MDSeq_871_962_P_F | GCTCAGCAGAGCTGCCC | MDSec_871_962_P_R | GCCCTGCTAGGAACTTTAATGC |
| 962 | Q | MDSeq_872_962_Q_F | TTTCCCTCTCTGTCTGCCC | MDSeq_872_962_O_R | GTCTCAGGCTCCCAGCAC |
| 962 | R | MDSeq_873_962_R_F | GATGAGGCCAGGCAAGGT | MDSeq_873_962_R_R | CCGACTTAGGATTCTCCCTCC |
| 962 | S | MDSeq_874_962_S_F | GTGGGTCCTCAAAGCCAGAG | MDSeq_874_962_S_R | GCTGAGCAGAGGGACAGGTT |
| 962 | T | MDSeq_875_962_T_F | GTCGTGGCACAGAGGAAAC | MDSeq_875_962_T_R | TGTCCCTTCCTGGCTGTGA |
| 962 | U | MDSeq_876_962_U_F | GCTCACAGGAACCTCACCCT | MDSeq_876_962_U_R | GGTCCCTGCTGTGGCT |
| 962 | V | MDSeq_877_962_V_F | CCTGCCACCCTTATTGT | MDSeq_877_962_V_R | CTGCCCTGCAGCATCTTA |
| 962 | W | MDSeq_878_962_W_F | TAAGAGAAGGTGCACCGGG | MDSeq_878_962_W_R | GTCATTGCAGTGCTTGGCAT |
| 962 | W | MDSeq_908_962_W_F | GGTTCTCCAGGTGCTGCG | MDSeq_908_962_W_R | GCCAATGATCAGGGCAGAG |
| 962 | X | MDSeq_879_962_X_F | AGAGAGACAGGCATGTGGG | MDSeq_879_962_X_R | CGCTTCTCCATGCTCACAGA |
| 962 | Z | MDSeq_880_962_Y_F | CAGCGGAGGACCCTTTGTC | MDSeq_880_962_Y_R | CCACGACAGATCTTTGCCC |
| 962 | Z | MDSeq_881_962_Z_F | CTTTCTGCCTGGGCTCACTT | MDSeq_881_962_Z_R | GATTAGGTTGGGTGGCTGGA |
| 962 | Z | MDSeq_882_962_Z_F | GCTGTGCTGCAAGTCCTG | MDSeq_882_962_Z_R | GTATTTGGTCGCTCCTGGG |

Single nucleotide polymorphisms (SNPs) that were identified in Gene 803, Gene 845, Gene 847, Gene 874 and Gene 962 are shown in Table 5. The first and second columns list SNP identifier and gene names, respectively. The third column lists the exons that either contain the SNPs or are flanked by intronic sequences that contain the SNPs. The fourth column lists the PMP sites for the SNPs. The “−” symbols denote polymorphisms which are 5′ of the exon and are within the intronic region. The “−” polymorphisms are numbered going from the 3′ to 5′ direction. The “+” symbols denote polymorphisms which are 3′ of the exon and are within the intronic region. The “+” polymorphisms are numbered going from the 5′ to 3′ direction. The second, third, and fourth columns, combined, correspond to the SNP names as described herein, e.g., 845_D_{—}+1, 845_D_{—}−1 etc. It should be noted that the disclosed SNPs are referred to herein using both short (e.g., SNP D+1 of Gene 845 or 845_D+1) and long (e.g., Gene 845 D+1) nomenclature. The fifth column lists the localization of the SNPs to exon, intron, or UTR sequences. The sixth column lists the SNP reference sequences and illustrates the SNP nucleotide changes in boldface. The seventh column lists the base changes of the SNP sequences. If applicable, the eighth column lists the amino acid changes resulting from the SNP sequences. The coordinates of the SNP as it corresponds to the genomic sequence are contained in the ninth and ten columns. More particularly, the ninth lists the coordinate of the particular SNP in relation to the single genomic contig reference sequence. The genomic contigs used to create the reference sequence are listed in the tenth column of Tables 5. The genomic sequences and contig sequences with their respective accession numbers are listed in Table 2 and provided in SEQ ID. NOs. 1-9. Column eleven lists the coordinates of the SNP as it corresponds to the genomic contig and sequence listed in column 10. The SNPs identified in the cDNA contain a coordinate listed in the twelfth column. In some instances, alternate splice variants for Gene 803, Gene 847 and Gene 962 contain different coordinates for each. Thus, the respective SNP and coordinate for each splice variant is noted in Table 5. In addition, FIGS. 1-12 show the respective cDNA sequence and SNP location relating to Table 5. One skilled in the art could also take the reference sequence listed in column 6 in Table 5 and compare to the related sequence described in Table 2 using the appropriate Accession number. For example, one could use the program BLAST or ClustalW to perform an alignment and comparison to identify the specific location. One could identify the location of exonic SNPs using FIGS. 1-12 to locate the particular SNP location in the genes and proteins provided herein. One could identify the location of intronic SNPs using the relevant SEQ ID NO: 1-9 and the appropriate coordinates from column 9 of Table 5. For example, to find the location of SNP 803 E+1, one could look at SEQ ID NO:1 at the position indicated by Table 5, in this case coordinate 276365. Alternatively, one could use the coordinate given in column 11 of Table 5 and the appropriate sequence from Table 2 to find the location of a particular SNP.

SEQ ID NOs: 1-9 contain the genomic sequence of Gene 803, Gene 845, Gene 847, Gene 874 and Gene 962. The corresponding accession numbers for these sequences are located within Table 2. SEQ ID NO: 1 contains the genomic sequence of Gene 803. SEQ ID NOs: 2-5 taken together contain the genomic sequence of Gene 845. SEQ ID NOs: 6 and 7 taken together contain the genomic sequence of Gene 847. SEQ ID NO: 8 contains the genomic sequence of Gene 874. SEQ ID NO: 9 contains the genomic sequence of Gene 962.

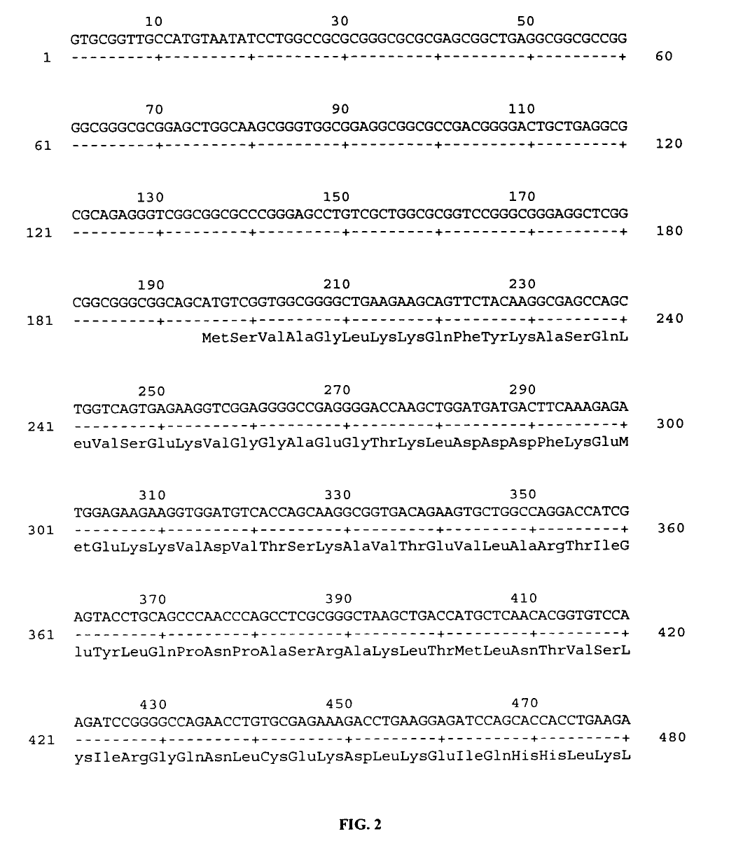

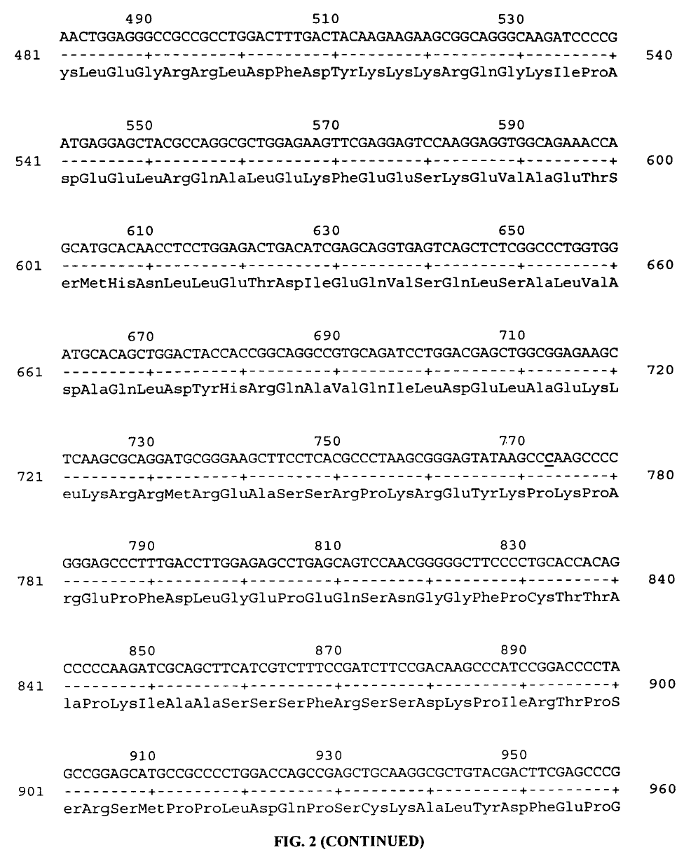

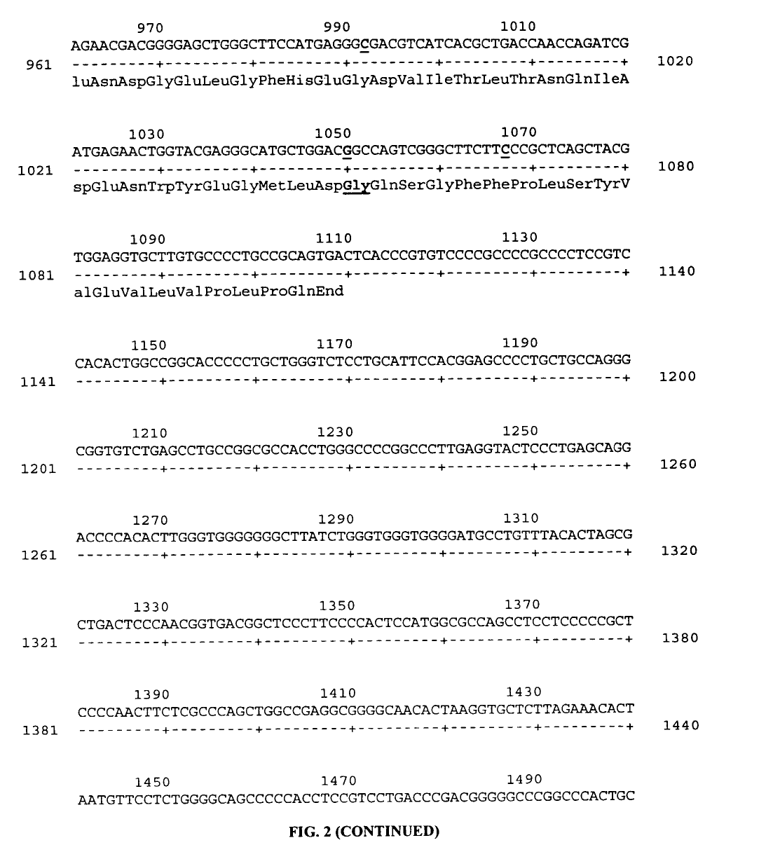

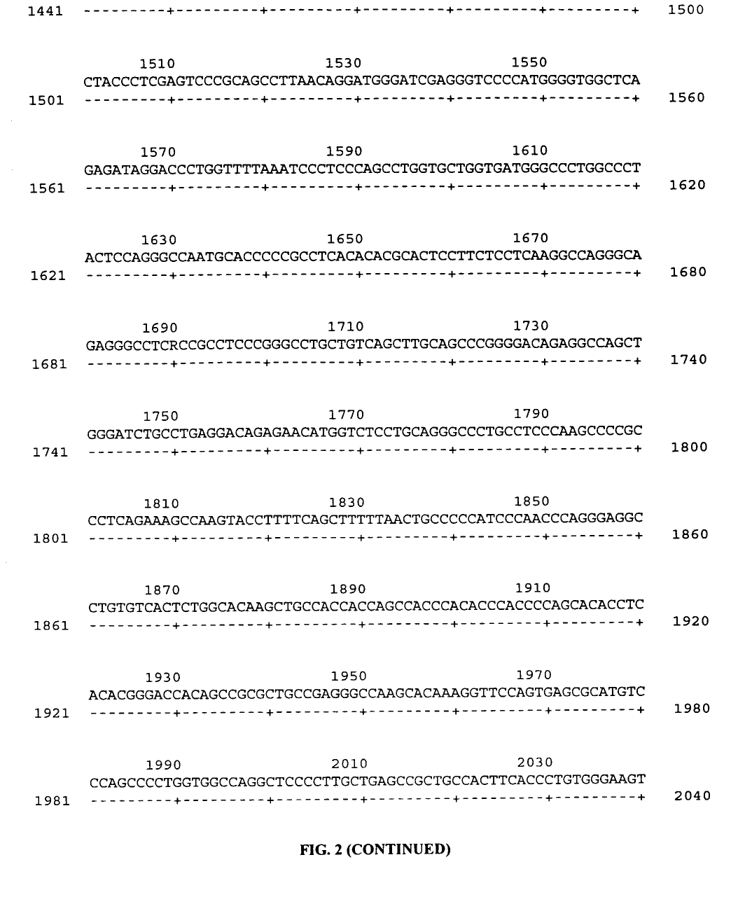

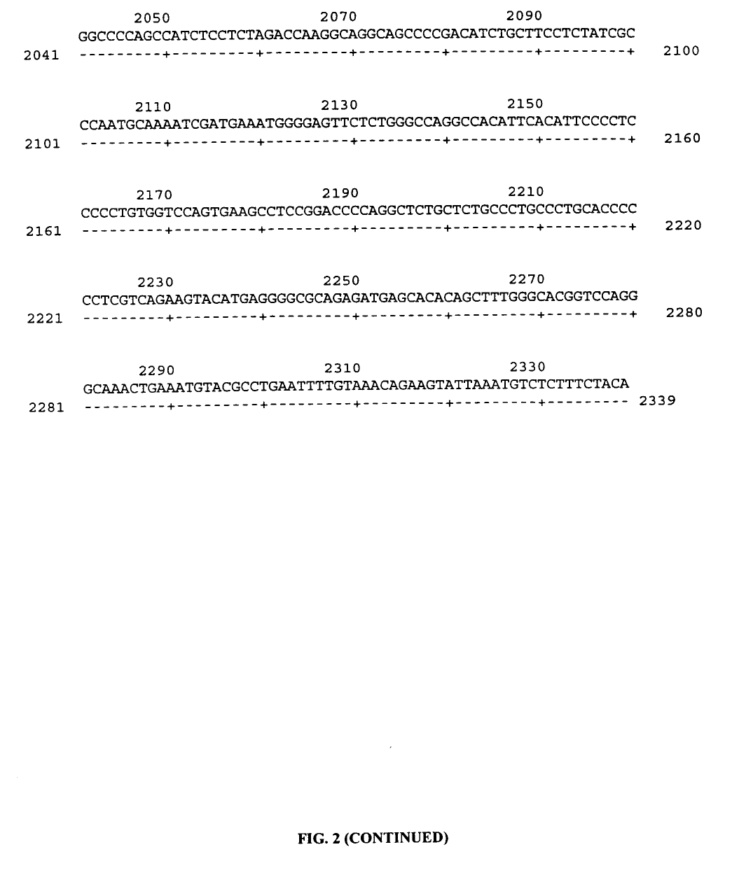

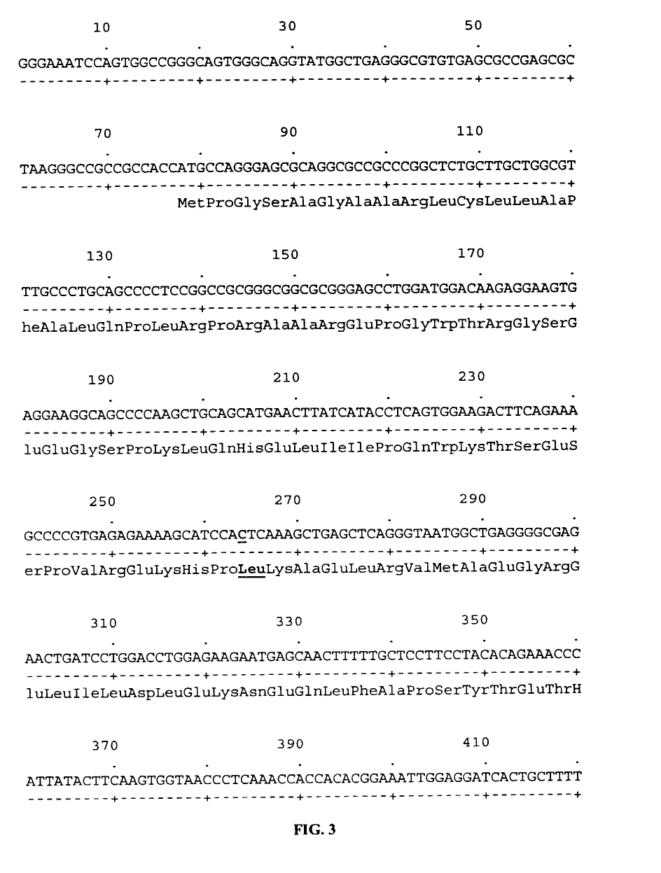

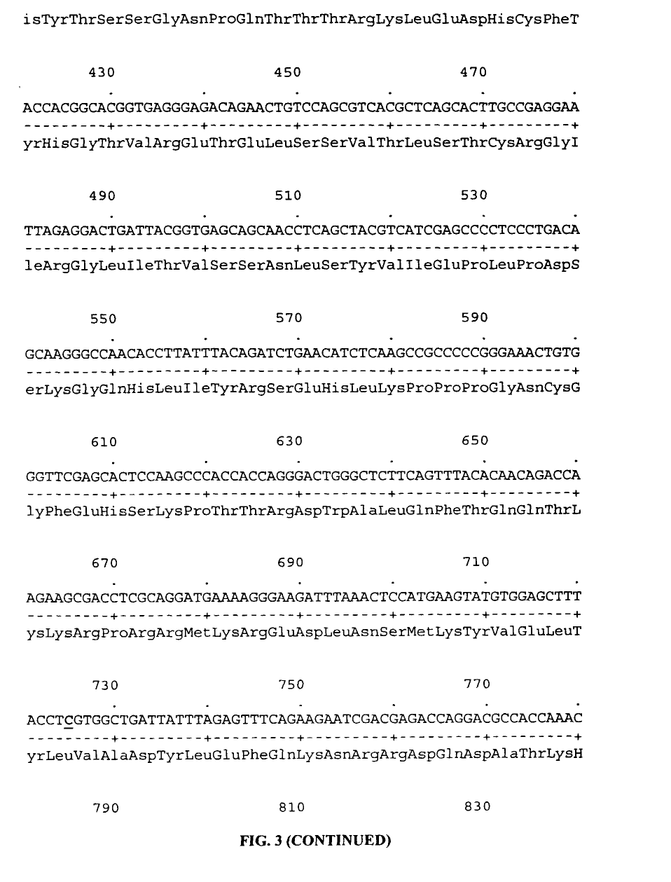

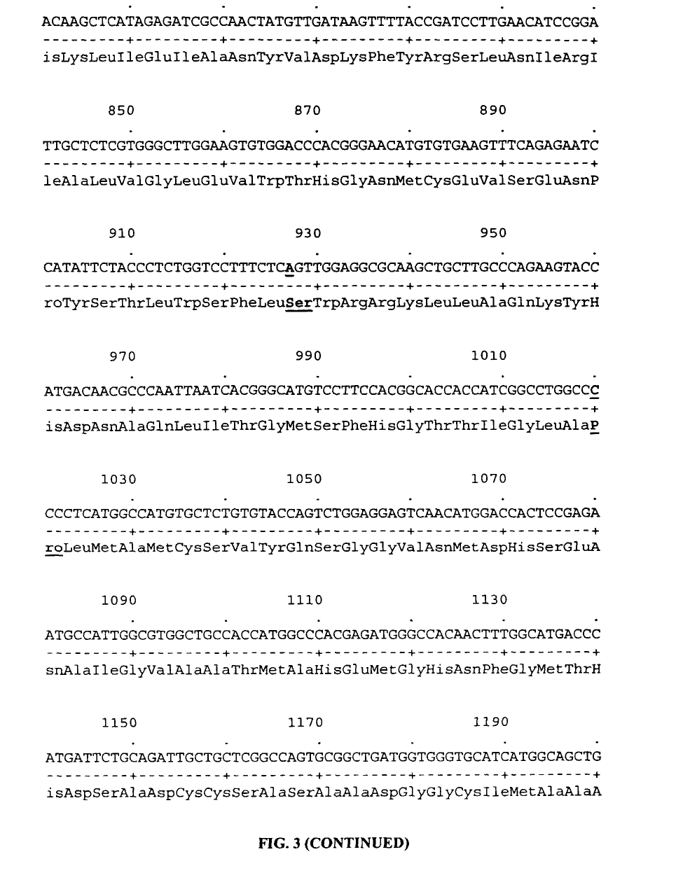

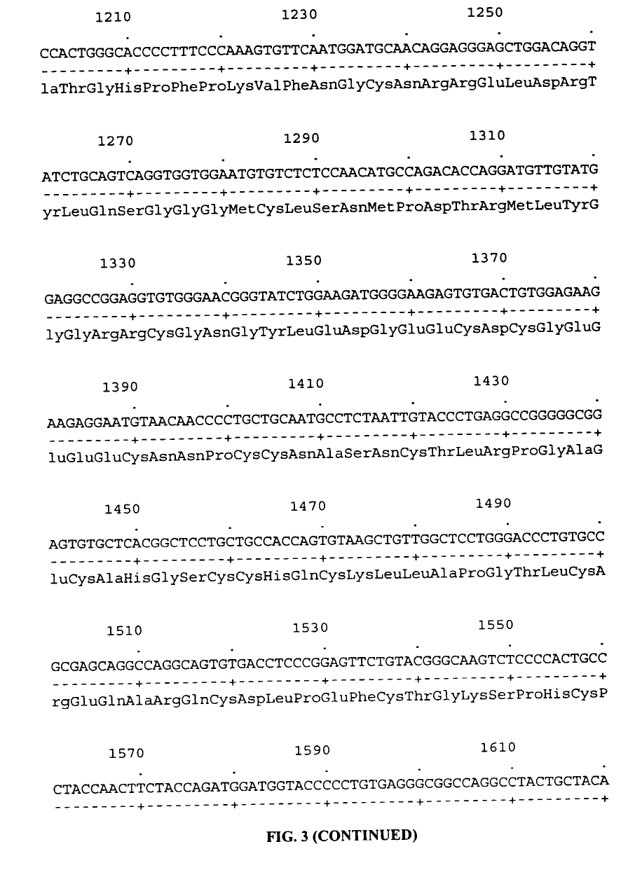

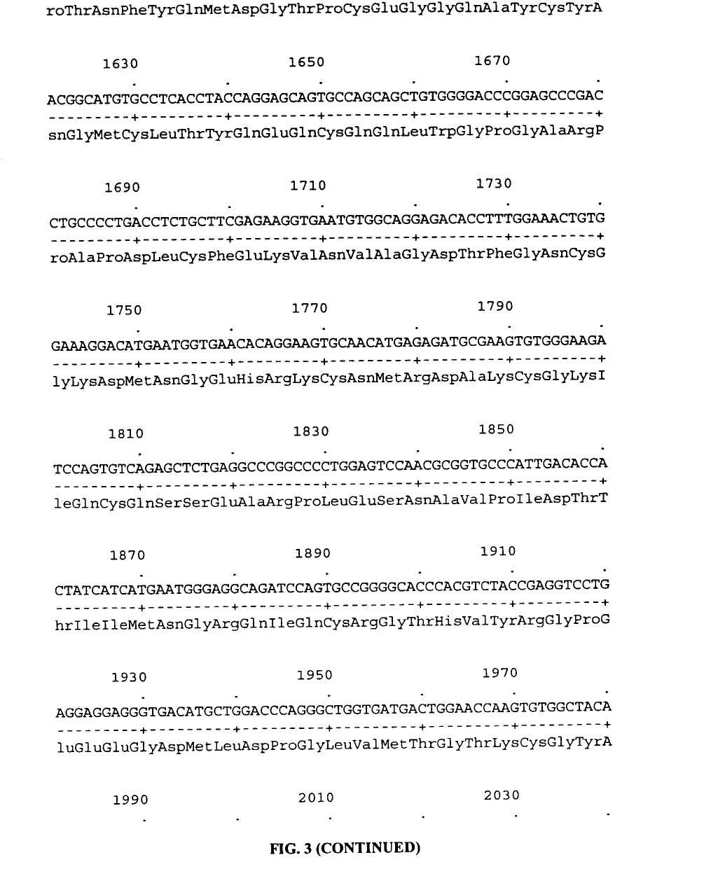

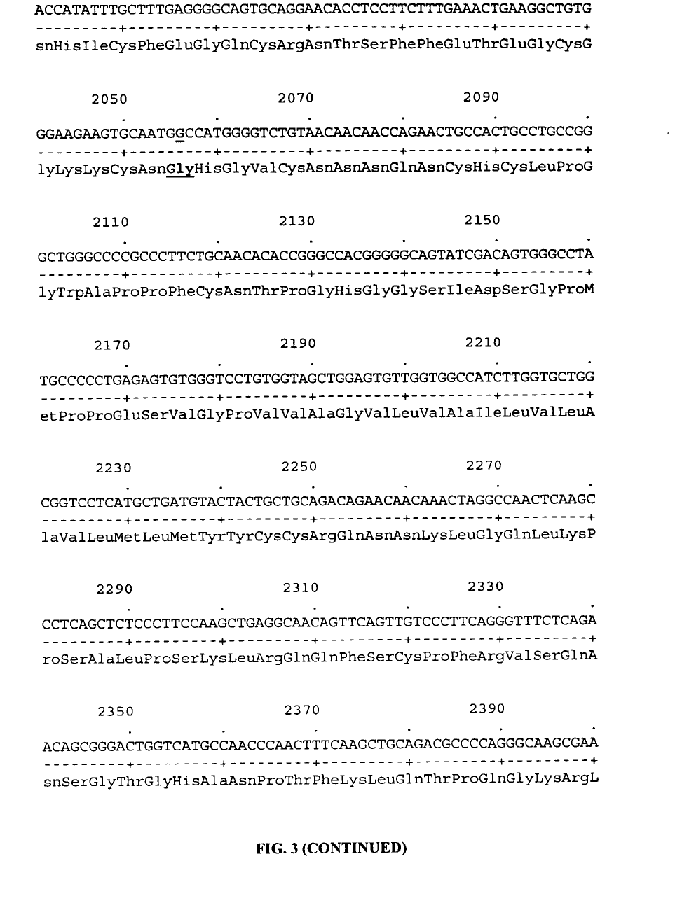

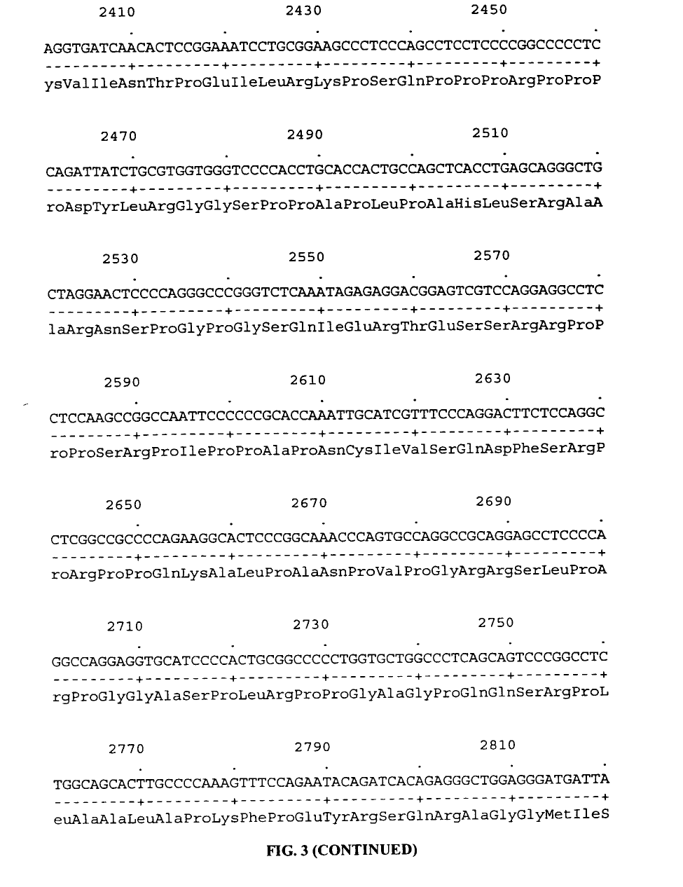

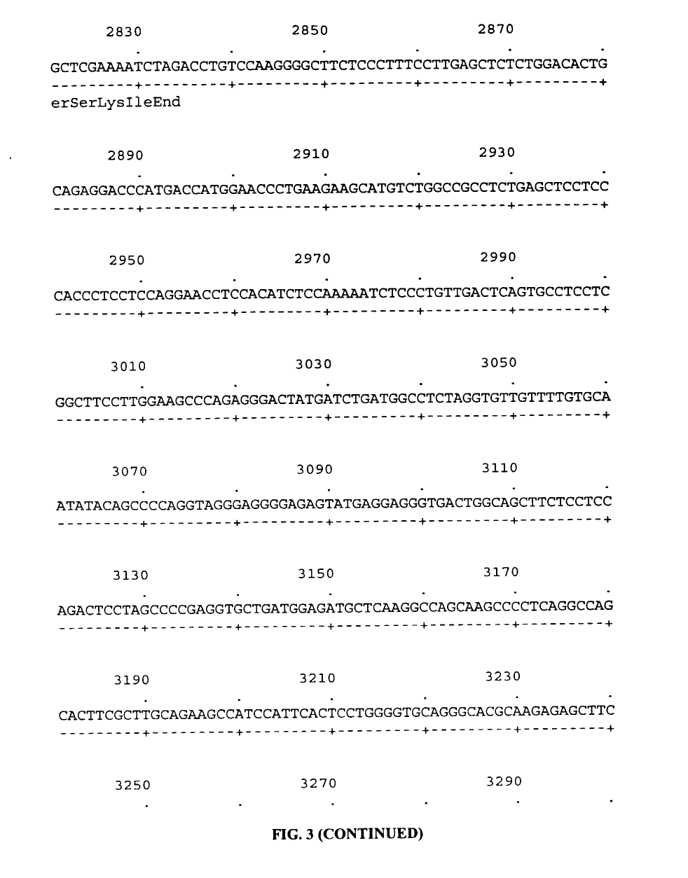

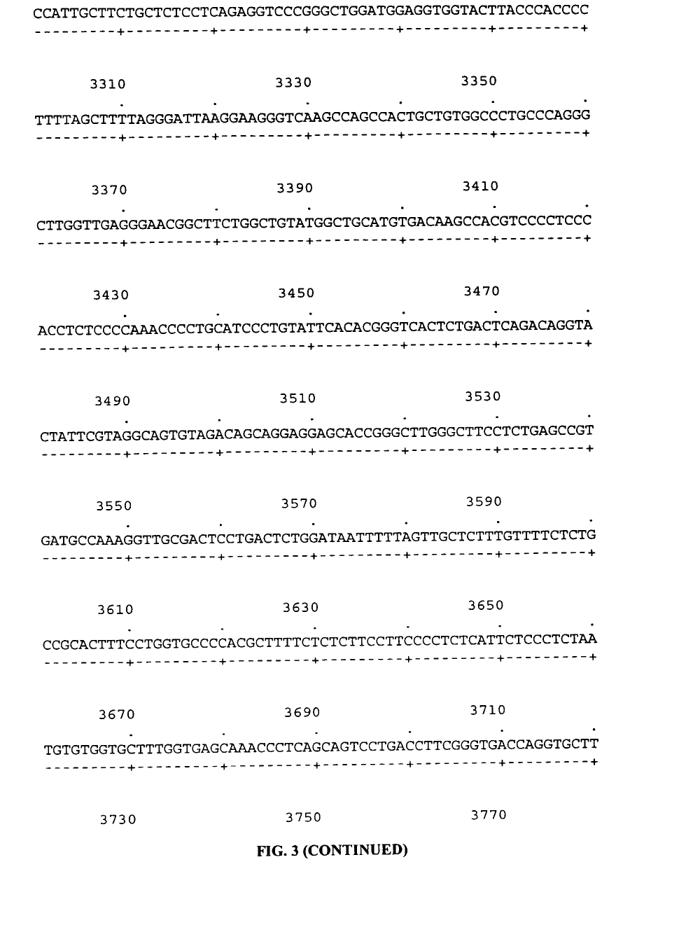

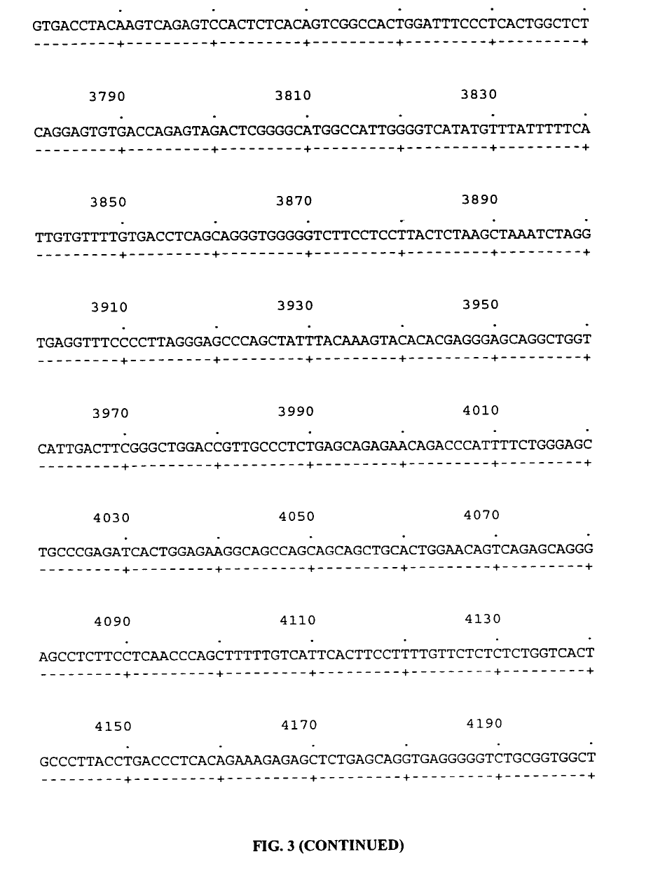

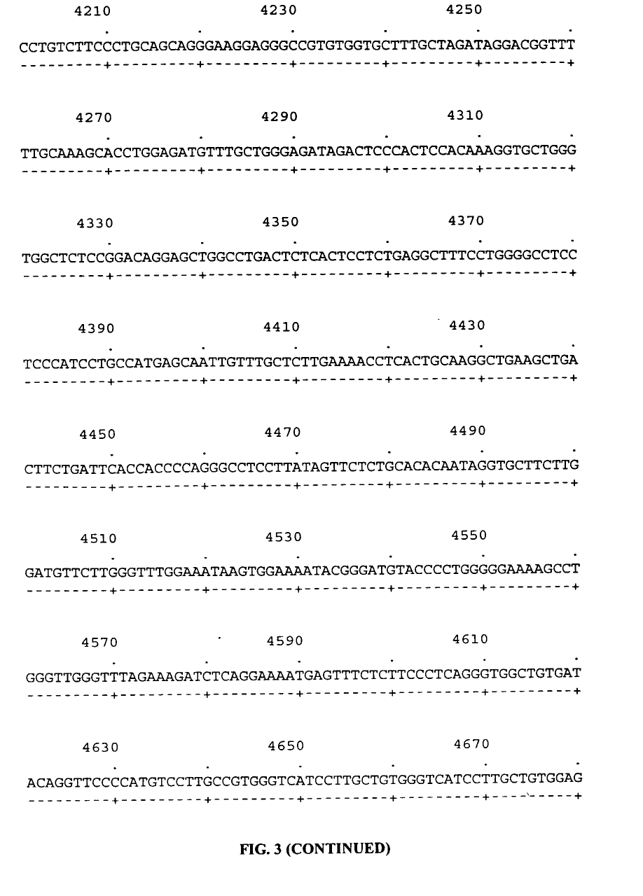

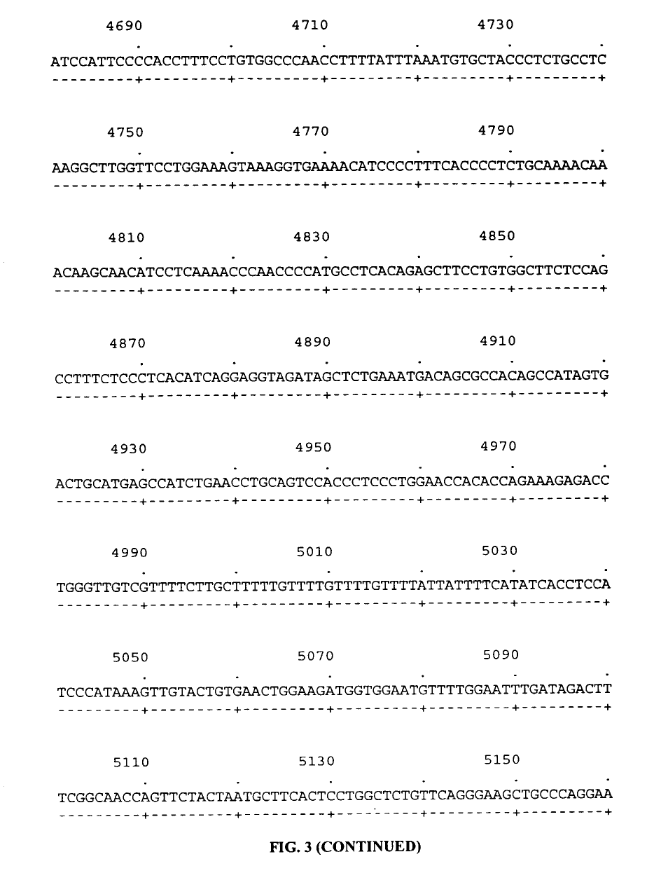

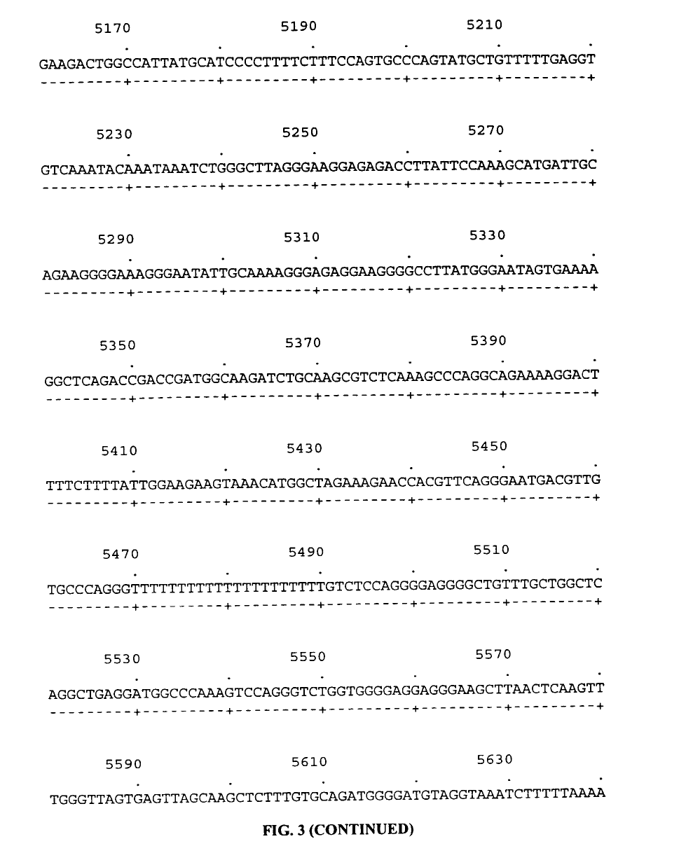

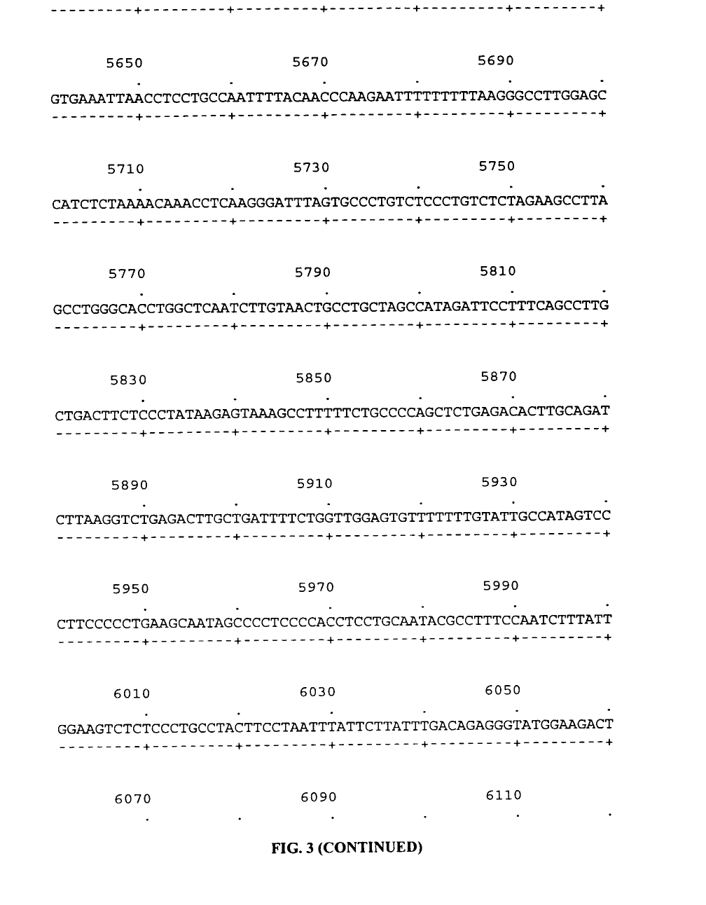

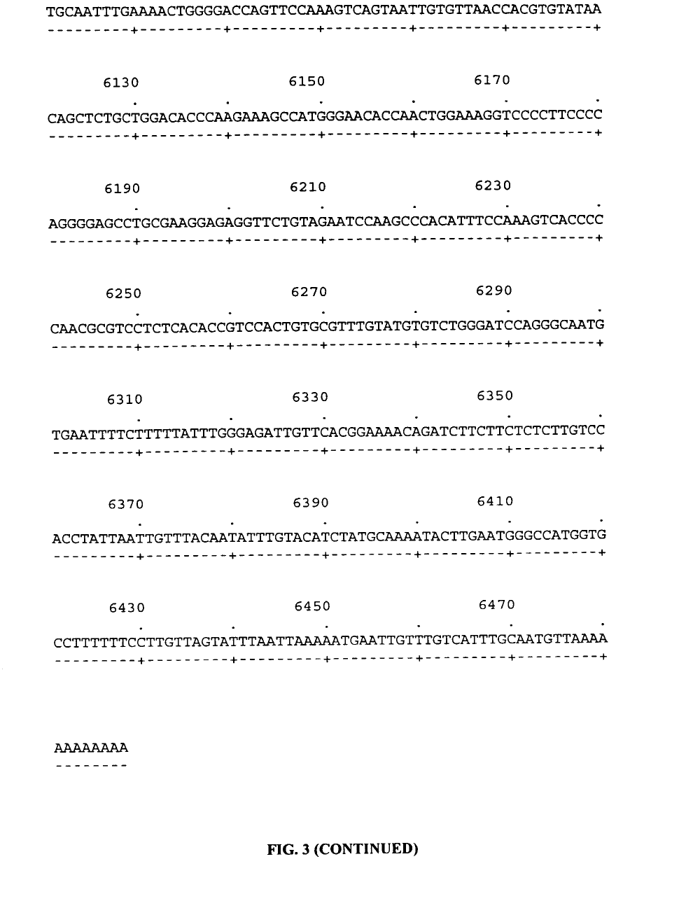

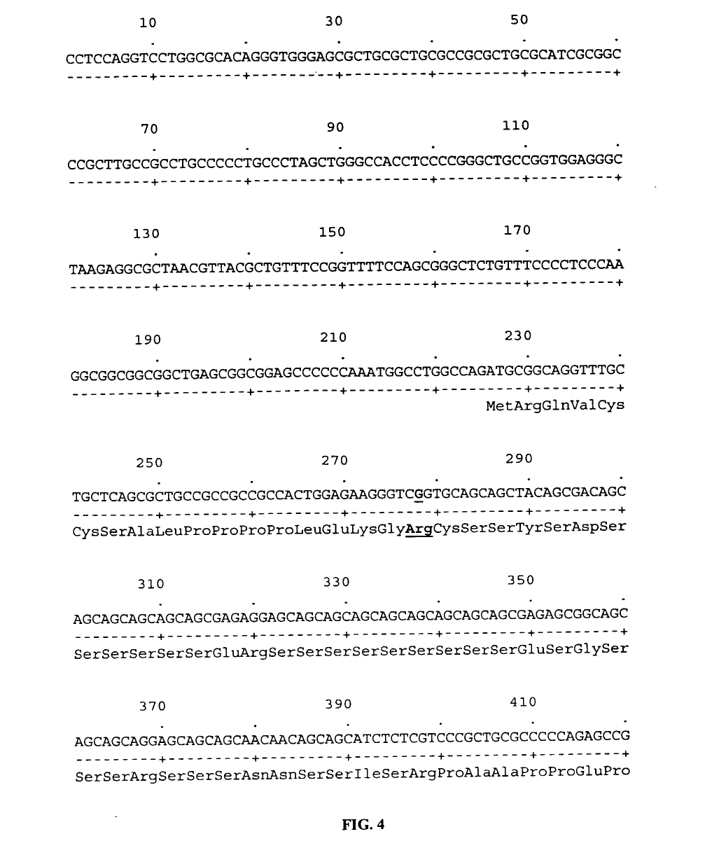

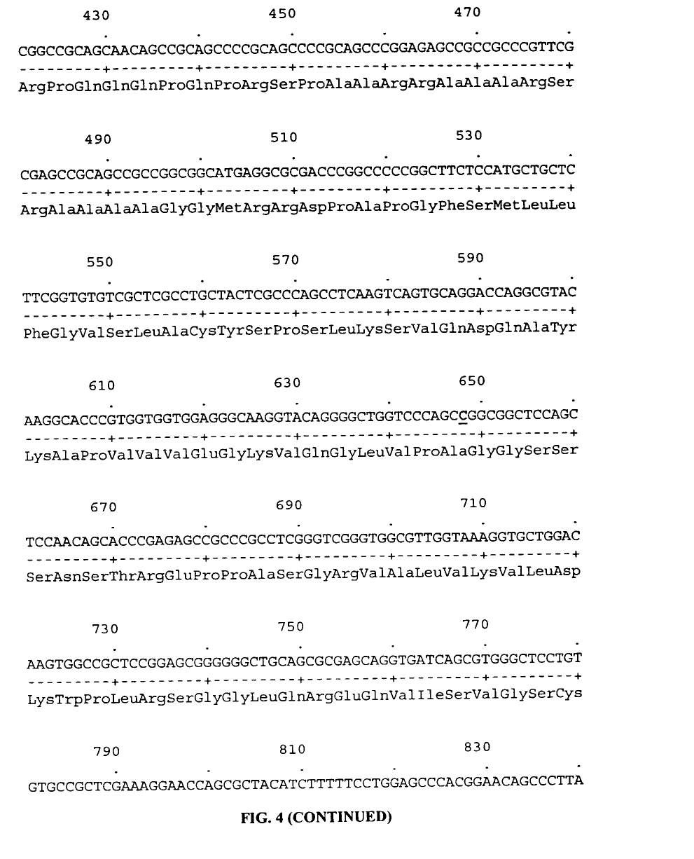

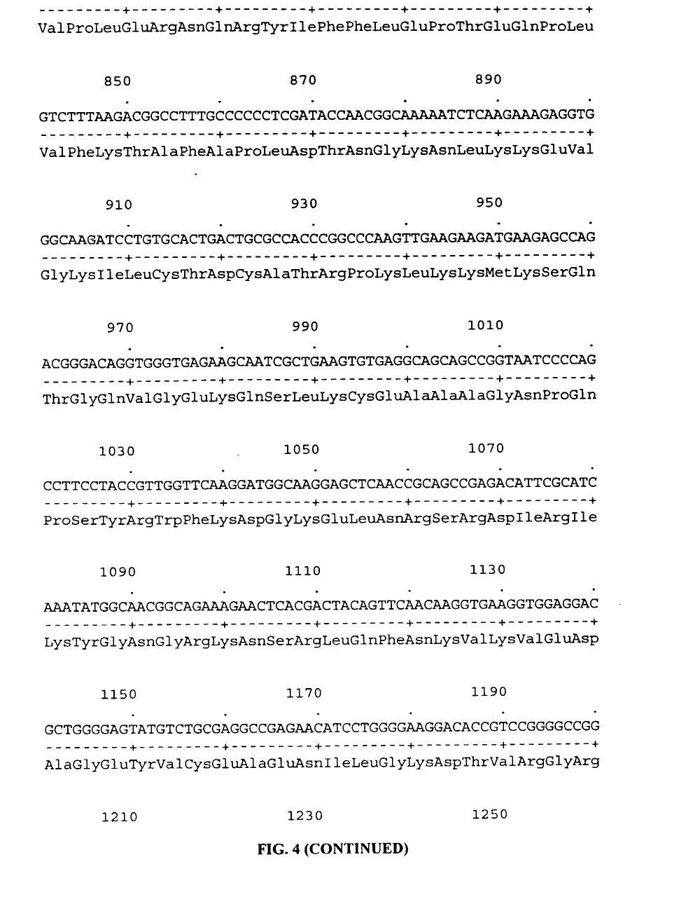

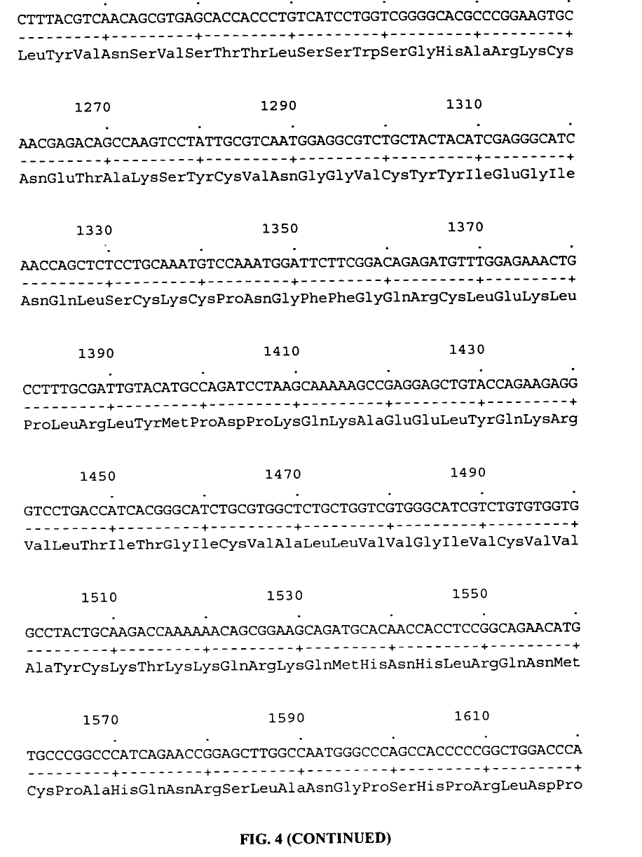

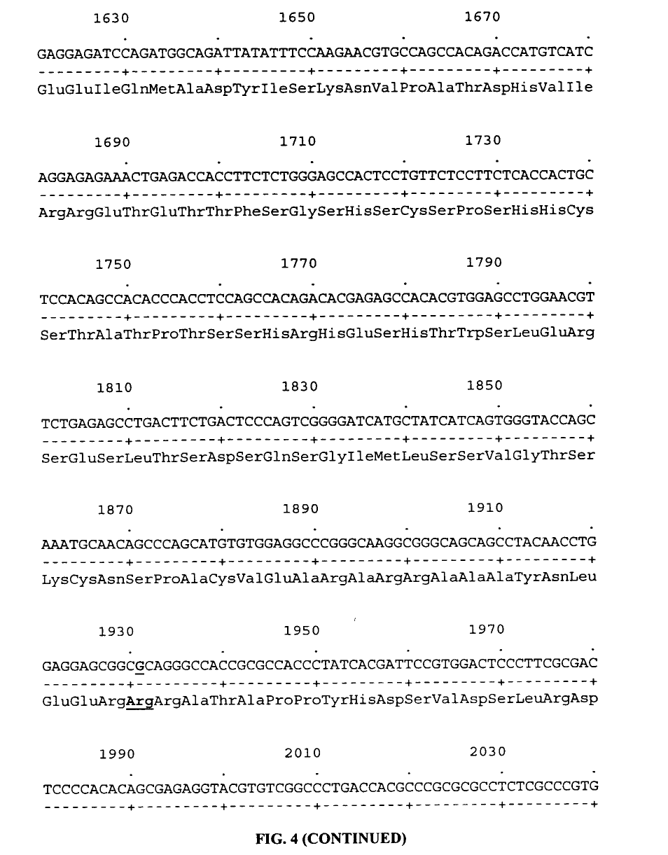

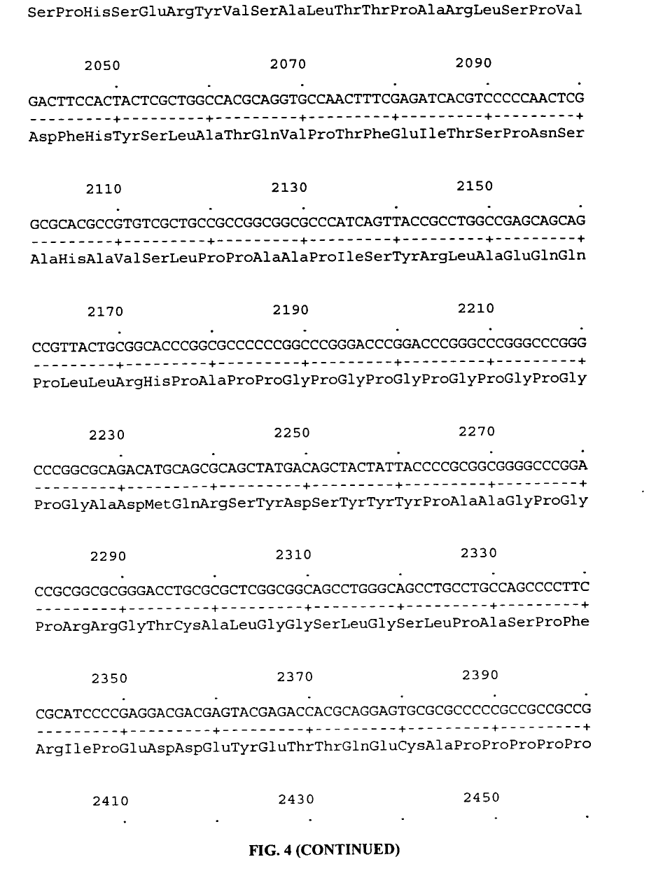

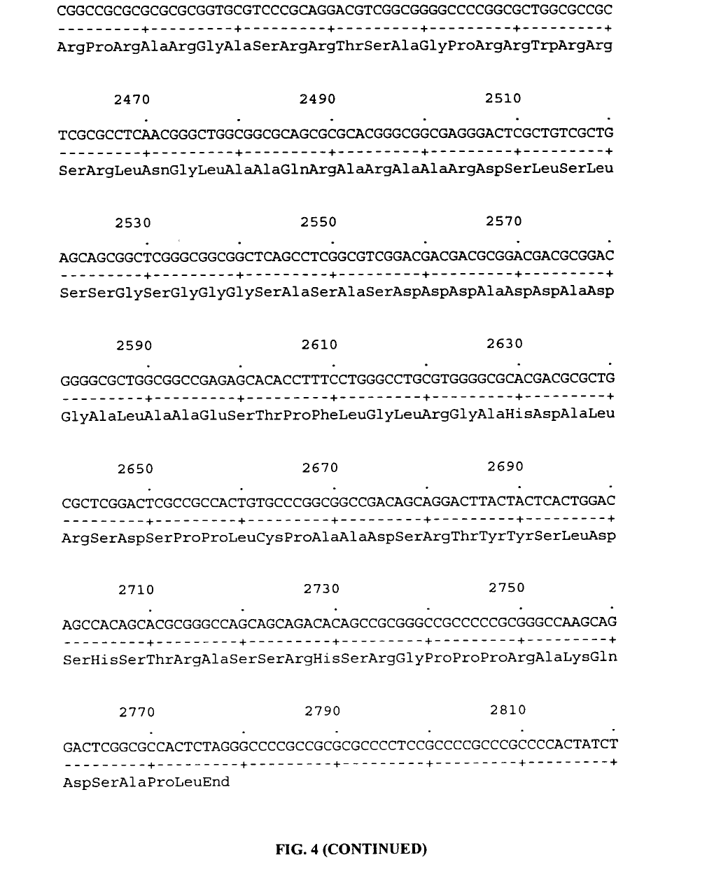

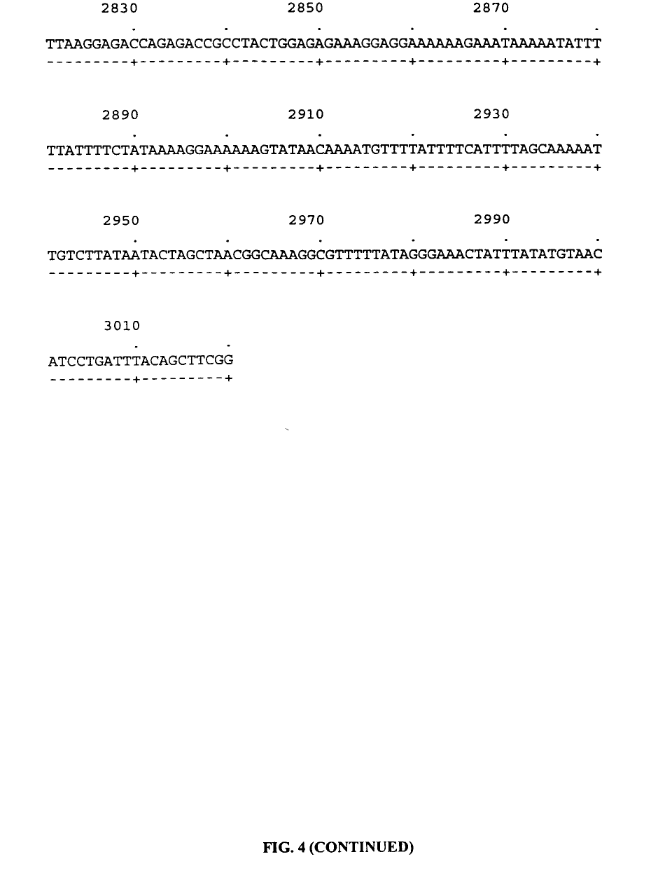

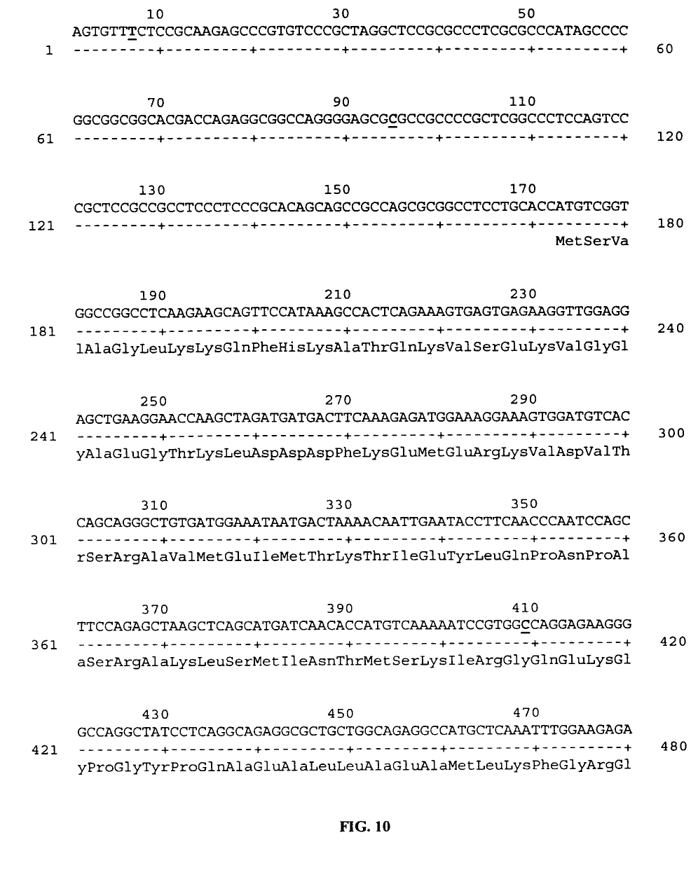

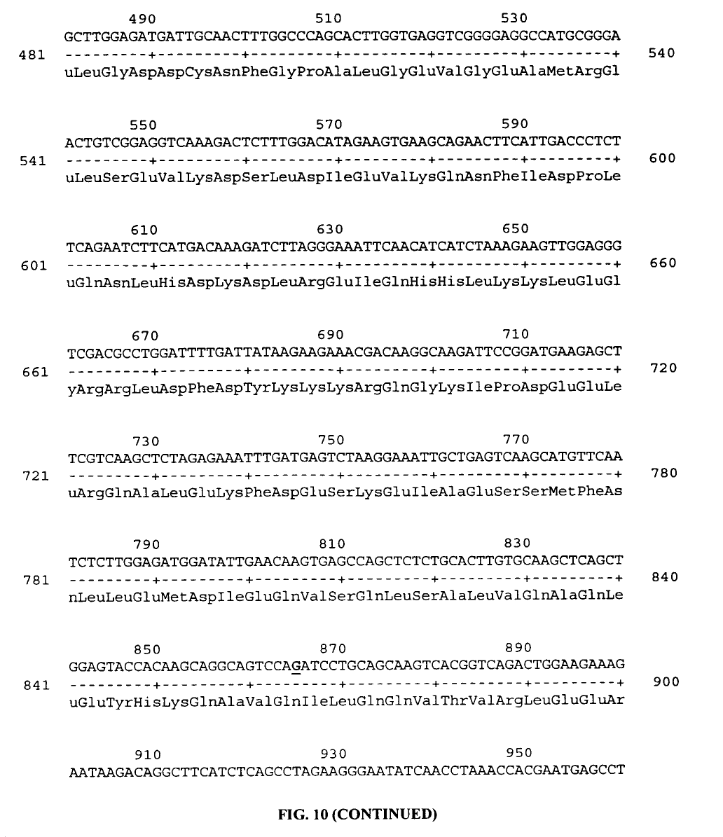

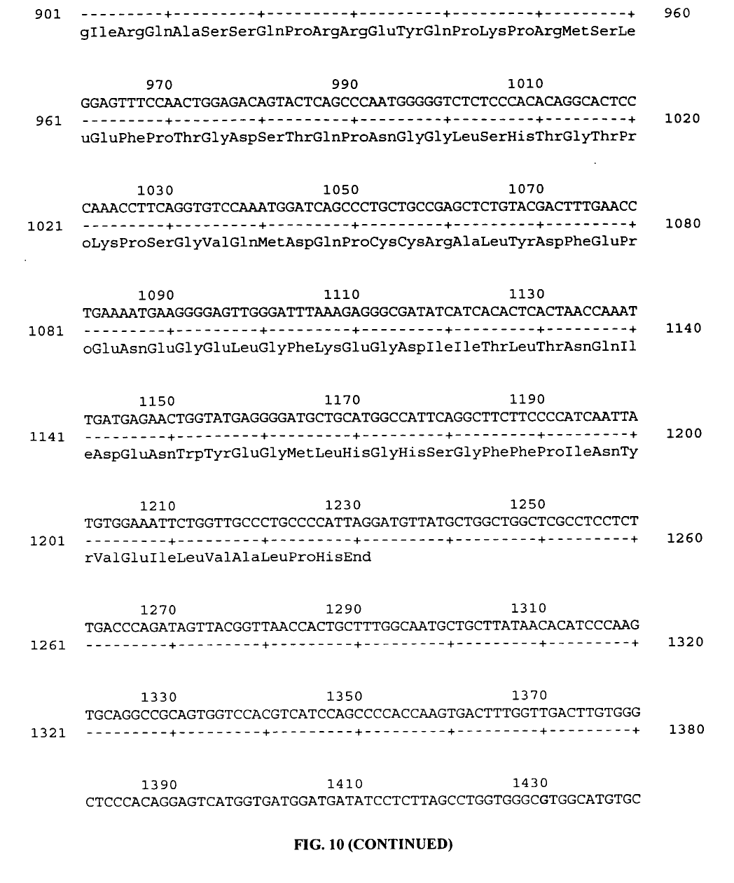

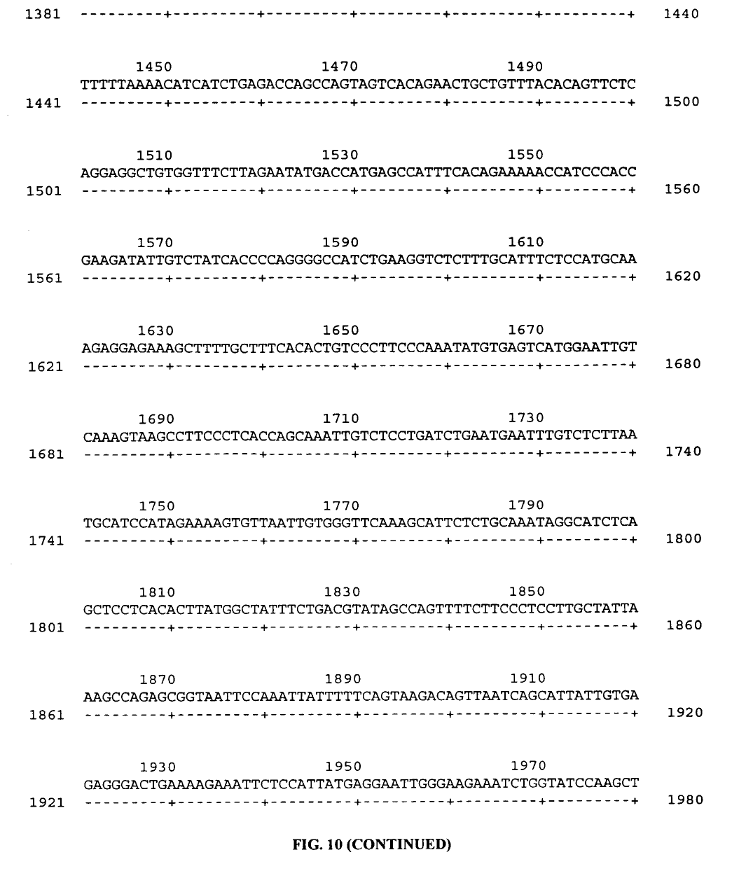

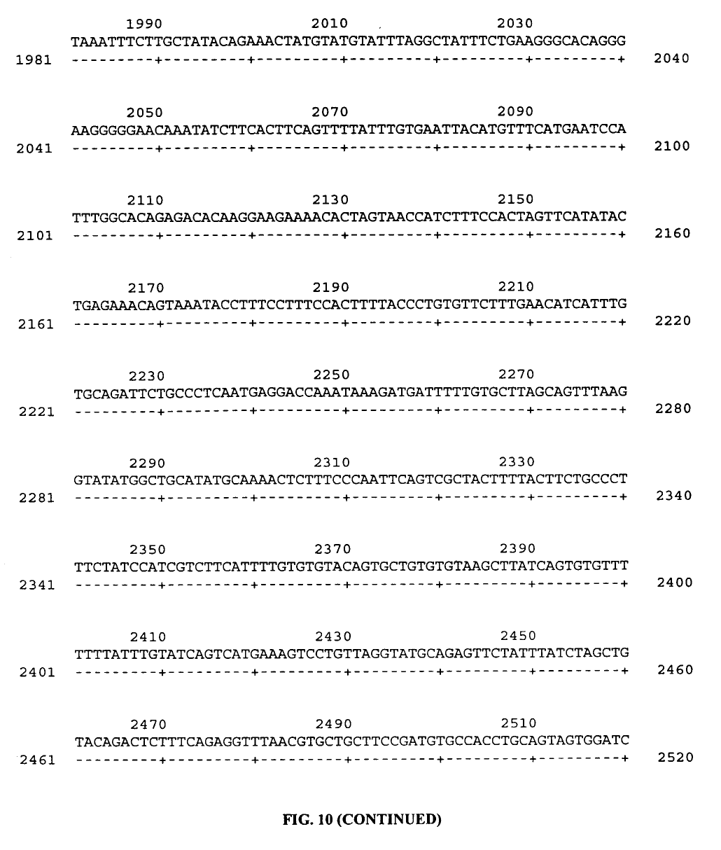

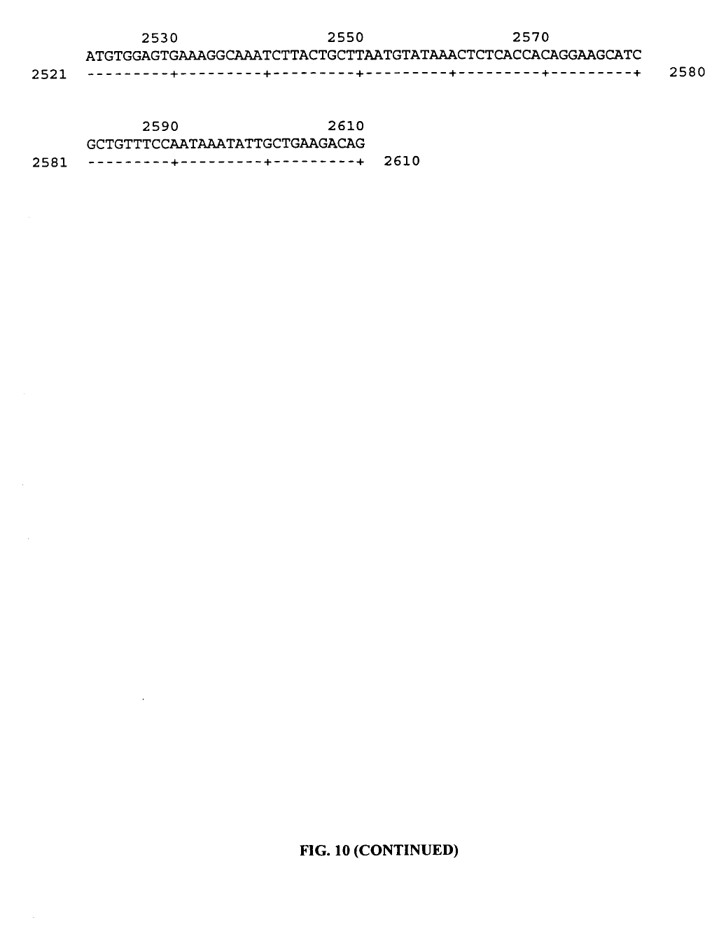

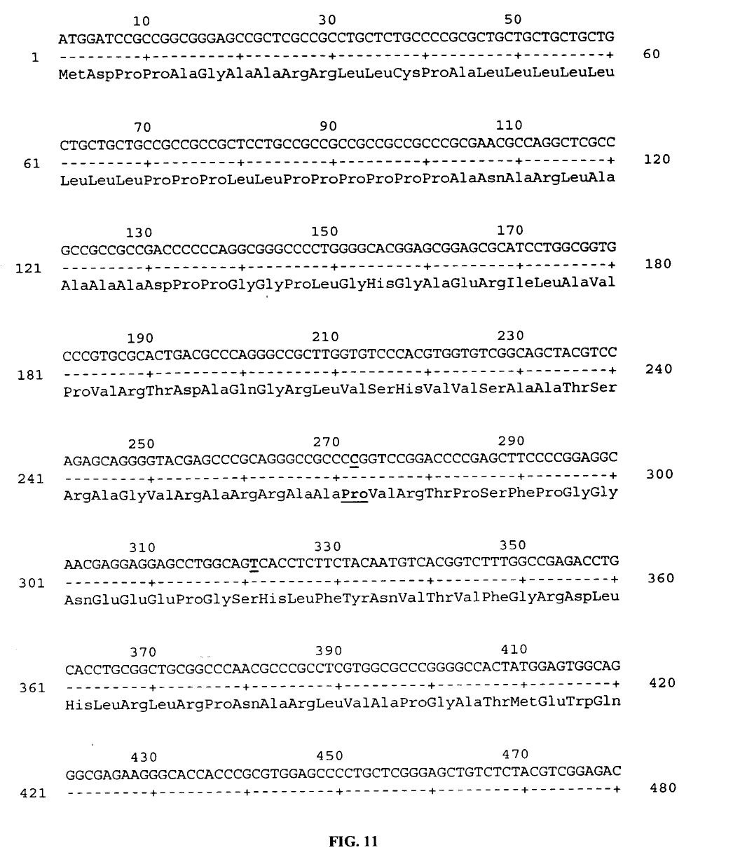

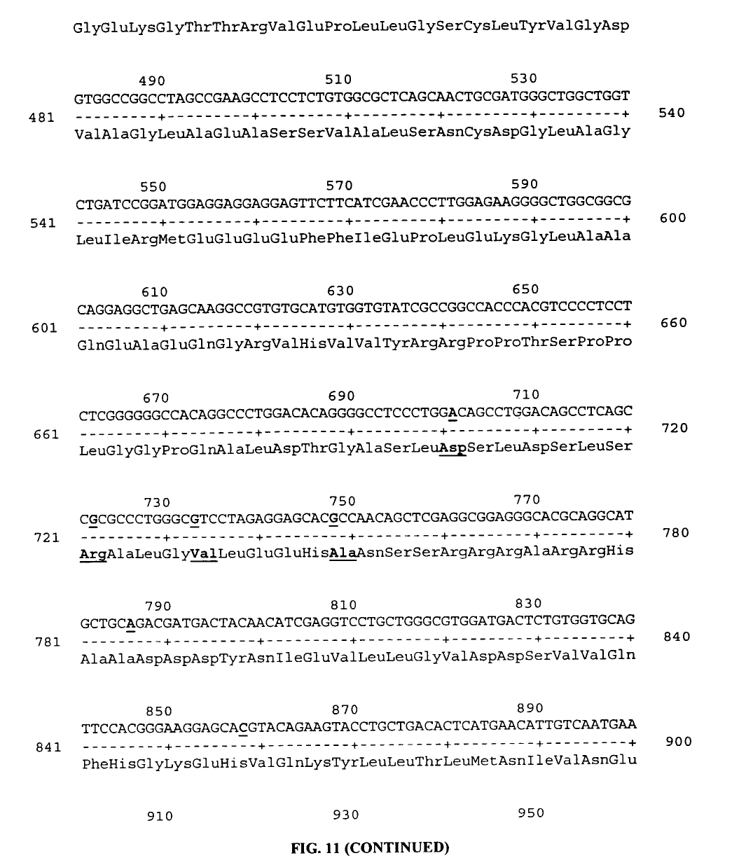

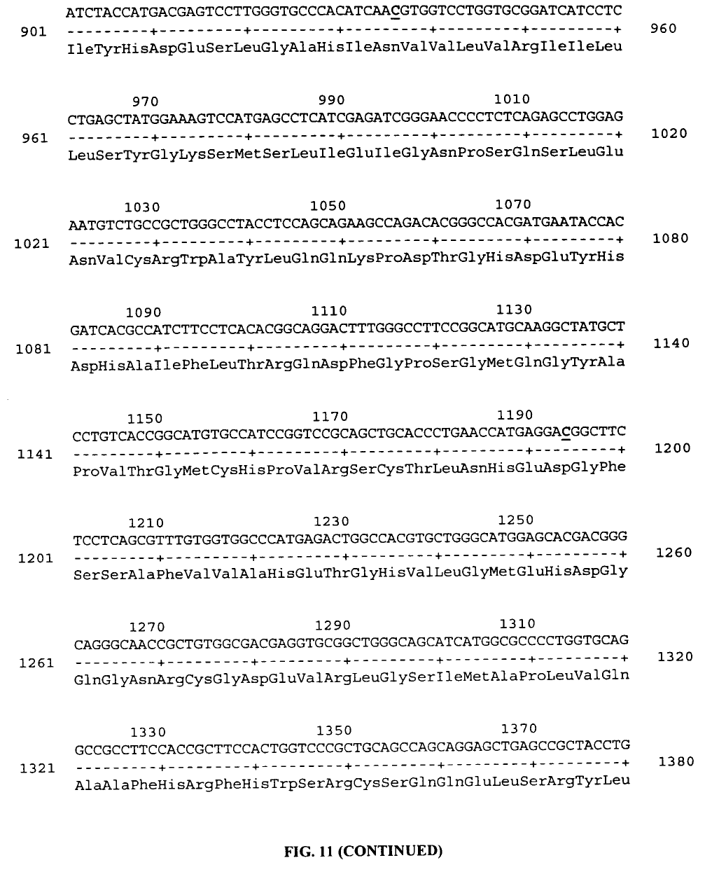

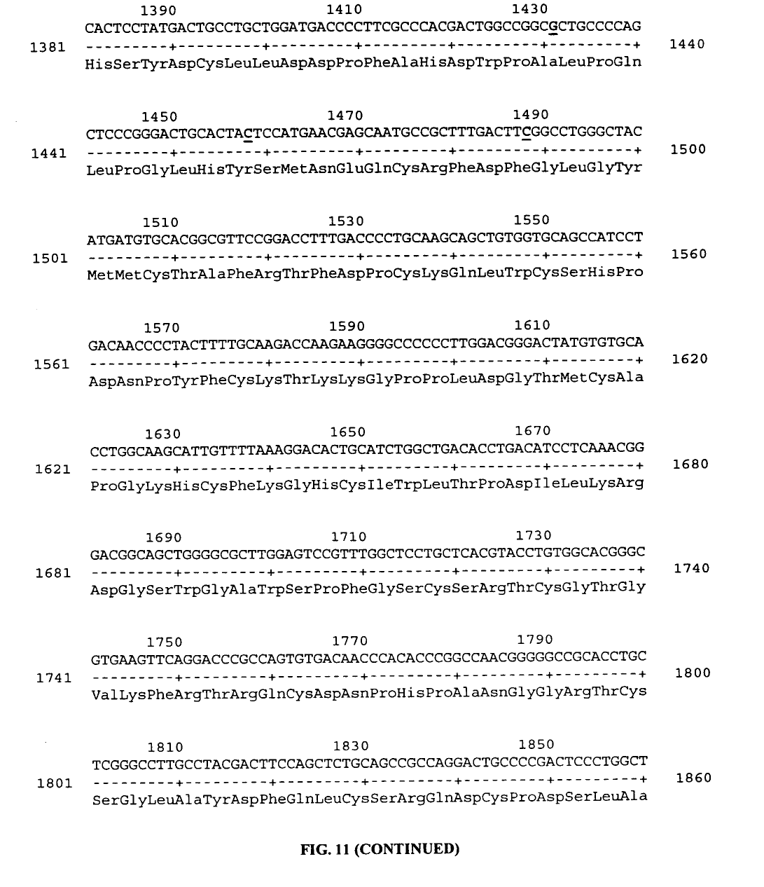

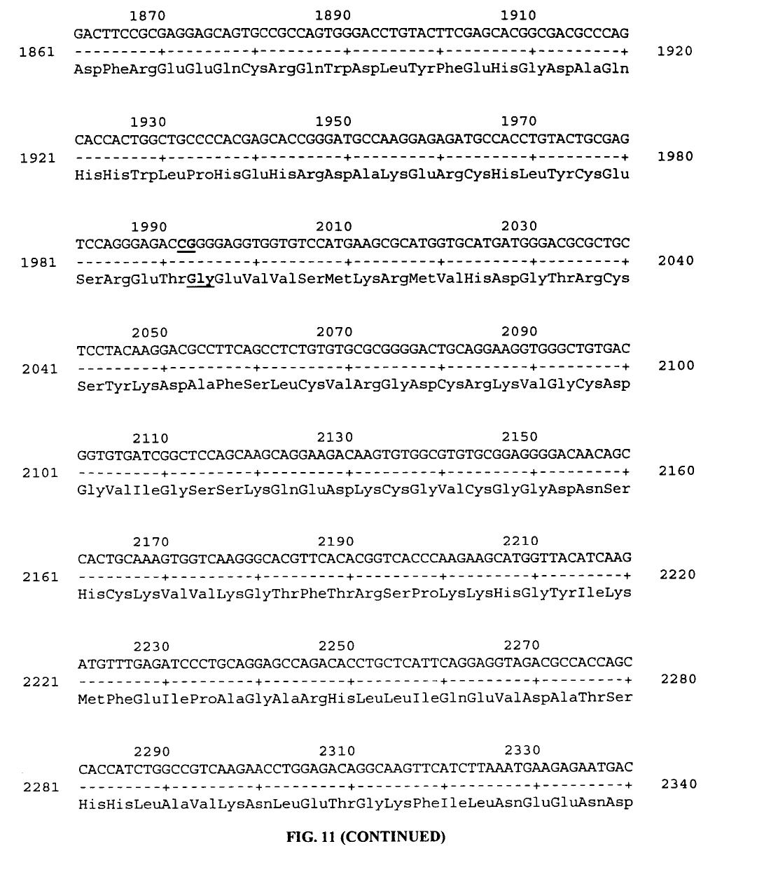

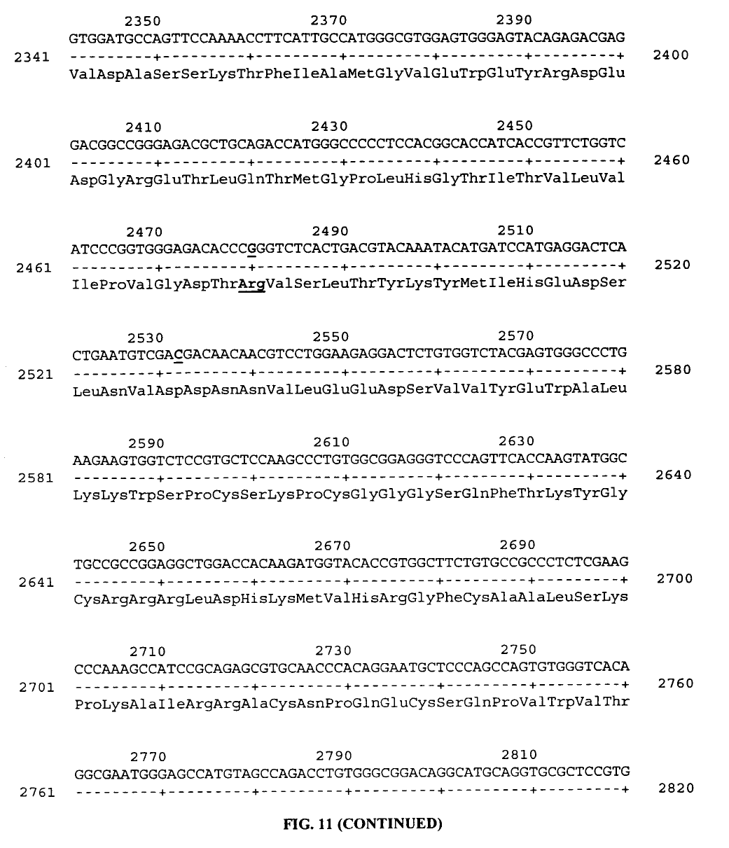

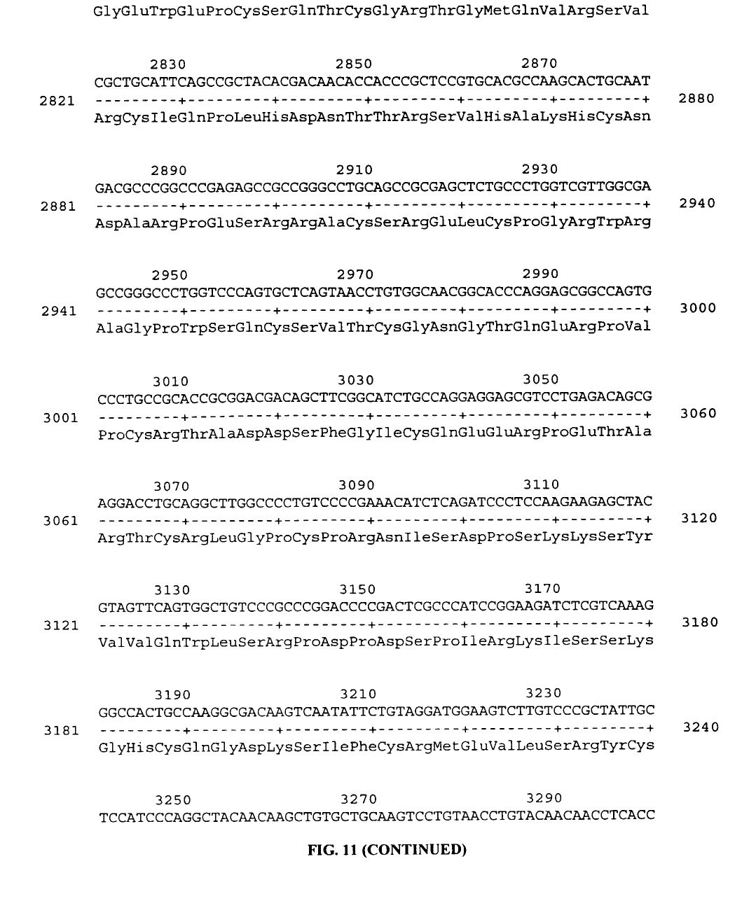

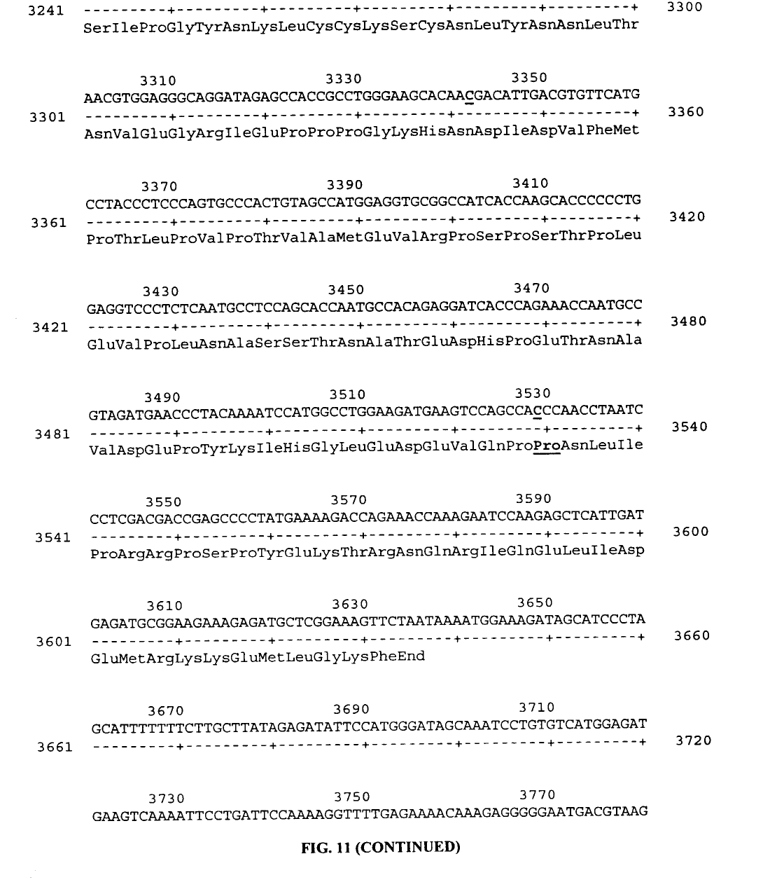

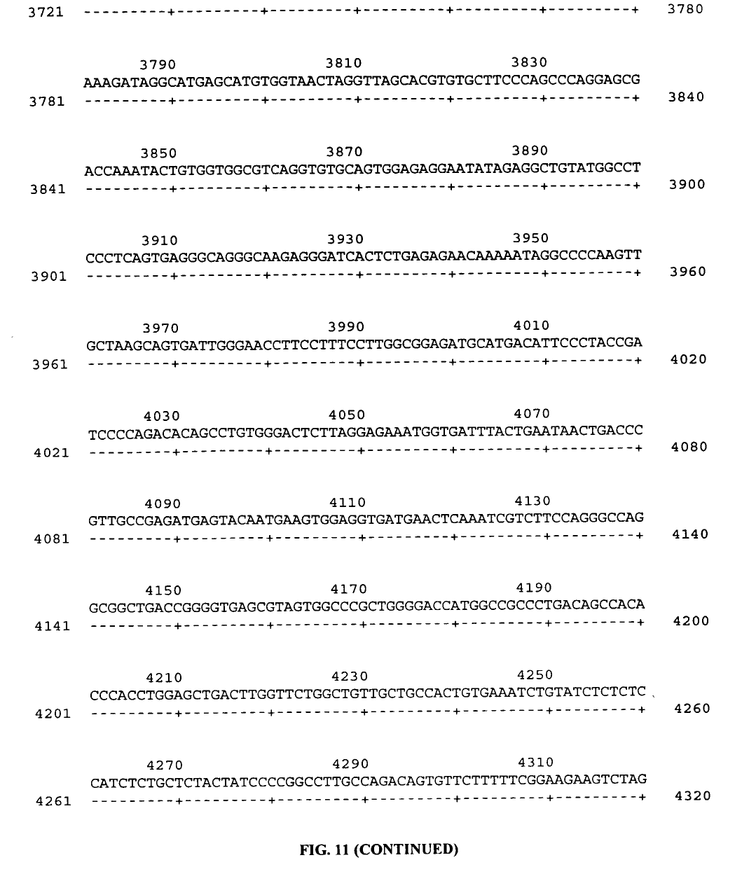

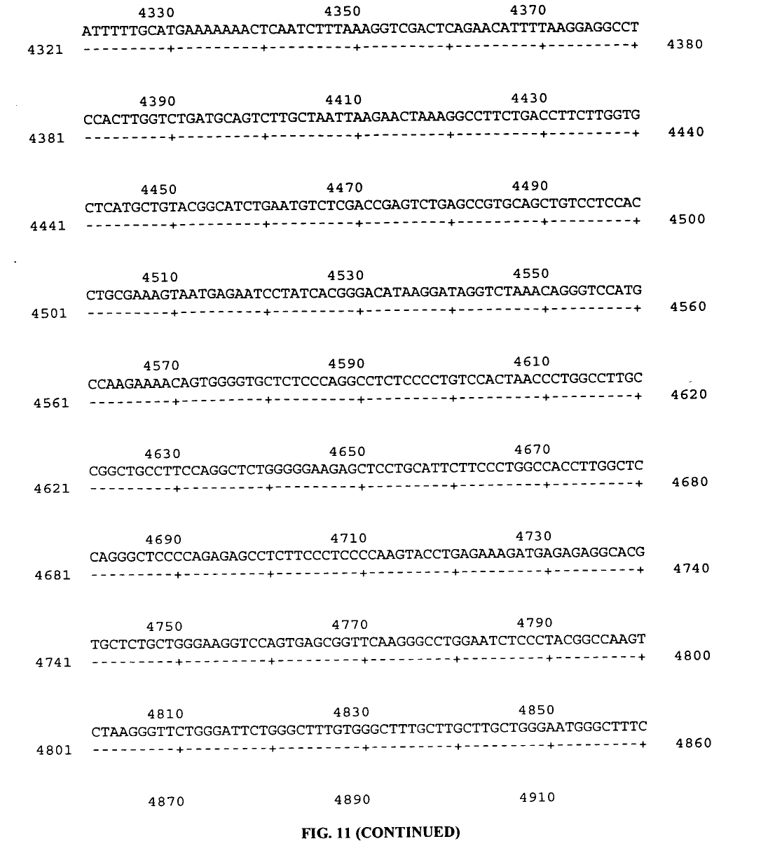

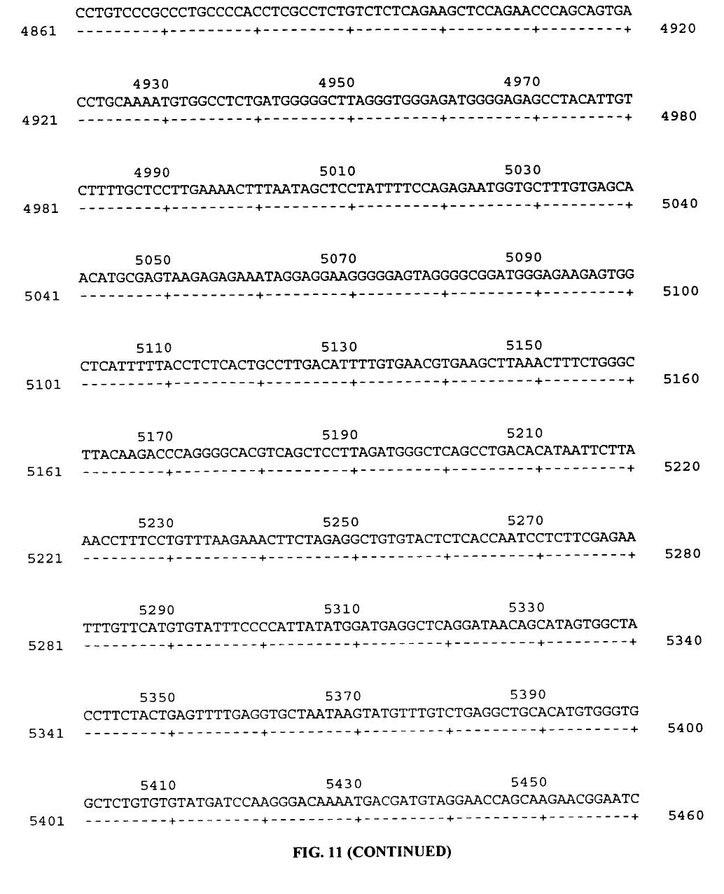

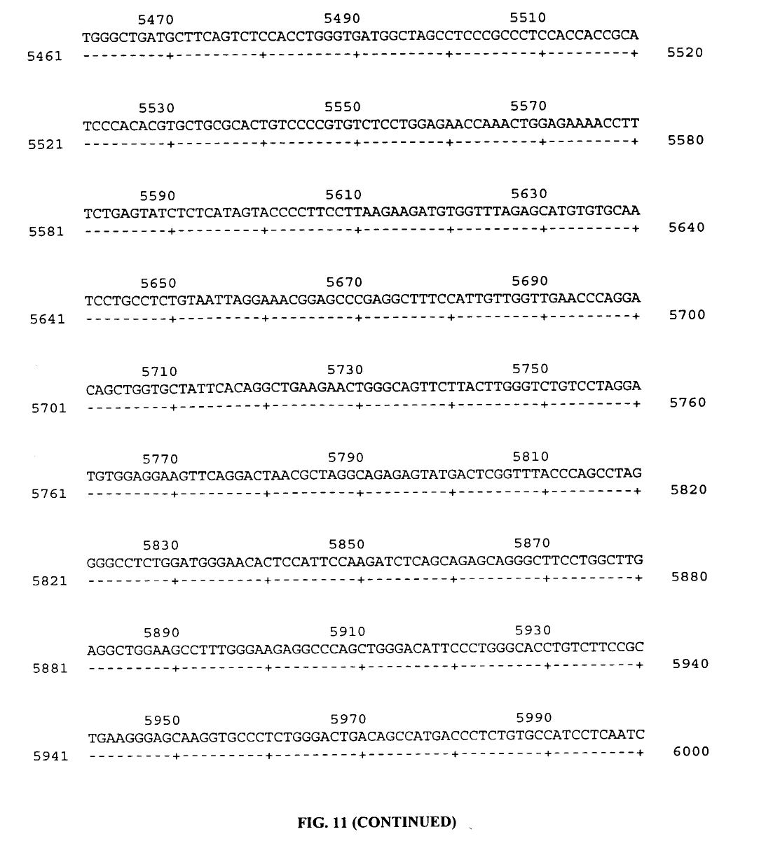

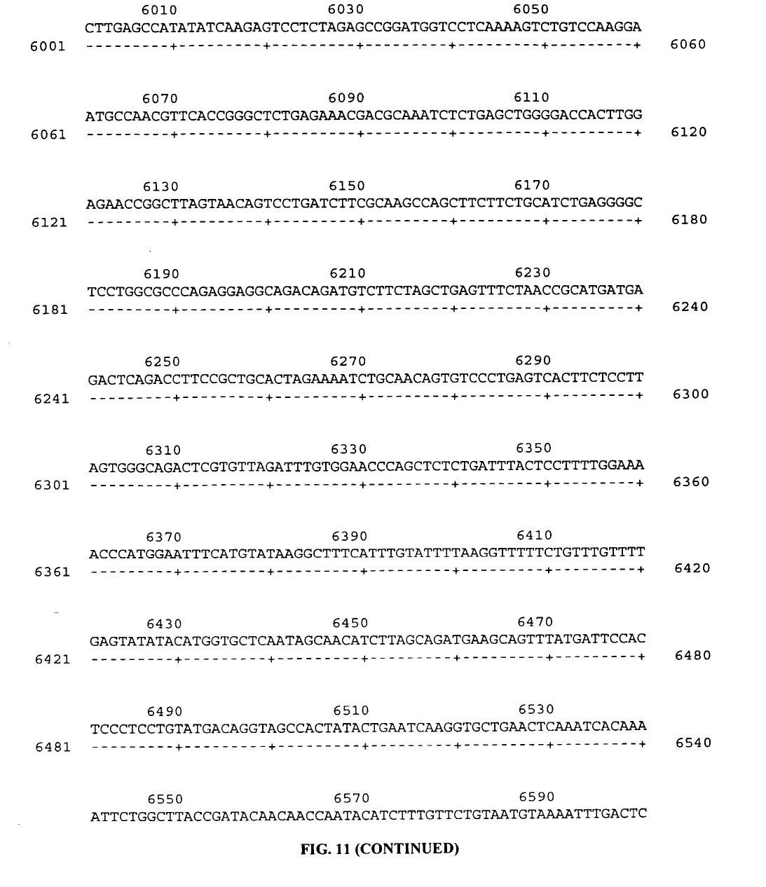

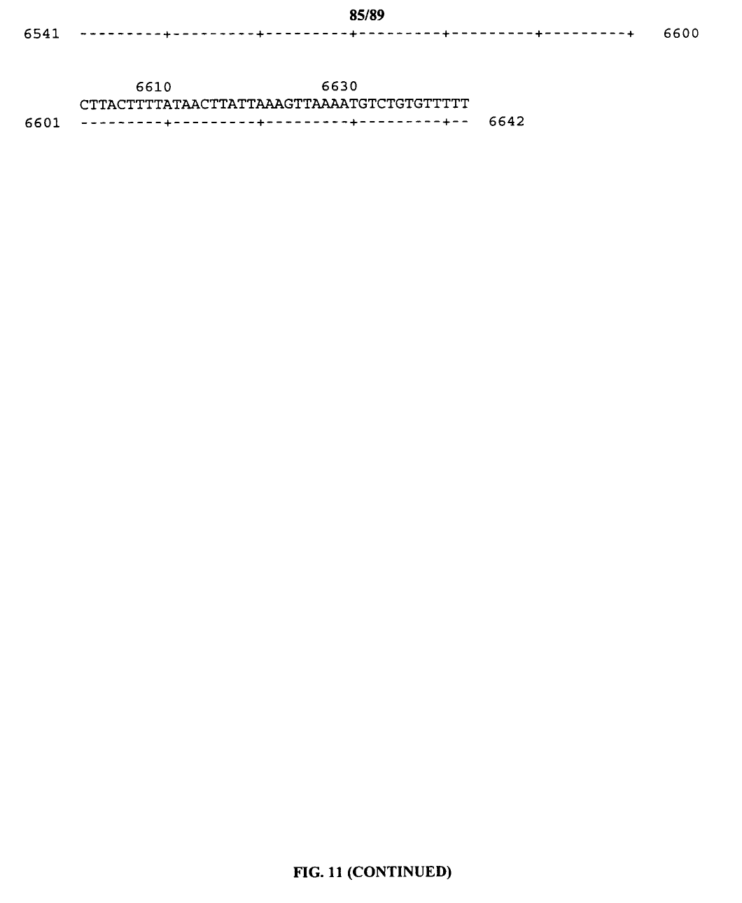

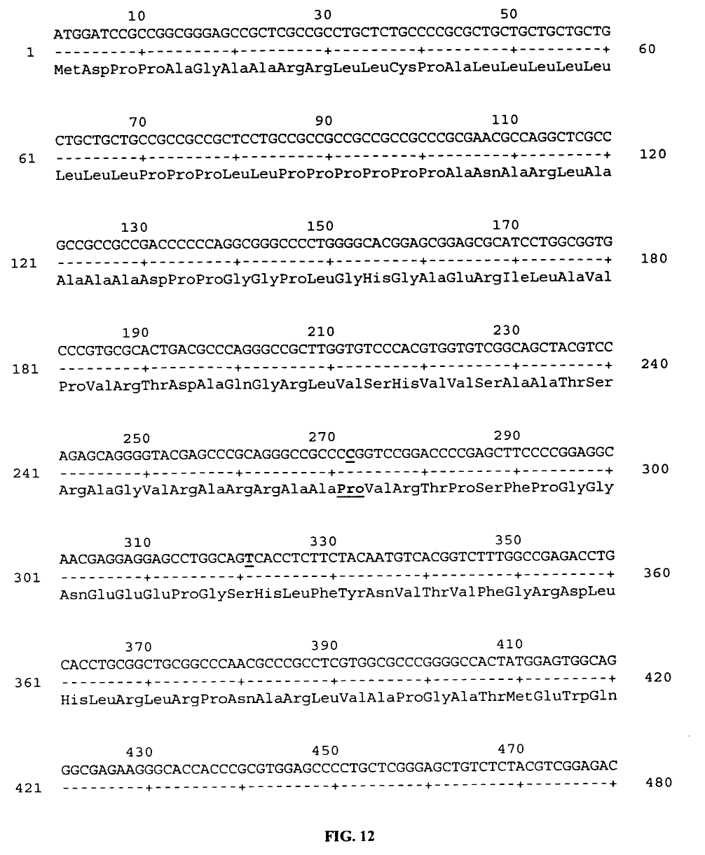

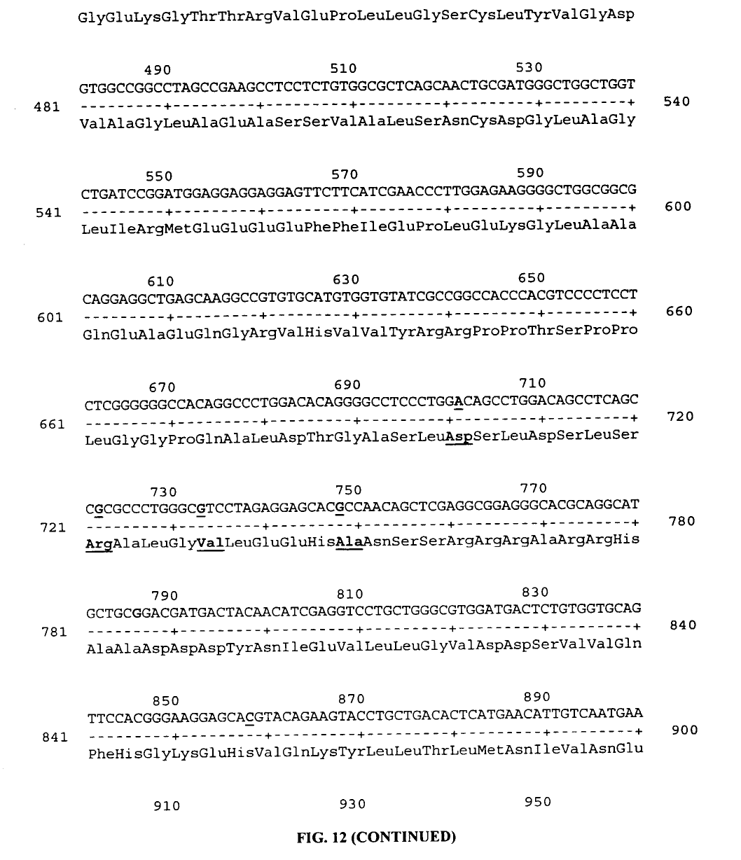

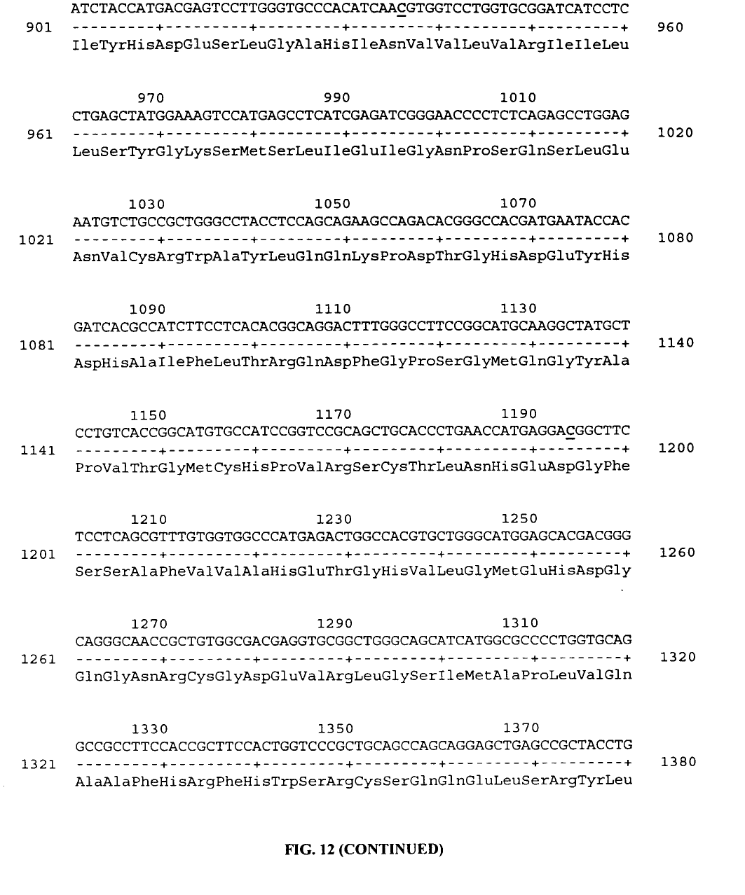

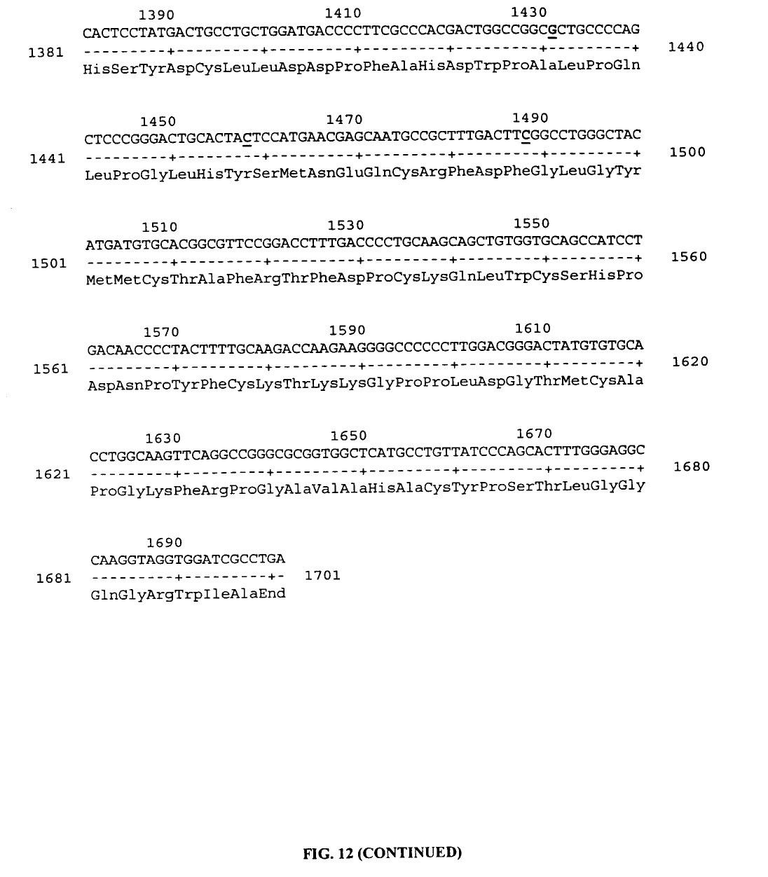

FIGS. 1-12 contain the cDNA and protein sequences with the corresponding SNP locations boldfaced and underlined for Gene 803, Gene 845, Gene 847, Gene 874 and Gene 962. FIGS. 1 and 2 contain the cDNA sequence and protein for two alternate splice variants of Gene 803. FIG. 3 contains the cDNA sequence and protein of Gene 845. FIGS. 4-9 contain the six alternate splice variants of Gene 847. FIG. 10 contains the cDNA and protein sequence of Gene 874. FIGS. 11 and 12 contain the cDNA and protein sequence of two alternate splice variants of Gene 962. Table 2 also contains the corresponding accession numbers to the cDNA and protein sequences relating to FIGS. 1-12.

TABLE 5
Gene SNPs

| | | | | | | | | Single | | | |
|---|---|---|---|---|---|---|---|---|---|---|---|
| | | | | | | | | contig | | | |
| | | | PMP | | | | AA | Genomic | | Genomic | |
| SNP | Gene | Exon | Site | Location | Sequence | PMP | change | coord | Contig used for coords | Coord | cDNA coord |
| 1 | 803 | E | +1 | intron | CAGGGCAGGTCCAGGCCCTGCATGTGCTGAGTGCCGGGGAC | C>T | N/A | 276365 | R31167 AC007292 R/C | 11130 | |
| 2 | 803 | E | +2 | intron | AGGGCAGGTCCAGGCCCTGCATGTGCTGAGTGCCGGGGACG | M>G | N/A | 276366 | R31167 | 11131 | |
| 3 | 803 | H | −1 | intron | GGCCCCAGTCCCCTGTCCCCTCTCAGCCCCCTTGCCTCCTG | TC | N/A | 278195 | R31167 | 12960-12961 | |
| | | | | | | del | | | | | |
| 4 | 803 | H | +1 | intron | GCAAGTGGGCAGCACACCTCGCTGTGGGGTGAGCACATGGC | G>A | N/A | 278474 | R31167 | 13239 | |
| 5 | 803 | I | −1 | intron | AGAGTGGGGCTGCCTCGGCCGTGACACGGGCTCGGTCCACT | G>C | N/A | 278990 | R31167 | 13755 | |
| 6 | 803 | I | 1 | Exon | CCTAAGCGGGAGTATAAGCCCAAGCCCCGGGAGCCCTTTGA | C>G | None | 279072 | R31167 | 13837 | 965(v1):629(v2) |
| 7 | 803 | K | 1 | Exon | GAGCTGGGCTTCCATGAGGGCGAOGTCATCACGCTGACCAA | C>T | None | 280049 | R31167 | 14814 | 1184(v1),848(v2) |
| 8 | 803 | K | 2 | Exon | GGTACGAGGGCATGCTGGACGGCCAGTCGGGCTTCTTCCCG | G>A | Gly>Ser | 280107 | R31167 | 14872 | 1242(v1),906(v2) |
| 9 | 803 | K | 3 | Exon | GACGGCCAGTCGGGCTTCTTCCCGCTCAGCTACGTGGAGGT | C>T | None | 280124 | R31167 | 14889 | 1259(v1),923(v2) |
| 10 | 845 | D | +1 | intron | ATAACTTCTGTGTCAACGCCAGACGCAGGTGTCGCTGTTCT | A>G | | N/A | 6E24AC008694 | 11667 | |
| 11 | 845 | D | −1 | intron | GTGCTAGATCCTGTGCCCGACCCAGGGAGCCTGGTGCCGGC | C>T | | N/A | 6E24AC008694 R/C | 11484 | |
| 12 | 845 | D | 1 | Exon | GTTGTTGCTTTGAGCATCCACTCAAAGCTGAGCTCAGGGTA | C>T | Leu>Phe | N/A | 6E24AC008694 R/C | 11547 | 264 |
| 13 | 845 | F | +1 | intron | CTGAGGCTGCAGGTGGCACCGGGCACTCTCCCCAGGAATGG | G>T | | N/A | 47B1136AC008676 | 5389 | |
| 14 | 845 | F | +2 | intron | GTTTCAAGATAGACAAGGCTGAGGCAAGGACCTTGGGAAGG | G>A | | N/A | 47B1136AC008676 | 5432 | |
| 15 | 845 | G | +1 | intron | AGAAATCAACCTGGGGACAGGCGGTCCCCTCTGAGGTTGC | G>T | | N/A | 47B1136AC008676 | 16370 | |
| 16 | 845 | H | +1 | intron | GCTCACGCCTGTAATCCCAGCAC1TTGGGTGGGCAAGGCGG | C>G | | N/A | 47B1136AC008676 | 17516 | |
| 17 | 845 | H | +2 | intron | TTTGGGTGGGCPAGGCGGGTGGATCACCTGAGGTCAGGAGT | G>A | | N/A | 4761136AC008676 | 17539 | |
| 18 | 845 | H | −1 | intron | GGAAGGTGTTATATGAGTGATGGCCACCACAGCCAAGAACA | T>C | | N/A | 47811.36 AC008676 | 17124 | |
| 19 | 845 | H | 1 | exon | AAGTATGTGGAGCTTTACCTCGTGGCTGATTATTTAGAGGT | C>T | | N/A | 47811.36 AC008676 | 17324 | 725 |
| 20 | 845 | I | −1 | intron | TTAGAGGMGGCAATTCTACTCCGTGCATMTTAUGCATG | T>C | | N/A | 4781129 AC008676 | 2204 | |
| 21 | 845 | J | −1 | intron | CTTTTTAATCTCAAGCTCCACCAGAATGAAAGGGGGGGCT | C>A | | N/A | 47811.29 AC008676 | 6416 | |
| 22 | 845 | J | 1 | exon | CTACCCTCTGGTCCTTTCTCAGTTGGAGGCGCAAGCTGCTT | A>G | Ser>Gly | N/A | 4781129 AC008676 | 6601 | 927 |
| 23 | 845 | K | −1 | intron | AGAGGTGTGGTGAGCTGGAAGTTGTCCAGTTGGCCTGGTTA | G>T | | N/A | 47811.29 AC008676 | 8658 | |
| 24 | 845 | K | −2 | intron | GTATATTGTGGGAGATAAACGACCTTTCTCTCTCTCTTTGT | G>A | | N/A | 47811.29 AC008676 | 8578 | |
| 25 | 845 | K | 1 | exon | GCACCACCATCGGCCTGGCCCCCCTCATGGCCATGTGCTCTCT | C>T | Pro>Ser | N/A | 47811.29 AC008676 | 8855 | 1020 |
| 26 | 845 | P | +1 | intron | ATGAAATGAGGATGAACAAGCAGTTTCTGCTCTGTCCTCAC | C>T | | N/A | 47811.40 AC008676 | 9688 | |
| 27 | 845 | R | −1 | intron | ATATGTTCCAAGTGCAAAATCTTGCCTCCTACTCCCAACC | C>T | | N/A | 47811.40 AC008676 | 12353 | |
| 28 | 845 | R | 1 | exon | CTGTGGGAAGAAGTGCAATGGCCATGGGGTGCGTGCTGGGT | G>A | Gly>Asp | N/A | 47811.40 AC008676 | 12490 | 2056 |
| 29 | 847 | A | −1 | intron | GGCTGAGCGGCGGAGCCCCCCAAATGGCCTGGCCAGATGCG | C>T | | N/A | 13185AC011379 | 20517 | |
| 30 | 847 | A | 1 | exon | GCCGCCACTGGAGAAGGGTCGGTGCAGCAGCTACAGCGACA | G>A | Arg>Gln | N/A | 13185 A0011379 | 20585 | 278(v1-v6) |
| 31 | 847 | A | 2 | exon | GTACAGGGGCTGGTCCCAGCCGGCGGCTCCAGCTCCMCAG | C>T | | N/A | 13185 AC011379 | 20955 | 648(v1-v6) |
| 32 | 847 | C | +1 | intron | CAACAGCGGTAGGTGGGCCCAGACAGAGGGAAGGGTCCTAG | A>G | | N/A | 35F21 AC008667 | 13697 | |
| 33 | 847 | O | −1 | intron | ATGATTCCTGGGGCCTAGGGATAGTCTCAGTGCGTCACTGG | A>C | | N/A | 35F21 AC008667 | 22708 | |
| 34 | 847 | E | +1 | intron | CCTGCTCCT1AGAAAGCTTCTGGGTGCAGTCCCCCCAATGT | T>C | | N/A | 35F21 AC008667 | 29093 | |
| 35 | 847 | J | +1 | intron | CAGCCACAGGTAGGCACCACCAAGGCCCATGGTAACTTGTA | C>T | | N/A | 35F21 AC008667 | 42223 | |
| 36 | 847 | J | −1 | intron | GAAGGGGTGGGGGTTGATTTGCTGGAGCCATCGCTGCCCCA | G>T | | N/A | 35F21 AC008667 | 41929 | |
| 37 | 847 | K | −1 | intron | CGAGGTGGAAGAAAGAGCCAGGGAGGTCCATGGGACCACACC | G>A | | N/A | 35F21 AC008667 | 42735 | |
| 38 | 847 | K | 1 | exon | CTACAACCTGGAGGAGCGGCGCAGGGCCACCGCGCCACCCT | G>A | Arg>His | N/A | 35F21 AC008667 | 42974 | 1931(v1), 1913(v2), |
| | | | | | | | | | | | 1955(v3),1937(v4), |
| | | | | | | | | | | | N/A(v5),N/A(v6) |
| 39 | 874 | R | +1 | intron | GCTGGCCGTCCGGCGGCAGCTTGGGGAGTTCGGCACCGAGC | T>C | N/A | 82285 | RP11-163F8 | 82285 | N/A |
| 40 | 874 | R | 1 | exon | GCCCTTGACGTCAGAGTGTTTCTCCGCAGAGCCCGTGTCCTG | T>G | Phe>Leu | 81990 | RP11-163F8 | 81990 | 7 |
| 41 | 874 | R | 2 | exon | CAGAGGCGGCCAGGGGAGCGCGCCGCCCCGCTCGGCCOTCC | C>T | Arg>Cys | 82078 | RP11-163F8 | 82078 | 95 |
| 42 | 874 | S | +5 | intron | TTTTCATGTATGTTTTAAAACATAAAATGTAAAATATTCTG | C>G | N/A | 250203 | RP11-335L15 | 95682 | N/A |
| 43 | 874 | S | +4 | intron | TACTTTTCATGTATGTTTTAAAACATAAAATGTAAATATTA | A>G | N/A | 250200 | RP11-335L15 | 95679 | N/A |
| 44 | 874 | S | +3 | intron | TAAAACTACTTTTCATGTATGTTTTAAAACATAAATGTA | G>A | N/A | 250194 | RP11-335L15 | 95673 | N/A |
| 45 | 874 | S | 2 | intron | GGAGAGGGCAACTGTTTTCCACTGGTCTCTGAGAATACTAC | A>G | N/A | 250142 | RP11-335L15 | 95621 | N/A |
| 46 | 874 | S | 1 | intron | AGTGTTCCCTTGGACATAAACATGTCTACCATATTAGAGG | C>A | N/A | 250088 | RP11-335L15 | 95567 | N/A |
| 47 | 874 | S | −1 | intron | GCTGCTATAAAAATGAGACTCTCCACCTAAGTCAGGGAATG | C>G | N/A | 249852 | RP11-335L15 | 95331 | N/A |
| 48 | 874 | T | 1 | intron | TACTTTTAGTTTCTCTTTGATAGACATTTTAAGTTGGGTG | A>G | N/A | 264482 | RP11-335L15 | 109961 | N/A |
| 49 | 874 | T | −1 | intron | ATACAGACTCAACCAAAAACCGGTATTCTAAAGCTCATCAT | C>T | N/A | 264294 | RP11-335L15 | 109773 | N/A |
| 50 | 874 | U | −2 | intron | TCCTGCTTCATCCAGAACAGAATTGCTGTAATTCATTTTAA | A>T | N/A | 289067 | RP11-335L15 | 134546 | N/A |
| 51 | 874 | U | −1 | intron | CCTGCTTCATCCAGAACAGAATTGCTGTAATTCA1TFrAAG | A>G | N/A | 289068 | RP11-335L15 | 134547 | N/A |
| 52 | 874 | U | 1 | exon | ACCATGTCAAAAATCCGTGGCCAGGAGAAGGGGCCAGGCTA | C>T | None | 289343 | RP11-335L15 | 134822 | 409 |
| 53 | 874 | V | −3 | intron | TAAAGGAAAAAGTAACTGTTCCATTCTTGATGAGAGGTATT | C>G | N/A | 290091 | RP11-335L15 | 135570 | N/A |
| 54 | 874 | V | −2 | intron | GGTATTTACCCTCTTAGGGGGCATTGAGTCTGTTGCCTGGA | G>A | N/A | 290126 | RP11-335L15 | 135605 | N/A |
| 55 | 874 | V | −1 | intron | CCTCTTAGGGGGCATTGAGTCTGTTGCCTGGAGTGAACTGA | C>G | N/A | 290135 | RP11-335L15 | 135614 | N/A |
| 56 | 874 | X | −1 | intron | TATGTATGAAACAGTAGTGGCTGTTTAGGGATGGTAACGTG | C>A | N/A | 294088 | RP11-335L15 | 139567 | N/A |
| 57 | 874 | X | 1 | exon | TACCACAAGCAGGCAGTCCAGATCCTGCAGCAAGTCACGGT | G>A | None | 294212 | RP11-335L15 | 139691 | 865 |
| 58 | 874 | Y | 1 | intron | TGTCAATCATCAGCACACACGAACACATTTTGTTTTGACCC | G>A | N/A | 296674 | RP11-335L15 | 142153 | N/A |
| 59 | 874 | Y | 2 | intron | TTTTGACCCTCCTTTGTGGTCGTAAATCGCCTTTCCTTGTC | C>T | N/A | 296706 | RP11-335L15 | 142185 | N/A |
| 60 | 874 | Z | 1 | exon | ATCCTCTTAGCCTGGTGGGCGTGGCATGTGCTTTTTAAAAC | G>A | None | 298855 | RP11-335L15 | 144334 | 1430 |
| 61 | 962 | E | +2 | intron | CTCCGCTGCTCTCGCCTGGGTTFTGGAAAAGGGTTACCTGG | T>C | N/A | 7805 | CTC-500G13 | 7805 | N/A |
| | | | | | | | | | AC008544 | | |
| 62 | 962 | E | +1 | intron | TGCGATGGGCTGGTGAGTACGCACTTCTCTAGCTCCTTCT | G>C | N/A | 7764 | CTC-500G13 | 7764 | N/A |
| 63 | 962 | E | 2 | Exon | ACGAGCCCGCAGGGCCGCCCCGGTCCGGACCCCGAGCTTCC | C>T | Pro>Leu | 7493 | CTC-500G13 | 7493 | 272(v1-v2) |
| 64 | 962 | E | 3 | Exon | AACGAGGAGGAGCCTGGCAGTCACCTCTTCTACAATGTCAC | T>C | None | 7542 | CTC-500G13 | 7542 | 321(v1-v2) |
| 65 | 962 | G | +1 | Intron | CTGAGGGGGCAGGGTGGGGCGGGGAGGGTCAACCAGGGGCT | G>A | N/A | 143815 | CTC-500G13 | 143815 | N/A |
| 66 | 962 | G | −1 | Intron | TTTGCCCTGCCGCTGAAGACGGTGTCCTCTCCTGGCAGGGG | G>A | N/A | 143561 | CTC-500G13 | 143561 | N/A |
| 67 | 962 | G | 1 | Exon | GCCTCAGCCGCGCCCTGGGCGTCCTAGAGGAGCACGCCAAC | G>A | Val>Ile | 143623 | CTC-500G13 | 143623 | 733(v1-v2) |
| 68 | 962 | G | 2 | Exon | AGGGCACGCAGGCATGCTGCGGACGATGACTACAACATCGA | G>A | None | 143676 | CTC-500G13 | 143676 | 786(v1-v2) |
| 69 | 962 | G | 3 | Exon | CCTTGCAGGGGCCTCCCTGGACAGCCTGGACAGCCTCAGCC | A>G | Asp>Gly | 143591 | CTC-500G13 | 143591 | 701(v1-v2) |
| 70 | 962 | G | 4 | Exon | CAGCCTGGACAGCCTCAGCCGCGCCCTGGGCGTCCTAGAGG | G>A | Arg>His | 143612 | CTC-500G13 | 143612 | 722(v1-v2) |
| 71 | 962 | G | 5 | Exon | TGGGCGTCCTAGAGGAGCACGCCAACAGCTCGAGGCGGAGG | G>A | Ala>Thr | 143638 | CTC-500G13 | 143638 | 748(v1-v2) |
| 72 | 962 | G | 6 | Exon | CAGTTCCACGGGAAGGAGCACGTACAGAAGTACCTGCTGAC | C>T | None | 143748 | CTC-500G13 | 143748 | 858(v1-v2) |
| 73 | 962 | H | −1 | Intron | CCCATTGGCAGAGCCAGAGCCAGTCCTAGGGCCTGTGGTTC | C>T | N/A | 170091 | CTC-202F10 | 4024 | N/A |
| 74 | 962 | H | 1 | Exon | TCCTTGGGTGCCCACATCAACGTGGTCCTGGTGCGGATCAT | C>T | None | 170183 | CTC-202F10 | 4116 | 936(v1-v2) |
| 75 | 962 | H | +1 | Intron | AGGTTCCTATAAGAAACAAGATCCCTGCTCCCTTTCACCCC | A>G | N/A | 170372 | CTC-202F10 | 4305 | N/A |
| 76 | 962 | H | 2 | Intron | GGTTCCTATAAGAAACAAGATCCCTGCTCCCTTTCACCCCT | T>A | N/A | 170373 | CTC-202F10 | 4306 | N/A |
| 77 | 962 | I | −1 | Intron | CACTACAGCCTGCTCGGCCCCCCACTGGGCCACCAGGCGCC | C>T | N/A | 192358 | CTC-202F10 | 26291 | N/A |
| 78 | 962 | J | +1 | Intron | CGTGTAAGTGGCCTGGGGAAGGGTGGGGCACAGAGGGGCCA | G>A | N/A | 196498 | CTC-202F10 | 30431 | N/A |
| 79 | 962 | J | 1 | Intron | GCCTGGGAGTTAGGCCAGGCCTCACCTTCCCGGCCAGGCTA | C>T | N/A | 196318 | CTC-202F10 | 30251 | N/A |
| 80 | 962 | J | 1 | Exon | TGCACCCTGAACCATGAGGACGGCTGCTCCTCAGCGTTTGT | C>T | None | 196436 | CTC-202F10 | 30369 | 1194(v1-v2) |
| 81 | 962 | L | −2 | Intron | GCCGCGCTGAAGGCTGCTCGCGGCACCGTGTGTCCCCCACA | C>T | N/A | 197649 | CTC-202F10 | 31582 | N/A |
| 82 | 962 | L | −1 | Intron | CTGAAGGCTGCTCGCGGCACCGTGTGTCCCCCACAGCTCCT | C>T | N/A | 197655 | CTC-202F10 | 31588 | N/A |
| 83 | 962 | L | 1 | exon | TTCGCCCACGACTGGCCGGCGOTGCCCCAGCTCCCGGGACT | G>A | None | 197719 | CTC-202F10 | 31652 | 1431(v1-v2) |
| 84 | 962 | L | 2 | Exon | CAGCTCCCGGGACTGCACTACTCCATGAACGAGCAATGCCG | C>T | None | 197746 | CTC-202F10 | 31679 | 1458(v1-v2) |
| 85 | 962 | L | 3 | exon | GAGCAATGCCGCTTTGACTTCGGCCTGGGCTACATGATGTG | C>T | None | 197776 | CTC-202F10 A0010216 | 31709 | 1488(v1-v2) |
| 86 | 962 | M | −1 | Intron | CTTCACCACCCCAGAATCACAACACCCACCAGCCTCACGGG | A>G | N/A | 198901 | CTC-202F10 | 32834 | N/A |
| 87 | 962 | M | +1 | Intron | GCACCTGGCAAGGTGAGGCAGCATCAAGGGCTCTTGGAGGG | G>A | N/A | 199161 | CTC-202F10 | 33094 | N/A |
| 88 | 962 | M | +2 | Intron | AGGCAGCATCAAGGGCTCTTGGAGGGCAGCAGGGCAGAGGA | G>C | N/A | 199176 | CTC-202F10 | 33109 | N/A |
| 89 | 962 | M | +3 | Intron | TAATTACCOCTCACACGCTGTACGCCGGAGCTGCCTCCACC | T>C | N/A | 199313 | CTC-202F10 | 33246 | N/A |
| 90 | 962 | O | −2 | Intron | GAGCGCAGGACTGTCCCCACGGCCCGGTGTGAGGGTGAGTG | G>A | N/A | 211213 | CTC-202F10 | 45146 | N/A |
| 91 | 962 | O | −1 | Intron | GTGAGGGTGAGTGGAGGGACTGGCTTCCTGTCTTTCAGCAT | T>C | N/A | 211241 | CTC-202F10 | 45174 | N/A |
| 92 | 962 | O | +1 | Intron | GCAGCTGAGGGTCCAGGAGACCCTCTCCAGCCAGCCCTGTC | C>T | N/A | 211462 | CTC-202F10 | 45395 | N/A |
| 93 | 962 | P | −3 | Intron | GCTCAGCAGAGCTGCCCCCCGGACACCTGAGACTTAGGGAT | G>A | N/A | 213243 | CTC-202F10 | 47176 | N/A |
| 94 | 962 | P | −2 | Intron | AAGATCTCACAGGGCACCCGGGTGCTGCCTCTTTCCATG | G>A | N/A | 213294 | CTC-202F10 | 47227 | N/A |
| 95 | 962 | P | −1 | Intron | TCTTTCCAATGGCACGGAGCGGCAAGGCCTTTGCTTTCTCC | G>A | N/A | 213324 | CTC-202F10 | 47257 | N/A |
| 96 | 962 | P | +1 | Intron | TTCTGGGAGGCATCAGTGGGGGCTCAGCAGGCAGGCCCTAG | G>A | N/A | 213555 | CTC-202F10 | 47488 | N/A |
| 97 | 962 | Q | +1 | Intron | ATGAAGCGCATGGTGCATGACGGGACGCGCTGCTCCTACAA | C>T | N/A | 215329 | CTC-202F10 | 49262 | N/A |
| 98 | 962 | Q | +2 | Intron | GTACTGACCTCCCCCTTTTCGGGGTATTGGCAAGATGCATG | G>A | N/A | 215462 | CTC-202P10 | 49395 | N/A |
| 99 | 962 | Q | −1 | Intron | GTGGGGACCCTTGTGGAAATTTCTCCTGCTTGGTGCCTCCT | T>A | N/A | 215171 | CTC-202F10 | 49104 | N/A |
| 100 | 962 | Q | 1 | Exon | TACTGCGAGTCCAGGGAGACCGGGGAGGTGGTGTCCATGAA | C>T | None | 215293 | CTC-202F10 | 49226 | 1992(v1), N/A(v2) |
| 101 | 962 | Q | 2 | Exon | ACTGCGAGTCCAGGGAGACCGGGGAGGTGGTGTCCATGAAG | G>A | Gly>Arg | 215294 | CTC-202F10 | 49227 | 1993(v1), N/A(v2) |
| 102 | 962 | S | −1 | Intron | AGGGTCCTGGGAGAGCCTCCGGAGGAGCTGCCTTCAAGAGC | G>C | N/A | 218885 | CTC-202F10 | 52818 | N/A |
| 103 | 962 | T | −2 | Intron | CCAGTGGGCCTGGGTCCTGCTCTTGGGTGACCACAACGGGG | T>C | N/A | 221154 | CTC-202F10 | 55087 | N/A |
| 104 | 962 | T | −1 | Intron | ACGGGGGACTTGGTCGGCCATTCTCAGCCGTCAAGAACCT | A>G | N/A | 221189 | CTC-202F10 | 55122 | N/A |
| 105 | 962 | U | 1 | Exon | CATCCCGGTGGGAGACACCCGGGTCTCACTGACGTACAAAT | G>A | Arg>Gln | 223199 | CTC-202F10 | 57132 | 2480(v1), N/A(v2) |
| 106 | 962 | U | 2 | Exon | GAGGACTCACTGAATGTCGACGACACAACGTCCTGGAAGA | C>T | None | 223251 | CTC-202F10 | 57184 | 2532(v1), N/A(v2) |
| 107 | 962 | V | +1 | Intron | GCAGCCCACCCCTCCTTGCACCCTCGGGCAGGGCATGCTGC | C>T | N/A | 225111 | CTC-202F10 | 59044 | N/A |
| 108 | 962 | V | +1 | Intron | CCCACTAGAGGAGACAGGCCAGGGGCCACCAGGGGCTCCCG | A>G | N/A | 225340 | CTC-202F10 | 59273 | N/A |
| 109 | 962 | V | +2 | Intron | GCCTGGGCCTGGCATCATCCGAGGCATTTGACCAAGTCTCT | G>A | N/A | 225397 | CTC-202F10 | 59330 | N/A |
| 110 | 962 | Y | +1 | Intron | GATCTCGTCAAGTAACCGACCCGTTTATAACTCTGCCTCTG | C>T | N/A | 229644 | CTC-202F10 | 63577 | N/A |
| 111 | 962 | Y | +2 | intron | GTGACCGTTTTCTCCCGGGCCTCTGAGCTCGGCGTCCGCTC | C>G | N/A | 229782 | CTC-202F10 | 63715 | N/A |
| 112 | 962 | Z | 1 | Exon | TGGAAGATGAAGTCCAGCCACCCAACCTATCCCTCGACGA | C>T | Pro>Ser | 237321 | CTC-202F10 | 71254 | 3529(v1), N/A(v2) |
| 113 | 962 | Z | 2 | exon | CCACCGCCTGGGAAGCACAACGACAGGACGTGTTCATGCC | C>T | None | 237134 | CTC-202F10 | 71067 | 3342(v1), N/A(v2) |

### Example 6

Allele Specific Assay

Once variants were confirmed by sequencing, rapid allele specific assays were designed to type and diagnose more than 400 individuals (>200 cases and >200 controls) for use in the association studies. All coding SNPs (cSNPs) that resulted in an amino acid change were typed. Neutral polymorphisms were typed if: 1) the polymorphism was present in an exon lacking a cSNP; 2) the polymorphism was present in an exon containing a cSNP, but the two polymorphisms were observed to have different frequencies; or 3) the polymorphism was in an intronic region adjacent to an exon without a cSNP. If results from the association studies appeared positive, additional neutral polymorphisms were typed.

Three types of allele specific assays (ASAs) were used. If the SNP resulted in a mutation that created or abolished a restriction site, RFLPs were obtained from PCR products that spanned the variants, and were subsequently analyzed. If the polymorphism did not result in an RFLP, allele-specific oligonucleotide or exonuclease proofreading assays were used. For the allele-specific oligonucleotide assays, PCR products that spanned the polymorphism were electrophoresed on agarose gels and transferred to nylon membranes by Southern blotting. Oligomers 16-20 bp in length were designed such that the middle base was specific for each variant. The oligomers were labeled and successively hybridized to the membrane in order to determine genotypes.

Table 7, below, shows the information for the ASAs. The first column lists the SNP names. The second column lists the specific assays used (RFLP, ASO, an alternate method). The third column lists the enzymes used in the RFLP assay (described below). The fourth and fifth columns list the sequences of the oligos used in the ASO assay (described below). In addition, Table 7 contains the nucleic acid base change at the SNP location and if applicable, the corresponding amino acid change of the resulting protein.

TABLE 7
Allele Specific Assays

| SNP | ASA Type | RFLP Enzyme | ASO Oligo1 | ASO Oligo2 | Base change | A.A. change |
|---|---|---|---|---|---|---|
| 845_D_+1 | RFLP | Mspl | | | A>G | |
| 845_D_−1 | ASO | | GTGCCCGACCCAGGGA | GTGCCCGATCCAGGGAGC | C>T | |
| 845_D_1 | ASO | | AGCATCCACTCAAAGCTG | AGCATCCATTCAAAGCTGA | C>T | Leu>Phe |
| 845_F_+1 | RFLP | Bsp12861 | | | G>T | |
| 845_G_+1 | ASO | | GGGGACAGGCTTGTCC | CCTGGGGACAGTCTTGTCCCCT | G>T | |
| 845_H_+1 | RFLP | BsaJL | | | C>G | |
| 845_H_+2 | RFLP | Alwl | | | G>A | |
| 845_H_−1 | RFLP | Mscl | | | T>C | |
| 845_I_−1 | ASO | | GCAATTCTACTCCGTGCATAAT | CAATTCTACCCCGTGCATA | T>C | |
| 845_J_−1 | RFLP | Msll | | | C>A | |
| 845_J_1 | RFLP | Odel | | | A>G | Ser>Gly |
| 845_K_−1 | RFLP | Tsp5091 | | | G>T | |
| 845_K_−2 | ASO | | GAGATAAACAACCTTTCTCT | GAGATAAACGACCTTTCTCT | G>A | |
| 845_K_1 | RFLP | Sau961 | | | C>T | Pro>Ser |
| 845_P_+1 | RFLP | AlwNl | | | C>T | |
| 845_R_−1 | RFLP | Tsp5091 | | | C>T | |
| 845_R_1 | RFLP | Mscl | | | G>A | Gly>Asp |
| 847_A_1 | ASO | | GAAGGGTCGGTGCAGCA | GAAGGGTCAGTGCAGCAGC | G>A | Arg>Gln |
| 847_A_2 | RFLP | Pvull | | | C>T | |
| 847_C_+1 | RFLP | Ncil | | | A>G | |
| 847_D_−1 | ASO | | GGGCCTAGGGATAGTCTCAG | CCTAGGGCTAGTCTCAGT | A>C | |
| 847_E_+1 | RFLP | Ncil | | | T>C | |
| 847_J_+1 | ASO | | GCACCACCAAGGCCCAT | AGGCACCACTAAGGCCCAT | C>T | |
| 847_K_1 | ASO | | GAGCGGCGCAGGGCC | GGAGCGGCACAGGGCCA | G>A | Arg>His |
| 803_E_+2 | RFLP | Nlalll | | | A>G | |
| 803_H_+1 | ASO | | ACACCTCGCTGTGGGGT | GCACACCTCACTGTGGGG | G>A | |
| 803_H_−1 | ASO | | GTCCCCTCTCAGCCCCC | GTCCCCTCAGCCCCC | TC del | |
| 803_I_−1 | RFLP BslEl | | | G>C | | |
| 803_I_1 | ASO | | GTATAAGCCCAAGCCCC | GTATAAGCCGAAGCCCC | C>G | |
| 803_K_3 | RFLP | Hboll | | | G>A | Gly>Ser |
| 803_K_3 | Mboll | | | | C>T | |
| 962_E_+2 | ASO | | CGCCTGGGTTTTGGAAAAG | CGCCTGGGCTTTGGAAAA | T>C | |
| 962_E_2 | At Meth | | | C>T Pro>Leu | | |
| 962_E_3 | RFLP | Hphl | | | T>C | |
| 962_G_1 | RFLP | BsaHl | | | G>A | Val>Ile |
| 962_G_2 | RFLP | Pstl | | | G>A | |
| 962_G_4 | RFLP | BstUl | | | G>A | Arg>His |
| 962_G_5 | RFLP | Cac8l | | | G>A | Ala>Thr |
| 962_G_6 | RFLP | HpyCH4lV | | | C>T | |
| 962_H_+2 | RFLP | Dpnll | | | T>A | |
| 962_J_1 | Alt Meth | | | C>T | | |
| 962_L_2 | Alt Meth | | | C>T | | |
| 962_M_+2 | RFLP | Taq | | | G>C | |
| 962_P_−2 | RFLP | AlwNl | | | G>A | |
| 962_Q_−1 | RFLP | Asp5091 | | | T>A | G>C |
| 962_S_−2 | RFLP | Slyl | | | T>C | |
| 962_U_1 | RFLP | Aval | | | G>A | Arg>Gln |
| 962_U_2 | RFLP | Hincll | | | C>T | |
| 962_V_+2 | RFLP | Styl | | | G>A | |
| 962_V_−1 | ASO | | CCCGAGGGTGCAAGGA | GCCCGAGGATGCAAGGAG | C>T | |
| 962_Z_1 | Alt Meth | | | C>T | Pro>Ser | |
| 874_R_+1 | RFLP | BstNl | | | T>C | |
| 874_R_1 | Alt Meth | | | T>G | Phe>Leu | |
| 874_R_2 | ASO | | GGGAGCGCGCC | GCCGGGGAGCGTGCCGCCC | C>T | Arg>Cys |
| 874_S_+1 | RFLP | Nlalll | | | C>A | |
| 874_S_+3 | Alt Meth | | | G>A | | |
| 874_T_−1 | RFLP | Hpall | | | C>T | |
| 874_U_−2 | Alt Meth | | | A>T | | |
| 874_V_−1 | RFLP | Hinfl | | | C>G | |
| 874_X_1 | RFLP | Dpnll | | | G>A | |
| 874_Y_+2 | It Meth | | | C>T | | |
| 874_Z_1 | It Meth | | | G>A | | |

1. RFLP Assay: The amplicon containing the polymorphism was PCR amplified using primers that generated fragments for sequencing (sequencing primers) or SSCP (SSCP primers). The appropriate population of individuals was PCR amplified in 96-well microtiter plates. Enzymes were purchased from NEB. The restriction cocktail containing the appropriate enzyme for the particular polymorphism was added to the PCR product. The reaction was incubated at the appropriate temperature according to the manufacturer's recommendations for 2-3 hr, followed by a 4° C. incubation. After digestion, the reactions were size fractionated using the appropriate agarose gel depending on the assay specifications (2.5%, 3%, or Metaphor, FMC Bioproducts). Gels were electrophoresed in 1× TBE buffer at 170 V for approximately 2 hr. The gel was illuminated using UV, and the image was saved as a Kodak 1D file. Using the Kodak 1D image analysis software, the images were scored and the data was exported to Microsoft® Excel (http://www.microsoft.com).

2. ASO assay: The amplicon containing the polymorphism was PCR amplified using primers that generated fragments for sequencing (sequencing primers) or SSCP (SSCP primers). The appropriate population of individuals was PCR amplified in 96-well microtiter plates and re-arrayed into 384-well microtiter plates using a Tecan Genesis RSP200. The amplified products were loaded onto 2% agarose gels and size fractionated at 150V for 5 min. The DNA was transferred from the gel to Hybond N+ nylon membrane (Amersham-Pharmacia) using a Vacuum blotter (Bio-Rad). The filter containing the blotted PCR products was transferred to a dish containing 300 ml pre-hybridization solution (5×SSPE (pH 7.4), 2% SDS, 5× Denhardt's). The filter was incubated in pre-hybridization solution at 40° C. for over 1 hr. After pre-hybridization, 10 ml of the pre-hybridization solution and the filter were transferred to a washed glass bottle. The allele-specific oligonucleotides (ASO) were designed to contain the polymorphism in the middle of the nucleotide sequence. The size of the oligonucleotide was dependent upon the GC content of the sequence around the polymorphism. Those ASOs that had a G or C polymorphism were designed so that the Tₘ was between 54-56° C. Those ASOs that had an A or T polymorphism were designed so that the Tₘ was between 60-64° C. All oligonucleotides were phosphate-free at the 5′ ends and purchased from GibcoBRL. For each polymorphism, 2 ASOs were designed to yield one ASO for each strand.

The ASOs that represented each polymorphism were resuspended at a concentration of 1 μg/μl. Each ASO was end-labeled with γ-ATP³² (6000 Ci/mmol) (NEN) using T4 polynucleotide kinase according to manufacturer recommendations (NEB). The end-labeled products were removed from the unincorporated γ-ATP³² using a Sephadex G-25 column according to the manufacturer's instructions (Amersham-Pharmacia). The entire end-labeled product of one ASO was added to the bottle containing the appropriate filter and 10 ml hybridization solution. The hybridization reaction was placed in a rotisserie oven (Hybaid) and left at 40° C. for a minimum of 4 hr. The other ASO was stored at −20° C.

After the prerequisite hybridization time had elapsed, the filter was removed from the bottle and transferred to 1 L of wash solution (0.1×SSPE (pH 7.4) and 0.1% SDS) pre-warmed to 45° C. After 15 min, the filter was transferred to another liter of wash solution (0.1×SSPE (pH 7.4) and 0.1% SDS) pre-warmed to 50° C. After 15 min, the filter was wrapped in Saran Wrap®, placed in an autoradiograph cassette, and an X-ray film (Kodak) was placed on top of the filter. Typically, an image was visible within 1 hr. After an image was captured on film following the 50° C. wash, images were captured following wash steps at 55° C., 60° C. and 65° C. The best image was selected.

The ASO was removed from the filter by adding 1 L of boiling strip solution (0.1×SSPE (pH 7.4) and 0.1% SDS). This was repeated two more times. After removing the ASO, the filter was pre-hybridized in 300 ml pre-hybridization solution (5×SSPE (pH 7.4), 2% SDS, and 5× Denhardt's) at 40° C. for over 1 hr. The second end-labeled ASO corresponding to the other strand was removed from storage at −20° C. and thawed at RT. The filter was placed into a glass bottle along with 10 ml hybridization solution and the entire end-labeled product of the second ASO. The hybridization reaction was placed in a rotisserie oven (Hybaid, http://www.hybaid.co.uk) and left at 40° C. for a minimum of 4 hr. After the hybridization, the filter was washed at various temperatures and images captured on film as described above. The best image for each ASO was converted into a digital image by scanning the film into Adobe® Photoshop®. These images were overlaid using Graphic Converter, and the overlaid images were scored.

3. Exonuclease Proofreading Assay: Exonuclease Proofreading Assays (EPAs) were also employed (see U.S. Pat. No. 5,391,480). Briefly, primers corresponding to the polymorphisms of interest were designed to contain fluorescent tags at the 3′ ends. The primers were designed such that the 3′ ends contained the variant or consensus nucleotides. Mismatched bases at the 3′ ends were removed by an exonuclease proof-reading enzyme (Pwo DNA polymerase; Roche, Germany; Cat. No. 1-644-855) in the PCR reaction. Where bases were matched, the resulting PCR products contained the tagged bases. The tagged bases were detected by gel electrophoresis or florescent polarization

### Example 7

Association Study Analysis

1. Case-Control Study All the genes listed in Tables 1 and 2 are involved in asthma and related disorders however, in order to determine which polymorphisms in candidate genes are strongly associated with the asthma phenotype, association studies were performed using a case-control design. In a well-matched design, the case-control approach is more powerful than the family based transmission disequilibrium test (TDT) (N. E. Morton and A. Collins, 1998, Proc. Natl. Acad. Sci. USA 95:11389-93). Case-control studies are, however, sensitive to population admixture.

To avoid issues of population admixture, which can bias case-control studies, unaffected controls were collected in both the US and the UK. A total of three hundred controls were collected, 200 in the UK and 100 in the US. Inclusion into the study required that the control individual was 1) negative for asthma (as determined by self-report of never having asthma); 2) had no first-degree relatives with asthma; and 3) was negative for eczema and symptoms indicative of atopy for the past 12 months. Data from an abbreviated questionnaire similar to that administered to the affected sib pair families were collected. Results from skin prick tests to 4 common allergens were also collected. The results of the skin prick tests were used to select a subset of controls that were most likely to be asthma and atopy negative.

A subset of unrelated cases was selected from the affected sib pair families based on the evidence for linkage at the chromosomal location near a given gene. One affected sib demonstrating identity-by-descent (IBD) at the appropriate marker loci was selected from each family. As the appropriate cases may vary for each gene in the region, a larger collection of individuals who were IBD across a larger interval was genotyped. A subset of this collection was used in the analyses. Over 100 IBD affected individuals and 200 controls were compared for allele and genotype frequencies.

For each polymorphism, the frequency of the alleles in the control and case populations was compared using a Fisher's exact test. A mutation that increased susceptibility to the disease was expected to be more prevalent in the cases than in the controls, while a protective mutation was expected to be more prevalent in the control group. Similarly, the genotype frequencies of the SNPs were compared between cases and controls. P-values for the allele and genotype tests are tabulated. A small p-value was deemed indicative of an association between the SNPs and the disease phenotype. The analysis was repeated for the US and UK populations, separately, to correct for genetic heterogeneity. The association tables under this section show the least frequent base or allele in the control population. Table 5 above shows all base changes for the particular SNP location. Therefore, a particular allele or base may be discussed as significant in the text under this section but the particular base is not reported in the tables below. Thus, the base at the particular location can be identified using Table 5.

2. Association test with individual SNPs: Fourteen SNPs in Gene 845, seven SNPs in Gene 847, four SNPs in Gene 874, six SNPs in Gene 803 and 16 SNPs in Gene 962 were typed. Four separate phenotypes were used in these analyses: asthma, bronchial hyper-responsiveness, total IgE, and specific IgE.

a. Asthma Phenotype: Frequencies and p-values for all typed SNPs are shown in Tables 9, 10, and 11 for the combined population and for the UK and US populations, separately. Column 1 lists the SNP names, which were derived from the gene numbers and closest exons. Column 2 lists the allele name. Columns 3 and 4 list the control (“CNTL”) allele frequencies and sample sizes (“N”), respectively. Columns 5 and 6 list the affected individuals (“CASE”) allele frequencies and sample sizes (“N”), respectively. Columns 7 and 8 list the p-values for the comparison between the case and control allele and genotype frequencies, respectively. A single SNP in Gene 845 reached statistical significance in the US population alone for the allele test: SNP P+1. For this SNP, 17.4% of the cases were carriers of the T allele, whereas the T allele was observed in only 6.5% of the controls (allele test p=0.0366). A single SNP in Gene 803 reached statistical significance in the combined and the US population alone for both the allele and the genotype tests: SNP K 2. For this SNP, 2.1% of the cases in the combined population were carriers of the A allele, whereas the A allele was observed in only 0.2% of the controls (combined: allele test p=0.0242, genotype test p=0.0237; US: allele test p=0.0475, genotype test p=0.0467). Five SNPs in Gene 962 reached statistical significance in the combined and the US population alone for both the allele and the genotype tests: 15.2% of the cases were carriers of the C allele in SNP M+2, whereas the C allele was observed in only 13.2% of the controls (US: genotype test p=0.0336), 40.5% of the cases were carriers of the A allele in SNP P−2, whereas the A allele was observed in only 22.4% of the controls (US: allele test p=0.0286, genotype test p=0.0227), 39.1% of the cases were carriers of the A allele in SNP Q−1, whereas the A allele was observed in only 21.7% of the controls (US: allele test p=0.0218, genotype test p=0.0375), 39.6% of the cases were carriers of the T allele in SNP U 2, whereas the T allele was observed in only 21.7% of the controls (US: allele test p=0.0225, genotype test p=0.0284) and 73.5% of the cases were carriers of the C allele in SNP V−1, whereas the C allele was observed in only 65.2% of the controls SNP V−1 (combined: allele test p=0.0383).

TABLE 9
ASSOCIATION ANALYSIS OF ASTHMA PHENOTYPE
COMBINED US/UK POPULATION
Combined US & UK

| | | | FREQUENCIES | | | ALLELE | GENOTYPE |
|---|---|---|---|---|---|---|---|
| GENE_EXON | ALLELE | CNTL | N | CASE | N | P-VALUE | P-VALUE |
| 845_R_1 | A | 14.8% | 216 | 12.5% | 100 | 0.4634 | 0.7361 |
| 845_R_−1 | T | 28.7% | 214 | 26.0% | 102 | 0.5070 | 0.7697 |
| 845_P_+1 | T | 6.5% | 217 | 9.5% | 105 | 0.1998 | 0.3027 |
| 845_K_1 | T | 0.2% | 210 | 0.0% | 96 | 1.0000 | 1.0000 |
| 845_K_−2 | A | 28.9% | 216 | 24.0% | 98 | 0.2103 | 0.4160 |
| 845_J_1 | G | 34.5% | 210 | 38.8% | 103 | 0.3296 | 0.5664 |
| 845_J_−1 | C | 37.1% | 217 | 38.5% | 104 | 0.7940 | 0.4453 |
| 845_I_−1 | C | 12.8% | 191 | 11.5% | 104 | 0.6962 | 0.6932 |
| 845_H_+1 | G | 19.6% | 217 | 14.9% | 104 | 0.1559 | 0.3133 |
| 845_H_−1 | T | 45.2% | 217 | 44.2% | 103 | 0.8650 | 0.7396 |
| 845_G_+1 | T | 13.2% | 216 | 13.9% | 104 | 0.8053 | 0.5846 |
| 845_F_+1 | T | 18.4% | 215 | 17.6% | 105 | 0.9130 | 0.5565 |
| 845_D_1 | T | 0.2% | 216 | 1.0% | 99 | 0.2339 | 0.2335 |
| 845_D_−1 | T | 9.9% | 217 | 11.1% | 99 | 0.6727 | 0.4308 |
| 847_K_1 | A | 4.9% | 214 | 5.7% | 97 | 0.6976 | 0.6900 |
| 847_J_+1 | T | 4.9% | 216 | 2.3% | 109 | 0.1394 | 0.1312 |
| 847_E_+1 | C | 12.6% | 210 | 13.1% | 107 | 0.9001 | 0.4982 |
| 847_D_−1 | C | 16.9% | 210 | 18.3% | 93 | 0.7272 | 0.8153 |
| 847_C_+1 | G | 17.9% | 209 | 20.8% | 108 | 0.3932 | 0.1700 |
| 847_A_2 | T | 6.7% | 217 | 7.3% | 109 | 0.7457 | 0.7365 |
| 847_A_1 | A | 1.0% | 192 | 0.5% | 103 | 0.6625 | 0.6610 |
| 874_R_+1 | T | 39.9% | 202 | 35.6% | 94 | 0.3645 | 0.2706 |
| 874_S_+1 | A | 39.5% | 214 | 41.9% | 99 | 0.5993 | 0.8366 |
| 874_T_−1 | T | 48.6% | 213 | 50.0% | 100 | 0.7971 | 0.8135 |
| 874_V_−1 | G | 17.6% | 216 | 19.5% | 100 | 0.5803 | 0.3894 |
| 803_K_3 | T | 0.9% | 218 | 1.2% | 121 | 0.7046 | 0.7035 |
| 803_K_2 | A | 0.2% | 217 | 2.1% | 121 | 0.0242 | 0.0237 |
| 803_I_1 | G | 28.2% | 195 | 26.9% | 119 | 0.7829 | 0.4504 |
| 803_I_−1 | C | 0.2% | 217 | 0.0% | 118 | 1.0000 | 1.0000 |
| 803_H_+1 | A | 25.2% | 218 | 24.4% | 119 | 0.8524 | 0.9029 |
| 803_E_+2 | A | 44.2% | 208 | 45.3% | 118 | 0.8060 | 0.8313 |
| 962_E_3 | C | 35.6% | 212 | 38.7% | 115 | 0.4455 | 0.4859 |
| 962_E_+2 | C | 12.7% | 217 | 10.4% | 120 | 0.4558 | 0.3772 |
| 962_G_4 | A | 13.6% | 202 | 9.6% | 114 | 0.1641 | 0.2877 |
| 962_G_1 | A | 27.2% | 217 | 34.3% | 121 | 0.0541 | 0.0932 |
| 962_G_2 | A | 7.6% | 217 | 7.6% | 118 | 1.0000 | 0.4312 |
| 962_G_6 | T | 20.4% | 194 | 17.8% | 115 | 0.4632 | 0.7068 |
| 962_H_+2 | A | 41.5% | 217 | 39.5% | 119 | 0.6236 | 0.6019 |
| 962_M_+2 | C | 12.7% | 213 | 9.3% | 113 | 0.2455 | 0.1865 |
| 962_P_−2 | A | 23.8% | 214 | 25.4% | 114 | 0.7026 | 0.4231 |
| 962_Q_−1 | A | 23.7% | 215 | 25.7% | 115 | 0.6345 | 0.7013 |
| 962_S_−1 | C | 11.4% | 215 | 7.6% | 119 | 0.1388 | 0.3757 |
| 962_U_1 | A | 3.0% | 214 | 3.5% | 115 | 0.8173 | 0.8144 |
| 962_U_2 | T | 23.8% | 212 | 25.0% | 118 | 0.7763 | 0.8883 |
| 962_V_−1 | T | 34.8% | 187 | 26.5% | 115 | 0.0383 | 0.0954 |
| 962_V_+2 | A | 4.5% | 209 | 2.5% | 119 | 0.2117 | 0.5991 |
| 962_Z_1 | T | 31.9% | 216 | 33.1% | 121 | 0.7970 | 0.1515 |

TABLE 10
ASSOCIATION ANALYSIS OF ASTHMA
PHENOTYPE UK POPULATION
UK population

| | | | FREQUENCIES | | | ALLELE | GENOTYPE |
|---|---|---|---|---|---|---|---|
| GENE_EXON | ALLELE | CNTL | N | CASE | N | P-VALUE | P-VALUE |
| 845_R_1 | A | 13.6% | 140 | 13.9% | 79 | 1.0000 | 0.9021 |
| 845_R_−1 | T | 26.6% | 137 | 25.3% | 79 | 0.8205 | 0.9047 |
| 845_P_+1 | T | 6.4% | 140 | 7.3% | 82 | 0.7005 | 0.6394 |
| 845_K_I | T | 0.4% | 135 | 0.0% | 73 | 1.0000 | 1.0000 |
| 845_K_−2 | A | 27.0% | 139 | 24.0% | 77 | 0.5669 | 0.8306 |
| 845_J_1 | G | 36.6% | 134 | 37.5% | 80 | 0.9176 | 0.9804 |
| 845_J_−1 | C | 37.5% | 140 | 40.1% | 81 | 0.6126 | 0.5300 |
| 845_I_−1 | C | 12.6% | 127 | 11.1% | 81 | 0.7575 | 0.9186 |
| 845_H_+1 | G | 19.3% | 140 | 16.0% | 81 | 0.4428 | 0.5503 |
| 845_H_−1 | T | 43.6% | 140 | 42.6% | 81 | 0.9207 | 0.9647 |
| 845_G_+1 | T | 3.2% | 140 | 14.8% | 81 | 0.6688 | 0.6235 |
| 845_F_+1 | T | 8.8% | 138 | 18.3% | 82 | 1.0000 | 0.9315 |
| 845_D_1 | T | 0.0% | 140 | 1.3% | 78 | 0.1275 | 0.1270 |
| 845_D−—1 | T | 9.3% | 140 | 10.3% | 78 | 0.7377 | 0.7237 |
| 847_K_1 | A | 3.6% | 139 | 4.8% | 73 | 0.6053 | 0.5980 |
| 847_J_+1 | T | 2.9% | 139 | 2.4% | 85 | 1.0000 | 1.0000 |
| 847_E_+1 | C | 12.8% | 133 | 14.1% | 85 | 0.7727 | 0.2313 |
| 847_D_−1 | C | 16.5% | 136 | 21.2% | 73 | 0.2351 | 0.2901 |
| 847_C_+1 | G | 17.8% | 132 | 22.9% | 85 | 0.2175 | 0.0894 |
| 847_A_2 | T | 5.4% | 140 | 7.1% | 85 | 0.5400 | 0.5266 |
| 847_A_1 | A | 1.3% | 120 | 0.6% | 83 | 0.6479 | 0.6462 |
| 874_R_+1 | T | 41.5% | 129 | 35.8% | 74 | 0.2918 | 0.4463 |
| 874_S_+1 | A | 38.0% | 137 | 42.3% | 78 | 0.4122 | 0.5428 |
| 874_T_−1 | T | 48.9% | 136 | 50.6% | 79 | 0.7645 | 0.9496 |
| 874_V_−1 | G | 16.9% | 139 | 19.6% | 79 | 0.5164 | 0.5248 |
| 803_K_3 | T | 1.1% | 140 | 1.5% | 99 | 0.6955 | 0.6940 |
| 803_K_2 | A | 0.4% | 139 | 1.5% | 99 | 0.3124 | 0.3104 |
| 803_I_1 | G | 28.6% | 117 | 24.7% | 97 | 0.3827 | 0.2487 |
| 803_I_−1 | C | 0.4% | 139 | 0.0% | 96 | 1.0000 | 1.0000 |
| 803_H_+1 | A | 25.7% | 140 | 25.3% | 97 | 1.0000 | 0.8747 |
| 803_E_+2 | A | 42.4% | 132 | 46.9% | 96 | 0.3903 | 0.5906 |
| 962_E_3 | C | 36.8% | 136 | 36.4% | 92 | 1.0000 | 0.9824 |
| 962_E_+2 | C | 12.5% | 140 | 9.9% | 96 | 0.4621 | 0.7476 |
| 962_G_4 | A | 14.0% | 132 | 10.6% | 90 | 0.3105 | 0.4407 |
| 962_G_1 | A | 25.2% | 139 | 33.5% | 97 | 0.0502 | 0.1164 |
| 962_G_2 | A | 6.4% | 140 | 7.4% | 94 | 0.7106 | 0.5925 |
| 962_G_6 | T | 21.7% | 129 | 16.8% | 92 | 0.2261 | 0.3013 |
| 962_H_+2 | A | 43.6% | 140 | 39.5% | 95 | 0.3926 | 0.5214 |
| 962_M_+2 | C | 12.4% | 137 | 7.8% | 90 | 0.1221 | 0.3225 |
| 962_P_−2 | A | 24.6% | 136 | 22.0% | 93 | 0.5757 | 0.7940 |
| 962_Q_−1 | A | 24.8% | 139 | 22.3% | 92 | 0.5776 | 0.8022 |
| 962_S_−1 | C | 10.9% | 137 | 6.3% | 95 | 0.1006 | 0.2430 |
| 962_U_1 | A | 3.7% | 136 | 3.7% | 94 | 1.0000 | 1.0000 |
| 962_U_2 | T | 25.0% | 136 | 21.3% | 94 | 0.3727 | 0.7464 |
| 962_V_−1 | T | 34.4% | 122 | 27.2% | 92 | 0.1154 | 0.0909 |
| 962_V_+2 | A | 4.5% | 134 | 2.6% | 95 | 0.4526 | 0.7627 |
| 962_Z_1 | T | 30.0% | 140 | 35.1% | 97 | 0.2713 | 0.0735 |

TABLE 11
ASSOCIATION ANALYSIS OF ASTHMA
PHENOTYPE US POPULATION
US population

| | | | FREQUENCIES | | | ALLELE | GENOTYPE |
|---|---|---|---|---|---|---|---|
| GENE_EXON | ALLELE | CNTL | N | CASE | N | P-VALUE | P-VALUE |
| 845_R_1 | A | 17.1% | 76 | 7.1% | 21 | 0.1433 | 0.3391 |
| 845_R_−1 | T | 32.5% | 77 | 28.3% | 23 | 0.7180 | 0.5080 |
| 845_P_+1 | T | 6.5% | 77 | 17.4% | 23 | 0.0366 | 0.1001 |
| 845_K_1 | T | 0.0% | 75 | 0.0% | 23 | 1.0000 | 1.0000 |
| 845_K_−2 | A | 32.5% | 77 | 23.8% | 21 | 0.3465 | 0.5335 |
| 845_J_1 | G | 30.9% | 76 | 43.5% | 23 | 0.1542 | 0.2183 |
| 845_J_−1 | C | 36.4% | 77 | 32.6% | 23 | 0.7267 | 0.8775 |
| 845_I_−1 | C | 13.3% | 64 | 13.0% | 23 | 1.0000 | 0.8761 |
| 845_H_+1 | G | 20.1% | 77 | 10.9% | 23 | 0.1915 | 0.3921 |
| 845_H_−1 | T | 48.1% | 77 | 50.0% | 22 | 0.8652 | 0.3922 |
| 845_G_+1 | T | 13.2% | 76 | 10.9% | 23 | 0.8037 | 1.0000 |
| 845_F_+1 | T | 17.5% | 77 | 15.2% | 23 | 0.8253 | 0.6755 |
| 845_D_1 | T | 0.7% | 76 | 0.0% | 21 | 1.0000 | 1.0000 |
| 845_D_−1 | T | 11.0% | 77 | 14.3% | 21 | 0.5906 | 0.2681 |
| 847_K_1 | A | 7.3% | 75 | 8.3% | 24 | 0.7617 | 0.7545 |
| 847_J_+1 | T | 8.4% | 77 | 2.1% | 24 | 0.1949 | 0.1777 |
| 847_E_+1 | C | 12.3% | 77 | 9.1% | 22 | 0.7899 | 0.7751 |
| 847_D_−1 | C | 17.6% | 74 | 7.5% | 20 | 0.1431 | 0.3321 |
| 847_C_+1 | G | 18.2% | 77 | 13.0% | 23 | 0.5067 | 0.9089 |
| 847_A_2 | T | 9.1% | 77 | 8.3% | 24 | 1.0000 | 1.0000 |
| 847_A_1 | A | 0.7% | 72 | 0.0% | 20 | 1.0000 | 1.0000 |
| 874_R_+1 | T | 37.0% | 73 | 35.0% | 20 | 0.8552 | 0.4180 |
| 874_S_+1 | A | 42.2% | 77 | 40.5% | 21 | 0.8620 | 0.7119 |
| 874_T_−1 | T | 48.1% | 77 | 47.6% | 21 | 1.0000 | 0.8466 |
| 874_V_−1 | G | 18.8% | 77 | 19.1% | 21 | 1.0000 | 0.7254 |
| 803_K_3 | T | 0.6% | 78 | 0.0% | 22 | 1.0000 | 1.0000 |
| 803_K_2 | A | 0.0% | 78 | 4.6% | 22 | 0.0475 | 0.0467 |
| 803_I_1 | G | 27.6% | 78 | 36.4% | 22 | 0.2666 | 0.4829 |
| 803_I_−1 | C | 0.0% | 78 | 0.0% | 22 | 1.0000 | 1.0000 |
| 803_H_+1 | A | 24.4% | 78 | 20.5% | 22 | 0.6895 | 0.9220 |
| 803_E_+2 | A | 47.4% | 76 | 38.6% | 22 | 0.3902 | 0.6005 |
| 962_E_3 | C | 33.6% | 76 | 47.8% | 23 | 0.0842 | 0.0784 |
| 962_E_+2 | C | 13.0% | 77 | 12.5% | 24 | 1.0000 | 0.2140 |
| 962_G_4 | A | 12.9% | 70 | 6.3% | 24 | 0.2910 | 0.5390 |
| 962_G_1 | A | 30.8% | 78 | 37.5% | 24 | 0.3839 | 0.5552 |
| 962_G_2 | A | 9.7% | 77 | 8.3% | 24 | 1.0000 | 1.0000 |
| 962_G_6 | T | 17.7% | 65 | 21.7% | 23 | 0.5195 | 0.7849 |
| 962_H_+2 | A | 37.7% | 77 | 39.6% | 24 | 0.8654 | 1.0000 |
| 962_M_+2 | C | 13.2% | 76 | 15.2% | 23 | 0.8065 | 0.0336 |
| 962_P_−2 | A | 22.4% | 78 | 40.5% | 21 | 0.0286 | 0.0227 |
| 962_Q_−1 | A | 21.7% | 76 | 39.1% | 23 | 0.0218 | 0.0375 |
| 962_S_−1 | C | 12.2% | 78 | 12.5% | 24 | 1.0000 | 0.8999 |
| 962_U_1 | A | 1.9% | 78 | 2.4% | 21 | 1.0000 | 1.0000 |
| 962_U_2 | T | 21.7% | 76 | 39.6% | 24 | 0.0225 | 0.0284 |
| 962_V_−1 | T | 35.4% | 65 | 23.9% | 23 | 0.1995 | 0.1120 |
| 962_V_+2 | A | 4.7% | 75 | 2.1% | 24 | 0.6823 | 0.6754 |
| 962_Z_1 | T | 35.5% | 76 | 25.0% | 24 | 0.2185 | 0.2520 |

b. Bronchial Hyper-responsiveness: The analyses were repeated using asthmatic children with borderline to severe BHR (PC₂₀≦16 mg/ml) or PC₂₀(16), as described in the Linkage Analysis section. (Example 3). First, sibling pairs were identified where both sibs were affected and satisfied this new criterion. Of these pairs, one sib was included in the case/control analyses if they showed evidence of linkage at the gene of interest. This phenotype was more restrictive than the Asthma yes/no criteria; hence the number of cases included in the analyses was reduced by approximately 57%. Where the PC₂₀(16) subgroup represented a more genetically homogeneous sample, one could expect an increase in the effect size compared to the one observed in the original set of cases. However, the reduction in sample size could result in estimates that were less accurate. This, in turn, could obscure a trend in allele frequencies in the control group, the original set of cases, and the PC₂₀(16) subgroup. In addition, the reduction in sample size could induce a reduction in power (and increase in p-values) in spite of the larger effect size.

The significance levels (p-values) for allele and genotype association of all typed SNPs to the BHR phenotype are shown in Tables 12, 13, and 14 for the combined population and for the UK and US populations separately. Allele frequencies are also included in the tables.

TABLE 12
ASSOCIATION ANALYSIS OF BHR
PHENOTYPE COMBINED US/UK POPULATION
Combined US & UK

| | | | FREQUENCIES | | | ALLELE | GENOTYPE |
|---|---|---|---|---|---|---|---|
| GENE_EXON | ALLELE | CNTL | N | CASE | N | P-VALUE | P-VALUE |
| 845_R_1 | A | 14.8% | 216 | 8.3% | 42 | 0.1227 | 0.3809 |
| 845_R_−1 | T | 28.7% | 214 | 23.8% | 42 | 0.4252 | 0.5421 |
| 845_P_+1 | T | 6.5% | 217 | 12.5% | 44 | 0.0715 | 0.0821 |
| 845_K_1 | T | 0.2% | 210 | 0.0% | 42 | 1.0000 | 1.0000 |
| 845_K_−2 | A | 28.9% | 216 | 20.9% | 43 | 0.1469 | 0.3532 |
| 845_J_1 | G | 34.5% | 210 | 50.0% | 43 | 0.0098 | 0.0151 |
| 845_J_−1 | C | 37.1% | 217 | 31.8% | 44 | 0.3952 | 0.5677 |
| 845_I_−1 | C | 12.8% | 191 | 12.5% | 44 | 1.0000 | 0.9160 |
| 845_H_+1 | G | 19.6% | 217 | 12.5% | 44 | 0.1325 | 0.2182 |
| 845_H_−1 | T | 45.2% | 217 | 37.5% | 44 | 0.1969 | 0.1876 |
| 845_G_+1 | T | 13.2% | 216 | 14.8% | 44 | 0.7318 | 0.4613 |
| 845_F_+1 | T | 18.4% | 215 | 18.2% | 44 | 1.0000 | 0.3208 |
| 845_D_1 | T | 0.2% | 216 | 1.2% | 42 | 0.2993 | 0.2996 |
| 845_D_−1 | T | 9.9% | 217 | 20.2% | 42 | 0.0139 | 0.0083 |
| 847_K_1 | A | 4.9% | 214 | 3.6% | 42 | 0.7810 | 0.7754 |
| 847_J_+1 | T | 4.9% | 216 | 1.1% | 47 | 0.1502 | 0.1413 |
| 847_E_+1 | C | 12.6% | 210 | 12.0% | 46 | 1.0000 | 0.9162 |
| 847_D_−1 | C | 16.9% | 210 | 15.1% | 43 | 0.7526 | 0.7296 |
| 847_C_+1 | G | 17.9% | 209 | 17.7% | 48 | 1.0000 | 1.0000 |
| 847_A_2 | T | 6.7% | 217 | 4.2% | 48 | 0.4852 | 0.4699 |
| 847_A_1 | A | 1.0% | 192 | 1.0% | 48 | 1.0000 | 1.0000 |
| 874_R_+1 | T | 39.9% | 202 | 29.3% | 46 | 0.0738 | 0.1586 |
| 874_S_+1 | A | 39.5% | 214 | 42.6% | 47 | 0.6422 | 0.8454 |
| 874_T_−1 | T | 48.6% | 213 | 46.9% | 48 | 0.8214 | 0.4498 |
| 874_V_−1 | G | 17.6% | 216 | 18.8% | 48 | 0.7695 | 0.6737 |
| 803_K_3 | T | 0.9% | 218 | 2.6% | 58 | 0.1647 | 0.1635 |
| 803_K_2 | A | 0.2% | 217 | 0.9% | 58 | 0.3776 | 0.3779 |
| 803_I_1 | G | 28.2% | 195 | 28.5% | 58 | 1.0000 | 1.0000 |
| 803_I_−1 | C | 0.2% | 217 | 0.0% | 56 | 1.0000 | 1.0000 |
| 803_H_+1 | A | 25.2% | 218 | 21.9% | 57 | 0.5414 | 0.6420 |
| 803_E_+2 | A | 44.2% | 208 | 46.5% | 57 | 0.6722 | 0.8478 |
| 962_E_3 | C | 35.6% | 212 | 34.7% | 49 | 0.9071 | 0.9013 |
| 962_E_+2 | C | 12.7% | 217 | 8.7% | 52 | 0.3128 | 0.5841 |
| 962_G_4 | A | 13.6% | 202 | 8.3% | 48 | 0.1756 | 0.2010 |
| 962_G_1 | A | 27.2% | 217 | 29.8% | 52 | 0.6260 | 0.5278 |
| 962_G_2 | A | 7.6% | 217 | 8.8% | 51 | 0.6830 | 0.6707 |
| 962_G_6 | T | 20.4% | 194 | 15.3% | 49 | 0.3165 | 0.4404 |
| 962_H_+2 | A | 41.5% | 217 | 35.3% | 51 | 0.2641 | 0.2847 |
| 962_M_+2 | C | 12.7% | 213 | 8.2% | 49 | 0.2968 | 0.2530 |
| 962_P_−2 | A | 23.8% | 214 | 21.4% | 49 | 0.6921 | 0.4539 |
| 962_Q_−1 | A | 23.7% | 215 | 21.9% | 48 | 0.7901 | 0.6686 |
| 962_S_−1 | C | 11.4% | 215 | 7.8% | 51 | 0.3741 | 0.4768 |
| 962_U_1 | A | 3.0% | 214 | 4.9% | 51 | 0.3617 | 0.3548 |
| 962_U_2 | T | 23.8% | 212 | 20.0% | 50 | 0.5095 | 0.7424 |
| 962_V_−1 | T | 34.8% | 187 | 26.5% | 49 | 0.1476 | 0.2075 |
| 962_V_+2 | A | 4.5% | 209 | 2.0% | 51 | 0.3973 | 0.5039 |
| 962_Z_1 | T | 31.9% | 216 | 37.5% | 52 | 0.2968 | 0.2436 |

TABLE 13
ASSOCIATION ANALYSIS OF BHR
PHENOTYPE UK POPULATION
UK population

| | | | FREQUENCIES | | | ALLELE | GENOTYPE |
|---|---|---|---|---|---|---|---|
| GENE_EXON | ALLELE | CNTL | N | CASE | N | P-VALUE | P-VALUE |
| 845_R_1 | A | 13.6% | 140 | 9.7% | 36 | 0.4358 | 0.9112 |
| 845_R_−1 | T | 26.6% | 137 | 21.4% | 35 | 0.4437 | 0.3471 |
| 845_P_+1 | T | 6.4% | 140 | 10.8% | 37 | 0.2120 | 0.2939 |
| 845_K_1 | T | 0.4% | 135 | 0.0% | 35 | 1.0000 | 1.0000 |
| 845_K_−2 | A | 27.0% | 139 | 18.1% | 36 | 0.1299 | 0.3673 |
| 845_J_1 | G | 36.6% | 134 | 51.4% | 36 | 0.0296 | 0.0475 |
| 845_J_−1 | C | 37.5% | 140 | 33.8% | 37 | 0.5899 | 0.8093 |
| 845_I_−1 | C | 12.6% | 127 | 12.2% | 37 | 1.0000 | 1.0000 |
| 845_H_+1 | G | 19.3% | 140 | 14.9% | 37 | 0.4994 | 0.7871 |
| 845_H_−1 | T | 43.6% | 140 | 33.8% | 37 | 0.1454 | 0.1454 |
| 845_G_+1 | T | 13.2% | 140 | 14.9% | 37 | 0.7047 | 0.5094 |
| 845_F_+1 | T | 18.8% | 138 | 18.9% | 37 | 1.0000 | 0.5839 |
| 845_D_1 | T | 0.0% | 140 | 1.4% | 36 | 0.2045 | 0.2045 |
| 845_D_−1 | T | 9.3% | 140 | 20.8% | 36 | 0.0120 | 0.0069 |
| 847_K_1 | A | 3.6% | 139 | 4.4% | 34 | 0.7250 | 0.7214 |
| 847_J_+1 | T | 2.9% | 139 | 1.3% | 39 | 0.6900 | 0.6859 |
| 847_E_+1 | C | 12.8% | 133 | 14.1% | 39 | 0.8486 | 0.4668 |
| 847_D_−1 | C | 16.5% | 136 | 18.6% | 35 | 0.7215 | 0.8339 |
| 847_C_+1 | G | 17.8% | 132 | 21.3% | 40 | 0.5130 | 0.6295 |
| 847_A_2 | T | 5.4% | 140 | 5.0% | 40 | 1.0000 | 1.0000 |
| 847_A_1 | A | 1.3% | 120 | 1.3% | 40 | 1.0000 | 1.0000 |
| 874_R_+1 | T | 41.5% | 129 | 30.8% | 39 | 0.1116 | 0.2579 |
| 874_S_+1 | A | 38.0% | 137 | 44.9% | 39 | 0.2947 | 0.4876 |
| 874_T_−1 | T | 48.9% | 136 | 47.5% | 40 | 0.8989 | 0.7399 |
| 874_V_−1 | G | 16.9% | 139 | 18.8% | 40 | 0.7379 | 0.8269 |
| 803_K_3 | T | 1.1% | 140 | 3.1% | 49 | 0.1828 | 0.1812 |
| 803_K_2 | A | 0.4% | 139 | 0.0% | 49 | 1.0000 | 1.0000 |
| 803_I_1 | G | 28.6% | 117 | 25.5% | 49 | 0.5933 | 0.8653 |
| 803_I_−1 | C | 0.4% | 139 | 0.0% | 47 | 1.0000 | 1.0000 |
| 803_H_+1 | A | 25.7% | 140 | 21.9% | 48 | 0.4953 | 0.7481 |
| 803_E_+2 | A | 42.4% | 132 | 49.0% | 48 | 0.2821 | 0.4597 |
| 962_E_3 | C | 36.8% | 136 | 32.1% | 42 | 0.5150 | 0.7793 |
| 962_E_+2 | C | 12.5% | 140 | 10.0% | 45 | 0.5802 | 0.9068 |
| 962_G_4 | A | 14.0% | 132 | 8.5% | 41 | 0.2549 | 0.2572 |
| 962_G_1 | A | 25.2% | 139 | 28.9% | 45 | 0.4922 | 0.3482 |
| 962_G_2 | A | 6.4% | 140 | 10.2% | 44 | 0.2444 | 0.2270 |
| 962_G_6 | T | 21.7% | 129 | 16.7% | 42 | 0.3540 | 0.2230 |
| 962_H_+2 | A | 43.6% | 140 | 35.2% | 44 | 0.1748 | 0.2934 |
| 962_M_+2 | C | 12.4% | 137 | 7.1% | 42 | 0.2352 | 0.5149 |
| 962_P_−2 | A | 24.6% | 136 | 19.3% | 44 | 0.3843 | 0.6062 |
| 962_Q_−1 | A | 24.8% | 139 | 20.2% | 42 | 0.4650 | 0.5108 |
| 962_S_−1 | C | 10.9% | 137 | 6.8% | 44 | 0.3107 | 0.7140 |
| 962_U_1 | A | 3.7% | 136 | 4.5% | 44 | 0.7524 | 0.7481 |
| 962_U_2 | T | 25.0% | 136 | 17.4% | 43 | 0.1868 | 0.3713 |
| 962_V_−1 | T | 34.4% | 122 | 25.0% | 42 | 0.1357 | 0.1353 |
| 962_V_+2 | A | 4.5% | 134 | 2.3% | 44 | 0.5311 | 0.7988 |
| 962_Z_1 | T | 30.0% | 140 | 40.0% | 45 | 0.0924 | 0.0467 |

TABLE 14
ASSOCIATION ANALYSIS OF
BHR PHENOTYPE US POPULATION
US population

| | | | FREQUENCIES | | | ALLELE | GENOTYPE |
|---|---|---|---|---|---|---|---|
| GENE_EXON | ALLELE | CNTL | N | CASE | N | P-VALUE | P-VALUE |
| 845_R_1 | A | 17.1% | 76 | 0.0% | 6 | 0.2168 | 0.2965 |
| 845_R_−1 | T | 32.5% | 77 | 35.7% | 7 | 0.7742 | 0.6790 |
| 845_P_+1 | T | 6.5% | 77 | 21.4% | 7 | 0.0801 | 0.1254 |
| 845_K_1 | T | 0.0% | 75 | 0.0% | 7 | 1.0000 | 1.0000 |
| 845_K_−2 | A | 32.5% | 77 | 35.7% | 7 | 0.7742 | 0.6790 |
| 845_J_1 | G | 30.9% | 76 | 42.9% | 7 | 0.3787 | 0.5066 |
| 845_J_−1 | C | 36.4% | 77 | 21.4% | 7 | 0.3830 | 0.5888 |
| 845_I_−1 | C | 13.3% | 64 | 14.3% | 7 | 1.0000 | 0.7087 |
| 845_H_+1 | G | 20.1% | 77 | 0.0% | 7 | 0.0752 | 0.1660 |
| 845_H_−1 | T | 48.1% | 77 | 57.1% | 7 | 0.5841 | 0.7769 |
| 845_G_+1 | T | 13.2% | 76 | 14.3% | 7 | 1.0000 | 0.7054 |
| 845_F_+1 | T | 17.5% | 77 | 14.3% | 7 | 1.0000 | 1.0000 |
| 845_D_1 | T | 0.7% | 76 | 0.0% | 6 | 1.0000 | 1.0000 |
| 845_D_−1 | T | 11.0% | 77 | 16.7% | 6 | 0.6307 | 0.6162 |
| 847_K_1 | A | 7.3% | 75 | 0.0% | 8 | 0.6027 | 0.5892 |
| 847_J_+1 | T | 8.4% | 77 | 0.0% | 8 | 0.6146 | 0.3488 |
| 847_E_+1 | C | 12.3% | 77 | 0.0% | 7 | 0.3721 | 0.3417 |
| 847_D_−1 | C | 17.6% | 74 | 0.0% | 8 | 0.0779 | 0.2076 |
| 847_C_+1 | G | 18.2% | 77 | 0.0% | 8 | 0.0770 | 0.1869 |
| 847_A_2 | T | 9.1% | 77 | 0.0% | 8 | 0.3669 | 0.3423 |
| 847_A_1 | A | 0.7% | 72 | 0.0% | 8 | 1.0000 | 1.0000 |
| 874_R_+1 | T | 37.0% | 73 | 21.4% | 7 | 0.3818 | 0.6663 |
| 874_S_+1 | A | 42.2% | 77 | 31.3% | 8 | 0.4382 | 0.6119 |
| 874_T_−1 | T | 48.1% | 77 | 43.8% | 8 | 0.7977 | 0.6137 |
| 874_V_−1 | G | 18.8% | 77 | 18.8% | 8 | 1.0000 | 0.7807 |
| 803_K_3 | T | 0.6% | 78 | 0.0% | 9 | 1.0000 | 1.0000 |
| 803_K_2 | A | 0.0% | 78 | 5.6% | 9 | 0.1034 | 0.1034 |
| 803_I_1 | G | 27.6% | 78 | 44.4% | 9 | 0.1714 | 0.2111 |
| 803_I_−1 | C | 0.0% | 78 | 0.0% | 9 | 1.0000 | 1.0000 |
| 803_H_+1 | A | 24.4% | 78 | 22.2% | 9 | 1.0000 | 0.8463 |
| 803_E_+2 | A | 47.4% | 76 | 33.3% | 9 | 0.3214 | 0.6636 |
| 962_E_3 | C | 33.6% | 76 | 50.0% | 7 | 0.2478 | 0.1841 |
| 962_E_+2 | C | 13.0% | 77 | 0.0% | 7 | 0.3791 | 0.1892 |
| 962_G_4 | A | 12.9% | 70 | 7.1% | 7 | 1.0000 | 1.0000 |
| 962_G_1 | A | 30.8% | 78 | 35.7% | 7 | 0.7656 | 0.6406 |
| 962_G_2 | A | 9.7% | 77 | 0.0% | 7 | 0.6172 | 0.3425 |
| 962_G_6 | T | 17.7% | 65 | 7.1% | 7 | 0.4658 | 1.0000 |
| 962_H_+2 | A | 37.7% | 77 | 35.7% | 7 | 1.0000 | 0.2819 |
| 962_M_+2 | C | 13.2% | 76 | 14.3% | 7 | 1.0000 | 0.1122 |
| 962_P_−2 | A | 22.4% | 78 | 40.0% | 5 | 0.2469 | 0.2330 |
| 962_D_−1 | A | 21.7% | 76 | 33.3% | 6 | 0.4707 | 0.3338 |
| 962_S_−1 | C | 12.2% | 78 | 14.3% | 7 | 0.6849 | 0.1564 |
| 962_U_1 | A | 1.9% | 78 | 7.1% | 7 | 0.2932 | 0.2955 |
| 962_U_2 | T | 21.7% | 76 | 35.7% | 7 | 0.3150 | 0.2666 |
| 962_V_−1 | T | 35.4% | 65 | 35.7% | 7 | 1.0000 | 1.0000 |
| 962_V_+2 | A | 4.7% | 75 | 0.0% | 7 | 1.0000 | 1.0000 |
| 962_Z_1 | T | 35.5% | 76 | 21.4% | 7 | 0.3845 | 0.7311 |

For the BHR phenotype, two SNPs in Gene 845 reached statistical significance in the combined and the UK population alone for both the allele and the genotype tests: 50.0% of the cases in the combined population were carriers of the G allele in SNP J 1, whereas the G allele was observed in only 34.5% of the controls (combined: allele test p=0.0098, genotype test p=0.0151; UK: allele test p=0.0296, genotype test p=0.0475) and 20.2% of the cases in the combined population were carriers of the T allele in SNP D−1, whereas the T allele was observed in only 9.9% of the controls (combined: allele test p=0.0139, genotype test p=0.0083; UK: allele test p=0.0120, genotype test p=0.0069).

c. Total IqE: The analyses were performed using asthmatic children with elevated total IgE levels, as described in the Linkage Analysis section (Example 3). First, sibling pairs were identified where both sibs were affected and satisfied this new criterion. Of these pairs, one sib was included in the case/control analyses if they showed evidence of linkage at the gene of interest. This phenotype was more restrictive than the Asthma yes/no criteria; hence the number of cases included in the analyses was reduced by approximately 42%.

The significance levels (p-values) for allele and genotype association of all typed SNPs to the IgE phenotype are shown in Tables 15, 16, and 17 for the combined population and for the UK and US populations, separately. Allele frequencies are also included in the tables.

TABLE 15
ASSOCIATION ANALYSIS OF TOTAL IgE
PHENOTYPE COMBINED US/UK POPULATION
Combined US & UK

| | | | FREQUENCIES | | | ALLELE | GENOTYPE |
|---|---|---|---|---|---|---|---|
| GENE_EXON | ALLELE | CNTL | N | CASE | N | P-VALUE | P-VALUE |
| 845_R_1 | A | 14.8% | 216 | 14.1% | 64 | 0.8877 | 1.0000 |
| 845_R_−1 | T | 28.7% | 214 | 21.1% | 64 | 0.0901 | 0.2230 |
| 845_P_+1 | T | 6.5% | 217 | 10.8% | 65 | 0.1257 | 0.2366 |
| 845_K_1 | T | 0.2% | 210 | 0.0% | 59 | 1.0000 | 1.0000 |
| 845_K_−2 | A | 28.9% | 216 | 22.1% | 61 | 0.1677 | 0.3144 |
| 845_J_1 | G | 34.5% | 210 | 40.0% | 65 | 0.2952 | 0.4631 |
| 845_J_−1 | C | 37.1% | 217 | 40.0% | 65 | 0.6062 | 0.7606 |
| 845_I_−1 | C | 12.8% | 191 | 11.5% | 65 | 0.7609 | 0.8852 |
| 845_H_+1 | G | 19.6% | 217 | 16.2% | 65 | 0.4431 | 0.5034 |
| 845_H_−1 | T | 45.2% | 217 | 41.4% | 64 | 0.4795 | 0.7050 |
| 845_G_+1 | T | 13.2% | 216 | 14.6% | 65 | 0.6628 | 0.8129 |
| 845_F_+1 | T | 18.4% | 215 | 18.5% | 65 | 1.0000 | 0.5492 |
| 845_D_1 | T | 0.2% | 216 | 0.8% | 62 | 0.3966 | 0.3969 |
| 845_D_−1 | T | 9.9% | 217 | 12.1% | 62 | 0.5050 | 0.4794 |
| 847_K_1 | A | 4.9% | 214 | 5.0% | 60 | 1.0000 | 1.0000 |
| 847_J_+1 | T | 4.9% | 216 | 3.0% | 67 | 0.4732 | 0.4625 |
| 847_E_+1 | C | 12.6% | 210 | 15.2% | 66 | 0.4629 | 0.3564 |
| 847_D_−1 | C | 16.9% | 210 | 17.9% | 56 | 0.7795 | 0.6697 |
| 847_C_+1 | G | 17.9% | 209 | 21.5% | 65 | 0.3696 | 0.0887 |
| 847_A_2 | T | 6.7% | 217 | 7.5% | 67 | 0.7007 | 0.8391 |
| 847_A_1 | A | 1.0% | 192 | 0.8% | 62 | 1.0000 | 1.0000 |
| 874_R_+1 | T | 39.9% | 202 | 32.0% | 64 | 0.1181 | 0.3281 |
| 874_S_+1 | A | 39.5% | 214 | 40.4% | 68 | 0.8413 | 0.9644 |
| 874_T_−1 | T | 48.6% | 213 | 50.0% | 69 | 0.8447 | 0.9345 |
| 874_V_−1 | G | 17.6% | 216 | 21.0% | 69 | 0.3785 | 0.1676 |
| 803_K_3 | T | 0.9% | 218 | 2.2% | 67 | 0.3637 | 0.3609 |
| 803_K_2 | A | 0.2% | 217 | 1.5% | 67 | 0.1402 | 0.1397 |
| 803_I_1 | G | 28.2% | 195 | 28.8% | 66 | 0.9113 | 0.5678 |
| 803_I_−1 | C | 0.2% | 217 | 0.0% | 64 | 1.0000 | 1.0000 |
| 803_H_+1 | A | 25.2% | 218 | 23.5% | 66 | 0.7311 | 0.7690 |
| 803_E_+2 | A | 44.2% | 208 | 45.4% | 65 | 0.8402 | 0.9639 |
| 962_E_3 | C | 35.6% | 212 | 32.1% | 70 | 0.4753 | 0.4122 |
| 962_E_+2 | C | 12.7% | 217 | 6.8% | 73 | 0.0677 | 0.1672 |
| 962_G_4 | A | 13.6% | 202 | 7.1% | 70 | 0.0487 | 0.0749 |
| 962_G_1 | A | 27.2% | 217 | 37.0% | 73 | 0.0280 | 0.0472 |
| 962_G_2 | A | 7.6% | 217 | 7.7% | 71 | 1.0000 | 1.0000 |
| 962_G_6 | T | 20.4% | 194 | 16.2% | 71 | 0.3205 | 0.0794 |
| 962_H_+2 | A | 41.5% | 217 | 34.0% | 72 | 0.1170 | 0.2501 |
| 962_M_+2 | C | 12.7% | 213 | 10.7% | 70 | 0.6553 | 0.5397 |
| 962_P_−2 | A | 23.8% | 214 | 25.4% | 69 | 0.7323 | 0.4327 |
| 962_Q_−1 | A | 23.7% | 215 | 24.6% | 69 | 0.8195 | 0.8627 |
| 962_S_−1 | C | 11.4% | 215 | 8.3% | 72 | 0.3507 | 0.6768 |
| 962_U_1 | A | 3.0% | 214 | 3.6% | 70 | 0.7821 | 0.7789 |
| 962_U_2 | T | 23.8% | 212 | 23.9% | 71 | 1.0000 | 0.8648 |
| 962_V_−1 | T | 34.8% | 187 | 30.4% | 69 | 0.3994 | 0.2858 |
| 962_V_+2 | A | 4.5% | 209 | 3.5% | 72 | 0.8112 | 1.0000 |
| 962_Z_1 | T | 31.9% | 216 | 31.5% | 73 | 1.0000 | 0.5168 |

TABLE 16
ASSOCIATION ANALYSIS OF TOTAL IgE
PHENOTYPE UK POPULATION
UK population

| | | | FREQUENCIES | | | ALLELE | GENOTYPE |
|---|---|---|---|---|---|---|---|
| GENE_EXON | ALLELE | CNTL | N | CASE | N | P-VALUE | P-VALUE |
| 845_R_1 | A | 13.6% | 140 | 15.4% | 52 | 0.6245 | 0.8181 |
| 845_R_−1 | T | 26.6% | 137 | 20.2% | 52 | 0.2308 | 0.3992 |
| 845_P_+1 | T | 6.4% | 140 | 9.4% | 53 | 0.3784 | 0.3276 |
| 845_K_1 | T | 0.4% | 135 | 0.0% | 47 | 1.0000 | 1.0000 |
| 845_K_−2 | A | 27.0% | 139 | 21.0% | 50 | 0.2842 | 0.4390 |
| 845_J_1 | G | 36.6% | 134 | 40.6% | 53 | 0.4798 | 0.7186 |
| 845_J_−1 | C | 37.5% | 140 | 40.6% | 53 | 0.6392 | 0.8105 |
| 845_I_−1 | C | 12.6% | 127 | 11.3% | 53 | 0.8603 | 1.0000 |
| 845_H_+1 | G | 19.3% | 140 | 16.0% | 53 | 0.5564 | 0.4068 |
| 845_H_−1 | T | 43.6% | 140 | 39.6% | 53 | 0.4918 | 0.8043 |
| 845_G_+1 | T | 13.2% | 140 | 16.0% | 53 | 0.5116 | 0.7663 |
| 845_F_+1 | T | 18.8% | 138 | 18.9% | 53 | 1.0000 | 0.8226 |
| 845_D_1 | T | 0.0% | 140 | 1.0% | 51 | 0.2670 | 0.2670 |
| 845_D_−1 | T | 9.3% | 140 | 11.8% | 51 | 0.4474 | 0.5389 |
| 847_K_1 | A | 3.6% | 139 | 3.2% | 47 | 1.0000 | 1.0000 |
| 847_J_+1 | T | 2.9% | 139 | 2.8% | 54 | 1.0000 | 1.0000 |
| 847_E_+1 | C | 12.8% | 133 | 17.9% | 53 | 0.2494 | 0.0776 |
| 847_D_−1 | C | 16.5% | 136 | 21.6% | 44 | 0.3355 | 0.1746 |
| 847_C_+1 | G | 17.8% | 132 | 24.5% | 53 | 0.1502 | 0.0228 |
| 847_A_2 | T | 5.4% | 140 | 7.4% | 54 | 0.4740 | 0.4606 |
| 847_A_1 | A | 1.3% | 120 | 0.9% | 53 | 1.0000 | 1.0000 |
| 874_R_+1 | T | 41.5% | 129 | 33.3% | 54 | 0.1597 | 0.3907 |
| 874_S_+1 | A | 38.0% | 137 | 39.5% | 57 | 0.8191 | 0.5707 |
| 874_T_−1 | T | 48.9% | 136 | 52.6% | 58 | 0.5794 | 0.7577 |
| 874_V_−1 | G | 16.9% | 139 | 19.8% | 58 | 0.4743 | 0.4157 |
| 803_K_3 | T | 1.1% | 140 | 2.6% | 58 | 0.3645 | 0.3612 |
| 803_K_2 | A | 0.4% | 139 | 1.7% | 58 | 0.2083 | 0.2077 |
| 803_I_1 | G | 28.6% | 117 | 26.3% | 57 | 0.7033 | 0.3961 |
| 803_I_−1 | C | 0.4% | 139 | 0.0% | 55 | 1.0000 | 1.0000 |
| 803_H_+1 | A | 25.7% | 140 | 23.7% | 57 | 0.7028 | 0.8814 |
| 803_E_+2 | A | 42.4% | 132 | 48.2% | 56 | 0.3091 | 0.5632 |
| 962_E_3 | C | 36.8% | 136 | 31.0% | 58 | 0.2974 | 0.5165 |
| 962_E_+2 | C | 12.5% | 140 | 7.4% | 61 | 0.1645 | 0.3555 |
| 962_G_4 | A | 14.0% | 132 | 7.8% | 58 | 0.0904 | 0.1867 |
| 962_G_1 | A | 25.2% | 139 | 34.4% | 61 | 0.0695 | 0.1331 |
| 962_G_2 | A | 6.4% | 140 | 7.6% | 59 | 0.6660 | 0.6550 |
| 962_G_6 | T | 21.7% | 129 | 14.4% | 59 | 0.1218 | 0.0239 |
| 962_H_+2 | A | 43.6% | 140 | 35.8% | 60 | 0.1834 | 0.2912 |
| 962_M_+2 | C | 12.4% | 137 | 8.6% | 58 | 0.3811 | 0.6573 |
| 962_P_−2 | A | 24.6% | 136 | 21.2% | 59 | 0.5173 | 0.8032 |
| 962_Q_−1 | A | 24.8% | 139 | 20.7% | 58 | 0.4355 | 0.7382 |
| 962_S_−1 | C | 10.9% | 137 | 6.7% | 60 | 0.2008 | 0.4682 |
| 962_U_1 | A | 3.7% | 136 | 3.4% | 59 | 1.0000 | 1.0000 |
| 962_U_2 | T | 25.0% | 136 | 19.5% | 59 | 0.2970 | 0.6158 |
| 962_V_−1 | T | 34.4% | 122 | 31.0% | 58 | 0.5516 | 0.2373 |
| 962_V_+2 | A | 4.5% | 134 | 3.3% | 60 | 0.7845 | 1.0000 |
| 962_Z_1 | T | 30.0% | 140 | 33.6% | 61 | 0.4837 | 0.2793 |

TABLE 17
ASSOCIATION ANALYSIS OF TOTAL IgE
PHENOTYPE US POPULATION
US population

| | | | FREQUENCIES | | | ALLELE | GENOTYPE |
|---|---|---|---|---|---|---|---|
| GENE_EXON | ALLELE | CNTL | N | CASE | N | P-VALUE | P-VALUE |
| 845_R_1 | A | 17.1% | 76 | 8.3% | 12 | 0.3763 | 0.6251 |
| 845_R_−1 | T | 32.5% | 77 | 25.0% | 12 | 0.6371 | 0.8973 |
| 845_P_+1 | T | 6.5% | 77 | 16.7% | 12 | 0.1002 | 0.1818 |
| 845_K_1 | T | 0.0% | 75 | 0.0% | 12 | 1.0000 | 1.0000 |
| 845_K_−2 | A | 32.5% | 77 | 27.3% | 11 | 0.8075 | 1.0000 |
| 845_J_1 | G | 30.9% | 76 | 37.5% | 12 | 0.6376 | 0.7377 |
| 845_J_−1 | C | 36.4% | 77 | 37.5% | 12 | 1.0000 | 1.0000 |
| 845_I_−1 | C | 13.3% | 64 | 12.5% | 12 | 1.0000 | 0.7982 |
| 845_H_+1 | G | 20.1% | 77 | 16.7% | 12 | 1.0000 | 1.0000 |
| 845_H_−1 | T | 48.1% | 77 | 50.0% | 11 | 1.0000 | 0.7010 |
| 845_G_+1 | T | 13.2% | 76 | 8.3% | 12 | 0.7426 | 1.0000 |
| 845_F_+1 | T | 17.5% | 77 | 16.7% | 12 | 1.0000 | 0.8486 |
| 845_D_1 | T | 0.7% | 76 | 0.0% | 11 | 1.0000 | 1.0000 |
| 845_D_−1 | T | 11.0% | 77 | 13.6% | 11 | 0.7202 | 0.7075 |
| 847_K_1 | A | 7.3% | 75 | 11.5% | 13 | 0.4387 | 0.4273 |
| 847_J_+1 | T | 8.4% | 77 | 3.8% | 13 | 0.6961 | 0.6829 |
| 847_E_+1 | C | 12.3% | 77 | 3.8% | 13 | 0.3158 | 0.2829 |
| 847_D_−1 | C | 17.6% | 74 | 4.2% | 12 | 0.1308 | 0.3152 |
| 847_C_+1 | G | 18.2% | 77 | 8.3% | 12 | 0.3780 | 0.6757 |
| 847_A_2 | T | 9.1% | 77 | 7.7% | 13 | 1.0000 | 1.0000 |
| 847_A_1 | A | 0.7% | 72 | 0.0% | 9 | 1.0000 | 1.0000 |
| 874_R_+1 | T | 37.0% | 73 | 25.0% | 10 | 0.3317 | 0.7405 |
| 874_S_+1 | A | 42.2% | 77 | 45.5% | 11 | 0.8201 | 0.4213 |
| 874_T_−1 | T | 48.1% | 77 | 36.4% | 11 | 0.3647 | 0.3947 |
| 874_V_−1 | G | 18.8% | 77 | 27.3% | 11 | 0.3924 | 0.2974 |
| 803_K_3 | T | 0.6% | 78 | 0.0% | 9 | 1.0000 | 1.0000 |
| 803_K_2 | A | 0.0% | 78 | 0.0% | 9 | 1.0000 | 1.0000 |
| 803_I_1 | G | 27.6% | 78 | 44.4% | 9 | 0.1714 | 0.2111 |
| 803_I_−1 | C | 0.0% | 78 | 0.0% | 9 | 1.0000 | 1.0000 |
| 803_H_+1 | A | 24.4% | 78 | 22.2% | 9 | 1.0000 | 0.8463 |
| 803_E_+2 | A | 47.4% | 76 | 27.8% | 9 | 0.1375 | 0.3483 |
| 962_E_3 | C | 33.6% | 76 | 37.5% | 12 | 0.8172 | 0.5320 |
| 962_E_+2 | C | 13.0% | 77 | 4.2% | 12 | 0.3160 | 0.2803 |
| 962_G_4 | A | 12.9% | 70 | 4.2% | 12 | 0.3133 | 0.5253 |
| 962_G_1 | A | 30.8% | 78 | 50.0% | 12 | 0.1014 | 0.0734 |
| 962_G_2 | A | 9.7% | 77 | 8.3% | 12 | 1.0000 | 1.0000 |
| 962_G_6 | T | 17.7% | 65 | 25.0% | 12 | 0.4013 | 0.5214 |
| 962_H_+2 | A | 37.7% | 77 | 25.0% | 12 | 0.2614 | 0.6315 |
| 962_M_+2 | C | 13.2% | 76 | 20.8% | 12 | 0.3451 | 0.0838 |
| 962_P_−2 | A | 22.4% | 78 | 50.0% | 10 | 0.0130 | 0.0116 |
| 962_Q_−1 | A | 21.7% | 76 | 45.5% | 11 | 0.0309 | 0.0261 |
| 962_S_−1 | C | 12.2% | 78 | 16.7% | 12 | 0.5172 | 0.5073 |
| 962_U_1 | A | 1.9% | 78 | 4.5% | 11 | 0.4129 | 0.4158 |
| 962_U_2 | T | 21.7% | 76 | 45.8% | 12 | 0.0201 | 0.0233 |
| 962_V_−1 | T | 35.4% | 65 | 27.3% | 11 | 0.6278 | 0.8097 |
| 962_V_+2 | A | 4.7% | 75 | 4.2% | 12 | 1.0000 | 1.0000 |
| 962_Z_1 | T | 35.5% | 76 | 20.8% | 12 | 0.2438 | 0.4274 |

For the total IgE phenotype, a single SNP in Gene 847 reached statistical significance in the UK population alone: SNP C+1. For this SNP, 24.5% of the cases were carriers of the G allele, whereas the G allele was observed in only 17.8% of the controls (genotype test p=0.0228). Six SNPs in Gene 962 reached statistical significance in the combined and the US population alone for both allele and genotype tests: 92.9% of the cases were carriers of the G allele in SNP G 4, whereas the G allele was observed in only 86.4% of the controls (combined: allele test p=0.0487), 37.0% of the cases were carriers of the A allele in SNP G 1, whereas the A allele was observed in only 27.2% of the controls (combined: allele test p=0.0280, genotype test p=0.0472), 85.6% of the cases were carriers of the C allele in SNP G 6, whereas the C allele was observed in only 78.3% of the controls (UK: genotype test p=0.0239), 50.0% of the cases were carriers of the A allele in SNP P−2, whereas the A allele was observed in only 22.4% of the controls (US: allele test p=0.0130, genotype test p=0.0116), 45.5% of the cases were carriers of the A allele in SNP Q−1, whereas the A allele was observed in only 21.7% of the controls (US: allele test p=0.0309, genotype test p=0.0261) and 45.8% of the cases were carriers of the T allele in SNP U 2, whereas the T allele was observed in only 21.7% of the controls (US: allele test p=0.0201 and genotype test p=0.0233).

d. Specific IgE: The analyses were performed using asthmatic children with elevated specific IgE levels for at least one allergen, as described in the Linkage Analysis section (Example 3). First, sibling pairs were identified where both sibs were affected and satisfied this new criterion. Of these pairs, one sib was included in the case/control analyses if they showed evidence of linkage at the gene of interest. This phenotype was more restrictive than the Asthma yes/no criteria; hence the number of cases included in the analyses was reduced by approximately 38%.

Frequencies and p-values for all typed SNPs are shown in Tables 18, 19 and 20 or the combined population and for the UK and US populations, separately.

TABLE 18
ASSOCIATION ANALYSIS OF SPECIFIC IgE
PHENOTYPE COMBINED US/UK POPULATION
Combined US & UK

| | | | FREQUENCIES | | | ALLELE | GENOTYPE |
|---|---|---|---|---|---|---|---|
| GENE_EXON | ALLELE | CNTL | N | CASE | N | P-VALUE | P-VALUE |
| 845_R_1 | A | 14.8% | 216 | 10.2% | 59 | 0.2296 | 0.3695 |
| 845_R_−1 | T | 28.7% | 214 | 24.2% | 60 | 0.3572 | 0.5546 |
| 845_P_+1 | T | 6.5% | 217 | 14.2% | 60 | 0.0126 | 0.0172 |
| 845_K_1 | T | 0.2% | 210 | 0.0% | 57 | 1.0000 | 1.0000 |
| 845_K_−2 | A | 28.9% | 216 | 24.6% | 57 | 0.4121 | 0.6113 |
| 845_J_1 | G | 34.5% | 210 | 43.3% | 60 | 0.0856 | 0.1956 |
| 845_J_−1 | C | 37.1% | 217 | 32.5% | 60 | 0.3910 | 0.5375 |
| 845_I_−1 | C | 12.8% | 191 | 11.7% | 60 | 0.8746 | 1.0000 |
| 845_H_+1 | G | 19.6% | 217 | 13.3% | 60 | 0.1414 | 0.2742 |
| 845_H_−1 | T | 45.2% | 217 | 40.7% | 59 | 0.4045 | 0.7192 |
| 845_G_+1 | T | 13.2% | 216 | 14.2% | 60 | 0.7635 | 0.8468 |
| 845_F_+1 | T | 18.4% | 215 | 19.2% | 60 | 0.8944 | 0.8931 |
| 845_D_1 | T | 0.2% | 216 | 0.9% | 57 | 0.3743 | 0.3746 |
| 845_D_−1 | T | 9.9% | 217 | 15.8% | 57 | 0.0931 | 0.0646 |
| 847_K_1 | A | 4.9% | 214 | 4.3% | 58 | 1.0000 | 1.0000 |
| 847_J_+1 | T | 4.9% | 216 | 1.6% | 64 | 0.1282 | 0.1200 |
| 847_E_+1 | C | 12.6% | 210 | 15.1% | 63 | 0.4567 | 0.3427 |
| 847_D_−1 | C | 16.9% | 210 | 17.6% | 54 | 0.8861 | 0.6933 |
| 847_C_+1 | G | 17.9% | 209 | 21.4% | 63 | 0.4345 | 0.1027 |
| 847_A_2 | T | 6.7% | 217 | 7.8% | 64 | 0.6925 | 0.6816 |
| 847_A_1 | A | 1.0% | 192 | 0.8% | 60 | 1.0000 | 1.0000 |
| 874_R_+1 | T | 39.9% | 202 | 32.5% | 60 | 0.1645 | 0.3165 |
| 874_S_+1 | A | 39.5% | 214 | 41.4% | 64 | 0.7578 | 0.8934 |
| 874_T_−1 | T | 48.6% | 213 | 49.2% | 65 | 0.9204 | 0.8841 |
| 874_V_−1 | G | 17.6% | 216 | 18.5% | 65 | 0.7951 | 0.5295 |
| 803_K_3 | T | 0.9% | 218 | 0.8% | 67 | 1.0000 | 1.0000 |
| 803_K_2 | A | 0.2% | 217 | 1.5% | 67 | 0.1402 | 0.1397 |
| 803_I_1 | G | 28.2% | 195 | 27.3% | 66 | 0.9108 | 0.6849 |
| 803_I_−1 | C | 0.2% | 217 | 0.0% | 64 | 1.0000 | 1.0000 |
| 803_H_+1 | A | 25.2% | 218 | 25.8% | 66 | 0.9094 | 0.8564 |
| 803_E_+2 | A | 44.2% | 208 | 44.6% | 65 | 1.0000 | 0.9816 |
| 962_E_3 | C | 35.6% | 212 | 33.1% | 65 | 0.6744 | 0.4216 |
| 962_E_+2 | C | 12.7% | 217 | 8.7% | 69 | 0.2273 | 0.2212 |
| 962_G_4 | A | 13.6% | 202 | 7.7% | 65 | 0.0891 | 0.0249 |
| 962_G_1 | A | 27.2% | 217 | 37.7% | 69 | 0.0245 | 0.0218 |
| 962_G_2 | A | 7.6% | 217 | 8.1% | 68 | 0.8546 | 0.3300 |
| 962_G_6 | T | 20.4% | 194 | 18.2% | 66 | 0.6158 | 0.5068 |
| 962_H_+2 | A | 41.5% | 217 | 35.3% | 68 | 0.2288 | 0.2945 |
| 962_M_+2 | C | 12.7% | 213 | 6.2% | 65 | 0.0389 | 0.0079 |
| 962_P_−2 | A | 23.8% | 214 | 28.0% | 66 | 0.3568 | 0.4360 |
| 962_Q_−1 | A | 23.7% | 215 | 28.1% | 64 | 0.3505 | 0.4884 |
| 962_S_−1 | C | 11.4% | 215 | 3.7% | 68 | 0.0068 | 0.0046 |
| 962_U_1 | A | 3.0% | 214 | 4.6% | 65 | 0.4086 | 0.4012 |
| 962_U_2 | T | 23.8% | 212 | 25.4% | 67 | 0.7291 | 0.8795 |
| 962_V_−1 | T | 34.8% | 187 | 31.1% | 66 | 0.4560 | 0.5715 |
| 962_V_+2 | A | 4.5% | 209 | 3.7% | 68 | 0.8108 | 1.0000 |
| 962_Z_1 | T | 31.9% | 216 | 28.3% | 69 | 0.4601 | 0.2792 |

TABLE 19
ASSOCIATION ANALYSIS OF SPECIFIC IgE
PHENOTYPE UK POPULATION
UK population

| | | | FREQUENCIES | | | ALLELE | GENOTYPE |
|---|---|---|---|---|---|---|---|
| GENE_EXON | ALLELE | CNTL | N | CASE | N | P-VALUE | P-VALUE |
| 845_R_1 | A | 13.6% | 140 | 11.6% | 43 | 0.7179 | 0.8612 |
| 845_R_−1 | T | 26.6% | 137 | 22.7% | 44 | 0.4875 | 0.8214 |
| 845_P_+1 | T | 6.4% | 140 | 12.5% | 44 | 0.0724 | 0.0938 |
| 845_K_1 | T | 0.4% | 135 | 0.0% | 41 | 1.0000 | 1.0000 |
| 845_K_−2 | A | 27.0% | 139 | 23.3% | 43 | 0.5747 | 0.8190 |
| 845_J_1 | G | 36.6% | 134 | 43.2% | 44 | 0.3119 | 0.5431 |
| 845_J_−1 | C | 37.5% | 140 | 34.1% | 44 | 0.6131 | 0.7816 |
| 845_I_−1 | C | 12.6% | 127 | 11.4% | 44 | 0.8520 | 1.0000 |
| 845_H_+1 | G | 19.3% | 140 | 13.6% | 44 | 0.2667 | 0.4230 |
| 845_H_−1 | T | 43.6% | 140 | 38.6% | 44 | 0.4590 | 0.4785 |
| 845_G_+1 | T | 13.2% | 140 | 15.9% | 44 | 0.5956 | 0.7510 |
| 845_F_+1 | T | 18.8% | 138 | 20.5% | 44 | 0.7570 | 0.8585 |
| 845_D_1 | T | 0.0% | 140 | 1.2% | 43 | 0.2350 | 0.2350 |
| 845_D_−1 | T | 9.3% | 140 | 15.1% | 43 | 0.1601 | 0.1350 |
| 847_K_1 | A | 3.6% | 139 | 2.5% | 40 | 1.0000 | 1.0000 |
| 847_J_+1 | T | 2.9% | 139 | 1.1% | 46 | 0.4610 | 0.4549 |
| 847_E_+1 | C | 12.8% | 133 | 18.9% | 45 | 0.1653 | 0.0766 |
| 847_D_−1 | C | 16.5% | 136 | 23.1% | 39 | 0.1853 | 0.1186 |
| 847_C_+1 | G | 17.8% | 132 | 26.1% | 46 | 0.0964 | 0.0143 |
| 847_A_2 | T | 5.4% | 140 | 6.5% | 46 | 0.6132 | 0.7884 |
| 847_A_1 | A | 1.3% | 120 | 1.1% | 46 | 1.0000 | 1.0000 |
| 874_R_+1 | T | 41.5% | 129 | 32.3% | 48 | 0.1412 | 0.2670 |
| 874_S_+1 | A | 38.0% | 137 | 40.2% | 51 | 0.7215 | 0.9056 |
| 874_T_−1 | T | 48.9% | 136 | 51.0% | 52 | 0.7309 | 0.9130 |
| 874_V_−1 | G | 16.9% | 139 | 18.3% | 52 | 0.7623 | 0.6537 |
| 803_K_3 | T | 1.1% | 140 | 0.9% | 53 | 1.0000 | 1.0000 |
| 803_K_2 | A | 0.4% | 139 | 0.9% | 53 | 0.4764 | 0.4769 |
| 803_I_1 | G | 28.6% | 117 | 24.0% | 52 | 0.4281 | 0.5565 |
| 803_I_−1 | C | 0.4% | 139 | 0.0% | 50 | 1.0000 | 1.0000 |
| 803_H_+1 | A | 25.7% | 140 | 26.0% | 52 | 1.0000 | 0.8447 |
| 803_E_+2 | A | 42.4% | 132 | 48.0% | 51 | 0.3491 | 0.5519 |
| 962_E_3 | C | 36.8% | 136 | 30.0% | 50 | 0.2697 | 0.3867 |
| 962_E_+2 | C | 12.5% | 140 | 8.5% | 53 | 0.3692 | 0.3657 |
| 962_G_4 | A | 14.0% | 132 | 8.2% | 49 | 0.1539 | 0.0449 |
| 962_G_1 | A | 25.2% | 139 | 34.9% | 53 | 0.0742 | 0.0681 |
| 962_G_2 | A | 6.4% | 140 | 8.7% | 52 | 0.5009 | 0.3792 |
| 962_G_6 | T | 21.7% | 129 | 15.0% | 50 | 0.1841 | 0.1382 |
| 962_H_+2 | A | 43.6% | 140 | 36.5% | 52 | 0.2445 | 0.2880 |
| 962_M_+2 | C | 12.4% | 137 | 3.0% | 50 | 0.0056 | 0.0165 |
| 962_P_−2 | A | 24.6% | 136 | 24.0% | 52 | 1.0000 | 1.0000 |
| 962_Q_−1 | A | 24.8% | 139 | 24.5% | 49 | 1.0000 | 0.9376 |
| 962_S_−1 | C | 10.9% | 137 | 1.0% | 52 | 0.0006 | 0.0025 |
| 962_U_1 | A | 3.7% | 136 | 4.9% | 51 | 0.5642 | 0.5575 |
| 962_U_2 | T | 25.0% | 136 | 20.6% | 51 | 0.4151 | 0.7659 |
| 962_V_−1 | T | 34.4% | 122 | 29.4% | 51 | 0.3829 | 0.1817 |
| 962_V_+2 | A | 4.5% | 134 | 3.8% | 52 | 1.0000 | 1.0000 |
| 962_Z_1 | T | 30.0% | 140 | 32.1% | 53 | 0.7114 | 0.1780 |

TABLE 20
ASSOCIATION ANALYSIS OF SPECIFIC IgE
PHENOTYPE US POPULATION
US population

| | | | FREQUENCIES | | | ALLELE | GENOTYPE |
|---|---|---|---|---|---|---|---|
| GENE_EXON | ALLELE | CNTL | N | CASE | N | P-VALUE | P-VALUE |
| 845_R_1 | A | 17.1% | 76 | 6.3% | 16 | 0.1750 | 0.3956 |
| 845_R_−1 | T | 32.5% | 77 | 28.1% | 16 | 0.6822 | 0.7490 |
| 845_P_+1 | T | 6.5% | 77 | 18.8% | 16 | 0.0362 | 0.0814 |
| 845_K_1 | T | 0.0% | 75 | 0.0% | 16 | 1.0000 | 1.0000 |
| 845_K_−2 | A | 32.5% | 77 | 28.6% | 14 | 0.8264 | 0.7366 |
| 845_J_1 | G | 30.9% | 76 | 43.8% | 16 | 0.2144 | 0.3882 |
| 845_J_−1 | C | 36.4% | 77 | 28.1% | 16 | 0.4212 | 0.6502 |
| 845_I_−1 | C | 13.3% | 64 | 12.5% | 16 | 1.0000 | 0.8344 |
| 845_H_+1 | G | 20.1% | 77 | 12.5% | 16 | 0.4562 | 0.5019 |
| 845_H_−1 | T | 48.1% | 77 | 46.7% | 15 | 1.0000 | 0.4033 |
| 845_G_+1 | T | 13.2% | 76 | 9.4% | 16 | 0.7705 | 1.0000 |
| 845_F_+1 | T | 17.5% | 77 | 15.6% | 16 | 1.0000 | 0.8876 |
| 845_D_1 | T | 0.7% | 76 | 0.0% | 14 | 1.0000 | 1.0000 |
| 845_D_−1 | T | 11.0% | 77 | 17.9% | 14 | 0.3432 | 0.2166 |
| 847_K_1 | A | 7.3% | 75 | 8.3% | 18 | 0.7364 | 1.0000 |
| 847_J_+1 | T | 8.4% | 77 | 2.8% | 18 | 0.4757 | 0.2939 |
| 847_E_+1 | C | 12.3% | 77 | 5.6% | 18 | 0.3765 | 0.3445 |
| 847_D_−1 | C | 17.6% | 74 | 3.3% | 15 | 0.0516 | 0.1501 |
| 847_C_+1 | G | 18.2% | 77 | 8.8% | 17 | 0.2134 | 0.5739 |
| 847_A_2 | T | 9.1% | 77 | 11.1% | 18 | 0.7523 | 0.7410 |
| 847_A_1 | A | 0.7% | 72 | 0.0% | 14 | 1.0000 | 1.0000 |
| 874_R_+1 | T | 37.0% | 73 | 33.3% | 12 | 0.8215 | 1.0000 |
| 874_S_+1 | A | 42.2% | 77 | 46.2% | 13 | 0.8308 | 0.7845 |
| 874_T_−1 | T | 48.1% | 77 | 42.3% | 13 | 0.6733 | 0.8501 |
| 874_V_−1 | G | 18.8% | 77 | 19.2% | 13 | 1.0000 | 0.8434 |
| 803_K_3 | T | 0.6% | 78 | 0.0% | 14 | 1.0000 | 1.0000 |
| 803_K_2 | A | 0.0% | 78 | 3.6% | 14 | 0.1522 | 0.1522 |
| 803_I_1 | G | 27.6% | 78 | 39.3% | 14 | 0.2596 | 0.3122 |
| 803_I_−1 | C | 0.0% | 78 | 0.0% | 14 | 1.0000 | 1.0000 |
| 803_H_+1 | A | 24.4% | 78 | 25.0% | 14 | 1.0000 | 1.0000 |
| 803_E_+2 | A | 47.4% | 76 | 32.1% | 14 | 0.1529 | 0.3421 |
| 962_E_3 | C | 33.6% | 76 | 43.3% | 15 | 0.3050 | 0.3833 |
| 962_E_+2 | C | 13.0% | 77 | 9.4% | 16 | 0.7706 | 0.7526 |
| 962_G_4 | A | 12.9% | 70 | 6.3% | 16 | 0.3748 | 0.5960 |
| 962_G_1 | A | 30.8% | 78 | 46.9% | 16 | 0.0999 | 0.1238 |
| 962_G_2 | A | 9.7% | 77 | 6.3% | 16 | 0.7414 | 0.7273 |
| 962_G_6 | T | 17.7% | 65 | 28.1% | 16 | 0.2160 | 0.3921 |
| 962_H_+2 | A | 37.7% | 77 | 31.3% | 16 | 0.5501 | 0.8134 |
| 962_M_+2 | C | 13.2% | 76 | 16.7% | 15 | 0.5704 | 0.1201 |
| 962_P_−2 | A | 22.4% | 78 | 42.9% | 14 | 0.0329 | 0.0244 |
| 962_Q_−1 | A | 21.7% | 76 | 40.0% | 15 | 0.0398 | 0.0619 |
| 962_S_−1 | C | 12.2% | 78 | 12.5% | 16 | 1.0000 | 0.4985 |
| 962_U_1 | A | 1.9% | 78 | 3.6% | 14 | 0.4864 | 0.4895 |
| 962_U_2 | T | 21.7% | 76 | 40.6% | 16 | 0.0411 | 0.0456 |
| 962_V_−1 | T | 35.4% | 65 | 36.7% | 15 | 1.0000 | 0.4874 |
| 962_V_+2 | A | 4.7% | 75 | 3.1% | 16 | 1.0000 | 1.0000 |
| 962_Z_1 | T | 35.5% | 76 | 15.6% | 16 | 0.0362 | 0.0826 |

For the specific IgE phenotype, a single SNP in Gene 845 reached statistical significance in the combined and the US population alone for both the allele and the genotype tests: SNP P+1. For this SNP, 14.2% of the cases in the combined population were carriers of the T allele, whereas the T allele was observed in only 6.5% of the controls (combined:

allele test p=0.0126, genotype test p=0.0172; US: allele test p=0.0362). A single SNP in Gene 847 reached statistical significance in the UK population alone: SNP C+1. For this SNP, 26.1% of the cases were carriers of the G allele, whereas the G allele was observed in only 17.8% of the controls (UK: genotype test p=0.0143). Eight SNPs in Gene 962 reached statistical significance in the combined, the UK population alone and the US population alone for the allele and the genotype tests: 92.3% of the cases in the combined population were carriers of the G allele in SNP G 4, whereas the G allele was observed in only 86.4% of the controls (combined: genotype test p=0.0249; UK: genotype test p=0.0449), 37.7% of the cases were carriers of the A allele in SNP G 1, whereas the A allele was observed in only 27.2% of the controls (combined: allele p=0.0245, genotype p=0.0218), 93.8% of the cases in the combined population were carriers of the G allele in SNP M+2, whereas the G allele was observed in only 87.3% of the controls (combined: allele test p=0.0389, genotype test p=0.0079; UK: allele test p=0.0056, genotype test p=0.0165), 42.9% of the cases were carriers of the A allele in SNP P−2, whereas the A allele was observed in only 22.4% of the controls (US: allele test p=0.0329, genotype test p=0.0244), 40.0% of the cases were carriers of the A allele in SNP Q−1, whereas the A allele was observed in only 21.7% of the controls (US: allele test p=0.0398), 96.3% of the cases in the combined population were carriers of the G allele in SNP S−1, whereas the G allele was observed in only 88.6% of the controls (combined: allele test p=0.0068, genotype test p=0.0046; UK: allele test p=0.0006, genotype test p=0.0025), 40.6% of the cases were carriers of the T allele in SNP U 2, whereas the T allele was observed in only 21.7% of the controls (US: allele p=0.0411, genotype p=0.0456) and 84.4% of the cases were carriers of the C allele in SNP Z 1, whereas the C allele was observed in only 64.5% of the controls (US: allele test p=0.0362).

3. Association Test with SNP Combinations:

In addition to the analysis of individual SNPs, haplotype frequencies between the case and control groups were also compared. The haplotypes were constructed using a maximum likelihood approach. Existing software for predicting haplotypes was unable to utilize individuals with missing data. Accordingly, a program was developed to make use of all individuals. This allowed more accurate estimates of haplotype frequency. Haplotype analysis based on multiple SNPs in a gene was expected to provide increased evidence for an association between a given phenotype and that gene, if all haplotyped SNPs were involved in the characterization of the phenotype. Otherwise, allelic variation involving those haplotyped SNPs would not be associated more significantly with different risks or susceptibilities toward the phenotype.

a. Asthma Phenotype:

The estimated frequencies of each haplotype for cases and controls were compared using a permutation test. An overall comparison of the distribution of all haplotypes between the two groups was also performed. In Tables 21, 22 and 23 the haplotype analysis (2-at-a-time) is presented for the combined, the UK and the US populations, respectively. The diagonal entries represent the single SNP p-values, while the other entries are the p-values for a test of association between the asthma phenotype and the haplotypes defined by the 2 SNPs listed on the horizontal and vertical axes. The frequencies of the individual SNPs in the cases and controls are shown at the bottom of the tables. Colored cells indicate p-values that were statistically significant (light gray: 0.01 to 0.05, dark gray: 0.001 to 0.0099, black: <0.001). We highlight those combinations that are significant at the 0.05 level and that are more significant than the two tests involving each of the constituent SNPs alone (diagonal entries). One SNP combination in Gene 845 is significant in the US population: SNPs R 1 & K−2 (p=0.0443). Four SNP combinations in Gene 803 are significant in the US population: SNPs K 3 & K 2 (p=0.0409), SNPs K 2 & I 1 (p=0.0146), SNPs K 2 & I−1 (p=0.0383), SNPs K 2 & E+2 (p=0.0197). Thirteen SNP combinations in Gene 962 are significant in the combined, the UK and the US population alone: SNPs E+2 & V−1 (UK p=0.0362), SNPs G 4 & G 1 (combined p=0.0472), SNPs G 4 & P−2 (US p=0.0174), SNPs G 4 & Q−1 (US p=0.016), SNPs G 4 & U 2 (US p=0.013) SNPs G 4 & V+2 (US p=0.0188), SNPs G 1 & G 6 (UK p=0.0369), SNPs G 1 & Q−1 (combined p=0.0441; US p=0.0197), SNPs G 1 & U 2 (US p=0.016), SNPs G 1 & V−1 (combined p=0.0311), SNPs G 6 & S−1 (UK p=0.038), SNPs H+2 & S−1 (combined p=0.0492) and SNPs U 2 & V+2 (US p=0.0212).

TABLE 21
HAPLOTYPE ANALYSIS OF ASTHMA PHENOTYPE COMBINED US/UK POPULATION

| | 845_R_1 | 845_R_−1 | 845_P_+1 | 845_K_1 | 845_K_−2 | 845_J_1 | 845_J_−1 | 845_l_−1′ | 845_H_+1 | 845_H_−1 | 845_G_+1 | 845_F_+1 | 845_D_1 | 845_D_−1 | |
|---|---|---|---|---|---|---|---|---|---|---|---|---|---|---|---|
| 845_{—} | 0.4634 | 0.4245 | 0.3666 | 0.619 | 0.2116 | 0.5313 | 0.555 | 0.6457 | 0.3764 | 0.7213 | 0.6732 | 0.6692 | 0.3245 | 0.7027 | 845_R_1 |
| R_1 | | | | | | | | | | | | | | | |
| 845_{—} | — | 0.507 | 0.387 | 0.665 | 0.1628 | 0.2303 | 0.721 | 0.8127 | 0.1361 | 0.17 | 0.846 | 0.8137 | 0.5012 | 0.6587 | 845_R_−1 |
| R_−1 | | | | | | | | | | | | | | | |
| 845_{—} | — | — | 0.1998 | 0.3458 | 0.2262 | 0.646 | 0.6124 | 0.3427 | 0.1955 | 0.5843 | 0.4044 | 0.4398 | 0.2971 | 0.5948 | 845_P_+1 |
| P_+1 | | | | | | | | | | | | | | | |
| 845_{—} | — | — | — | 1 | 0.3967 | 0.5143 | 0.8472 | 0.7931 | 0.2278 | 0.9613 | 0.8108 | 0.9055 | 0.4205 | 0.7815 | 845_K_1 |
| K_1 | | | | | | | | | | | | | | | |
| 845_{—} | — | — | — | — | 0.2103 | 0.3312 | 0.4067 | 0.2975 | 0.0555 | 0.1066 | 0.438 | 0.33 | 0.2143 | 0.4 | 845_K_−2 |
| K_−2 | | | | | | | | | | | | | | | |
| 845_{—} | — | — | — | — | — | 0.3296 | 0.2547 | 0.3243 | 0.3114 | 0.6043 | 0.4215 | 0.6475 | 0.2175 | 0.3693 | 845_J_1 |
| J_1 | | | | | | | | | | | | | | | |
| 845_{—} | — | — | — | — | — | — | 0.794 | 0.89 | 0.0977 | 0.4639 | 0.8126 | 0.9791 | 0.5486 | 0.8222 | 845_J_−1 |
| J_−1 | | | | | | | | | | | | | | | |
| 845_{—} | — | — | — | — | — | — | — | 0.6962 | 0.2455 | 0.8919 | 0.5241 | 0.4525 | 0.5393 | 0.3813 | 845_I_−1 |
| I_−1 | | | | | | | | | | | | | | | |
| 845_{—} | — | — | — | — | — | — | — | — | 0.1559 | 0.3291 | 0.356 | 0.5486 | 0.1114 | 0.3007 | 845_H_+1 |
| H_+1 | | | | | | | | | | | | | | | |
| 845_{—} | — | — | — | — | — | — | — | — | — | 0.865 | 0.9607 | 0.8998 | 0.6117 | 0.9736 | 845_H_−1 |
| H_−1 | | | | | | | | | | | | | | | |
| 845_{—} | — | — | — | — | — | — | — | — | — | — | 0.8053 | 0.5316 | 0.5733 | 0.5129 | 845_G_+1 |
| G_+1 | | | | | | | | | | | | | | | |
| 845_{—} | — | — | — | — | — | — | — | — | — | — | — | 0.913 | 0.5852 | 0.8162 | 845_F_+1 |
| F_+1 | | | | | | | | | | | | | | | |
| 845_{—} | — | — | — | — | — | — | — | — | — | — | — | — | 0.2339 | 0.5455 | 845_D_1 |
| D_1 | | | | | | | | | | | | | | | |
| 845_{—} | — | — | — | — | — | — | — | — | — | — | — | — | — | 0.6727 | 845_D_−1 |
| D_−1 | | | | | | | | | | | | | | | |
| CNTL | 14.8% | 28.7% | 6.5% | 0.2% | 28.9% | 34.5% | 37.1% | 12.8% | 19.6% | 45.2% | 13.2% | 18.4% | 0.2% | 9.9% | CNTL |
| CASE | 12.5% | 26.0% | 9.5% | 0.0% | 24.0% | 38.8% | 38.5% | 11.5% | 14.9% | 44.2% | 13.9% | 17.6% | 1.0% | 11.1% | CASE |

| | 847_K_1 | 847_J_+1 | 847_E_+1 | 847_D_−1 | 847_C_+1 | 847_A_2 | 847_A_1 | |
|---|---|---|---|---|---|---|---|---|
| 847_K_1 | 0.6976 | 0.2577 | 0.5741 | 0.8309 | 0.6644 | 0.5754 | 0.6198 | 847_K_1 |
| 847_J_+1 | — | 0.1394 | 0.2436 | 0.263 | 0.2015 | 0.2544 | 0.2522 | 847_J_+1 |
| 847_E_+1 | — | — | 0.9001 | 0.3793 | 0.7355 | 0.9908 | 0.8391 | 847_E_+1 |
| 847_D_−1 | — | — | — | 0.7272 | 0.6646 | 0.9053 | 0.816 | 847_D_−1 |
| 847_C_+1 | — | — | — | — | 0.3932 | 0.8445 | 0.6336 | 847_C_+1 |
| 847_A_2 | — | — | — | — | — | 0.7457 | 0.6891 | 847_A_2 |
| 847_A_1 | — | — | — | — | — | — | 0.6625 | 847_A_1 |
| CNTL | 4.9% | 4.9% | 12.6% | 16.9% | 17.9% | 6.7% | 1.0% | CNTL |
| CASE | 5.7% | 2.3% | 13.1% | 18.3% | 20.8% | 7.3% | 0.5% | CASE |

| | 874_R_+1 | 874_S_+1 | 874_T_−1 | 874_V_−1 | |
|---|---|---|---|---|---|
| 874_R_+1 | 0.3645 | 0.6882 | 0.7952 | 0.5905 | 874_R_+1 |
| 874_S_+1 | — | 0.5993 | 0.3209 | 0.9142 | 874_S_+1 |
| 874_T_−1 | — | — | 0.7971 | 0.8707 | 874_T_−1 |
| 874_V_−1 | — | — | — | 0.5803 | 874_V_−1 |
| CNTL | 39.9% | 39.5% | 48.6% | 17.6% | CNTL |
| CASE | 35.6% | 41.9% | 50.0% | 19.5% | CASE |

| | 803_K_3 | 803_K_2 | 803_I_1 | 803_1_−1 | 803_H_+1 | 803_E_+2 | |
|---|---|---|---|---|---|---|---|
| 803_K_3 | 0.7046 | 0.104 | 0.7655 | 0.9881 | 0.905 | 0.9005 | 803_K_3 |
| 803_K_2 | — | 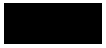 | 0.074 | 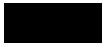 | 0.1057 | 0.1142 | 803_K_2 |
| 803_I_1 | — | — | 0.7829 | 0.82 | 0.835 | 0.5279 | 803_I_1 |
| 803_I_−1 | — | — | — | 1 | 0.9137 | 0.8473 | 803_I_−1 |
| 803_H_+1 | — | — | — | — | 0.8524 | 0.7543 | 803_H_+1 |
| 803_E_+2 | — | — | — | — | — | 0.806 | 803_E_+2 |
| CNTL | 0.9% | 0.2% | 28.2% | 0.2% | 25.2% | 44.2% | CNTL |
| CASE | 1.2% | 2.1% | 26.9% | 0.0% | 24.4% | 45.3% | CASE |

| | 962_E_3 | 962_E_+2 | 962_G_4 | 962_G_1 | 962_G_2 | 962_G_6 | 962_H_+2 | 962_M_+2 | 962_P_−2 | 962_Q_−1 | 962_S_−1 | 962_U_1 | |
|---|---|---|---|---|---|---|---|---|---|---|---|---|---|
| 962_E_3 | 0.4455 | 0.2588 | 0.4189 | 0.0937 | 0.6231 | 0.7666 | 0.6764 | 0.5219 | 0.8744 | 0.8667 | 0.3369 | 0.8593 | 962_E_3 |
| 962_E_+2 | — | 0.4558 | 0.4429 | 0.2074 | 0.6609 | 0.6666 | 0.774 | 0.4971 | 0.3414 | 0.4857 | 0.3437 | 0.81 | 962_E_+2 |
| 962_G_4 | — | — | 0.1641 | 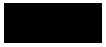 | 0.3472 | 0.4503 | 0.4306 | 0.1435 | 0.4478 | 0.4527 | 0.0606 | 0.4802 | 962_G_4 |
| 962_G_1 | — | — | — | 0.0541 | 0.3005 | 0.0609 | 0.1946 | 0.1569 | 0.0625 | 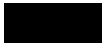 | 0.0992 | 0.2891 | 962_G_1 |
| 962_G_2 | — | — | — | | 1 | 0.863 | 0.9481 | 0.6477 | 0.9326 | 0.9491 | 0.3253 | 0.2915 | 962_G_2 |
| 962_G_6 | | — | — | — | | 0.4632 | 0.8269 | 0.3438 | 0.6522 | 0.6812 | 0.19 | 0.0953 | 962_G_6 |
| 962_H_+2 | | — | — | — | | | 0.6236 | 0.2445 | 0.9325 | 0.8592 | 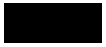 | 0.9522 | 962_H_+2 |
| 962_M_+2 | | — | — | — | — | | | 0.2455 | 0.439 | 0.4377 | 0.4435 | 0.4248 | 962_M_+2 |
| 962_P_−2 | | — | — | | — | | | | 0.7026 | 0.9337 | 0.3309 | 0.8719 | 962_P_+2 |
| 962_Q_−1 | | — | — | | — | | | | | 0.6345 | 0.3378 | 0.9307 | 962_Q_−1 |
| 962_S_−1 | — | — | — | — | — | — | — | | | | 0.1388 | 0.2844 | 962_S_−1 |
| 962_U_1 | — | — | — | — | — | | | | | — | | 0.8173 | 962_U_1 |
| 962_U_2 | | | — | — | — | | | — | — | — | — | | 962_U_2 |
| 962_V_−1 | | | — | — | — | | | | — | | — | | 962_V_−1 |
| 962_V_+2 | — | — | — | — | — | | | — | — | | — | | 962_V_+2 |
| 962_Z_1 | — | | — | — | — | | — | | — | — | — | — | 962_Z_1 |
| CNTL | 35.6% | 12.7% | 13.6% | 27.2% | 7.6% | 20.4% | 41.5% | 12.7% | 23.8% | 23.7% | 11.4% | 3.0% | CNTL |
| CASE | 38.7% | 10.4% | 9.6% | 34.3% | 7.6% | 17.8% | 39.5% | 9.3% | 25.4% | 25.7% | 7.6% | 3.5% | CASE |

| | 962_U_2 | 962_V_−1 | 962_V_−2 | 962_Z_1 | |
|---|---|---|---|---|---|
| 962_E_3 | 0.8966 | 0.0994 | 0.2232 | 0.7448 | 962_E_3 |
| 962_E_+2 | 0.4599 | 0.0563 | 0.3217 | 0.6337 | 962_E_+2 |
| 962_G_4 | 0.4129 | 0.0772 | 0.3147 | 0.2586 | 962_G_4 |
| 962_G_1 | 0.0902 | 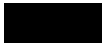 | 0.1532 | 0.2534 | 962_G_1 |
| 962_G_2 | 0.7658 | 0.2209 | 0.6273 | 0.9931 | 962_G_2 |
| 962_G_6 | 0.8694 | 0.1544 | 0.3544 | 0.8695 | 962_G_6 |
| 962_H_+2 | 0.6692 | 0.0854 | 0.2691 | 0.7072 | 962_H_+2 |
| 962_M_+2 | 0.3992 | 0.0824 | 0.1662 | 0.6105 | 962_M_+2 |
| 962_P_−2 | 0.8843 | 0.2614 | 0.4837 | 0.4783 | 962_P_+2 |
| 962_Q_−1 | 0.8431 | 0.2369 | 0.5754 | 0.5458 | 962_Q_−1 |
| 962_S_−1 | 0.5332 | 0.0578 | 0.1023 | 0.3447 | 962_S_−1 |
| 962_U_1 | 0.9205 | 0.1002 | 0.3899 | 0.6979 | 962_U_1 |
| 962_U_2 | 0.7763 | 0.112 | 0.6047 | 0.9367 | 962_U_2 |
| 962_V_−1 | | 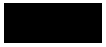 | 0.17 | 0.2122 | 962_V_−1 |
| 962_V_+2 | — | — | 0.2117 | 0.3953 | 962_V_+2 |
| 962_Z_1 | — | | | 0.797 | 962_Z_1 |
| CNTL | 23.8% | 34.8% | 4.5% | 31.9% | CNTL |
| CASE | 25.0% | 26.5% | 2.5% | 33.1% | CASE |

TABLE 22
HAPLOTYPE ANALYSIS OF ASTHMA PHENOTYPE UK POPULATION

| | 845_R_1 | 845_R_−1 | 845_P_+1 | 845_K_1 | 845_K_−2 | 845_J_1 | 845_J_−1 | 845_I_−1 | 845_H_+1 | 845_H_−1 | 845_G_1+1 | 845_F_1+1 | 845_D_1 | 845_D_−1 | |
|---|---|---|---|---|---|---|---|---|---|---|---|---|---|---|---|
| 845_{—} | 1 | 0.9397 | 0.9453 | 0.9951 | 0.7734 | 0.976 | 0.8431 | 0.8827 | 0.4507 | 0.7585 | 0.7084 | 0.7086 | 0.1635 | 0.9311 | 845_R_1 |
| R_1 | | | | | | | | | | | | | | | |
| 845_{—} | — | 0.8205 | 0.9313 | 0.9699 | 0.6056 | 0.2624 | 0.8569 | 0.8283 | 0.5498 | 0.1328 | 0.8756 | 0.9152 | 0.17 | 0.9508 | 845_R_−1 |
| R_−1 | | | | | | | | | | | | | | | |
| 845_{—} | — | — | 0.7005 | 0.9103 | 0.7895 | 0.9834 | 0.8405 | 0.8749 | 0.6972 | 0.932 | 0.841 | 0.9488 | 0.3189 | 0.6164 | 845_P_+1 |
| P_+1 | | | | | | | | | | | | | | | |
| 845_{—} | — | — | — | 1 | 0.7384 | 0.9153 | 0.7644 | 0.8366 | 0.6246 | 0.9887 | 0.7308 | 0.9604 | 0.0582 | 0.912 | 845_K_1 |
| K_1 | | | | | | | | | | | | | | | |
| 845_{—} | — | — | — | — | 0.5669 | 0.7827 | 0.7547 | 0.6488 | 0.434 | 0.2897 | 0.7535 | 0.7374 | 0.1262 | 0.7782 | 845_K_−2 |
| K_−2 | | | | | | | | | | | | | | | |
| 845_{—} | — | — | — | — | — | 0.9176 | 0.6353 | 0.8137 | 0.674 | 0.9811 | 0.7349 | 0.9898 | 0.1654 | 0.9809 | 845_J_1 |
| J_1 | | | | | | | | | | | | | | | |
| 845_{—} | — | — | — | — | — | — | 0.6126 | 0.8198 | 0.2988 | 0.6821 | 0.6903 | 0.9269 | 0.1298 | 0.7766 | 845_J_−1 |
| J_−1 | | | | | | | | | | | | | | | |
| 845_{—} | — | — | — | — | — | — | — | 0.7575 | 0.5333 | 0.9108 | 0.2364 | 0.8438 | 0.1247 | 0.769 | 845_I_−1 |
| I_−1 | | | | | | | | | | | | | | | |
| 845_{—} | — | — | — | — | — | — | — | — | 0.4428 | 0.6175 | 0.6713 | 0.8652 | 0.0632 | 0.6706 | 845_H_+1 |
| H_+1 | | | | | | | | | | | | | | | |
| 845_{—} | — | — | — | — | — | — | — | — | — | 0.9207 | 0.9052 | 0.9291 | 0.1835 | 0.7567 | 845_H_−1 |
| H_−1 | | | | | | | | | | | | | | | |
| 845_{—} | — | — | — | — | — | — | — | — | — | — | 0.6688 | 0.4125 | 0.1321 | 0.7828 | 845_G_+1 |
| G_+1 | | | | | | | | | | | | | | | |
| 845_{—} | — | — | — | — | — | — | — | — | — | — | — | 1 | 0.1697 | 0.9427 | 845_F_+1 |
| F_+1 | | | | | | | | | | | | | | | |
| 845_{—} | — | — | — | — | — | — | — | — | — | — | — | — | 0.1275 | 0.2245 | 845_D_1 |
| D_−1 | | | | | | | | | | | | | | | |
| 845_{—} | — | | — | — | — | — | — | — | — | — | — | — | — | 0.7377 | 845_D_−1 |
| D_−1 | | | | | | | | | | | | | | | |
| CNTL | 13.6% | 26.6% | 6.4% | 0.4% | 27.0% | 36.6% | 37.5% | 12.6% | 19.3% | 43.6% | 13.2% | 18.8% | 0.0% | 9.3% | CNTL |
| CASE | 13.9% | 25.3% 7.3% | 0.0% | 24.0% | 37.5% | 40.1% | 11.1% | 16.1% | 42.6% | 14.8% | 18.3% | 1.3% | 10.3% | CASE | |

| | 847_K_1 | 847_J_+1 | 847_E_+1 | 847_D_−1 | 847_C_+1 | 847_A_2 | 847_A_1 | |
|---|---|---|---|---|---|---|---|---|
| 847_K_1 | 0.6053 | 0.8162 | 0.4353 | 0.4016 | 0.327 | 0.3663 | 0.678 | 847_K_1 |
| 847_J_+1 | — | 1 | 0.858 | 0.4861 | 0.4485 | 0.8585 | 0.6844 | 847_J_+1 |
| 847_E_+1 | — | — | 0.7727 | 0.1972 | 0.434 | 0.8528 | 0.729 | 847_E_+1 |
| 847_D_−1 | — | — | — | 0.2351 | 0.2354 | 0.6545 | 0.5647 | 847_D_−1 |
| 847_C_+1 | — | — | — | — | 0.2175 | 0.5544 | 0.5157 | 847_C_+1 |
| 847_A_2 | — | — | — | — | — | 0.54 | 0.5849 | 847_A_2 |
| 847_A_1 | — | — | — | — | — | — | 0.6479 | 847_A_1 |
| CNTL | 3.6% | 2.9% | 12.8% | 16.5% | 17.8% | 5.4% | 1.3% | CNTL |
| CASE | 4.8% | 2.3% | 14.1% | 21.2% | 22.9% | 7.1% | 0.6% | CASE |

| | 874_R_+1 | 874_S_+1 | 874_T_−1 | 874_V_−1 | |
|---|---|---|---|---|---|
| 874_R_+1 | 0.2918 | 0.5308 | 0.7188 | 0.5775 | 874_R_+1 |
| 874_S_+1 | — | 0.4122 | 0.1976 | 0.5377 | 874_S_+1 |
| 874_T_−1 | — | — | 0.7645 | 0.6776 | 874_T_−1 |
| 874_V_−1 | — | — | — | 0.5164 | 874_V_−1 |
| CNTL | 41.5% | 38.0% | 48.9% | 16.9% | CNTL |
| CASE | 35.8% | 42.3% | 50.6% | 19.6% | CASE |

| | 803_K_3 | 803_K_2 | 803_I_1 | 803_I_−1 | 803_H_+1 | 803_E_+2 | |
|---|---|---|---|---|---|---|---|
| 803_K_3 | 0.6955 | 0.3719 | 0.5379 | 0.9555 | 0.9532 | 0.7228 | 803_K_3 |
| 803_K_2 | — | 0.3124 | 0.3076 | 0.3062 | 0.5566 | 0.4253 | 803_K_2 |
| 803_I_1 | — | — | 0.3827 | 0.5052 | 0.5243 | 0.597 | 803_I_1 |
| 803_I_−1 | — | — | — | 1 | 0.9649 | 0.4211 | 803_I_−1 |
| 803_H_+1 | — | — | — | — | 1 | 0.5717 | 803_H_+1 |
| 803_E_+2 | — | — | — | — | — | 0.3903 | 803_E_+2 |
| CNTL | 1.1% | 0.4% | 28.6% | 0.4% | 25.7% | 42.4% | CNTL |
| CASE | 1.5% | 1.5% | 24.7% | 0.0% | 25.3% | 46.9% | CASE |

| | 962_E_3 | 962_E_+2 | 962_G_4 | 962_G_1 | 962_G_2 | 962_G_6 | 962_H_+2 | 962_M_+2 | 962_P_=2 | 962_Q_−1 | 962_S_−1 | 962_U_1 | 962_U_2 | |
|---|---|---|---|---|---|---|---|---|---|---|---|---|---|---|
| 962_E_3 | 1 | 0.6202 | 0.6817 | 0.1354 | 0.931 | 0.6669 | 0.8392 | 0.4729 | 0.8903 | 0.9042 | 0.3877 | 0.6387 | 0.82 | 962_E_3 |
| 962_E_+2 | | 0.4621 | 0.409 | 0.1993 | 0.4089 | 0.1655 | 0.5307 | 0.3457 | 0.323 | 0.423 | 0.322 | 0.7633 | 0.3004 | 962_E_+2 |
| 962_G_4 | — | | 0.3105 | 0.0711 | 0.5288 | 0.2277 | 0.5171 | 0.2912 | 0.4569 | 0.5166 | 0.1901 | 0.6237 | 0.3428 | 962_G_4 |
| 962_G_1 | — | | — | 0.0502 | 0.2925 | 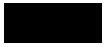 | 0.175 | 0.1113 | 0.1215 | 0.1506 | 0.1123 | 0.1071 | 0.1606 | 962_G_1 |
| 962_G_2 | | — | — | — | 0.7106 | 0.5809 | 0.7814 | 0.4259 | 0.8995 | 0.9066 | 0.2037 | 0.3868 | 0.5857 | 962_G_2 |
| 962_G_6 | — | | — | — | — | 0.2261 | 0.4569 | 0.1416 | 0.1459 | 0.1754 | 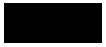 | 0.0718 | 0.507 | 962_G_6 |
| 962_H_+2 | — | | — | — | — | — | 0.3926 | 0.301 | 0.7112 | 0.7315 | 0.1376 | 0.7039 | 0.5272 | 962_H_+2 |
| 962_M_+2 | | | — | — | — | — | | 0.1221 | 0.2005 | 0.2046 | 0.309 | 0.2791 | 0.1253 | 962_M_+2 |
| 962_P_−2 | | | | — | — | | — | | 0.5757 | 0.7311 | 0.21 | 0.9297 | 0.891 | 962_P_−2 |
| 962_Q_−1 | | | | — | — | | — | | | 0.5776 | 0.2338 | 0.882 | 0.8633 | 962_Q_−1 |
| 962_S_−1 | — | | — | | — | | | | — | | 0.1006 | 0.2301 | 0.2779 | 962_S_−1 |
| 962_U_1 | | | — | — | — | | | | — | | | 1 | 0.7188 | 962_U_1 |
| 962_U_2 | | | — | — | | — | — | | — | — | — | | 0.3727 | 962_U_2 |
| 962_V_−1 | — | | — | — | | | | — | — | | — | — | | 962_V_−1 |
| 962_V_+2 | — | — | — | — | | | | | — | | — | — | | 962_V_+2 |
| 962_Z_1 | | — | — | — | | | — | | — | x | — | — | | 962_Z_1 |
| CNTL | 36.8% | 12.5% | 14.0% | 25.2% | 6.4% | 21.7% | 43.6% | 12.4% | 24.6% | 24.8% | 10.9% | 3.7% | 25.0% | CNTL |
| CASE | 36.4% | 9.9% | 10.6% | 33.5% | 7.4% | 16.8% | 39.5% | 7.8% | 22.0% | 22.3% | 6.3% | 3.7% | 21.3% | CASE |

| | 962_V_−1 | 962_V_+2 | 962_Z_1 | |
|---|---|---|---|---|
| 962_E_3 | 0.3109 | 0.5899 | 0.5887 | 962_E_3 |
| 962_E_+2 | 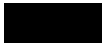 | 0.4364 | 0.5318 | 962_E_+2 |
| 962_G_4 | 0.262 | 0.2455 | 0.2256 | 962_G_4 |
| 962_G_1 | 0.0671 | 0.2155 | 0.1322 | 962_G_1 |
| 962_G_2 | 0.4558 | 0.6792 | 0.7051 | 962_G_2 |
| 962_G_6 | 0.0978 | 0.2839 | 0.3739 | 962_G_6 |
| 962_H_+2 | 0.3122 | 0.2422 | 0.5073 | 962_H_+2 |
| 962_M_+2 | 0.1441 | 0.1237 | 0.1854 | 962_M_+2 |
| 962_P_−2 | 0.1815 | 0.515 | 0.5684 | 962_P_−2 |
| 962_Q_−1 | 0.2645 | 0.724 | 0.5896 | 962_Q_−1 |
| 962_S_−1 | 0.1283 | 0.0951 | 0.0898 | 962_S_−1 |
| 962_U_1 | 0.2327 | 0.6153 | 0.5005 | 962_U_1 |
| 962_U_2 | 0.1 | 0.6232 | 0.734 | 962_U_2 |
| 962_V_−1 | 0.1154 | 0.2506 | 0.5107 | 962_V_−1 |
| 962_V_+2 | | 0.4526 | 0.3607 | 962_V_+2 |
| 962_Z_1 | — | 0.2713 | 962_Z_1 | |
| CNTL | 34.4% | 4.5% | 30.0% | CNTL |
| CASE | 27.2% | 2.6% | 35.1% | CASE |

TABLE 23
HAPLOTYPE ANALYSIS OF ASTHMA PHENOTYPE US POPULATION

| | 845_R_1 | 845_R_−1 | 845_P_+1 | 845_K_1 | 845_K_−2 | 845_J_1 | 845_J_−1 | 845_I_−1 | 845_H_+1 | 845_H_−1 | 845_G_+1 | 845_F_+1 | 845_D_1 | 845_D_−1 | |
|---|---|---|---|---|---|---|---|---|---|---|---|---|---|---|---|
| 845_{—} | 0.1433 | 0.0751 | 0.0677 | 0.0985 | 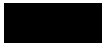 | 0.1403 | 0.2377 | 0.2748 | 0.3446 | 0.1361 | 0.3212 | 0.3334 | 0.236 | 0.289 | 845_R_1 |
| R_1 | | | | | | | | | | | | | | | |
| 845_{—} | | 0.718 | 0.1286 | 0.6516 | 0.3598 | 0.3854 | 0.5459 | 0.9798 | 0.1447 | 0.7334 | 0.9033 | 0.9244 | 0.6754 | 0.7863 | 845_R_−1 |
| R_−1 | | | | | | | | | | | | | | | |
| 845_{—} | | — | 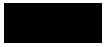 | 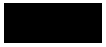 | 0.0855 | 0.1031 | 0.1415 | 0.0766 | 0.0661 | 0.1272 | 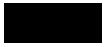 | 0.1122 | 0.0741 | 0.1412 | 845_P_+1 |
| P_+1 | | | | | | | | | | | | | | | |
| 845_{—} | — | — | — | 1 | 0.2461 | 0.1405 | 0.6341 | 0.9495 | 0.1157 | 0.8569 | 0.6691 | 0.7337 | 0.8977 | 0.5857 | 845_K_1 |
| K_−1 | | | | | | | | | | | | | | | |
| 845_{—} | — | — | — | — | 0.3465 | 0.2259 | 0.3114 | 0.574 | 0.0596 | 0.3323 | 0.5334 | 0.4471 | 0.406 | 0.524 | 845_K_−2 |
| K_−2 | | | | | | | | | | | | | | | |
| 845_{—} | — | — | — | — | — | 0.1542 | 0.2328 | 0.2346 | 0.1707 | 0.2033 | 0.2717 | 0.3959 | 0.282 | 0.2671 | 845_J_1 |
| J_1 | | | | | | | | | | | | | | | |
| 845_{—} | — | — | — | — | — | — | 0.7267 | 0.8971 | 0.2452 | 0.5322 | 0.7891 | 0.9281 | 0.7726 | 0.7919 | 845_J_−1 |
| J_−1 | | | | | | | | | | | | | | | |
| 845_{—} | — | — | — | — | — | — | — | 1 | 0.4022 | 0.9652 | 0.9853 | 0.6398 | 0.9932 | 0.6283 | 845_I_−1 |
| 1_−1 | | | | | | | | | | | | | | | |
| 845_{—} | — | — | — | — | — | — | — | — | 0.1915 | 0.2811 | 0.3217 | 0.5231 | 0.3226 | 0.3268 | 845_H_+1 |
| H_+1 | | | | | | | | | | | | | | | |
| 845_{—} | — | — | — | — | — | — | — | — | — | 0.8652 | 0.9207 | 0.933 | 0.9655 | 0.7683 | 845_H_−1 |
| H_−1 | | | | | | | | | | | | | | | |
| 845_{—} | — | — | — | — | — | — | — | — | — | — | 0.8037 | 0.9146 | 0.7918 | 0.4422 | 845_G_+1 |
| G_+1 | | | | | | | | | | | | | | | |
| 845_{—} | — | — | — | — | — | — | — | — | — | — | — | 0.8253 | 0.836 | 0.698 | 845_F_+1 |
| F_+1 | | | | | | | | | | | | | | | |
| 845_{—} | — | — | — | — | — | — | — | — | — | — | — | — | 1 | 0.6294 | 845_D_1 |
| D_1 | | | | | | | | | | | | | | | |
| 845_{—} | — | | — | — | — | — | — | — | — | — | — | — | — | 0.5906 | 845_D_−1 |
| D_−1 | | | | | | | | | | | | | | | |
| CNTL | 17.1% | 32.5% | 6.5% | 0.0% | 32.5% | 30.9% | 36.4% | 13.3% | 20.1% | 48.1% | 13.2% | 17.5% | 0.7% | 11.0% | CNTL |
| CASE | 7.1% | 28.3% | 17.4% | 0.0% | 23.8% | 43.5% | 32.6% | 13.0% | 10.9% | 50.0% | 10.9% | 15.2% | 0.0% | 14.3% | CASE |

| | 847_K_1 | 847_J_+1 | 847_E_+1 | 847_D_−1 | 847_C_+1 | 847_A_2 | 847_A_1 | |
|---|---|---|---|---|---|---|---|---|
| 847_K_1 | 0.7617 | 0.3082 | 0.8693 | 0.2469 | 0.7319 | 0.9941 | 0.9725 | 847_K_1 |
| 847_J_+1 | — | 0.1949 | 0.1683 | 0.0636 | 0.1634 | 0.0917 | 0.1948 | 847_J_+1 |
| 847_E_+1 | — | — | 0.7899 | 0.6313 | 0.6794 | 0.8897 | 0.6777 | 847_E_+1 |
| 847_D_+1 | — | — | — | 0.1431 | 0.4248 | 0.2657 | 0.1912 | 847_D_−1 |
| 847_C_+1 | — | — | — | — | 0.5067 | 0.8066 | 0.5641 | 847_C_+1 |
| 847_A_2 | — | — | — | — | — | 1 | 0.9553 | 847_A_2 |
| 847_A_1 | — | — | — | — | — | — | 1 | 847_A_1 |
| CNTL | 7.3% | 8.4% | 12.3% | 17.6% | 18.2% | 9.1% | 0.7% | CNTL |
| CASE | 8.3% | 2.1% | 9.1% | 7.5% | 13.0% | 8.3% | 0.0% | CASE |

| | 874_R_+1 | 874_S_+1 | 874_T_−1 | 874_V_−1 | |
|---|---|---|---|---|---|
| 874_R_+1 | 0.8552 | 0.8248 | 0.9906 | 0.9913 | 874_R_+1 |
| 874_S_+1 | — | 0.862 | 0.9128 | 0.9877 | 874_S_+1 |
| 874_T_−1 | — | — | 1 | 0.9906 | 874_T_−1 |
| 874_V_−1 | — | — | — | 1 | 874_V_−1 |
| CNTL | 37.0% | 42.2% | 48.1% | 18.8% | CNTL |
| CASE | 35.0% | 40.5% | 47.6% | 19.1% | CASE |

| | 803_K_3 | 803_K_2 | 803_I_1 | 803_I_−1 | 803_H_+1 | 803_E_+2 | |
|---|---|---|---|---|---|---|---|
| 803_K_3 | 1 | 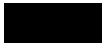 | 0.3618 | 0.8151 | 0.6769 | 0.5253 | 803_K_3 |
| 803_K_2 | — | 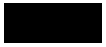 | 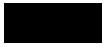 | 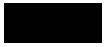 | 0.0581 | 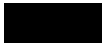 | 803_K_2 |
| 803_I_1 | — | — | 0.2666 | 0.3065 | 0.5303 | 0.4402 | 803_I_1 |
| 803_I_−1 | — | — | — | 1 | 0.6784 | 0.3286 | 803_I_−1 |
| 803_H_+1 | — | — | — | — | 0.6895 | 0.4468 | 803_H_+1 |
| 803_E_+2 | — | — | — | — | — | 0.3902 | 803_E_+2 |
| CNTL | 0.6% | 0.0% | 27.6% | 0.0% | 24.4% | 47.4% | CNTL |
| CASE | 0.0% | 4.5% | 36.4% | 0.0% | 20.5% | 38.6% | CASE |

| | 962_E_3 | 962_E_+2 | 962_G_4 | 962_G_1 | 962_G_2 | 962_G_6 | 962_H_+2 | 296_M_+2 | 296_P_−2 | 962_Q_−1 | 962_S_−1 | 962_U_1 | |
|---|---|---|---|---|---|---|---|---|---|---|---|---|---|
| 962_E_3 | 0.0842 | 0.116 | 0.0783 | 0.0837 | 0.2803 | 0.3026 | 0.2041 | 0.3918 | 0.0681 | 0.0605 | 0.3617 | 0.1492 | 962_E_3 |
| 962_E_+2 | | 1 | 0.5883 | 0.8796 | 0.9873 | 0.6633 | 0.9931 | 0.9613 | 0.1674 | 0.1752 | 0.9814 | 0.9603 | 962_E_+2 |
| 962_G_4 | | | 0.291 | 0.1488 | 0.5145 | 0.5225 | 0.6364 | 0.2885 | 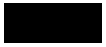 | 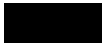 | 0.2983 | 0.4429 | 962_G_4 |
| 962_G_1 | | | | 0.3839 | 0.8397 | 0.7321 | 0.4268 | 0.7269 | 0.0502 | 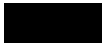 | 0.587 | 0.5489 | 962_G_1 |
| 962_G_2 | | | | | 1 | 0.9314 | 0.9849 | 0.954 | 0.0577 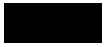 | 0.9935 | 0.8216 | 962_G_2 | |
| 962_G_6 | | | | | | 0.5195 | 0.946 | 0.9703 | 0.1488 | 0.1326 | 0.8997 | 0.8463 | 962_G_6 |
| 962_H_+2 | | | | | | | 0.8654 | 0.6608 | 0.2227 | 0.1321 | 0.4193 | 0.8572 | 962_H_+2 |
| 962_M_+2 | | | — | | | — | | 0.8065 | 0.0529 | 0.059 | 0.1433 | 0.8703 | 962_M_+2 |
| 962_P_+2 | | — | | — | | — | — | — | 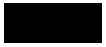 | 0.0864 | 0.0605 | 0.1057 | 962_P_−2 |
| 962_Q_−1 | — | — | — | — | | — | — | — | — | 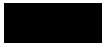 | 0.0651 | 0.0846 | 962_Q_−1 |
| 962_S_−1 | | — | | | — | — | — | — | — | | 1 | 0.9244 | 962_S_−1 |
| 962_U_1 | | | | | | — | — | — | — | — | | 1 | 962_U_1 |
| 962_U_2 | | — | | | | — | — | — | — | — | | | 962_V_2 |
| 962_V_−1 | | | — | | | — | — | — | — | — | | — | 962_V_−1 |
| 962_V_+2 | | | | | — | — | — | — | — | — | | | 962_V_+2 |
| 962_Z_1 | | — | | | — | — | — | — | — | — | | | 962_Z_1 |
| CNTL | 33.6% | 13.0% | 129% | 30.8% | 9.7% | 17.7% | 37.7% | 13.2% | 22.4% | 21.7% | 12.2% | 1.9% | CNTL |
| CASE | 47.8% | 12.5% | 6.3% | 37.5% | 8.3% | 21.7% | 39.6% | 15.2% | 40.5% | 39.1% | 12.5% | 2.4% | CASE |

| | 962_U_2 | 962_V_−1 | 962_V_+2 | 296_Z_1 | |
|---|---|---|---|---|---|
| 962_E_3 | 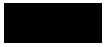 | 0.2377 | 0.2088 | 0.1554 | 962_E_3 |
| 962_E_+2 | 0.1617 | 0.6046 | 0.8233 | 0.5705 | 962_E_+2 |
| 962_G_4 | 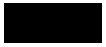 | 0.2715 | 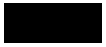 | 0.2321 | 962_G_4 |
| 962_G_1 | 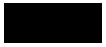 | 0.3085 | 0.3786 | 0.2794 | 962_G_1 |
| 962_G_2 | 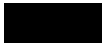 | 0.2836 | 0.432 | 0.4668 | 962_G_2 |
| 962_G_6 | 0.1362 | 0.3672 | 0.599 | 0.6065 | 962_G_6 |
| 962_H_+2 | 0.1034 | 0.2646 | 0.7828 | 0.5467 | 962_H_+2 |
| 962_M_+2 | 0.056 | 0.5608 | 0.7893 | 0.5495 | 962_M_+2 |
| 962_P_+2 | 0.0849 | 0.1147 | 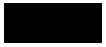 | 0.0938 | 962_P_−2 |
| 962_Q_−1 | 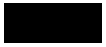 | 0.0563 | 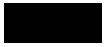 | 0.0875 | 962_Q_−1 |
| 962_S_−1 | 0.0551 | 0.5854 | 0.8282 | 0.7192 | 962_S_−1 |
| 962_U_1 | 0.064 | 0.5148 | 0.6903 | 0.5005 | 962_U_1 |
| 962_U_2 | 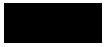 | 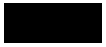 | 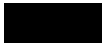 | 0.0587 | 962_V_2 |
| 962_V_−1 | | 0.1995 | 0.0821 | 0.076 | 962_V_−1 |
| 962_V_+2 | — | | 0.6823 | 0.2958 | 962_V_+2 |
| 962_Z_1 | | | | 0.2185 | 962_Z_1 |
| CNTL | 21.7% | 35.4% | 4.7% | 35.5% | CNTL |
| CASE | 39.6% | 23.9% | 2.1% | 25.0% | CASE |

All SNP combinations in Tables 21, 22, and 23 that demonstrated a significant difference (p≦0.05) in the distribution of frequencies of the four haplotypes between the cases and the control populations were further analyzed to identify individual haplotypes that were also significant. Table 24 presents the haplotypes that were significantly associated, at the 0.05 level of significance, with the asthma phenotype. Haplotypes with higher allele frequency in the case population than in the control population acted as risk factors that increased the susceptibility to asthma. Haplotypes with lower allele frequencies in the case population than in the control population acted as protective factors that decreased the susceptibility to asthma. For Gene 962, three haplotypes involving allele A at SNP G1 were susceptibility haplotypes, associated with an increased risk of asthma at the 0.05 level of significance in the combined population. They were haplotypes A/A (SNPs G1/Q−1, p=0.0084), A/C (SNPs G1/V−1, p=0.0142) and G/A (SNPs G4/G1, p=0.045). Haplotype A/C was a protective haplotype (SNPs H+2/S−1, p=0.0097). In the UK population, three haplotypes were protective. They were haplotypes C/T (SNPs E+2/V−1, p=0.0149), G/T (SNPs G1/G6, p=0.0164) and C/C (SNPs G6/S−1, p=0.0308). In the US population, six haplotypes were susceptibility haplotypes. They were G/A (SNPs G4/Q-1, p=0.0466), G/T (SNPs G4/U2, p=0.0363), A/A (SNPs G4/V+2, p=0.0428), A/A (SNPs G1/Q−1, p=0.0024), A/T (SNPs G1/U2, p=0.0027) and T/G (SNPs U2/V+2, p=0.0216). For Gene 845, haplotype G/G (SNPs R1/K−2, p=0.01 16) was a susceptibility haplotype in the US population and haplotype A/G (SNPs R1/K−2, p=0.0367) was protective in the US population. For Gene 803, three haplotypes involving allele A at SNP K2 were susceptibility haplotypes in the US population. They were haplotypes C/A (SNPs K3/K2, p=0.0451), A/C (SNPs K2/I1, p=0.0453) and A/A (SNPs K2/E+2, p=0.0442).

TABLE 24

| | | | | | |
|---|---|---|---|---|---|
| | SNP | | | | |
| | COMBI- | HAPLO- | FREQUENCIES | | P- |
| GENE | NATION | TYPE | CNTL | CASE | VALUE |

Asthma Yes/No
Combined US and UK

| | | | | | |
|---|---|---|---|---|---|
| 962 | G4/G1 | GA | 0.208523 | 0.278141 | 0.045 |
| 962 | G1/Q−1 | AA | 0.049187 | 0.12121 | 0.0084 |
| 962 | G1/V−1 | AC | 0.157636 | 0.256126 | 0.0142 |
| 962 | H+2/S−1 | AC | 0.05552 | 0.000001 | 0.0097 |

Asthma Yes/No
UK Population

| | | | | | |
|---|---|---|---|---|---|
| 962 | E+2/V−1 | CT | 0.054678 | 0 | 0.0149 |
| 962 | G1/G6 | GT | 0.198435 | 0.099309 | 0.0164 |
| 962 | G6/S−1 | CC | 0.101659 | 0.040006 | 0.0308 |

Asthma Yes/No
US Population

| | | | | | |
|---|---|---|---|---|---|
| 845 | R1/K−2 | GG | 0.504855 | 0.731782 | 0.0116 |
| 845 | R1/K−2 | AG | 0.17047 | 0.032411 | 0.0367 |
| 803 | K3/K2 | CA | 0 | 0.045455 | 0.0451 |
| 803 | K2/I−1 | AG | 0 | 0.045455 | 0.0478 |
| 803 | K2/E+2 | AA | 0 | 0.045455 | 0.0442 |
| 962 | G4/Q−1 | GA | 0.192645 | 0.327035 | 0.0466 |
| 962 | G4/U2 | GT | 0.191643 | 0.333333 | 0.0363 |
| 962 | G4/V+2 | AA | 0 | 0.020833 | 0.0428 |
| 962 | G1/Q−1 | AA | 0.042798 | 0.25136 | 0.0024 |
| 962 | G1/U2 | AT | 0.039558 | 0.248019 | 0.0027 |
| 962 | U2/V+2 | TG | 0.217236 | 0.375 | 0.0216 |

b. Bronchial Hyper-Responsiveness:

In Tables 25, 26 and 27, the haplotype analysis (2-at-a-time) is presented for the combined, the UK and the US populations, respectively. Ten SNP combinations in Gene 845 are significant in the combined, the UK and the US population alone: SNPs R 1 & K−2 (combined p=0.0385), SNPs R−1 & J 1 (UK p=0.013), SNPs P+1 & H+1 (US p=0.0267), SNPs K 1 & H+1 (US p=0.0076), SNPs K 1 & D−1 (combined p=0.0134), SNPs K−2 & H+1 (combined p=0.0355), SNPs K−2 & D 1 (UK p=0.0428), SNPs J 1 & D 1 (UK p=0.0097), SNPs H−1 & D 1 (UK p=0.0422) and SNPs D 1 & D−1 (UK p=0.007). Nine SNP combinations in Gene 847 are significant in the US population: SNPs K 1 & D−1 (p=0.0118), SNPs K 1 & C+1 (p=0.0225), SNPs J+1 & E+1 (p=0.038), SNPs J+1 & D−1 (p=0.0081), SNPs J+1 & C+1 (p=0.0077), SNPs E+1 & C+1 (p=0.0296), SNPs D−1 & A 2 (p=0.0343), SNPs D−1 & A 1 (p=0.0483) and SNPs C+1 & A 2 (p=0.0328). Two SNP combinations in Gene 803 are significant in the US population: SNPs K 2 & I 1 (p=0.0212), and SNPs K 2 & E+2 (p=0.0281). Two SNP combinations in Gene 962 are significant in the UK and the US population alone: SNPs G 2 & S−1 (UK p=0.0491) and SNPs E 3 & E+2 (US p=0.0431).

TABLE 25
HAPLOTYPE ANALYSIS OF BHR PHENOTYPE COMBINED US/UK POPULATION

| | 845_R_1 | 845_R_−1 | 845_P_+1 | 845_K_1 | 845_K_2 | 845_J_1 | 845_J_−1 | 845_I_−1 | 845_H_+1 | 845_H_−1 | 845_G_+1 | 845_F_+1 | 845_D_1 | 845_D_−1 | |
|---|---|---|---|---|---|---|---|---|---|---|---|---|---|---|---|
| 845_{—} | 0.1227 | 0.093 | 0.1055 | 0.254 | 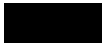 | 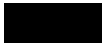 | 0.3268 | 0.2586 | 0.4918 | 0.0834 | 0.4205 | 0.418 | 0.0775 | 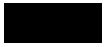 | 845_R_1 |
| R_1 | | | | | | | | | | | | | | | |
| 845_{—} | — | 0.4252 | 0.1888 | 0.4799 | 0.1658 | 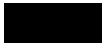 | 0.1544 | 0.7499 | 0.0753 | 0.1256 | 0.729 | 0.7624 | 0.2879 | 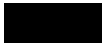 | 845_R_−1 |
| R_−1 | | | | | | | | | | | | | | | |
| 845_{—} | — | — | 0.0715 | 0.1912 | 0.1045 | 0.0511 | 0.3078 | 0.2192 | 0.1092 | 0.184 | 0.18 | 0.2121 | 0.1304 | 0.0807 | 845_P_+1 |
| P_+1 | | | | | | | | | | | | | | | |
| 845_{—} | | — | — | 1 | 0.2677 | 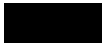 | 0.4404 | 0.9403 | 0.2549 | 0.3269 | 0.7194 | 0.9307 | 0.2135 | 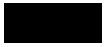 | 845_K_1 |
| K_1 | | | | | | | | | | | | | | | |
| 845_{—} | — | — | — | — | 0.1469 | 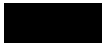 | 0.0665 | 0.2862 | 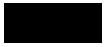 | 0.2709 | 0.3012 | 0.2844 | 0.1286 | 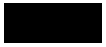 | 845_K_−2 |
| K_−2 | | | | | | | | | | | | | | | |
| 845_{—} | — | — | — | — | — | 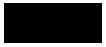 | 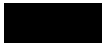 | 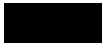 | 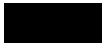 | 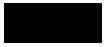 | 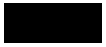 | 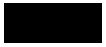 | 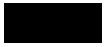 | 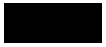 | 845_J_1 |
| J_1 | | | | | | | | | | | | | | | |
| 845_{—} | — | — | — | — | — | — | 0.3952 | 0.5902 | 0.2621 | 0.3141 | 0.8472 | 0.8055 | 0.197 | 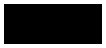 | 845_J_−1 |
| J_−1 | | | | | | | | | | | | | | | |
| 845_{—} | — | — | — | — | — | — | — | 1 | 0.2429 | 0.37 | 0.6965 | 0.7552 | 0.584 | 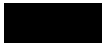 | 845_I_−1 |
| I_−1 | | | | | | | | | | | | | | | |
| 845_{—} | — | — | — | — | — | — | — | — | 0.1325 | 0.0718 | 0.2968 | 0.4653 | 0.0749 | 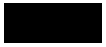 | 845_H_+1 |
| H_+1 | | | | | | | | | | | | | | | |
| 845_{—} | — | — | — | — | — | — | — | — | — | 0.1969 | 0.424 | 0.3396 | 0.192 | 0.0636 | 845_H_−1 |
| H_−1 | | | | | | | | | | | | | | | |
| 845_{—} | — | — | — | — | — | — | — | — | — | — | 0.7318 | 0.6831 | 0.4852 | 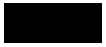 | 845_G_+1 |
| G_+1 | | | | | | | | | | | | | | | |
| 845_{—} | — | — | — | — | — | — | — | — | — | — | — | 1 | 0.5881 | 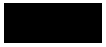 | 845_F_+1 |
| F_+1 | | | | | | | | | | | | | | | |
| CNTL | 14.8% | 28.7% | 6.5% | 0.2% | 28.9% | 34.5% | 37.1% | 12.8% | 19.6% | 45.2% | 13.2% | 18.4% | 0.2% | 9.9% | CNTL |
| CASE | 8.3% | 23.8% | 12.5% | 0.0% | 20.9% | 50.0% | 31.8% | 12.5% | 12.5% | 37.5% | 14.8% | 18.2% | 1.2% | 20.2% | CASE |

| | 847_K_1 | 847_J_+1 | 847_E_+1 | 847_D_−1 | 847_C_+1 | 847_A_2 | 847_A_1 | |
|---|---|---|---|---|---|---|---|---|
| 847_K_1 | 0.781 | 0.1716 | 0.8466 | 0.7351 | 0.93 | 0.6906 | 0.8334 | 847_K_1 |
| 847_J_+1 | — | 0.1502 | 0.1639 | 0.1579 | 0.2008 | 0.1666 | 0.1498 | 847_J_+1 |
| 847_E_+1 | — | — | 1 | 0.8405 | 0.964 | 0.3695 | 0.9439 | 847_E_+1 |
| 847_D_−1 | — | — | — | 0.7526 | 0.7406 | 0.617 | 0.9136 | 847_D_−1 |
| 847_C_+1 | — | — | — | — | 1 | 0.6384 | 0.9961 | 847_C_+1 |
| 847_A_2 | — | — | — | — | — | 0.4852 | 0.7284 | 847_A_2 |
| 847_A_1 | — | — | — | — | — | — | 1 | 847_A_1 |
| CNTL | 4.9% | 4.9% | 12.6% | 16.9% | 17.9% | 6.7% | 1.0% | CNTL |
| CASE | 3.6% | 1.1% | 12.0% | 15.1% | 17.7% | 4.2% | 1.0% | CASE |

| | 874_R_+1 | 874_S_+1 | 874_T_−1 | 874_V_−1 | |
|---|---|---|---|---|---|
| 874_R_+1 | 0.0738 | 0.1638 | 0.2993 | 0.2605 | 874_R_+1 |
| 874_S_+1 | — | 0.6422 | 0.3442 | 0.7898 | 874_S_+1 |
| 874_T_−1 | — | — | 0.8214 | 0.92 | 874_T_−1 |
| 874_V_−1 | — | — | — | 0.7695 | 874_V_−1 |
| CNTL | 39.9% | 39.5% | 48.6% | 17.6% | CNTL |
| CASE | 29.3% | 42.6% | 46.9% | 18.8% | CASE |

| | 803_K_3 | 803_K_2 | 803_I_1 | 803_I_−1 | 803_H_+1 | 803_E_+2 | |
|---|---|---|---|---|---|---|---|
| 803_K_3 | 0.1647 | 0.3798 | 0.4488 | 0.4937 | 0.3962 | 0.5099 | 803_K_3 |
| 803_K_2 | — | 0.3776 | 0.9758 | 0.9847 | 0.5573 | 0.8898 | 803_K_2 |
| 803_I_1 | — | — | 1 | 0.9954 | 0.7551 | 0.6129 | 803_I_1 |
| 803_I_−1 | — | — | — | 1 | 0.5881 | 0.7119 | 803_I_−1 |
| 803_H_+1 | — | — | — | — | 0.5414 | 0.8006 | 803_H_+1 |
| 803_E_+2 | — | — | — | — | — | 0.6722 | 803_E_+2 |
| CNTL | 0.9% | 0.2% | 28.2% | 0.2% | 25.2% | 44.2% | CNTL |
| CASE | 2.6% | 0.9% | 28.4% | 0.0% | 21.9% | 46.5% | CASE |

| | 962_E_3 | 962_E_+2 | 962_G_4 | 962_G_1 | 962_G_2 | 962_G_6 | 962_H_+2 | 962_M_+2 | 962_P_−2 | 962_Q_−1 | 962_S_−1 | 962_U_1 | |
|---|---|---|---|---|---|---|---|---|---|---|---|---|---|
| 962_E_3 | 0.9071 | 0.4317 | 0.516 | 0.4162 | 0.9572 | 0.7189 | 0.4794 | 0.6478 | 0.7666 | 0.8326 | 0.774 | 0.874 | 962_E_3 |
| 962_E_+2 | | 0.3128 | 0.3279 | 0.6247 | 0.2338 | 0.4261 | 0.4258 | 0.3215 | 0.2481 | 0.3352 | 0.2178 | 0.4443 | 962_E_+2 |
| 962_G_4 | — | | 0.1756 | 0.2176 | 0.365 | 0.3236 | 0.2693 | 0.1477 | 0.3337 | 0.3724 | 0.1964 | 0.3196 | 962_G_4 |
| 962_G_1 | — | | | 0.626 | 0.7488 | 0.6561 | 0.6164 | 0.5051 | 0.6091 | 0.4918 | 0.6264 | 0.7516 | 962_G_1 |
| 962_G_2 | | | | | 0.683 | 0.154 | 0.7557 | 0.3427 | 0.7201 | 0.7211 | 0.1784 | 0.5279 | 962_G_2 |
| 962_G_6 | — | | | | | 0.3165 | 0.2217 | 0.2693 | 0.0824 | 0.0752 | 0.2377 | 0.1175 | 962_G_6 |
| 962_H_+2 | — | — | — | | — | | 0.2641 | 0.0762 | 0.622 | 0.6895 | 0.1513 | 0.6198 | 962_H_+2 |
| 962_M_+2 | | | | | — | — | | 0.2968 | 0.3632 | 0.3917 | 0.6844 | 0.3581 | 962_M_+2 |
| 962_P_−2 | | | | | — | — | | | 0.6921 | 0.9484 | 0.5977 | 0.789 | 962_P_−2 |
| 962_Q_−1 | — | | | | — | — | — | — | — | 0.7901 | 0.6265 | 0.7198 | 962_Q_−1 |
| 962_S_−1 | — | — | | | — | — | — | — | — | | 0.3741 | 0.4508 | 962_S_−1 |
| 962_U_1 | — | | | | — | — | — | — | | — | | 0.3617 | 962_U_1 |
| 962_U_2 | — | | | | — | — | — | — | | — | — | | 962_U_2 |
| 962_V_−1 | | | | — | — | — | — | — | | — | | — | 962_V_−1 |
| 962_V_+2 | — | — | — | | — | — | — | — | — | — | | — | 962_V_+2 |
| 962_Z_1 | | | — | — | — | — | — | — | | — | | — | 962_Z_1 |
| CNTL | 35.6% | 12.7% | 13.6% | 27.2% | 7.6% | 20.4% | 41.5% | 12.7% | 23.8% | 23.7% | 11.4% | 3.0% | CNTL |
| CASE | 34.7% | 8.7% | 8.3% | 29.8% | 8.8% | 15.3% | 35.3% | 8.2% | 21.4% | 21.9% | 7.8% | 4.9% | CASE |

| | 962_U_2 | 962_V_−1 | 982_V_+2 | 962_Z_1 | |
|---|---|---|---|---|---|
| 962_E_3 | 0.7189 | 0.3361 | 0.6552 | 0.6083 | 962_E_3 |
| 962_E_+2 | 0.3618 | 0.1028 | 0.3315 | 0.3764 | 962_E_+2 |
| 962_G_4 | 0.2854 | 0.1625 | 0.2663 | 0.2801 | 962_G_4 |
| 962_G_1 | 0.7442 | 0.2441 | 0.6164 | 0.5673 | 962_G_1 |
| 962_G_2 | 0.8726 | 0.4279 | 0.6058 | 0.5691 | 962_G_2 |
| 962_G_6 | 0.6195 | 0.2712 | 0.3766 | 0.3552 | 962_G_6 |
| 962_H_+2 | 0.5144 | 0.2246 | 0.2681 | 0.4391 | 962_H_+2 |
| 962_M_+2 | 0.133 | 0.2053 | 0.0779 | 0.4261 | 962_M_+2 |
| 962_P_−2 | 0.8121 | 0.2414 | 0.471 | 0.5539 | 962_P_−2 |
| 962_Q_−1 | 0.2688 | 0.1939 | 0.5462 | 0.6022 | 962_Q_−1 |
| 962_S_−1 | 0.3359 | 0.2835 | 0.0991 | 0.5538 | 962_S_−1 |
| 962_U_1 | 0.693 | 0.1535 | 0.3288 | 0.5037 | 962_U_1 |
| 962_U_2 | 0.5095 | 0.1516 | 0.4574 | 0.6757 | 962_U_2 |
| 962_V_−1 | | 0.1476 | 0.2689 | 0.2726 | 962_V_−1 |
| 962_V_+2 | — | | 0.3973 | 0.3009 | 962_V_+2 |
| 962_Z_1 | — | — | | 0.2968 | 962_Z_1 |
| CNTL | 23.8% | 34.8% | 4.5% | 31.9% | CNTL |
| CASE | 20.0% | 26.5% | 2.0% | 37.5% | CASE |

TABLE 26
HAPLOTYPE ANALYSIS OF BHR PHENOTYPE UK POPULATION

| | 845_R_1 | 845_R_−1 | 845_P_+1 | 845_K_1 | 845_K_−2 | 845_J_1 | 845_J_−1 | 845_I_−1 | 845_H_+1 | 845_H_−1 | 845_G_+1 | 845_F_+1 | 845_D_1 | 845_D_−1 | |
|---|---|---|---|---|---|---|---|---|---|---|---|---|---|---|---|
| 845_{—} | 0.4358 | 0.3636 | 0.4243 | 0.5414 | 0.1445 | 0.0893 | 0.7328 | 0.6729 | 0.8754 | 0.1964 | 0.8488 | 0.8809 | 0.0859 | 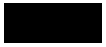 | 845_R_1 |
| R_1 | | | | | | | | | | | | | | | |
| 845_{—} | — | 0.4437 | 0.4676 | 0.5086 | 0.094 | 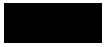 | 0.3194 | 0.6632 | 0.3213 | 0.0654 | 0.6852 | 0.6861 | 0.133 | 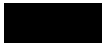 | 845_R_−1 |
| R_−1 | | | | | | | | | | | | | | | |
| 845_{—} | — | — | 0.212 | 0.4876 | 0.2276 | 0.1577 | 0.6931 | 0.5344 | 0.419 | 0.3042 | 0.4678 | 0.5175 | 0.1343 | 0.0725 | 845_P_+1 |
| P_+1 | | | | | | | | | | | | | | | |
| 845_{—} | — | — | | 1 | 0.3021 | 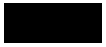 | 0.6043 | 0.9481 | 0.4873 | 0.3001 | 0.6846 | 0.9503 | 0.0978 | 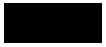 | 845_K_1 |
| K_1 | | | | | | | | | | | | | | | |
| 845_{—} | — | — | | | 0.1299 | 0.0504 | 0.1195 | 0.247 | 0.1486 | 0.1872 | 0.2942 | 0.2783 | 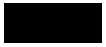 | 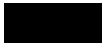 | 845_K_−2 |
| K_−2 | | | | | | | | | | | | | | | |
| 845_{—} | | — | — | | | 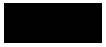 | 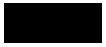 | 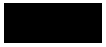 | 0.1209 | 0.0621 | 0.1018 | 0.0957 | 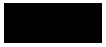 | 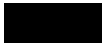 | 845_J_1 |
| J_1 | | | | | | | | | | | | | | | |
| 845_{—} | — | — | — | | | | 0.5899 | 0.7923 | 0.6445 | 0.3626 | 0.9382 | 0.9304 | 0.1438 | 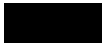 | 845_J_−1 |
| J_−1 | | | | | | | | | | | | | | | |
| R_1 | | | | | | | | | | | | | | | |
| 845_{—} | | — | — | — | — | — | | 1 | 0.6382 | 0.2665 | 0.7018 | 0.9058 | 0.2428 | 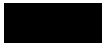 | 845_I_−1 |
| I_−1 | | | | | | | | | | | | | | | |
| 845_{—} | | | — | — | — | — | — | | 0.4994 | 0.1731 | 0.6698 | 0.8308 | 0.0868 | 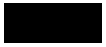 | 845_H_+1 |
| H_+1 | | | | | | | | | | | | | | | |
| 845_{—} | | — | — | — | — | | — | | | 0.1454 | 0.3025 | 0.2504 | 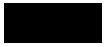 | 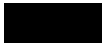 | 845_H_−1 |
| H_+1 | | | | | | | | | | | | | | | |
| 845_{—} | | — | — | — | — | | — | — | — | | 0.7047 | 0.8022 | 0.1862 | 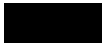 | 845_G_+1 |
| G_+1 | | | | | | | | | | | | | | | |
| 845_{—} | | — | — | — | — | | — | — | | — | | 1 | 0.2476 | 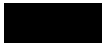 | 845_F_+1 |
| F_+1 | | | | | | | | | | | | | | | |
| 845_{—} | | — | — | — | — | | — | — | — | — | | | 0.2045 | 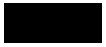 | 845_D_1 |
| D_1 | | | | | | | | | | | | | | | |
| 845_{—} | | — | — | — | — | | | | | — | | — | | 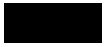 | 845_D_−1 |
| D_−1 | | | | | | | | | | | | | | | |
| CNTL | 13.6% | 26.6% | 6.4% | 0.4% | 27.0% | 36.6% | 37.5% | 12.6% | 19.3% | 43.6% | 13.2% | 18.8% | 0.0% | 9.3% | CNTL |
| CASE | 9.7% | 21.4% | 10.8% | 0.0% | 18.1% | 51.4% | 33.8% | 12.2% | 14.9% | 33.8% | 14.9% | 18.9% | 1.4% | 20.8% | CASE |

| | 847_K_1 | 847_J_+1 | 847_E_+1 | 847_D_−1 | 847_C_+1 | 847_A_2 | 847_A_1 | |
|---|---|---|---|---|---|---|---|---|
| 847_K_1 | 0.725 | 0.6597 | 0.704 | 0.9645 | 0.9194 | 0.9621 | 0.8974 | 847_K_1 |
| 847_J_+1 | — | 0.69 | 0.7023 | 0.6844 | 0.5625 | 0.8319 | 0.7757 | 847_J_+1 |
| 847_E_+1 | — | — | 0.8486 | 0.6266 | 0.8008 | 0.6334 | 0.8728 | 847_E_+1 |
| 847_D_−1 | — | — | — | 0.7215 | 0.5405 | 0.699 | 0.9003 | 847_D_−1 |
| 847_C_+1 | — | — | — | | 0.513 | 0.6701 | 0.8359 | 847_C_+1 |
| 847_A_2 | — | — | — | — | | 1 | 0.9999 | 847_A_2 |
| 847_A_1 | — | — | — | — | — | | 1 | 847_A_1 |
| CNTL | 3.6% | 2.9% | 12.8% | 16.5% | 17.8% | 5.4% | 1.3% | CNTL |
| CASE | 4.4% | 1.3% | 14.1% | 18.6% | 21.3% | 5.0% | 1.3% | CASE |

| | 874_R_+1 | 874_S_+1 | 874_T_−1 | 874_V_−1 | |
|---|---|---|---|---|---|
| 874_R_+1 | 0.1116 | 0.2338 | 0.3081 | 0.4057 | 874_R_+1 |
| 874_S_+1 | — | 0.2947 | 0.233 | 0.56 | 874_S_+1 |
| 874_T_−1 | — | — | 0.8989 | 0.9168 | 874_T_−1 |
| 874_V_−1 | — | — | — | 0.7379 | 874_V_−1 |
| CNTL | 41.5% | 38.0% | 48.9% | 16.9% | CNTL |
| CASE | 30.8% | 44.9% | 47.5% | 18.8% | CASE |

| | 803_K_3 | 803_K_2 | 803_I_1 | 803_I_−1 | 803_H_+1 | 803_E_+2 | |
|---|---|---|---|---|---|---|---|
| 803_K_3 | 0.1828 | 0.4394 | 0.3288 | 0.5003 | 0.3875 | 0.356 | 803_K_3 |
| 803_K_2 | | 1 | 0.6552 | 0.9425 | 0.5862 | 0.4151 | 803_K_2 |
| 803_I_1 | — | — | 0.5933 | 0.6726 | 0.4824 | 0.511 | 803_I_1 |
| 803_I_−1 | — | — | | 1 | 0.6153 | 0.4267 | 803_I_−1 |
| 803_H_+1 | — | — | — | | 0.4953 | 0.487 | 803_H_+1 |
| 803_E_+2 | — | | — | — | | 0.2821 | 803_E_+2 |
| CNTL | 1.1% | 0.4% | 28.6% | 0.4% | 25.7% | 42.4% | CNTL |
| CASE | 3.1% | 0.0% | 25.5% | 0.0% | 21.9% | 49.0% | CASE |

| | 962_E_3 | 962_E_+2 | 962_G_4 | 962_G_1 | 962_G_2 | 962_G_6 | 962_H_+2 | 962_M_+2 | 962_P_−2 | 962_Q_−1 | 962_S_−1 | 962_U_1 | |
|---|---|---|---|---|---|---|---|---|---|---|---|---|---|
| 962_E_3 | 0.515 | 0.7464 | 0.5152 | 0.3509 | 0.5785 | 0.6404 | 0.4499 | 0.4264 | 0.3429 | 0.4711 | 0.5547 | 0.6799 | 962_E_3 |
| 962_E_+2 | | 0.5802 | 0.5161 | 0.7985 | 0.1852 | 0.6109 | 0.4938 | 0.451 | 0.1519 | 0.2682 | 0.3983 | 0.7012 | 962_E_+2 |
| 962_G_4 | — | | 0.2549 | 0.2797 | 0.2378 | 0.2417 | 0.3093 | 0.2107 | 0.235 | 0.2861 | 0.2708 | 0.3498 | 962_G_4 |
| 962_G_1 | | — | | 0.4922 | 0.531 | 0.492 | 0.4817 | 0.4536 | 0.4058 | 0.4423 | 0.5459 | 0.432 | 962_G_1 |
| 962_G_2 | | | | | 0.2444 | 0.2212 | 0.4306 | 0.1508 | 0.6403 | 0.6035 | 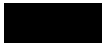 | 0.4173 | 962_G_2 |
| 962_G_6 | | — | | — | | 0.354 | 0.2355 | 0.2219 | 0.0881 | 0.0733 | 0.1759 | 0.198 | 962_G_6 |
| 962_H_+2 | | — | — | — | — | | 0.1748 | 0.0984 | 0.2908 | 0.4093 | 0.2204 | 0.483 | 962_H_+2 |
| 962_M_+2 | | — | | | | — | | 0.2352 | 0.1972 | 0.2338 | 0.6025 | 0.4056 | 962_M_+2 |
| 962_P_−2 | | — | | | | | — | | 0.3843 | 0.7457 | 0.4412 | 0.7518 | 962_P_−2 |
| 962_Q_−1 | | — | | | | — | — | — | — | 0.465 | 0.5054 | 0.8408 | 962_Q_−1 |
| 962_S_−1 | | — | — | | | — | — | — | — | | 0.3107 | 0.5315 | 962_S_−1 |
| 962_U_1 | | — | — | — | — | — | — | — | — | — | | 0.7524 | 962_U_1 |
| 962_U_2 | | — | | | | — | — | — | — | — | — | | 962_U_2 |
| 962_V_−1 | | — | | | | — | — | — | — | — | | — | 962_V_−1 |
| 962_V_+2 | | | — | | | — | — | — | — | | — | | 962_V_+2 |
| 962_Z_1 | | | | | | | | | — | — | | — | 962_Z_1 |
| CNTL | 36.8% | 12.5% | 14.0% | 25.2% | 6.4% | 21.7% | 43.6% | 12.4% | 24.6% | 24.8% | 10.9% | 3.7% | CNTL |
| CASE | 32.1% | 10.0% | 85% | 28.9% | 10.2% | 16.7% | 35.2% | 7.1% | 19.3% | 20.2% | 6.8% | 4.5% | CASE |

| | 962_U_2 | 962_V_−1 | 962_V_+2 | 962_Z_1 | |
|---|---|---|---|---|---|
| 962_E_3 | 0.3051 | 0.2308 | 0.7642 | 0.2543 | 962_E_3 |
| 962_E_+2 | 0.1706 | 0.1462 | 0.6373 | 0.3188 | 962_E_+2 |
| 962_G_4 | 0.1569 | 0.1482 | 0.4131 | 0.2011 | 962_G_4 |
| 962_G_1 | 0.4464 | 0.1672 | 0.7516 | 0.178 | 962_G_1 |
| 962_G_2 | 0.2471 | 0.2202 | 0.5097 | 0.3565 | 962_G_2 |
| 962_G_6 | 0.462 | 0.2207 | 0.4478 | 0.1923 | 962_G_6 |
| 962_H_+2 | 0.1388 | 0.191 | 0.2337 | 0.2066 | 962_H_+2 |
| 962_M_+2 | 0.0629 | 0.1486 | 0.087 | 0.159 | 962_M_+2 |
| 962_P_−2 | 0.8329 | 0.1438 | 0.4439 | 0.3371 | 962_P_−2 |
| 962_Q_−1 | 0.3176 | 0.1324 | 0.6132 | 0.3637 | 962_Q_−1 |
| 962_S_−1 | 0.1474 | 0.2006 | 0.1081 | 0.2071 | 962_S_−1 |
| 962_U_1 | 0.5151 | 0.067 | 0.5727 | 0.3179 | 962_U_1 |
| 962_U_2 | 0.1868 | 0.0501 | 0.3777 | 0.2338 | 962_U_2 |
| 962_V_−1 | | 0.1357 | 0.3014 | 0.2074 | 962_V_−1 |
| 962_V_+2 | — | | 0.5311 | 0.1916 | 962_V_+2 |
| 962_Z_1 | | — | | 0.0924 | 962_Z_1 |
| CNTL | 25.0% | 34.4% | 4.5% | 30.0% | CNTL |
| CASE | 17.4% | 25.0% | 2.3% | 40.0% | CASE |

TABLE 27
HAPLOTYPE ANALYSIS OF BHR PHENOTYPE US POPULATION

| | 845_R_1 | 845_R_−1 | 845_P_+1 | 845_K_1 | 845_K_−2 | 845_J_1 | 845_J_−1 | 845_I_−1 | 845_H_−1 | 845_H_−1 | 845_G_+1 | 845_F_+1 | 845_D_1 | 845_D_−1 | |
|---|---|---|---|---|---|---|---|---|---|---|---|---|---|---|---|
| 845_{—} | 0.2168 | 0.1594 | 0.0827 | 0.0855 | 0.1679 | 0.2103 | 0.1745 | 0.2628 | 0.1094 | 0.1574 | 0.2903 | 0.2857 | 0.1568 | 0.2542 | 845_R_1 |
| R_1 | | | | | | | | | | | | | | | |
| 845_{—} | | 0.7742 | 0.1844 | 0.9763 | 0.9421 | 0.4599 | 0.4528 | 0.9788 | 0.0539 | 0.9001 | 0.9422 | 0.9765 | 0.9697 | 0.7545 | 845_R_−1 |
| R_−1 | | | | | | | | | | | | | | | |
| 845_{—} | — | | 0.0801 | 0.0886 | 0.1795 | 0.3146 | 0.1941 | 0.2962 | 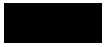 | 0.1209 | 0.2818 | 0.3014 | 0.1605 | 0.2485 | 845_P_+1 |
| P_+1 | | | | | | | | | | | | | | | |
| 845_{—} | — | | — | 1 | 0.966 | 0.5089 | 0.2612 | 0.928 | 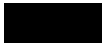 | 0.5848 | 0.8566 | 0.8208 | 0.8841 | 0.681 | 845_K_1 |
| K_1 | | | | | | | | | | | | | | | |
| 845_{—} | — | — | — | | 0.7742 | 0.4363 | 0.4784 | 0.9915 | 0.0651 | 0.8203 | 0.9969 | 0.9798 | 0.9669 | 0.7489 | 845_K_−2 |
| K_−2 | | | | | | | | | | | | | | | |
| 845_{—} | — | — | | | | 0.3787 | 0.4458 | 0.6187 | 0.0897 | 0.0573 | 0.744 | 0.6551 | 0.4122 | 0.7182 | 845_J_1 |
| J_1 | | | | | | | | | | | | | | | |
| 845_{—} | — | | | | — | | 0.383 | 0.5758 | 0.0656 | 0.2691 | 0.5926 | 0.5584 | 0.394 | 0.6422 | 845_J_−1 |
| J_−1 | | | | | | | | | | | | | | | |
| 845_{—} | — | | | | — | — | — | 1 | 0.0879 | 0.7864 | 0.8033 | 0.6122 | 0.9962 | 0.5973 | 845_I_−1 |
| I_−1 | | | | | | | | | | | | | | | |
| 845_{—} | — | | — | | | — | — | | 0.0752 | 0.0751 | 0.0988 | 0.133 | 0.0521 | 0.061 | 845_H_+1 |
| H_+1 | | | | | | | | | | | | | | | |
| 845_{—} | — | | | — | | — | — | — | — | 0.5841 | 0.7955 | 0.8275 | 0.6282 | 0.5876 | 845_H_−1 |
| H_−1 | | | | | | | | | | | | | | | |
| 845_{—} | | | — | | | — | — | | | | 1 | 0.684 | 0.9967 | 0.5614 | 845_G_+1 |
| G_+1 | | | | | | | | | | | | | | | |
| 845_{—} | | | | — | | — | — | — | | | | 1 | 0.9645 | 0.6021 | 845_F_+1 |
| F_+1 | | | | | | | | | | | | | | | |
| 845_{—} | — | | — | | | — | — | — | | — | — | | 1 | 0.4079 | 845_D_1 |
| D_1 | | | | | | | | | | | | | | | |
| 845_{—} | — | | — | — | — | — | — | — | | | — | — | | 0.6307 | 845_D_−1 |
| D_−1 | | | | | | | | | | | | | | | |
| CNTL | 17.1% | 32.5% | 6.5% | 0.0% | 32.5% | 30.9% | 36.4% | 13.3% | 20.1% | 48.1% | 13.2% | 17.5% | 0.7% | 11.0% | CNTL |
| CASE | 0.0% | 35.7% | 21.4% | 0.0% | 35.7% | 42.9% | 21.4% | 14.3% | 0.0% | 57.1% | 14.3% | 14.3% | 0.0% | 16.7% | CASE |

| | 847_K_1 | 847_J_+1 | 847_E_+1 | 847_D_−1 | 847_C_+1 | 847_A_2 | 847_A_1 | |
|---|---|---|---|---|---|---|---|---|
| 847_K_1 | 0.6027 | 0.1218 | 0.0756 | 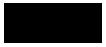 | 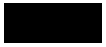 | 0.0971 | 0.219 | 847_K_1 |
| 847_J_+1 | — | 0.6146 | 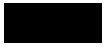 | 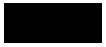 | 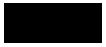 | 0.0668 | 0.2001 | 847_J_+1 |
| 847_E_+1 | — | — | 0.3721 | 0.11 | 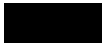 | 0.1021 | 0.1828 | 847_E_+1 |
| 847_D_−1 | — | — | — | 0.0779 | 0.0563 | 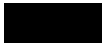 | 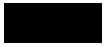 | 847_D_−1 |
| 847_C_+1 | — | — | — | — | 0.077 | 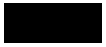 | 0.0514 | 847_C_+1 |
| 847_A_2 | — | — | — | — | — | 0.3669 | 0.117 | 847_A_2 |
| 847_A_1 | — | — | — | — | — | — | 1 | 847_A_1 |
| CNTL | 7.3% | 8.4% | 12.3% | 17.6% | 18.2% | 9.1% | 0.7% | CNTL |
| CASE | 0.0% | 0.0% | 0.0% | 0.0% | 0.0% | 0.0% | 0.0% | CASE |

| | 874_R_+1 | 874_S_+1 | 874_T_−1 | 874_V_−1 | |
|---|---|---|---|---|---|
| 874_R_+1 | 0.3818 | 0.0621 | 0.4429 | 0.115 | 874_R_+1 |
| 874_S_+1 | — | 0.4382 | 0.2736 | 0.5583 | 874_S_+1 |
| 874_T_−1 | — | — | 0.7977 | 0.9652 | 874_T_−1 |
| 874_V_−1 | — | — | — | 1 | 874_V_−1 |
| CNTL | 37.0% | 42.2% | 48.1% | 18.8% | CNTL |
| CASE | 21.4% | 31.3% | 43.8% | 18.8% | CASE |

| | 803_K_3 | 803_K_2 | 803_I_1 | 803_I_−1 | 803_H_+1 | 803_E_+2 | |
|---|---|---|---|---|---|---|---|
| 803_K_3 | 1 | 0.182 | 0.2121 | 0.782 | 0.8147 | 0.3784 | 803_K_3 |
| 803_K_2 | — | 0.1034 | 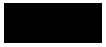 | 0.0804 | 0.1419 | 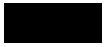 | 803_K_2 |
| 803_I_1 | — | — | 0.1714 | 0.1246 | 0.3257 | 0.3586 | 803_I_1 |
| 803_I_−1 | — | — | — | 1 | 0.9227 | 0.2631 | 803_I_−1 |
| 803_H_+1 | — | — | — | — | 1 | 0.5364 | 803_H_+1 |
| 803_E_+2 | — | — | — | — | — | 0.3214 | 803_E_+2 |
| CNTL | 0.6% | 0.0% | 27.6% | 0.0% | 24.4% | 47.4% | CNTL |
| CASE | 0.0% | 5.6% | 44.4% | 0.0% | 22.2% | 33.3% | CASE |

| | 962_E_3 | 962_E_+2 | 962_G_4 | 962_G_1 | 962_G_2 | 962_G_6 | 962_H_+2 | 962_M_+2 | 962_P_−2 | 962_Q_−1 | 962_S_−1 | 962_U_1 | |
|---|---|---|---|---|---|---|---|---|---|---|---|---|---|
| 962_E_3 | 0.2478 | 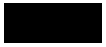 | 0.5963 | 0.2248 | 0.1369 | 0.5509 | 0.1992 | 0.65 | 0.3174 | 0.5385 | 0.6246 | 0.0854 | 962_E_3 |
| 962_E_+2 | | 0.3791 | 0.3768 | 0.2434 | 0.0858 | 0.229 | 0.3403 | 0.4079 | 0.1189 | 0.1552 | 0.3781 | 0.1263 | 962_E_+2 |
| 962_G_4 | | | 1 | 0.7616 | 0.4552 | 0.6851 | 0.6567 | 0.8356 | 0.529 | 0.5598 | 0.8179 | 0.4133 | 962_G_4 |
| 962_G_1 | | | | 0.7656 | 0.3307 | 0.6798 | 0.8991 | 0.9987 | 0.6566 | 0.5303 | 0.9899 | 0.3312 | 962_G_1 |
| 962_G_2 | | | | | 0.6172 | 0.3257 | 0.4824 | 0.557 | 0.1541 | 0.1814 | 0.6406 | 0.0754 | 962_G_2 |
| 962_G_6 | | | — | | — | 0.4658 | 0.7235 | 0.6973 | 0.3602 | 0.5274 | 0.6737 | 0.3596 | 962_G_6 |
| 962_H_+2 | | | | | — | — | 1 | 0.7252 | 0.287 | 0.8698 | 0.7496 | 0.4577 | 962_H_+2 |
| 962_M_+2 | — | | — | | — | — | — | 1 | 0.502 | 0.677 | 0.7965 | 0.755 | 962_M_+2 |
| 962_P_−2 | | | — | — | — | — | | | 0.2469 | 0.1296 | 0.4992 | 0.292 | 962_P_−2 |
| 962_Q_−1 | — | | — | — | — | — | | | — | 0.4707 | 0.6638 | 0.2631 | 962_Q_−1 |
| 962_S_−1 | | | — | | — | | | — | — | — | 0.6849 | 0.7862 | 962_S_−1 |
| 962_U_1 | | | | — | — | | | | | — | | 0.2932 | 962_U_1 |
| 962_U_2 | | | — | — | — | | | | | — | | — | 962_U_2 |
| 962_V_−1 | | | — | — | — | — | — | | | — | — | — | 962_V_−1 |
| 962_V_+2 | | | — | | — | — | | | | — | — | — | 962_V_+2 |
| 962_Z_1 | | | | — | — | | | | — | — | — | | 962_Z_1 |
| CNTL | 33.6% | 13.0% | 12.9% | 30.8% | 9.7% | 177% | 37.7% | 13.2% | 22.4% | 21.7% | 12.2% | 1.9% | CNTL |
| CASE | 50.0% | 0.0% | 7.1% | 35.7% | 0.0% | 7.1% | 35.7% | 14.3% | 40.0% | 33.3% | 14.3% | 7.1% | CASE |

| | 962_U_2 | 962_V_−1 | 962_V_+2 | 962_Z_1 | |
|---|---|---|---|---|---|
| 962_E_3 | 0.4772 | 0.6165 | 0.2995 | 0.1623 | 962_E_3 |
| 962_E_+2 | 0.1087 | 0.3523 | 0.0993 | 0.1578 | 962_E_+2 |
| 962_G_4 | 0.5146 | 0.874 | 0.4457 | 0.2758 | 962_G_4 |
| 962_G_1 | 0.4157 | 0.5854 | 0.6819 | 0.4627 | 962_G_1 |
| 962_G_2 | 0.1463 | 0.517 | 0.2801 | 0.2 | 962_G_2 |
| 962_G_6 | 0.4287 | 0.6038 | 0.4528 | 0.4868 | 962_G_6 |
| 962_H_+2 | 0.666 | 0.9967 | 0.7213 | 0.5899 | 962_H_+2 |
| 962_M_+2 | 0.588 | 0.9449 | 0.7439 | 0.5583 | 962_M_+2 |
| 962_P_−2 | 0.2324 | 0.3661 | 0.3176 | 0.2698 | 962_P_−2 |
| 962_Q_−1 | 0.3068 | 0.4758 | 0.4862 | 0.2157 | 962_Q_−1 |
| 962_S_−1 | 0.5596 | 0.946 | 0.6896 | 0.569 | 962_S_−1 |
| 962_U_1 | 0.2042 | 0.6576 | 0.2103 | 0.1691 | 962_U_1 |
| 962_U_2 | 0.315 | 0.4167 | 0.379 | 0.1139 | 962_U_2 |
| 962_V_−1 | | 1 | 0.6162 | 0.5854 | 962_V_−1 |
| 962_V_+2 | | | 1 | 0.2332 | 962_V_+2 |
| 962_Z_1 | | | — | 0.3845 | 962_Z_1 |
| CNTL | 21.7% | 35.4% | 4.7% | 35.5% | CNTL |
| CASE | 35.7% | 35.7% | 0.0% | 21.4% | CASE |

All SNP combinations in Tables 25, 26, and 27 that demonstrated a significant difference (p≦0.05) in the distribution of frequencies of the four haplotypes between the cases and the control populations were further analyzed to identify individual haplotypes that were also significant. Table 28 presents the haplotypes that were significantly associated, at the 0.05 level of significance, with the BHR phenotype. Haplotypes with higher allele frequency in the case population than in the control population acted as risk factors that increased the susceptibility to asthma. Haplotypes with lower allele frequencies in the case population than in the control population acted as protective factors that decreased the susceptibility to asthma. For Gene 845, three haplotypes were susceptibility haplotypes, associated with an increased risk of asthma at the 0.05 level of significance in the combined population. They were haplotypes G/G (SNPs R1/K−2, p=0.0144), C/T (SNPs K1/D−1, p=0.0035) and G/C (SNPs K−2/H+1, p=0.0153). One haplotype, C/C (SNPs K1/D−1, p=0.004), was protective in the combined population. In the UK population, seven haplotypes were susceptibility haplotypes. They were haplotypes T/G (SNPs R−1/J1, p=0.0209), G/T (SNPs K−2/D1 p=0.0378), G/C (SNPs J1/D1, p=0.0234), A/T (SNPs J1/D1, p=0.0003), C/T (SNPs H−1/D1, p=0.0389), C/T (SNPs D1/D-1, p=0.007) and T/T (SNPs D1/D−1, p=0.0326). There were two haplotypes that were protective in the UK population, A/C (SNPs J1/D1, p=0.0133) and C/C (SNPs D1/D−1, p=0.003). In the US population, haplotype C/G (SNPs K1/H+1, p=0.0494) was a protective haplotype. Two haplotypes were susceptibility haplotypes in the US population, T/C (SNPs P+1/H+1, p=0.0482) and C/C (SNPs K1/H+1, p=0.0329). For Gene 847, four haplotypes were protective in the US population. They were haplotypes G/C (SNPs K1/D−1, p=0.0393), G/G (SNPs K1/C+1, p=0.036), C/C (SNPs J+1/D−1, p=0.0386), C/G (SNPs J+1/C+1, p=0.0373). Seven haplotypes were susceptibility haplotypes in the US population. They were haplotypes G/C (SNPs K1/D−1, p=0.0164), G/A (SNPs K1/C+1, p=0.0217), C/T (SNPs J+1/E+1, p=0.0259), C/A (SNPs J+1/D−1, p=0.0165), C/A (SNPs J+1/C+1 0.0219), A/C (SNPs D−1/A2, p=0.0423) and A/C (SNPs C+1/A2, p=0.0495). For Gene 803, two haplotypes were protective in the US population. They were haplotypes G/C (SNPs K2/I1, p=0.047) and G/C (SNPs K2/E+2, p=0.047). For Gene 962, haplotype A/C (SNPs G2/S−1, p=0.0396) was a susceptibility haplotype in the UK population.

TABLE 28

| | | | | | |
|---|---|---|---|---|---|
| | SNP | | | | |
| | COMBI- | HAPLO- | FREQUENCIES | | P- |
| GENE | NATION | TYPE | CNTL | CASE | VALUE |

BHR Combined
US and UK

| | | | | | |
|---|---|---|---|---|---|
| 845 | R1/K−2 | GG | 0.56247 | 0.707124 | 0.0144 |
| 845 | K1/D−1 | CC | 0.898545 | 0.797619 | 0.004 |
| 845 | K1/D−1 | CT | 0.099078 | 0.202381 | 0.0035 |
| 845 | K−2/H+1 | GC | 0.514469 | 0.665 | 0.0153 |

BHR
UK Population

| | | | | | |
|---|---|---|---|---|---|
| 845 | R−1/J1 | TG | 0 | 0.039657 | 0.0209 |
| 845 | K−2/D1 | GT | 0 | 0.013974 | 0.0378 |
| 845 | J1/D1 | GC | 0.365672 | 0.514095 | 0.0234 |
| 845 | J1/D1 | AC | 0.634328 | 0.471596 | 0.0133 |
| 845 | J1/D1 | AT | 0 | 0.014308 | 0.0003 |
| 845 | H−1/D1 | CT | 0 | 0.013795 | 0.0389 |
| 845 | D1/D−1 | CC | 0.907143 | 0.791667 | 0.003 |
| 845 | D1/D−1 | CT | 0.092857 | 0.194444 | 0.007 |
| 845 | D1/D−1 | TT | 0 | 0.013889 | 0.0326 |
| 962 | G2/S−1 | AC | 0.010124 | 0.042804 | 0.0396 |

BHR
US Population

| | | | | | |
|---|---|---|---|---|---|
| 845 | P+1/H+1 | TC | 0.052119 | 0.214286 | 0.0482 |
| 845 | K1/H+1 | CC | 0.798701 | 1 | 0.0329 |
| 845 | K1/H+1 | CG | 0.201299 | 0 | 0.0494 |
| 847 | K1/D−1 | GA | 0.750602 | 1 | 0.0164 |
| 847 | K1/D−1 | GC | 0.176239 | 0 | 0.0393 |
| 847 | K1/C+1 | GA | 0.745012 | 1 | 0.0217 |
| 847 | K1/C+1 | GG | 0.181817 | 0 | 0.036 |
| 847 | J+1/E+1 | CT | 0.791905 | 1 | 0.0259 |
| 847 | J+1/D−1 | CA | 0.738791 | 1 | 0.0165 |
| 847 | J+1/D−1 | CC | 0.176558 | 0 | 0.0386 |
| 847 | J+1/C+1 | CA | 0.733303 | 1 | 0.0219 |
| 847 | J+1/C+1 | CG | 0.182036 | 0 | 0.0373 |
| 847 | D−1/A2 | AC | 0.772011 | 1 | 0.0423 |
| 847 | C+1/A2 | AC | 0.761836 | 1 | 0.0495 |
| 803 | K2/I1 | GC | 0.724359 | 0.5 | 0.047 |
| 803 | K2/E+2 | GC | 0.724359 | 0.5 | 0.047 |

c. Total IgE

In Tables 29, 30 and 31, the haplotype analysis (2-at-a-time) is presented for the combined, the UK and the US populations, respectively. A single SNP combination in Gene 845 is significant in the US population: SNPs R 1 & R−1 (p=0.0355). Fourteen SNP combinations in Gene 962 are significant in the combined and in the UK and US population alone: SNPs E 3 & G 1 (US p=0.0398), SNPs E+2 & G 1 (combined p=0.0249), SNPs E+2 & V−1 (combined p=0.0305), SNPs G 4 & G 1 (combined p=0.0089, UK p=0.0282), SNPs G 4 & G 6 (UK p=0.0376), SNPs G 4 & Q−1 (US p=0.0263), SNPs G 4 & U 2 (US p=0.0168), SNPs G 4 & V+2 (US p=0.0052), SNPs G 1 & P−2 (combined p=0.0268), SNPs G 1 & Q−1 (combined p=0.0069, UK p=0.0375, US p=0.025), SNPs G 1 & U 2 (US p=0.0194), SNPs G 6 & S−1 (UK p=0.0112), SNPs H+2 & S−1 (combined p=0.0426) and SNPs M+2 & P−2 (US p=0.0096).

TABLE 29
HAPLOTYPE ANALYSIS OF TOTAL IgE PHENOTYPE COMBINED US/UK POPULATION

| | 845_R_1 | 845_R_−1 | 845_P_+1 | 845_K_1 | 845_K_−2 | 845_J_1 | 845_J_−1 | 845_I_−1 | 845_H_+1 | 845_H_−1 | 845_G_+1 | 845_F_+1 | 845_D_1 | 845_D_−1 | |
|---|---|---|---|---|---|---|---|---|---|---|---|---|---|---|---|
| 845_{—} | 0.8877 | 0.1664 | 0.3567 | 0.9097 | 0.2478 | 0.5528 | 0.6568 | 0.9102 | 0.5597 | 0.7751 | 0.6319 | 0.699 | 0.921 | 0.7729 | 845_R_1 |
| R_1 | | | | | | | | | | | | | | | |
| 845_{—} | — | 0.0901 | 0.1208 | 0.1986 | 0.0797 | 0.288 | 0.2058 | 0.2832 | 0.0682 | 0.2866 | 0.3337 | 0.322 | 0.1405 | 0.2124 | 845_R_−1 |
| R_−1 | | | | | | | | | | | | | | | |
| 845_{—} | — | — | 0.1257 | 0.3338 | 0.1405 | 0.4922 | 0.321 | 0.3264 | 0.2647 | 0.4947 | 0.2845 | 0.318 | 0.275 | 0.3822 | 845_P_+1 |
| P_+1 | | | | | | | | | | | | | | | |
| 845_{—} | — | — | — | 1 | 0.3383 | 0.424 | 0.6671 | 0.8301 | 0.5338 | 0.6229 | 0.7208 | 0.938 | 0.7972 | 0.6038 | 845_K_1 |
| K_1 | | | | | | | | | | | | | | | |
| 845_{—} | — | — | — | — | 0.1677 | 0.2421 | 0.2833 | 0.2178 | 0.0993 | 0.3013 | 0.3282 | 0.2824 | 0.1951 | 0.2697 | 845_K_−2 |
| K_−2 | | | | | | | | | | | | | | | |
| 845_{—} | — | — | — | — | — | 0.2952 | 0.0942 | 0.3088 | 0.4593 | 0.681 | 0.4093 | 0.6186 | 0.3818 | 0.4658 | 845_J_1 |
| J_1 | | | | | | | | | | | | | | | |
| 845_{—} | — | — | — | — | — | — | 0.6062 | 0.815 | 0.2347 | 0.4244 | 0.6932 | 0.9265 | 0.7733 | 0.571 | 845_J_−1 |
| J_−1 | | | | | | | | | | | | | | | |
| 845_{—} | — | — | — | — | — | — | — | 0.7609 | 0.5615 | 0.6564 | 0.3315 | 0.7179 | 0.8779 | 0.4486 | 845_I_−1 |
| I_−1 | | | | | | | | | | | | | | | |
| 845_{—} | — | — | — | — | — | — | — | — | 0.4431 | 0.4406 | 0.7 | 0.8354 | 0.3571 | 0.5568 | 845_H_+1 |
| H_+1 | | | | | | | | | | | | | | | |
| 845_{—} | — | — | — | — | — | — | — | — | — | 0.4795 | 0.761 | 0.7321 | 0.6216 | 0.7728 | 845_H_−1 |
| H_−1 | | | | | | | | | | | | | | | |
| 845_{—} | — | — | — | — | — | — | — | — | — | — | 0.6628 | 0.7036 | 0.8653 | 0.6024 | 845_G_+1 |
| G_+1 | | | | | | | | | | | | | | | |
| 845_{—} | — | — | — | — | — | — | — | — | — | — | | 1 | 0.9464 | 0.7782 | 845_F_+1 |
| F_+1 | | | | | | | | | | | | | | | |
| 845_{—} | — | — | — | — | — | — | — | — | — | — | — | — | 0.3966 | 0.8431 | 845_D_1 |
| D_1 | | | | | | | | | | | | | | | |
| 845_{—} | — | — | — | | — | — | — | — | — | — | — | — | — | 0.505 | 845_D_−1 |
| D_−1 | | | | | | | | | | | | | | | |
| CNTL | 14.8% | 28.7% | 6.5% | 0.2% | 28.9% | 34.5% | 37.1% | 12.8% | 19.6% | 45.2% | 13.2% | 18.4% | 0.2% | 9.9% | CNTL |
| CASE | 14.1% | 21.1% | 10.8% | 0.0% | 22.1% | 40.0% | 40.0% | 11.5% | 16.2% | 41.4% | 14.6% | 18.5% | 0.8% | 12.1% | CASE |

| | 847_K_1 | 847_J_+1 | 847_E_+1 | 847_D_−1 | 847_C_+1 | 847_A_2 | 847_A_1 | |
|---|---|---|---|---|---|---|---|---|
| 847_K_1 | 1 | 0.7224 | 0.5704 | 0.9785 | 0.8175 | 0.4686 | 0.7671 | 847_K_1 |
| 847_J_+1 | — | 0.4732 | 0.4748 | 0.6465 | 0.4706 | 0.8183 | 0.5835 | 847_J_+1 |
| 847_E_+1 | — | — | 0.4629 | 0.5443 | 0.6655 | 0.8942 | 0.7853 | 847_E_+1 |
| 847_D_+1 | — | — | — | 0.7795 | 0.8749 | 0.9481 | 0.9318 | 847_D_−1 |
| 847_C_+1 | — | — | — | — | 0.3696 | 0.8263 | 0.7704 | 847_C_+1 |
| 847_A_2 | — | — | — | — | — | 0.7007 | 0.8952 | 847_A_2 |
| 847_A_1 | — | — | — | — | — | — | 1 | 847_A_1 |
| CNTL | 4.9% | 4.9% | 12.6% | 16.9% | 17.9% | 6.7% | 1.0% | CNTL |
| CASE | 5.0% | 3.0% | 15.2% | 17.9% | 21.5% | 7.5% | 0.8% | CASE |

| | 874_R_+1 | 874_S_+1 | 874_T_−1 | 874_V_−1 | |
|---|---|---|---|---|---|
| 874_R_+1 | 0.1181 | 0.476 | 0.4027 | 0.361 | 874_R_+1 |
| 874_S_+1 | — | 0.8413 | 0.4528 | 0.4627 | 874_S_+1 |
| 874_T_−1 | — | — | 0.8447 | 0.4184 | 874_T_−1 |
| 874_V_−1 | — | — | — | 0.3785 | 874_V_−1 |
| CNTL | 39.9% | 39.5% | 48.6% | 17.6% | CNTL |
| CASE | 32.0% | 40.4% | 50.0% | 21.0% | CASE |

| | 803_K_3 | 803_K_2 | 803_I_1 | 803_I_−1 | 803_H_+1 | 803_E_+2 | |
|---|---|---|---|---|---|---|---|
| 803_K_3 | 0.3637 | 0.234 | 0.5258 | 0.517 | 0.5151 | 0.5526 | 803_K_3 |
| 803_K_2 | — | 0.1402 | 0.3257 | 0.3147 | 0.3245 | 0.3302 | 803_K_2 |
| 803_I_1 | — | — | 0.9113 | 0.9885 | 0.9273 | 0.6626 | 803_I_1 |
| 803_I_−1 | — | — | — | 1 | 0.7904 | 0.8594 | 803_I_−1 |
| 803_H_+1 | — | — | — | — | 0.7311 | 0.9108 | 803_H_+1 |
| 803_E_+2 | — | — | — | — | — | 0.8402 | 803_E_+2 |
| CNTL | 0.9% | 0.2% | 28.2% | 0.2% | 25.2% | 44.2% | CNTL |
| CASE | 2.2% | 1.5% | 28.8% | 0.0% | 23.5% | 45.4% | CASE |

| | 962_E_3 | 962_E_+2 | 962_G_4 | 962_G_1 | 962_G_2 | 962_G_6 | 962_H_+2 | 962_M_+2 | 962_P_−2 | 962_Q_−1 | 962_S_−1 | 962_U_1 | |
|---|---|---|---|---|---|---|---|---|---|---|---|---|---|
| 962_E_3 | 0.4753 | 0.1281 | 0.1867 | 0.1135 | 0.6053 | 0.556 | 0.3468 | 0.6678 | 0.8706 | 0.8684 | 0.5581 | 0.8916 | 962_E_3 |
| 962_E_+2 | — | 0.0677 | 0.0562 | 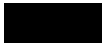 | 0.1588 | 0.1748 | 0.074 | 0.1769 | 0.1882 | 0.2181 | 0.1578 | 0.1515 | 962_E_+2 |
| 962_G_4 | — | — | 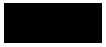 | 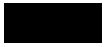 | 0.1223 | 0.081 | 0.0657 | 0.1223 | 0.2134 | 0.232 | 0.0512 | 0.1698 | 962_G_4 |
| 962_G_1 | — | — | — | 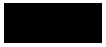 | 0.1715 | 0.0856 | 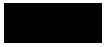 | 0.1718 | 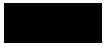 | 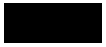 | 0.1315 | 0.1675 | 962_G_1 |
| 962_G_2 | — | — | — | — | 1 | 0.4859 | 0.5186 | 0.8108 | 0.9667 | 0.9888 | 0.4504 | 0.6718 | 962_G_2 |
| 962_G_6 | — | — | — | — | — | 0.3205 | 0.3003 | 0.2838 | 0.6461 | 0.6884 | 0.1011 | 0.168 | 962_G_6 |
| 962_H_+2 | — | — | — | — | — | — | 0.117 | 0.1524 | 0.4959 | 0.431 | 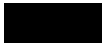 | 0.5122 | 962_H_+2 |
| 962_M_+2 | — | — | — | — | — | — | — | 0.6553 | 0.7965 | 0.8171 | 0.7357 | 0.8275 | 962_M_+2 |
| 962_P_−2 | — | — | — | — | — | — | — | — | 0.7323 | 0.6335 | 0.6199 | 0.9552 | 962_P_−2 |
| 962_Q_−1 | — | — | — | — | — | — | — | — | — | 0.8195 | 0.6502 | 0.9595 | 962_Q_−1 |
| 962_S_−1 | — | — | — | — | — | — | — | — | — | — | 0.3507 | 0.5865 | 962_S_−1 |
| 962_U_1 | — | — | — | — | — | — | — | — | — | — | — | 0.7821 | 962_U_1 |
| 962_U_2 | — | — | — | — | — | — | — | — | — | — | — | — | 962_U_2 |
| 962_V_−1 | — | — | — | — | — | — | — | — | — | — | — | — | 962_V_−1 |
| 962_V_+2 | — | — | — | — | — | — | — | — | — | — | — | — | 962_V_+2 |
| 962_Z_1 | — | — | — | — | — | — | — | — | — | — | — | — | 962_Z_1 |
| CNTL | 35.6% | 12.7% | 13.6% | 27.2% | 7.6% | 20.4% | 41.5% | 12.7% | 23.8% | 23.7% | 11.4% | 3.0% | CNTL |
| CASE | 32.1% | 6.8% | 7.1% | 37.0% | 7.7% | 16.2% | 34.0% | 10.7% | 25.4% | 24.6% | 8.3% | 3.6% | CASE |

| | 962_U_2 | 962_V_−1 | 962_V_+2 | 962_Z_1 | |
|---|---|---|---|---|---|
| 962_E_3 | 0.8737 | 0.2619 | 0.4308 | 0.45 | 962_E_3 |
| 962_E_+2 | 0.2713 | 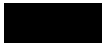 | 0.1741 | 0.184 | 962_E_+2 |
| 962_G_4 | 0.2206 | 0.1478 | 0.2075 | 0.2068 | 962_G_4 |
| 962_G_1 | 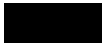 | 0.1068 | 0.1444 | 0.1705 | 962_G_1 |
| 962_G_2 | 0.9814 | 0.805 | 0.9191 | 0.9782 | 962_G_2 |
| 962_G_6 | 0.7122 | 0.5476 | 0.5849 | 0.6686 | 962_G_6 |
| 962_H_+2 | 0.2878 | 0.1857 | 0.2384 | 0.2674 | 962_H_+2 |
| 962_M_+2 | 0.4814 | 0.6648 | 0.6777 | 0.9384 | 962_M_+2 |
| 962_P_−2 | 0.9691 | 0.8527 | 0.7035 | 0.6705 | 962_P_−2 |
| 962_Q_−1 | 0.7251 | 0.7745 | 0.8439 | 0.832 | 962_Q_−1 |
| 962_S_−1 | 0.7682 | 0.473 | 0.4304 | 0.784 | 962_S_−1 |
| 962_U_1 | 0.9682 | 0.4878 | 0.8 | 0.8671 | 962_U_1 |
| 962_U_2 | 1 | 0.6732 | 0.8443 | 0.9975 | 962_U_2 |
| 962_V_−1 | — | 0.3994 | 0.8052 | 0.6563 | 962_V_−1 |
| 962_V_+2 | — | — | 0.8112 | 0.848 | 962_V_+2 |
| 962_Z_1 | — | — | — | 1 | 962_Z_1 |
| CNTL | 23.8% | 34.8% | 4.5% | 31.9% | CNTL |
| CASE | 23.9% | 30.4% | 3.5% | 31.5% | CASE |

TABLE 30
HAPLOTYPE ANALYSIS OF TOTAL IgE PHENOTYPE UK POPULATION

| | 845_R_1 | 845_R_−1 | 845_P_+1 | 845_K_1 | 845_K_−2 | 845_J_1 | 845_J_−1 | 845_I_−1 | 845_H_+1 | 845_H_−1 | 845_G_+1 | 845_F_+1 | 845_D_1 | 845_D_−1 | |
|---|---|---|---|---|---|---|---|---|---|---|---|---|---|---|---|
| 845_{—} | 0.6245 | 0.4023 | 0.5577 | 0.7546 | 0.4747 | 0.6289 | 0.864 | 0.8681 | 0.4819 | 0.6939 | 0.5258 | 0.5721 | 0.3188 | 0.6705 | 845_R_1 |
| R_1 | | | | | | | | | | | | | | | |
| 845_{—} | — | 0.2308 | 0.3701 | 0.4518 | 0.5377 | 0.3954 | 0.4301 | 0.3487 | 0.2214 | 0.4435 | 0.3928 | 0.3966 | 0.0989 | 0.4124 | 845_R_−1 |
| R_−1 | | | | | | | | | | | | | | | |
| 845_{—} | — | — | 0.3784 | 0.5805 | 0.382 | 0.8256 | 0.4869 | 0.6381 | 0.5489 | 0.7792 | 0.5012 | 0.6447 | 0.2661 | 0.4538 | 845_P_+1 |
| P_+1 | | | | | | | | | | | | | | | |
| 845_{—} | — | — | — | 1 | 0.4822 | 0.6095 | 0.726 | 0.9105 | 0.6231 | 0.6843 | 0.5994 | 0.9638 | 0.2114 | 0.5816 | 845_K_1 |
| K_1 | | | | | | | | | | | | | | | |
| 845_{—} | — | — | — | — | 0.2842 | 0.4374 | 0.4736 | 0.3916 | 0.262 | 0.4658 | 0.4576 | 0.4679 | 0.1238 | 0.4401 | 845_K_−2 |
| K_−2 | | | | | | | | | | | | | | | |
| 845_{—} | — | — | — | — | — | 0.4798 | 0.264 | 0.559 | 0.6743 | 0.8718 | 0.561 | 0.8951 | 0.2368 | 0.7503 | 845_J_1 |
| J_1 | | | | | | | | | | | | | | | |
| 845_{—} | — | — | — | — | — | — | 0.6392 | 0.8437 | 0.3453 | 0.5816 | 0.6365 | 0.9516 | 0.2641 | 0.5881 | 845_J_−1 |
| J_−1 | | | | | | | | | | | | | | | |
| 845_{—} | — | — | — | — | — | — | — | 0.8603 | 0.6469 | 0.6953 | 0.2242 | 0.7965 | 0.2795 | 0.7392 | 845_I_−1 |
| I_−1 | | | | | | | | | | | | | | | |
| 845_{—} | — | — | — | — | — | — | — | — | 0.5564 | 0.5493 | 0.68 | 0.9185 | 0.1306 | 0.6331 | 845_H_+1 |
| H_+1 | | | | | | | | | | | | | | | |
| 845_{—} | — | — | — | — | — | — | — | — | — | 0.4918 | 0.7011 | 0.7365 | 0.2415 | 0.7078 | 845_H_−1 |
| H_−1 | | | | | | | | | | | | | | | |
| 845_{—} | — | — | — | — | — | — | — | — | — | — | 0.5116 | 0.3714 | 0.218 | 0.5832 | 845_G_+1 |
| G_+1 | | | | | | | | | | | | | | | |
| 845_{—} | — | — | — | — | — | — | — | — | — | — | — | 1 | 0.3304 | 0.7673 | 845_F_+1 |
| F_+1 | | | | | | | | | | | | | | | |
| 845_{—} | — | — | — | — | — | — | — | — | — | — | — | — | 0.267 | 0.2239 | 845_D_1 |
| D_1 | | | | | | | | | | | | | | | |
| 845_{—} | — | — | — | — | — | — | — | — | — | — | — | — | — | 0.4474 | 845_D_−1 |
| D_−1 | | | | | | | | | | | | | | | |
| CNTL | 13.6% | 26.6% | 6.4% | 0.4% | 27.0% | 36.6% | 37.5% | 12.6% | 19.3% | 43.6% | 13.2% | 18.8% | 0.0% | 9.3% | CNTL |
| CASE | 15.4% | 20.2% | 9.4% | 0.0% | 21.0% | 40.6% | 40.6% | 11.3% | 16.0% | 39.6% | 16.0% | 18.9% | 1.0% | 11.8% | CASE |

| | 847_K_1 | 847_J_+1 | 847_E_+1 | 847_D_−1 | 847_C_+1 | 847_A_2 | 847_A_1 | |
|---|---|---|---|---|---|---|---|---|
| 847_K_1 | 1 | 0.9807 | 0.3563 | 0.7223 | 0.4804 | 0.2596 | 0.9246 | 847_K_1 |
| 847_J_+1 | — | 1 | 0.4687 | 0.5717 | 0.3727 | 0.7772 | 0.8857 | 847_J_+1 |
| 847_E_+1 | — | — | 0.2494 | 0.2263 | 0.3632 | 0.5525 | 0.6005 | 847_E_+1 |
| 847_D_−1 | — | — | — | 0.3355 | 0.2587 | 0.708 | 0.7655 | 847_D_−1 |
| 847_C_+1 | — | — | — | — | 0.1502 | 0.5014 | 0.5763 | 847_C_+1 |
| 847_A_2 | — | — | — | — | — | 0.474 | 0.7139 | 847_A_2 |
| 847_A_1 | — | — | — | — | — | — | 1 | 847_A_1 |
| CNTL | 3.6% | 2.9% | 12.8% | 16.5% | 17.8% | 5.4% | 1.3% | CNTL |
| CASE | 3.2% | 2.8% | 17.9% | 21.6% | 24.5% | 7.4% | 0.9% | CASE |

| | 874_R_+1 | 874_S_+1 | 874_T_−1 | 874_V_−1 | |
|---|---|---|---|---|---|
| 874_R_+1 | 0.1597 | 0.4766 | 0.3645 | 0.4563 | 874_R_+1 |
| 874_S_+1 | — | 0.8191 | 0.3188 | 0.6485 | 874_S_+1 |
| 874_T_−1 | — | — | 0.5794 | 0.5573 | 874_T_−1 |
| 874_V_−1 | — | — | — | 0.4743 | 874_V_−1 |
| CNTL | 41.5% | 38.0% | 48.9% | 16.9% | CNTL |
| CASE | 33.3% | 39.5% | 52.6% | 19.8% | CASE |

| | 803_K_3 | 803_K_2 | 803_I_1 | 803_I_−1 | 803_H_+1 | 803_E_+2 | |
|---|---|---|---|---|---|---|---|
| 803_K_3 | 0.3645 | 0.2827 | 0.3971 | 0.5477 | 0.5264 | 0.4218 | 803_K_3 |
| 803_K_2 | — | 0.2083 | 0.5861 | 0.6613 | 0.6176 | 0.2997 | 803_K_2 |
| 803_I_1 | — | — | 0.7033 | 0.753 | 0.6955 | 0.5913 | 803_I_1 |
| 803_I_−1 | — | — | — | 1 | 0.7974 | 0.4821 | 803_I_−1 |
| 803_H_+1 | — | — | — | — | 0.7028 | 0.5103 | 803_H_+1 |
| 803_E_+2 | — | — | — | — | — | 0.3091 | 803_E_+2 |
| CNTL | 1.1% | 0.4% | 28.6% | 0.4% | 25.7% | 42.4% | CNTL |
| CASE | 2.6% | 1.7% | 26.3% | 0.0% | 23.7% | 48.2% | CASE |

| | 962_E_3 | 962_E_+2 | 962_G_4 | 962_G_1 | 962_G_2 | 962_G_6 | 962_H_+2 | 962_M_+2 | 962_P_−2 | 962_Q_−1 | 962_S_−1 | 962_U_1 | |
|---|---|---|---|---|---|---|---|---|---|---|---|---|---|
| 962_E_3 | 0.2974 | 02915 | 0.204 | 0.087 | 0.6881 | 0.2484 | 0.311 | 0.5433 | 0.6727 | 0.6634 | 0.4786 | 0.6045 | 962_E_3 |
| 962_E_+2 | — | 0.1645 | 0.1231 | 0.1181 | 0.158 | 0.082 | 0.2014 | 0.2432 | 0.2642 | 0.2958 | 0.2729 | 0.2645 | 692_E_+2 |
| 962_G_4 | — | — | 0.0904 | 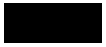 | 0.1929 | 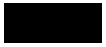 | 0.1096 | 0.2014 | 0.2795 | 0.2753 | 0.1095 | 0.2195 | 962_G_4 |
| 962_G_1 | — | — | — | 0.0695 | 0.3054 | 0.1099 | 0.1424 | 0.1657 | 0.0649 | 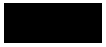 | 0.1516 | 0.1315 | 962_G_1 |
| 962_G_2 | — | — | | — | 0.666 | 0.2817 | 0.5762 | 0.5679 | 0.8178 | 0.774 | 0.3131 | 0.6239 | 962_G_2 |
| 962_G_6 | — | | | — | — | 0.1218 | 0.172 | 0.0727 | 0.0853 | 0.0695 | 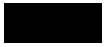 | 0.1155 | 962_G_6 |
| 962_H_+2 | — | — | | — | — | — | 0.1834 | 0.3028 | 0.4322 | 0.3945 | 0.1222 | 0.4941 | 962_H_+2 |
| 962_M_+2 | — | — | | — | — | — | — | 0.3811 | 0.3722 | 0.3156 | 0.6192 | 0.5613 | 962_M_+2 |
| 962_P_−2 | — | — | | — | — | — | — | — | 0.5173 | 0.33 | 0.3482 | 0.922 | 962_P_−2 |
| 962_Q_−1 | — | — | | — | — | — | — | — | — | 0.4355 | 0.3319 | 0.8763 | 962_Q_−1 |
| 962_S_−1 | — | — | | | — | | — | — | | | 0.2008 | 0.4341 | 962_S_−1 |
| 962_U_1 | — | | | — | — | — | — | — | | — | — | 1 | 962_U_1 |
| 962_U_2 | — | | | | — | — | — | | — | — | — | — | 962_U_2 |
| 962_V_−1 | — | | | — | — | — | — | | — | — | — | — | 962_V_−1 |
| 962_V_+2 | — | | | — | — | — | — | | — | — | | — | 962_V_+2 |
| 962_Z_1 | — | | | — | — | — | — | | — | — | | — | 962_Z_1 |
| CNTL | 36.8% | 12.5%140% | 14.0% | 25.2% | 6.4% | 21.7% | 43.6% | 12.4% | 24.6% | 24.8% | 10.9% | 3.7% | CNTL |
| CASE | 31.0% | 7.4% | 7.8% | 34.4% | 7.6% | 14.4% | 35.8% | 8.6% | 21.2% | 20.7% | 6.7% | 3.4% | CASE |

| | 962_U_2 | 962_V_−1 | 962_V_+2 | 962_Z_1 | |
|---|---|---|---|---|---|
| 962_E_3 | 0.5419 | 0.3712 | 0.5548 | 0.3789 | 962_E_3 |
| 962_E_+2 | 0.2659 | 0.0886 | 0.3613 | 0.3826 | 692_E_+2 |
| 962_G_4 | 0.2152 | 0.3258 | 0.2153 | 0.3197 | 962_G_4 |
| 962_G_1 | 0.1043 | 0.1972 | 0.3573 | 0.2492 | 962_G_1 |
| 962_G_2 | 0.6705 | 0.6619 | 0.5615 | 0.7564 | 962_G_2 |
| 962_G_6 | 0.3091 | 0.1323 | 0.2743 | 0.3044 | 962_G_6 |
| 962_H_+2 | 0.2633 | 0.4808 | 0.2801 | 0.4622 | 962_H_+2 |
| 962_M_+2 | 0.1131 | 0.6542 | 0.3507 | 0.5703 | 962_M_+2 |
| 962_P_−2 | 0.948 | 0.6796 | 0.7363 | 0.7975 | 962_P_−2 |
| 962_Q_−1 | 0.7428 | 0.6208 | 0.8437 | 0.7718 | 962_Q_−1 |
| 962_S_−1 | 0.3126 | 0.5109 | 0.2025 | 0.3561 | 962_S_−1 |
| 962_U_1 | 0.6355 | 0.3439 | 0.8724 | 0.7934 | 962_U_1 |
| 962_U_2 | 0.297 | 0.3151 | 0.7156 | 0.6601 | 962_U_2 |
| 962_V_−1 | — | 0.5516 | 0.7585 | 0.8999 | 962_V_−1 |
| 962_V_+2 | | — | 0.7845 | 0.7238 | 962_V_+2 |
| 962_Z_1 | | — | | 0.4837 | 962_Z_1 |
| CNTL | 25.0% | 34.4% | 4.5% | 30.0% | CNTL |
| CASE | 19.5% | 31.0% | 3.3% | 33.6% | CASE |

TABLE 31
HAPLOTYPE ANALYSIS OF TOTAL IgE PHENOTYPE US POPULATION

| | 845_R_1 | 845_R_−1 | 845_P_+1 | 845_K_1 | 845_K_−2 | 845_J_1 | 845_J_−1 | 845_H_+1 | 845_H_−1 | 845_G_+1 | 845_F_+1 | 845_D_1 | 845_D_{—1} | 845_D_+1 | |
|---|---|---|---|---|---|---|---|---|---|---|---|---|---|---|---|
| 845_{—} | 0.3763 | 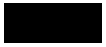 | 0.4291 | 0.2741 | 0.061 | 0.5114 | 0.3395 | 0.6041 | 0.5582 | 0.6003 | 0.5553 | 0.6409 | 0.341 | 0.5721 | 845_R_1 |
| R_1 | | | | | | | | | | | | | | | |
| 845_{—} | — | 0.6371 | 0.3579 | 0.4625 | 0.7658 | 0.7312 | 0.7173 | 0.8947 | 0.5329 | 0.7777 | 0.789 | 0.788 | 0.5082 | 0.698 | 845_R_−1 |
| R_−1 | | | | | | | | | | | | | | | |
| 845_{—} | — | — | 0.1002 | 0.2853 | 0.3643 | 0.3915 | 0.3745 | 0.3972 | 0.4378 | 0.4423 | 0.3215 | 0.4794 | 0.3904 | 0.5397 | 845_P_+1 |
| P_+1 | | | | | | | | | | | | | | | |
| 845_{—} | — | — | — | 1 | 0.6082 | 0.6006 | 0.8939 | 0.8958 | 0.6015 | 0.9926 | 0.454 | 0.8896 | 0.8236 | 0.7791 | 845_K_1 |
| K_1 | | | | | | | | | | | | | | | |
| 845_{—} | — | — | — | — | 0.8075 | 0.7627 | 0.8266 | 0.8464 | 0.3925 | 0.8264 | 0.7076 | 0.8454 | 0.6625 | 0.8512 | 845_K_−2 |
| K_−2 | | | | | | | | | | | | | | | |
| 845_{—} | — | — | — | — | — | 0.6376 | 0.689 | 0.7509 | 0.8291 | 0.7813 | 0.5999 | 0.8772 | 0.6346 | 0.7247 | 845_J_1 |
| J_1 | | | | | | | | | | | | | | | |
| 845_{—} | — | — | — | — | — | — | 1 | 0.9932 | 0.8628 | 0.8851 | 0.7794 | 0.9827 | 0.9724 | 0.9371 | 845_J_−1 |
| J_−1 | | | | | | | | | | | | | | | |
| 845_{—} | — | — | — | — | — | — | — | 1 | 0.8924 | 0.9739 | 0.533 | 0.9293 | 0.9712 | 0.7797 | 845_I_−1 |
| I_−1 | | | | | | | | | | | | | | | |
| 845_{—} | — | — | — | — | — | — | — | — | 1 | 0.8797 | 0.7679 | 0.9337 | 0.6599 | 0.908 | 845_H_+1 |
| H_+1 | | | | | | | | | | | | | | | |
| 845_{—} | — | — | — | — | — | — | — | — | — | 1 | 0.837 | 0.9597 | 0.9746 | 0.9407 | 845_H_−1 |
| H_−1 | | | | | | | | | | | | | | | |
| 845_{—} | | — | — | — | — | — | | — | — | — | 0.7426 | 0.6413 | 0.6061 | 0.4484 | 845_G_+1 |
| G_+1 | | | | | | | | | | | | | | | |
| 845_{—} | | — | — | — | — | — | | — | — | — | — | 1 | 0.9585 | 0.9481 | 845_F_+1 |
| F_+1 | | | | | | | | | | | | | | | |
| 845_{—} | | — | — | — | — | — | — | — | — | — | — | — | 1 | 0.725 | 845_D_1 |
| D_1 | | | | | | | | | | | | | | | |
| 845_{—} | — | — | — | — | — | — | — | — | — | — | — | — | — | 0.7202 | 845_D_−1 |
| D_−1 | | | | | | | | | | | | | | | |
| CNTL | 17.1% | 32.5% | 6.5% | 0.0% | 32.5% | 30.9% | 36.4% | 13.3% | 20.1% | 48.1% | 13.2% | 17.5% | 0.7% | 11.0% | CNTL |
| CASE | 8.3% | 25.0% | 16.7% | 0.0% | 27.3% | 37.5% | 37.5% | 12.5% | 16.7% | 50.0% | 8.3% | 16.7% | 0.0% | 13.6% | CASE |

| | 847_K_1 | 847_J_+1 | 847_E_+1 | 847_D_−1 | 847_C_+1 | 847_A_2 | 847_A_1 | |
|---|---|---|---|---|---|---|---|---|
| 847_K_1 | 0.4387 | 0.6112 | 0.3997 | 0.2007 | 0.3158 | 0.8474 | 0.7237 | 847_K_1 |
| 847_J_+1 | — | 0.6961 | 0.2419 | 0.1401 | 0.3176 | 0.207 | 0.5424 | 847_J_+1 |
| 847_E_+1 | — | — | 0.3158 | 0.1564 | 0.4302 | 0.5092 | 0.2996 | 847_E_+1 |
| 847_D_−1 | — | — | — | 0.1308 | 0.1493 | 0.2724 | 0.1859 | 847_D_−1 |
| 847_C_+1 | — | — | — | — | 0.378 | 0.5464 | 0.3744 | 847_C_+1 |
| 847_A_2 | — | — | — | — | — | 1 | 0.9789 | 847_A_2 |
| 847_A_1 | — | — | — | — | — | — | 1 | 847_A_1 |
| CNTL | 7.3% | 8.4% | 12.3% | 17.6% | 18.2% | 9.1% | 0.7% | CNTL |
| CASE | 11.5% | 3.8% | 3.8% | 4.2% | 8.3% | 7.7% | 0.0% | CASE |

| | 874_R_+1 | 874_S_+1 | 874_T_−1 | 874_V_−1 | |
|---|---|---|---|---|---|
| 874_R_+1 | 0.3317 | 0.4639 | 0.5461 | 0.533 | 874_R_+1 |
| 874_S_+1 | — | 0.8201 | 0.3993 | 0.6175 | 874_S_+1 |
| 874_T_−1 | — | — | 0.3647 | 0.5908 | 874_T_−1 |
| 874_V_−1 | — | — | — | 0.3924 | 874_V_−1 |
| CNTL | 37.0% | 42.2% | 48.1% | 18.8% | CNTL |
| CASE | 25.0% | 45.5% | 36.4% | 27.3% | CASE |

| | 803_K_3 | 803_K_2 | 803_I_1 | 803_I_−1 | 803_H_+1 | 803_E_+2 | |
|---|---|---|---|---|---|---|---|
| 803_K_3 | 1 | 0.7837 | 0.222 | 0.7897 | 0.8098 | 0.2268 | 803_K_3 |
| 803_K_2 | — | 1 | 0.1237 | 1 | 0.9255 | 0.117 | 803_K_2 |
| 803_I_1 | — | — | 0.1714 | 0.1213 | 0.3365 | 0.2921 | 803_I_1 |
| 803_I_−1 | — | — | — | 1 | 0.9248 | 0.11 | 803_I_−1 |
| 803_H_+1 | — | — | — | — | 1 | 0.2773 | 803_H_+1 |
| 803_E_+2 | — | — | — | — | — | 0.1375 | 803_E_+2 |
| CNTL | 0.6% | 0.0% | 27.6% | 0.0% | 24.4% | 47.4% | CNTL |
| CASE | 0.0% | 0.0% | 44.4% | 0.0% | 22.2% | 27.8% | CASE |

| | 962_E_3 | 962_E_+2 | 962_G_4 | 962_G_1 | 962_G_2 | 962_D_6 | 962_H_+2 | 962_M_+2 | 962_P_−2 | 962_Q_−1 | 962_S_−1 | 962_U_1 | |
|---|---|---|---|---|---|---|---|---|---|---|---|---|---|
| 962_E_3 | 0.8172 | 0.2115 | 0.2866 | 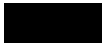 | 0.5867 | 0.8474 | 0.1143 | 0.5722 | 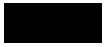 | 0.0848 | 0.6602 | 0.2048 | 692_E_3 |
| 962_E_+2 | — | 0.316 | 0.4477 | 0.1729 | 0.5175 | 0.2022 | 0.2203 | 0.3776 | 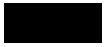 | 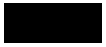 | 0.4254 | 0.3649 | 962_E_+2 |
| 962_G_4 | | — | 0.3133 | 0.0863 | 0.1481 | 0.3626 | 0.32 | 0.3242 | 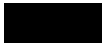 | 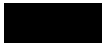 | 0.4093 | 0.2215 | 962_G_4 |
| 962_G_1 | — | — | | 0.1014 | 0.3663 | 0.2943 | 0.1246 | 0.2333 | 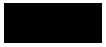 | 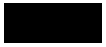 | 0.2458 | 0.0679 | 962_G_1 |
| 962_G_2 | | — | | — | 1 | 0.9185 | 0.5113 | 0.8406 | 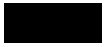 | 0.0728 | 0.9624 | 0.8903 | 962_G_2 |
| 962_G_6 | | | | — | — | 0.4013 | 0.6844 | 0.659 | 0.0884 | 0.0934 | 0.699 | 0.5045 | 962_Q_6 |
| 962_H_+2 | | — | | — | — | — | 0.2614 | 0.2184 | 0.0694 | 0.101 | 0.3392 | 0.3355 | 962_H_+2 |
| 962_M_+2 | | | — | | | — | — | 0.3451 | 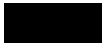 | 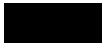 | 0.4484 | 0.5179 | 962_M_+2 |
| 962_P_−2 | | | | — | | — | — | — | 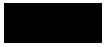 | 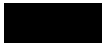 | 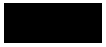 | 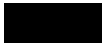 | 962_P_−2 |
| 962_Q_−1 | | | | | | | | — | | 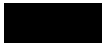 | 0.0676 | 0.0603 | 962_Q_−1 |
| 962_S_−1 | | | — | | | — | — | — | — | 0.5172 | 0.8519 | 962_S_−1 | |
| 962_U_1 | | — | — | | — | — | — | | — | — | 0.4129 | 962_U_1 | |
| 962_U_2 | | — | — | | | — | — | | — | — | — | 962_U_2 | |
| 962_V_−1 | | | | | — | — | — | | — | — | — | 962_V_−1 | |
| 962_V_+2 | | | — | | | | — | | — | — | — | 962_V_+2 | |
| 962_Z_1 | | | — | | | — | | | — | — | — | 962_Z_1 | |
| CNTL | 33.6% | 13.0% | 12.9% | 30.8% | 9.7% | 17.7% | 37.7% | 13.2% | 22.4% | 21.7% | 12.2% | 1.9% | CNTL |
| CASE | 37.5% | 4.2% | 4.2% | 50.0% | 8.3% | 25.0% | 25.0% | 20.8% | 50.0% | 45.5% | 16.7% | 4.5% | CASE |

| | 962_U_2 | 962_V_−1 | 962_V_+2 | 962_Z_1 | |
|---|---|---|---|---|---|
| 962_E_3 | 0.0641 | 0.5184 | 0.807 | 0.2894 | 692_E_3 |
| 962_E_+2 | 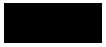 | 0.383 | 0.5794 | 0.1121 | 962_E_+2 |
| 962_G_4 | 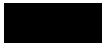 | 0.4162 | 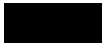 | 0.2008 | 962_G_4 |
| 962_G_1 | 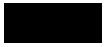 | 0.0625 | 0.1196 | 0.1409 | 962_G_1 |
| 962_G_2 | 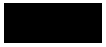 | 0.6958 | 0.4654 | 0.3824 | 962_G_2 |
| 962_G_6 | 0.093 | 0.0671 | 0.7585 | 0.4835 | 962_Q_6 |
| 962_H_+2 | 0.0822 | 0.074 | 0.5836 | 0.2512 | 962_H_+2 |
| 962_M_+2 | 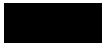 | 0.6632 | 0.7877 | 0.5007 | 962_M_+2 |
| 962_P_−2 | 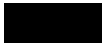 | 0.01951 | 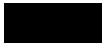 | 0.0672 | 962_P_−2 |
| 962_Q_−1 | 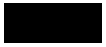 | 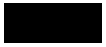 | 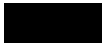 | 0.1148 | 962_Q_−1 |
| 962_S_−1 | 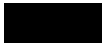 | 0.7916 | 0.8609 | 0.4923 | 962_S_−1 |
| 962_U_1 | 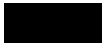 | 0.8537 | 0.8558 | 0.184 | 962_U_1 |
| 962_U_2 | 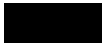 | 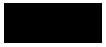 | 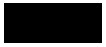 | 0.0705 | 962_U_2 |
| 962_V_−1 | — | 0.6278 | 0.1653 | 0.0863 | 962_V_−1 |
| 962_V_+2 | — | | 1 | 0.4925 | 962_V_+2 |
| 962_Z_1 | | | | 0.2483 | 962_Z_1 |
| CNTL | 21.7% | 35.4% | 4.7% | 35.5% | CNTL |
| CASE | 45.8% | 27.3% | 4.2% | 20.8% | CASE |

All SNP combinations in Tables 29, 30, and 31 that demonstrated a significant difference (p≦0.05) in the distribution of frequencies of the four haplotypes between the cases and the control populations were further analyzed to identify individual haplotypes that were also significant. Table 32 presents the haplotypes that were significantly associated, at the 0.05 level of significance, with the IgE phenotype. Haplotypes with higher allele frequency in the case population than in the control population acted as risk factors that increased the susceptibility to asthma. Haplotypes with lower allele frequencies in the case population than in the control population acted as protective factors that decreased the susceptibility to asthma. For Gene 845, a single susceptibility haplotype G/C (SNPs R1/R−1, p=0.0287) was significant in the US population. For Gene 962, four haplotypes were susceptibility haplotypes in the combined population. They were haplotypes T/A (SNPs E+2/G1, p=0.0163), G/A (SNPs G4/G1, p=0.0096), A/A (SNPs G1/P−2, p=0.0121) and A/A (SNPs G1/Q-1, p=0.0018). Two haplotypes were protective in the combined population. They were C/T (SNPs E+2/V−-1, p=0.0386) and G/A (SNPs G1/Q−1, p=0.0196). Four haplotypes were susceptibility haplotypes in the UK population. They were haplotypes G/A (SNPs G4/G1, p=0.0104), G/C (SNPs G4/G6, p=0.0156), A/A (SNPs G1/Q−1, p=0.041) and C/G (SNPs G6/S−1, p=0.0057). Three haplotypes were protective in the UK population. They were haplotypes G/A (SNPs G1/Q−1, p=0.0138), C/C (SNPs G6/S−1, p=0.0401) and T/G (SNPs G6/S−1, p=0.0255). Six haplotypes were susceptibility haplotypes in the US population. They were G/A (SNPs G4/Q−1, p=0.0096), G/T (SNPs G4/U2, p=0.0086), A/A (SNPs G4N+2, p=0.0305), A/A (SNPs G1/Q−1, p=0.0072), A/T (SNPs G1/U2, p=0.0062) and G/A (SNPs M+2/P−2, p=0.0009). The haplotypes T/G (SNPs E3/G1, p=0.0367) and G/G (SNPs M+2/P−2, p=0.0001) were protective haplotypes in the US population.

TABLE 32

| | | | | | |
|---|---|---|---|---|---|
| | SNP | | | | |
| | COMBI- | HAPLO- | FREQUENCIES | | P- |
| GENE | NATION | TYPE | CNTL | CASE | VALUE |

Total IgE Combined
US and UK

| | | | | | |
|---|---|---|---|---|---|
| 962 | E+2/G1 | TA | 0.237558 | 0.340794 | 0.0163 |
| 962 | E+2/V−1 | CT | 0.049664 | 0 | 0.0386 |
| 962 | G4/G1 | GA | 0.208523 | 0.317136 | 0.0096 |
| 962 | G1/P−2 | AA | 0.057605 | 0.140911 | 0.0121 |
| 962 | G1/Q−1 | AA | 0.049187 | 0.148707 | 0.0018 |
| 962 | G1/Q−1 | GA | 0.187956 | 0.094082 | 0.0196 |

Total IgE
UK Population

| | | | | | |
|---|---|---|---|---|---|
| 962 | G4/G1 | GA | 0.175073 | 0.289291 | 0.0104 |
| 962 | G4/G6 | GC | 0.640687 | 0.77847 | 0.0156 |
| 962 | G1/Q−1 | AA | 0.050665 | 0.116778 | 0.041 |
| 962 | G1/Q−1 | GA | 0.197425 | 0.088239 | 0.0138 |
| 962 | G6/S−1 | CC | 0.101659 | 0.031721 | 0.0401 |
| 962 | G6/S−1 | CG | 0.681786 | 0.821243 | 0.0057 |
| 962 | G6/S−1 | TG | 0.208433 | 0.11209 | 0.0255 |

Total IgE
US Population

| | | | | | |
|---|---|---|---|---|---|
| 845 | R1/R−1 | GC | 0.504855 | 0.75 | 0.0287 |
| 962 | E3/G1 | TG | 0.383347 | 0.125 | 0.0367 |
| 962 | G4/Q−1 | GA | 0.192645 | 0.410714 | 0.0096 |
| 962 | G4/U2 | GT | 0.191643 | 0.416667 | 0.0086 |
| 962 | G4/V+2 | AA | 0 | 0.041667 | 0.0305 |
| 962 | G1/Q−1 | AA | 0.042798 | 0.29779 | 0.0072 |
| 962 | G1/U2 | AT | 0.039558 | 0.286625 | 0.0068 |
| 962 | M+2/P−2 | GG | 0.643053 | 0.263889 | 0.0001 |
| 962 | M+2/P−2 | GA | 0.224358 | 0.527778 | 0.0009 |

d. Specific IgE

In Tables 33, 34 and 35, the haplotype analysis (2-at-a-time) is presented for the combined, the UK and the US populations, respectively. Two SNP combinations in Gene 845 are significant in the US population: SNPs R 1 & R−1 (p=0.0227) and SNPs R 1 & K−2 (p=0.0293). A single SNP combination in Gene 847 is significant in the US population: SNPs J+1 & D−1 (p=0.0341). Three SNP combinations in Gene 803 are significant in the US population: SNPs K 2 & I 1 (p=0.0469), SNPs K 2 & I−1 (p=0.0322) and SNPs K 2 & E+2 (p=0.0212). Sixteen SNP combinations in Gene 962 are significant in the combined and in the UK and US population alone: SNPs E 3 & G 1 (US p=0.0281), SNPs G 4 & G 1 (combined p=0.0047, UK p=0.0351), SNPs G 4 & S−1 (combined p=0.0064), SNPs G 4 & U 2 (US p=0.0386), SNPs G 4 & V+2 (US p=0.0366), SNPs G 1 & M+2 (combined p=0.0184, UK p=0.0049), SNPs G 1 & P−2 (combined p=0.0235), SNPs G 1 & Q−1 (combined p=0.0144, US p=0.0265), SNPs G 1 & S−1 (combined p=0.0051, UK p=0.00055), SNPs G 1 & U 2 (combined p=0.0213, US p=0.0256), SNPs G 6 & S−1 (UK p=0.00021), SNPs G 6 & V−1 (UK p=0.0143), SNPs Q−1 & V−1 (US p=0.023), SNPs U1 & Z1 (US p=0.0328), SNPs U 2 & V−1 (US p=0.0239) and SNPs U 2 & V+2 (US p=0.0387).

TABLE 33
HAPLOTYPE ANALYSIS OF SPECIFIC IgE PHENOTYPE COMBINED US/UK POPULATION

| | 845_R_1 | 845_R_−1 | 845_P_+1 | 845_K_1 | 845_K_−2 | 845_J_1 | 845_J_−1 | 845_I_−1 | 845_H_+1 | 845_H_−1 | 845_G_+1 | 845_F_+1 | 845_D_1 | 845_D_−1 | |
|---|---|---|---|---|---|---|---|---|---|---|---|---|---|---|---|
| 845_{—} | 0.2296 | 0.1518 | 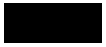 | 0.3714 | 0.1807 | 0.1629 | 0.3673 | 0.3834 | 0.1673 | 0.2708 | 0.3605 | 0.3813 | 0.1999 | 0.1281 | 845_R_1 |
| R_1 | | | | | | | | | | | | | | | |
| 845_{—} | — | 0.3572 | 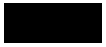 | 0.4822 | 0.3161 | 0.2909 | 0.1797 | 0.6834 | 0.0798 | 0.7266 | 0.7126 | 0.7033 | 0.3785 | 0.1726 | 845_R_−1 |
| R_31 1 | | | | | | | | | | | | | | | |
| 845_{—} | — | — | 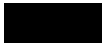 | 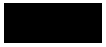 | 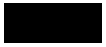 | 0.0745 | 0.0779 | 0.054 | 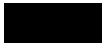 | 0.0731 | 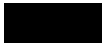 | 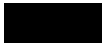 | 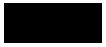 | 0.0768 | 845_P_+1 |
| P_+1 | | | | | | | | | | | | | | | |
| 845_{—} | — | — | — | 1 | 0.5052 | 0.1554 | 0.4826 | 0.791 | 0.2566 | 0.5345 | 0.7481 | 0.7973 | 0.7413 | 0.1938 | 845_K_1 |
| K_1 | | | | | | | | | | | | | | | |
| 845_{—} | | — | — | — | 0.4121 | 0.1831 | 0.226 | 0.5452 | 0.0913 | 0.6269 | 0.6766 | 0.6511 | 0.4105 | 0.17 | 845_K_−2 |
| K_−2 | | | | | | | | | | | | | | | |
| 845_{—} | — | — | — | — | — | 0.0856 | 0.1835 | 0.0905 | 0.1172 | 0.329 | 0.0883 | 0.1606 | 0.1449 | 0.106 | 845_J_1 |
| J_1 | | | | | | | | | | | | | | | |
| 845_{—} | — | — | — | — | — | — | 0.391 | 0.5301 | 0.2668 | 0.3866 | 0.5501 | 0.7273 | 0.3249 | 0.1999 | 845_J_−1 |
| J_−1 | | | | | | | | | | | | | | | |
| 845_{—} | — | — | — | — | — | — | — | 0.8746 | 0.2215 | 0.6014 | 0.5279 | 0.7317 | 0.8892 | 0.211 | 845_I_−1 |
| I_−1 | | | | | | | | | | | | | | | |
| 845_{—} | — | — | — | — | — | — | — | — | 0.1414 | 0.1415 | 0.3128 | 0.3392 | 0.1094 | 0.0821 | 845_H_+1 |
| H_+1 | | | | | | | | | | | | | | | |
| 845_{—} | — | — | — | — | — | — | — | — | — | 0.4045 | 0.7055 | 0.7036 | 0.4819 | 0.3251 | 845_H_−1 |
| H_−1 | | | | | | | | | | | | | | | |
| 845_{—} | — | — | — | — | — | — | — | — | — | — | 0.7635 | 0.9483 | 0.8866 | 0.2038 | 845_G_+1 |
| G_+1 | | | | | | | | | | | | | | | |
| 845_{—} | — | — | — | — | — | — | — | — | — | — | — | 0.8944 | 0.9094 | 0.2005 | 845_F_+1 |
| F_+1 | | | | | | | | | | | | | | | |
| 845_{—} | — | — | — | — | — | — | — | — | — | — | — | — | 0.3743 | 0.1541 | 845_D_1 |
| D_1 | | | | | | | | | | | | | | | |
| 845_{—} | — | — | — | — | — | — | — | — | — | — | — | — | — | 0.0931 | 845_D_−1 |
| D_−1 | | | | | | | | | | | | | | | |
| CNTL | 14.8% | 28.7% | 6.5% | 0.2% | 28.9% | 34.5% | 37.1% | 12.8% | 19.6% | 45.2% | 13.2% | 18.4% | 0.2% | 9.9% | CNTL |
| CASE | 10.2% | 24.2% | 14.2% | 0.0% | 24.6% | 43.3% | 32.5% | 11.7% | 13.3% | 40.7% | 14.2% | 19.2% | 0.9% | 15.8% | CASE |

| | 847_K_1 | 847_J_+1 | 847_E_+1 | 847_D_−1 | 847_C_+1 | 847_A_2 | 847_A_1 | |
|---|---|---|---|---|---|---|---|---|
| 847_K_1 | 1 | 0.2108 | 0.5992 | 0.9672 | 0.8047 | 0.4804 | 0.7265 | 847_K_1 |
| 847_J_+1 | — | 0.1282 | 0.1488 | 0.2185 | 0.1525 | 0.188 | 0.198 | 847_J_+1 |
| 847_E_+1 | | — | 0.4567 | 0.5769 | 0.6622 | 0.8442 | 0.7965 | 847_E_+1 |
| 847_D_−1 | | — | | 0.8861 | 0.8465 | 0.9751 | 0.943 | 847_D_−1 |
| 847_C_+1 | — | — | — | — | 0.4345 | 0.801 | 0.7925 | 847_C_+1 |
| 847_A_2 | — | — | — | — | — | 0.6925 | 0.833 | 847_A_2 |
| 847_A_1 | — | — | — | — | — | — | 1 | 847_A_1 |
| CNTL | 4.9% | 4.9% | 12.6% | 16.9% | 17.9% | 6.7% | 1.0% | CNTL |
| CASE | 4.3% | 1.6% | 15.1% | 17.6% | 21.4% | 7.8% | 0.8% | CASE |

| | 874_R_+1 | 874_S_+1 | 874_T_−1 | 874_V_−1 | |
|---|---|---|---|---|---|
| 874_R_+1 | 0.1645 | 0.52 | 0.4011 | 0.5423 | 874_R_+1 |
| 874_S_+1 | | 0.7578 | 0.4091 | 0.7481 | 874_S_+1 |
| 874_T_−1 | — | — | 0.9204 | 0.8202 | 874_T_−1 |
| 874_V_−1 | | — | — | 0.7951 | 874_V_−1 |
| CNTL | 39.9% | 39.5% | 48.6% | 17.6% | CNTL |
| CASE | 32.5% | 41.4% | 49.2% | 18.5% | CASE |

| | 803_K_3 | 803_K_2 | 803_I_1 | 803_I_−1 | 803_H_+1 | 803_E_+2 | |
|---|---|---|---|---|---|---|---|
| 803_K_3 | 1 | 0.4344 | 0.9062 | 0.9978 | 0.969 | 0.9789 | 803_K_3 |
| 803_K_2 | — | 0.1402 | 0.3256 | 0.3166 | 0.3845 | 0.3316 | 803_K_2 |
| 803_I_1 | — | — | 0.9108 | 0.8981 | 0.9528 | 0.8027 | 803_I_1 |
| 803_I_−1 | — | — | — | 1 | 0.9445 | 0.9941 | 803_I_−1 |
| 803_H_+1 | — | — | — | — | 0.9094 | 0.8957 | 803_H_+1 |
| 803_E_+2 | — | — | | — | — | 1 | 803_E_+2 |
| CNTL | 0.9% | 0.2% | 28.2% | 0.2% | 25.2% | 44.2% | CNTL |
| CASE | 0.7% | 1.5% | 27.3% | 0.0% | 25.8% | 44.6% | CASE |

| | 962_E_3 | 962_E_+2 | 962_G_4 | 962_G_1 | 962_G_2 | 962_G_6 | 962_H_+2 | 962_M_+2 | 962_P_−2 | 962_Q_−1 | 962_S_−1 | 962_U_1 | |
|---|---|---|---|---|---|---|---|---|---|---|---|---|---|
| 962_E_3 | 0.6744 | 0.4225 | 0.3457 | 0.1286 | 0.4144 | 0.7862 | 0.3675 | 0.112 | 0.7783 | 0.7556 | 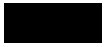 | 0.8311 | 962_E_3 |
| 962_E_+2 | — | 0.2273 | 0.1035 | 0.0705 | 0.6363 | 0.5865 | 0.3159 | 0.0889 | 0.2515 | 0.2958 | 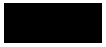 | 0.4087 | 962_E_+2 |
| 962_G_4 | — | — | 0.0891 | 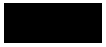 | 0.2229 | 0.2633 | 0.2078 | 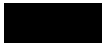 | 0.2325 | 0.2413 | 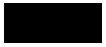 | 0.1943 | 962_G_4 |
| 962_G_1 | — | — | — | 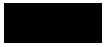 | 0.1604 | 0.0767 | 0.0529 | 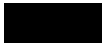 | 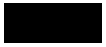 | 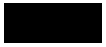 | 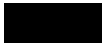 | 0.0854 | 962_G_1 |
| 962_G_2 | — | — | — | — | 0.8546 | 0.9507 | 0.5882 | 0.1948 | 0.7999 | 0.8257 | 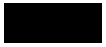 | 0.1274 | 962_G_2 |
| 962_G_6 | — | — | — | — | — | 0.6158 | 0.5057 | 0.1446 | 0.6043 | 0.6193 | 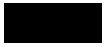 | 0.1287 | 962_G_6 |
| 962_H_+2 | — | — | — | — | — | — | 0.2288 | 0.0722 | 0.4866 | 0.4064 | 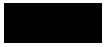 | 0.5704 | 962_H_+2 |
| 962_M_+2 | — | — | — | — | — | — | — | 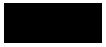 | 0.0925 | 0.1035 | 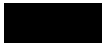 | 0.0938 | 962_M_+2 |
| 962_P_−2 | — | — | — | — | — | — | — | — | 0.3568 | 0.8098 | 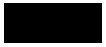 | 0.6846 | 962_P_−2 |
| 962_Q_−1 | — | — | — | — | — | — | — | — | — | 0.3505 | 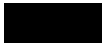 | 0.6508 | 962_Q_−1 |
| 962_S_−1 | — | — | — | — | — | — | — | — | — | — | 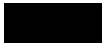 | 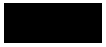 | 962_S_−1 |
| 962_U_1 | — | — | — | — | — | — | — | — | — | — | — | 0.4086 | 962_U_1 |
| 962_U_2 | — | — | | | — | — | — | — | — | — | — | — | 962_U_2 |
| 962_V_−1 | — | — | — | | — | — | — | — | — | — | — | — | 962_V_−1 |
| 962_V_+2 | — | — | — | — | — | — | — | — | — | — | — | — | 962_V_+2 |
| 962_Z_1 | — | — | — | — | — | — | — | — | — | — | — | — | 962_Z_1 |
| CNTL | 35.6% | 12.7% | 13.6% | 27.2% | 7.6% | 20.4% | 41.5% | 12.7% | 23.8% | 23.7% | 11.4% | 3.0% | CNTL |
| CASE | 33.1% | 8.7% | 7.7% | 37.7% | 8.1% | 18.2% | 35.3% | 6.2% | 28.0% | 28.1% | 3.7% | 4.6% | CASE |

| | 962_U_2 | 962_V_−1 | 962_V_+2 | 962_Z_1 | |
|---|---|---|---|---|---|
| 962_E_3 | 0.9381 | 0.2573 | 0.7005 | 0.7193 | 962_E_3 |
| 962_E_+2 | 0.419 | 0.3405 | 0.4616 | 0.4665 | 962_E_+2 |
| 962_G_4 | 0.3096 | 0.262 | 0.335 | 0.1846 | 962_G_4 |
| 962_G_1 | 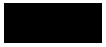 | 0.0946 | 0.1697 | 0.1218 | 962_G_1 |
| 962_G_2 | 0.9647 | 0.8981 | 0.9379 | 0.8667 | 962_G_2 |
| 962_G_6 | 0.9029 | 0.539 | 0.523 | 0.7072 | 962_G_6 |
| 962_H_+2 | 0.3779 | 0.1938 | 0.3487 | 0.4438 | 962_H_+2 |
| 962_M_+2 | 0.112 | 0.1275 | 0.0974 | 0.1731 | 962_M_+2 |
| 962_P_−2 | 0.3626 | 0.8466 | 0.6076 | 0.4826 | 962_P_−2 |
| 962_Q_−1 | 0.6619 | 0.6842 | 0.7026 | 0.553 | 962_Q_−1 |
| 962_S_−1 | 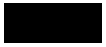 | 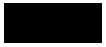 | 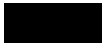 | 962_{—} | |
| | | | | S_−1 | |
| 962_U_1 | 0.8632 | 0.5495 | 0.6703 | 0.343 | 962_U_1 |
| 962_U_2 | 0.7291 | 0.7724 | 0.8708 | 0.7266 | 962_U_2 |
| 962_V_−1 | — | 0.456 | 0.8624 | 0.6423 | 962_V_−1 |
| 962_V_+2 | — | — | 0.8108 | 0.6306 | 962_V_+2 |
| 962_Z_1 | — | | — | 0.4601 | 962_Z_1 |
| CNTL | 23.8% | 34.8% | 4.5% | 31.9% | CNTL |
| CASE | 25.4% | 31.1% | 3.7% | 28.3% | CASE |

TABLE 34
HAPLOTYPE ANALYSIS OF SPECIFIC IgE PHENOTYPE UK POPULATION

| | 845_R_1 | 845_R_−1 | 845_P_+1 | 845_K_1 | 845_K_−2 | 845_J_1 | 845_J_−1 | | |
|---|---|---|---|---|---|---|---|---|---|
| 845_R_1 | 0.7179 | 0.6166 | 0.2644 | 0.7802 | 0.6552 | 0.5648 | 0.7964 | 845_R_1 | |
| 845_R_−1 | — | 0.4875 | 0.2588 | 0.6508 | 0.6762 | 0.5075 | 0.4611 | 845_R_−1 | |
| 845_P_+1 | | | 0.0724 | 0.2294 | 0.27 | 0.3931 | 0.2586 | 845_P_+1 | |
| 845_K_1 | | — | — | 1 | 0.6585 | 0.4304 | 0.6677 | 845_K_1 | |
| 845_K_−2 | | | | — | 0.5747 | 0.4925 | 0.5387 | 845_K_−2 | |
| 845_J_1 | | | | | — | 0.3119 | 0.5036 | 845_J_1 | |
| 845_J_−1 | | | — | — | — | — | 0.6131 | 845_J_−1 | |
| 845_I_−1 | — | — | — | — | — | — | — | 845_I_−1 | |
| 845_H_+1 | | — | — | — | — | — | — | 845_H_+1 | |
| 845_H_−1 | | — | — | — | — | — | — | 845_H_−1 | |
| 845_G_+1 | — | — | — | — | — | — | — | 845_G_+1 | |
| 845_F_+1 | — | — | — | — | — | — | — | 845_F_+1 | |
| 845_D_1 | — | | — | — | — | — | — | 845_D_1 | |
| 845_D_−1 | — | | — | — | — | — | — | 845_D_−1 | |
| CNTL | 13.6% | 26.6% | 6.4% | 0.4% | 27.0% | 36.6% | 37.5% | CNTL | |
| CASE | 11.6% | 22.7% | 12.5% | 0.0% | 23.3% | 43.2% | 34.1% | CASE | |
| | 845_I_−1 | 845_H_+1 | 845_H_−1 | 845_G_+1 | 845_F_+1 | 845_D_1 | 845_D_−1 | | |
| 845_R_1 | 0.8368 | 0.3811 | 0.6567 | 0.5634 | 0.5657 | 0.1807 | 0.3181 | 845_R_1 | |
| 845_R_−1 | 0.675 | 0.2615 | 0.7534 | 0.6647 | 0.7616 | 0.1752 | 0.3296 | 845_R_−1 | |
| 845_P_+1 | 0.2596 | 0.1602 | 0.348 | 0.2028 | 0.2206 | 0.0811 | 0.2774 | 845_P_+1 | |
| 845_K_1 | 0.8109 | 0.425 | 0.5828 | 0.5418 | 0.7533 | 0.1398 | 0.3064 | 845_K_1 | |
| 845_K_−2 | 0.7254 | 0.2937 | 0.7282 | 0.7126 | 0.7917 | 0.1835 | 0.3029 | 845_K_−2 | |
| 845_J_1 | 0.3359 | 0.3591 | 0.7922 | 0.3446 | 0.5693 | 0.132 | 0.3158 | 845_J_1 | |
| 845_J_−1 | 0.7402 | 0.4496 | 0.6547 | 0.5778 | 0.9237 | 0.1777 | 0.3385 | 845_J_−1 | |
| 845_I_−1 | 0.852 | 0.3828 | 0.6398 | 0.2937 | 0.7773 | 0.2669 | 0.3774 | 845_I_−1 | |
| 845_H_+1 | — | 0.2667 | 0.2899 | 0.4678 | 0.5499 | 0.0519 | 0.192 | 845_H_+1 | |
| 845_H_−1 | — | — | 0.459 | 0.6689 | 0.7392 | 0.1676 | 0.1996 | 845_H_−1 | |
| 845_G_+1 | — | — | — | 0.5956 | 0.7324 | 0.1832 | 0.2585 | 845_G_+1 | |
| 845_F_+1 | — | — | — | — | 0.757 | 0.2616 | 0.2757 | 845_F_+1 | |
| 845_D_1 | — | — | — | — | — | 0.235 | 0.0739 | 845_D_1 | |
| 845_D_−1 | — | — | — | — | — | — | 0.1601 | 845_D_−1 | |
| CNTL | 12.6% | 19.3% | 43.6% | 13.2% | 18.8% | 0.0% | 9.3% | CNTL | |
| CASE | 11.4% | 13.6% | 38.6% | 15.9% | 20.5% | 1.2% | 15.1% | CASE | |
| | 847_K_1 | 847_J_+1 | 847_E_+1 | 847_D_−1 | 847_C_+1 | 847_A_2 | 847_A_1 | | |
| 847_K_1 | 1 | 0.556 | 0.33 | 0.5504 | 0.3852 | 0.446 | 0.9021 | 847_K_1 | |
| 847_J_+1 | — | 0.461 | 0.2494 | 0.28 | 0.1971 | 0.6781 | 0.6582 | 847_J_+1 | |
| 847_E_+1 | — | — | 0.1653 | 0.1659 | 0.2652 | 0.4232 | 0.4364 | 847_E_+1 | |
| 847_D_−1 | | — | — | 0.1853 | 0.1276 | 0.5154 | 0.7086 | 847_D_−1 | |
| 847_C_+1 | — | — | — | — | 0.0964 | 0.272 | 0.33 | 847_C_+1 | |
| 847_A_2 | — | — | — | — | — | 0.6132 | 0.8312 | 847_A_2 | |
| 847_A_1 | — | — | — | — | — | — | 1 | 847_A_1 | |
| CNTL | 3.6% | 2.9% | 12.8% | 16.5% | 17.8% | 5.4% | 1.3% | CNTL | |
| CASE | 2.5% | 1.1% | 18.9% | 23.1% | 26.1% | 6.5% | 1.1% | CASE | |
| | 874_R_+1 | 874_S_+1 | 874_T_−1 | 874_V_−1 | | | | | |
| 874_R_+1 | 0.1412 | 0.332 | 0.2742 | 0.4802 | 874_R_+1 | | | | |
| 874_S_+1 | — | 0.7215 | 0.4266 | 0.8159 | 874_S_+1 | | | | |
| 874_T_−1 | | — | 0.7309 | 0.8326 | 874_T_−1 | | | | |
| 874_V_−1 | — | — | — | 0.7623 | 874_V_−1 | | | | |
| CNTL | 41.5% | 38.0% | 48.9% | 16.9% | CNTL | | | | |
| CASE | 32.3% | 40.2% | 51.0% | 18.3% | CASE | | | | |
| | 803_K_3 | 803_K_2 | 803_I_1 | 803_I_−1 | 803_H_+1 | 803_E_+2 | | | |
| 803_K_3 | 1 | 0.9362 | 0.6485 | 0.9952 | 0.9915 | 0.8117 | 803_K_3 | | |
| 803_K_2 | — | 0.4764 | 0.7517 | 0.993 | 0.5925 | 0.7175 | 803_K_2 | | |
| 803_I_1 | — | — | 0.4281 | 0.5416 | 0.5808 | 0.5877 | 803_I_1 | | |
| 803_I_−1 | — | — | — | 1 | 0.9705 | 0.4939 | 803_I_−1 | | |
| 803_H_+1 | — | — | — | — | 1 | 0.4115 | 803_H_+1 | | |
| 803_E_+2 | | — | — | — | — | 0.3491 | 803_E_+2 | | |
| CNTL | 1.1% | 0.4% | 28.6% | 0.4% | 25.7% | 42.4% | CNTL | | |
| CASE | 0.9% | 0.9% | 24.0% | 0.0% | 26.0% | 48.0% | CASE | | |
| | 962_E_3 | 962_E_+2 | 962_G_4 | 962_G_1 | 962_G_2 | 962_G_6 | 962_H_+2 | 962_M_+2 | |
| 962_E_3 | 0.2697 | 0.4471 | 0.2848 | 0.1582 | 0.5166 | 0.2632 | 0.3556 | 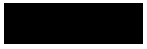 | 962_E_3 |
| 962_E_+2 | | 0.3692 | 0.1179 | 0.1643 | 0.5854 | 0.1661 | 0.4283 | 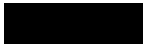 | 962_E_+2 |
| 962_G_4 | — | — | 0.1539 | 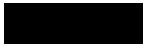 | 0.2729 | 0.098 | 0.3311 | 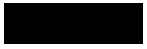 | 962_G_4 |
| 962_G_1 | | — | — | 0.0742 | 0.2966 | 0.0975 | 0.1596 | 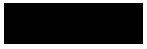 | 962_G_1 |
| 962_G_2 | — | — | — | — | 0.5009 | 0.4053 | 0.4236 | 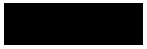 | 962_G_2 |
| 962_G_6 | — | — | — | — | — | 0.1841 | 0.1991 | 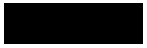 | 962_G_6 |
| 962_H_+2 | — | — | — | — | — | — | 0.2445 | 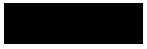 | 962_H_+2 |
| 962_M_+2 | — | — | — | — | — | — | — | 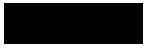 | 962_M_+2 |
| 962_P_−2 | — | — | — | — | — | — | — | — | 962_P_−2 |
| 962_Q_−1 | — | — | — | — | — | — | — | — | 962_Q_−1 |
| 962_S_−1 | — | — | — | — | — | — | — | — | 962_S_−1 |
| 962_U_1 | — | — | — | — | — | — | — | — | 962_U_1 |
| 962_U_2 | — | — | — | | — | — | — | — | 962_U_2 |
| 962_V_−1 | — | — | — | — | — | — | — | — | 962_V_−1 |
| 962_V_+2 | — | | | — | — | — | — | — | 962_V_+2 |
| 962_Z_1 | | — | — | — | — | — | — | — | 962_Z_1 |
| CNTL | 36.8% | 12.5% | 14.0% | 25.2% | 6.4% | 21.7% | 43.6% | 12.4% | CNTL |
| CASE | 30.0% | 8.5% | 8.2% | 34.9% | 8.7% | 15.0% | 36.5% | 3.0% | CASE |
| | 962_P_−2 | 962_Q_−1 | 962_S_−1 | 962_U_1 | 962_U_2 | 962_V_−1 | 962_V_+2 | 962_Z_1 | |
| 962_E_3 | 0.7001 | 0.7038 | 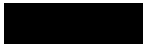 | 0.3907 | 0.5306 | 0.1475 | 0.7456 | 0.5462 | 962_E_3 |
| 962_E_+2 | 0.2666 | 0.3534 | 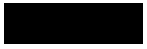 | 0.4799 | 0.2426 | 0.1067 | 0.5848 | 0.6605 | 962_E_+2 |
| 962_G_4 | 0.4604 | 0.484 | 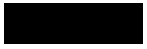 | 0.3027 | 0.366 | 0.3921 | 0.297 | 0.3495 | 962_G_4 |
| 962_G_1 | 0.1284 | 0.1732 | 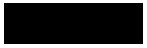 | 0.0626 | 0.1182 | 0.1849 | 0.2897 | 0.3231 | 962_G_1 |
| 962_G_2 | 0.9134 | 0.8569 | 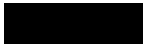 | 0.1854 | 0.766 | 0.5967 | 0.8865 | 0.8829 | 962_G_2 |
| 962_G_6 | 0.135 | 0.1258 | 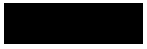 | 0.0962 | 0.2368 | 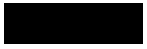 | 0.2299 | 0.4163 | 962_G_6 |
| 962_H_+2 | 0.5986 | 0.6454 | 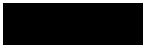 | 0.5194 | 0.4206 | 0.4917 | 0.3688 | 0.6572 | 962_H_+2 |
| 962_M_+2 | 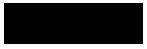 | 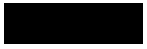 | 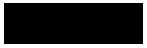 | 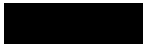 | 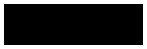 | 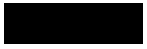 | 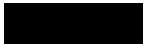 | 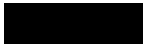 | 962_M_+2 |
| 962_P_−2 | 1 | 0.9998 | 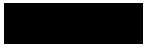 | 0.9493 | 0.6173 | 0.7626 | 0.9745 | 0.9291 | 962_P_−2 |
| 962_Q_−1 | — | 1 | 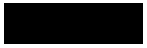 | 0.9707 | 0.7799 | 0.7163 | 0.9784 | 0.9584 | 962_Q_−1 |
| 962_S_−1 | — | — | 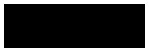 | 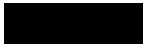 | 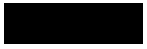 | 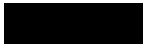 | 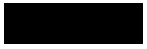 | 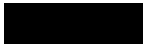 | 962_S_−1 |
| 962_U_1 | — | — | — | 0.5642 | 0.8007 | 0.4202 | 0.8658 | 0.729 | 962_U_1 |
| 962_U_2 | — | — | — | — | 0.4151 | 0.3504 | 0.8659 | 0.8125 | 962_U_2 |
| 962_V_−1 | — | — | — | — | — | 0.3829 | 0.6388 | 0.7466 | 962_V_−1 |
| 962_V_+2 | — | — | — | — | — | — | 1 | 0.8919 | 962_V_+2 |
| 962_Z_1 | — | — | — | — | — | — | — | 0.7114 | 962_Z_1 |
| CNTL | 24.6% | 24.8% | 10.9% | 3.7% | 25.0% | 34.4% | 4.5% | 30.0% | CNTL |
| CASE | 24.0% | 24.5% | 1.0% | 4.9% | 20.6% | 29.4% | 3.8% | 32.1% | CASE |

TABLE 35
HAPLOTYPE ANALYSIS OF SPECIFIC IgE PHENOTYPE US POPULATION

| | 845_R_1 | 845_R_−1 | 845_P_+1 | 845_K_1 | 845_K_−2 | 845_J_1 | 845_J_−1 | | |
|---|---|---|---|---|---|---|---|---|---|
| 845_R_1 | 0.175 | 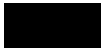 | 0.097 | 0.0804 | 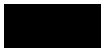 | 0.1689 | 0.2609 | 845_R_1 | |
| 845_R_−1 | | 0.6822 | 0.1594 | 0.6501 | 0.9435 | 0.4122 | 0.3452 | 845_R_−1 | |
| 845_P_+1 | — | — | 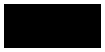 | 0.0909 | 0.1551 | 0.1498 | 0.1668 | 845_P_+1 | |
| 845_K_1 | — | — | — | 1 | 0.6613 | 0.1843 | 0.358 | 845_K_1 | |
| 845_K_−2 | | — | — | — | 0.8264 | 0.3347 | 0.4297 | 845_K_−2 | |
| 845_J_1 | — | — | — | — | — | 0.2144 | 0.3497 | 845_J_1 | |
| 845_J_−1 | | — | — | — | | — | 0.4212 | 845_J_−1 | |
| 845_I_−1 | | — | — | — | — | — | — | 845_I_−1 | |
| 845_H_+1 | | — | — | — | — | — | — | 845_H_+1 | |
| 845_H_−1 | | — | — | — | — | — | — | 845_H_−1 | |
| 845_G_+1 | — | — | — | — | — | — | — | 845_G_+1 | |
| 845_F_+1 | | — | — | — | — | — | — | 845_F_+1 | |
| 845_D_1 | | — | — | — | — | — | — | 845_D_1 | |
| 845_D_−1 | — | — | — | — | — | — | — | 845_D_−1 | |
| CNTL | 17.1% | 32.5% | 6.5% | 0.0% | 32.5% | 30.9% | 36.4% | CNTL | |
| CASE | 6.3% | 28.1% | 18.8% | 0.0% | 28.6% | 43.8% | 28.1% | CASE | |
| | 845_I_−1 | 845_H_+1 | 845_H_−1 | 845_G_+1 | 845_F_+1 | 845_D_1 | 845_D_−1 | | |
| 845_R_1 | 0.2989 | 0.4563 | 0.1511 | 0.3264 | 0.3726 | 0.2774 | 0.2491 | 845_R_1 | |
| 845_R_−1 | 0.9851 | 0.3784 | 0.8511 | 0.7938 | 0.9446 | 0.7015 | 0.6007 | 845_R_−1 | |
| 845_P_+1 | 0.197 | 0.1484 | 0.2216 | 0.1837 | 0.2313 | 0.1372 | 0.1265 | 845_P_+1 | |
| 845_K_1 | 0.9047 | 0.2098 | 0.8964 | 0.506 | 0.7327 | 0.9344 | 0.3654 | 845_K_1 | |
| 845_K_−2 | 0.6989 | 0.2679 | 0.9696 | 0.5982 | 0.8598 | 0.7101 | 0.6064 | 845_K_−2 | |
| 845_J_1 | 0.3023 | 0.3171 | 0.2419 | 0.3491 | 0.4717 | 0.3253 | 0.3068 | 845_J_1 | |
| 845_J_−1 | 0.6395 | 0.5232 | 0.6157 | 0.5865 | 0.7769 | 0.4739 | 0.549 | 845_J_−1 | |
| 845_I_−1 | 1 | 0.6081 | 0.9805 | 0.7185 | 0.816 | 0.9895 | 0.6621 | 845_I_−1 | |
| 845_H_+1 | — | 0.4562 | 0.4663 | 0.5249 | 0.7149 | 0.4445 | 0.4594 | 845_H_+1 | |
| 845_H_−1 | — | — | 1 | 0.8316 | 0.9391 | 0.9825 | 0.6571 | 845_H_−1 | |
| 845_G_+1 | — | — | — | 0.7705 | 0.8159 | 0.6574 | 0.5856 | 845_G_+1 | |
| 845_F_+1 | — | — | — | — | 1 | 0.9598 | 0.6606 | 845_F_+1 | |
| 845_D_1 | — | — | — | — | — | 1 | 0.4301 | 845_D_1 | |
| 845_D_−1 | — | — | — | — | — | — | 0.3432 | 845_D_−1 | |
| CNTL | 13.3% | 20.1% | 48.1% | 13.2% | 17.5% | 0.7% | 11.0% | CNTL | |
| CASE | 12.5% | 12.5% | 46.7% | 9.4% | 15.6% | 0.0% | 17.9% | CASE | |
| | 847_K_1 | 847_J_+1 | 847_E_+1 | 847_D_−1 | 847_C_+1 | 847_A_2 | 847_A_1 | | |
| 847_K_1 | 0.7364 | 0.5688 | 0.5756 | 0.1052 | 0.2831 | 0.9369 | 0.9841 | 847_K_1 | |
| 847_J_+1 | | 0.4757 | 0.1843 | 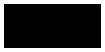 | 0.1836 | 0.1681 | 0.3925 | 847_J_+1 | |
| 847_E_+1 | | — | 0.3765 | 0.1293 | 0.3792 | 0.593 | 0.3756 | 847_E_+1 | |
| 847_D_−1 | — | — | — | 0.0516 | 0.1028 | 0.1118 | 0.0505 | 847_D_−1 | |
| 847_C_+1 | — | — | — | — | 0.2134 | 0.5643 | 0.3653 | 847_C_+1 | |
| 847_A_2 | — | — | — | — | — | 0.7523 | 0.8675 | 847_A_2 | |
| 847_A_1 | — | — | — | — | — | — | 1 | 847_A_1 | |
| CNTL | 7.3% | 8.4% | 12.3% | 17.6% | 18.2% | 9.1% | 0.7% | CNTL | |
| CASE | 8.3% | 2.8% | 5.6% | 3.3% | 8.8% | 11.1% | 0.0% | CASE | |
| | 874_R_+1 | 874_S_+1 | 874_T_−1 | 874_V_−1 | | | | | |
| 874_R_+1 | 0.8215 | 0.6966 | 0.9496 | 0.8969 | 874_R_+1 | | | | |
| 874_S_+1 | — | 0.8308 | 0.7872 | 0.9389 | 874_S_+1 | | | | |
| 874_T_−1 | — | — | 0.6733 | 0.8809 | 874_T_−1 | | | | |
| 874_V_−1 | | — | — | 1 | 874_V_−1 | | | | |
| CNTL | 37.0% | 42.2% | 48.1% | 18.8% | CNTL | | | | |
| CASE | 33.3% | 46.2% | 42.3% | 19.2% | CASE | | | | |
| | 803_K_3 | 803_K_2 | 803_I_1 | 803_I_−1 | 803_H_+1 | 803_E_+2 | | | |
| 803_K_3 | 1 | 0.2217 | 0.3284 | 0.7941 | 0.8564 | 0.2885 | 803_K_3 | | |
| 803_K_2 | — | 0.1522 | 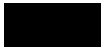 | 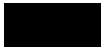 | 0.2229 | 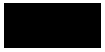 | 803_K_2 | | |
| 803_I_1 | — | — | 0.2596 | 0.2116 | 0.3967 | 0.3811 | 803_I_1 | | |
| 803_I_−1 | — | — | — | 1 | 0.9655 | 0.136 | 803_I_−1 | | |
| 803_H_+1 | — | — | — | — | 1 | 0.3741 | 803_H_+1 | | |
| 803_E_+2 | — | — | — | — | — | 0.1529 | 803_E_+2 | | |
| CNTL | 0.6% | 0.0% | 27.6% | 0.0% | 24.4% | 47.4% | CNTL | | |
| CASE | 0.0% | 3.6% | 39.3% | 0.0% | 25.0% | 32.1% | CASE | | |
| | 962_E_3 | 962_E_+2 | 962_G_4 | 962_G_1 | 962_G_2 | 962_G_6 | 962_H_+2 | 962_M_+2 | |
| 962_E_3 | 0.305 | 0.2406 | 0.2585 | 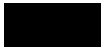 | 0.2389 | 0.3664 | 0.3253 | 0.6097 | 962_E_3 |
| 962_E_+2 | — | 0.7706 | 0.7148 | 0.26 | 0.6897 | 0.0917 | 0.2222 | 0.8358 | 962_E_+2 |
| 962_G_4 | — | — | 0.3748 | 0.1121 | 0.3078 | 0.2144 | 0.6699 | 0.389 | 962_G_4 |
| 962_G_1 | | — | — | 0.0999 | 0.3587 | 0.2778 | 0.2425 | 0.3823 | 962_G_1 |
| 962_G_2 | — | — | — | — | 0.7414 | 0.6277 | 0.6815 | 0.8657 | 962_G_2 |
| 962_G_6 | — | — | — | — | — | 0.216 | 0.6615 | 0.6239 | 962_G_6 |
| 962_H_+2 | — | — | — | — | — | — | 0.5501 | 0.3982 | 962_H_+2 |
| 962_M_+2 | — | — | — | — | — | — | — | 0.5704 | 962_M_+2 |
| 962_P_−2 | — | — | — | — | — | — | — | — | 962_P_−2 |
| 962_Q_−1 | — | — | — | — | — | — | — | — | 962_Q_−1 |
| 962_S_−1 | — | — | — | — | — | — | — | — | 962_S_−1 |
| 962_U_1 | — | — | — | — | — | — | — | — | 962_U_1 |
| 962_U_2 | — | — | — | — | — | — | — | — | 962_U_2 |
| 962_V_−1 | | — | — | — | — | — | — | — | 962_V_−1 |
| 962_V_+2 | | — | — | — | — | — | — | — | 962_V_+2 |
| 962_Z_1 | — | — | — | — | — | — | — | — | 962_Z_1 |
| CNTL | 33.6% | 13.0% | 12.9% | 30.8% | 9.7% | 17.7% | 37.7% | 13.2% | CNTL |
| CASE | 43.3% | 9.4% | 6.3% | 46.9% | 6.3% | 28.1% | 31.3% | 16.7% | CASE |
| | 962_P_−2 | 962_Q_−1 | 962_S_−1 | 962_U_1 | 962_U_2 | 962_V_−1 | 962_V_+2 | 962_Z_1 | |
| 962_E_3 | 0.0714 | 0.121 | 0.7172 | 0.1596 | 0.0999 | 0.7343 | 0.5741 | 0.0682 | 962_E_3 |
| 962_E_+2 | 0.0908 | 0.1311 | 0.8874 | 0.8644 | 0.0896 | 0.8613 | 0.7766 | 0.1191 | 962_E_+2 |
| 962_G_4 | 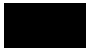 | 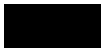 | 0.4842 | 0.4509 | 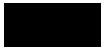 | 0.7467 | 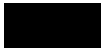 | 0.0531 | 962_G_4 |
| 962_G_1 | 0.0616 | 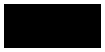 | 0.398 | 0.1067 | 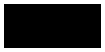 | 0.4599 | 0.1584 | 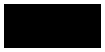 | 962_G_1 |
| 962_G_2 | 0.0905 | 0.1021 | 0.8738 | 0.7868 | 0.0725 | 0.6338 | 0.2843 | 0.1006 | 962_G_2 |
| 962_G_6 | 0.117 | 0.1176 | 0.6019 | 0.4484 | 0.1235 | 0.3756 | 0.5743 | 0.1 | 962_G_6 |
| 962_H_+2 | 0.2436 | 0.1996 | 0.4288 | 0.6756 | 0.1596 | 0.1278 | 0.7467 | 0.0525 | 962_H_+2 |
| 962_M_+2 | 0.0637 | 0.1021 | 0.5669 | 0.8508 | 0.0878 | 0.9423 | 0.8599 | 0.2111 | 962_M_+2 |
| 962_P_−2 | 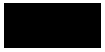 | 0.0791 | 0.1051 | 0.1038 | 0.0869 | 0.0677 | 0.0677 | 0.0514 | 962_P_−2 |
| 962_Q_−1 | — | 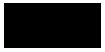 | 0.1482 | 0.1022 | 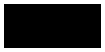 | 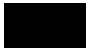 | 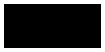 | 0.0558 | 962_Q_−1 |
| 962_S_−1 | — | — | 1 | 0.9234 | 0.1333 | 0.9917 | 0.9384 | 0.22 | 962_S_−1 |
| 962_U_1 | — | — | — | 0.4864 | 0.0912 | 0.9104 | 0.8463 | 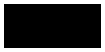 | 962_U_1 |
| 962_U_2 | — | — | — | — | 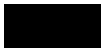 | 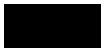 | 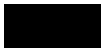 | 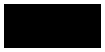 | 962_U_2 |
| 962_V_−1 | — | — | — | — | — | 1 | 0.1642 | 0.0526 | 962_V_−1 |
| 962_V_+2 | — | — | — | — | — | — | 1 | 0.0525 | 962_V_+2 |
| 962_Z_1 | — | — | — | — | — | — | — | 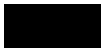 | 962_Z_1 |
| CNTL | 22.4% | 21.7% | 12.2% | 1.9% | 21.7% | 35.4% | 4.7% | 35.5% | CNTL |
| CASE | 42.9% | 40.0% | 12.5% | 3.6% | 40.6% | 36.7% | 3.1% | 15.6% | CASE |

All SNP combinations in Tables 33, 34, and 35 that demonstrated a significant difference (p≦0.05) in the distribution of frequencies of the four haplotypes between the cases and the control populations were further analyzed to identify individual haplotypes that were also significant. Table 36 presents the haplotypes that were significantly associated, at the 0.05 level of significance, with the Specific IgE phenotype. Haplotypes with higher allele frequency in the case population than in the control population acted as risk factors that increased the susceptibility to asthma. Haplotypes with lower allele frequencies in the case population than in the control population acted as protective factors that decreased the susceptibility to asthma. For Gene 845, two haplotypes were protective in the US population. They were haplotypes A/C (SNPs R1/R−1, p=0.0237) and A/G (SNPs R1/K−2, p=0.0268). Haplotypes G/C (SNPs R1/R−1, p=0.0308) and G/G (SNPs R1/K−2, p=0.0392) were susceptibility haplotypes in the US population. For Gene 847, two haplotypes were protective in the US population. They were haplotypes C/C (SNPs J+1/D−1, p=0.0409) and C/G (SNPs D−1/A1, p=0.0378). Haplotypes C/A (SNPs J+1/D−1, p=0.0113) and A/G (SNPs D−1/A1, p=0.0399) were susceptibility haplotypes in the US population. For Gene 962, seven haplotypes were susceptibility haplotypes in the combined population. They were haplotypes G/A (SNPs G4/G1, p=0.0175), G/G (SNPs G4/S−1, p=0.0066), A/G (SNPs G1/M+2, p=0.0107), A/A (SNPs G1/P−2, p=0.0054), A/A (SNPs G1/Q−1, p=0.0016), A/G (SNPs G1/S−1, p=0.0052) and ANT (SNPs G1/U2, p=0.004). The haplotype A/G (SNPs G4/G1, p=0.0211) was a protective haplotype in the combined population. Five haplotypes were susceptibility haplotypes in the UK population. They were G/A (SNPs G4/G1, p=0.0258), A/G (SNPs G1/M+2, p=0.0289), G/G (SNPs G1/S−1, p=0.02103), C/G (SNPs G6/S−1, p=0.0014) and C/C (SNPs G6/V−1, p=0.0084). Four haplotypes were protective haplotypes in the UK population. They were haplotypes G/C (SNPs G1/M+2, p=0.0171), G/C (SNPs G1/S−1, p=0.01065), C/C (SNPs G6/S−1, p=0.00239) and T/C (SNPs G6N-1, p=0.0085). Eight haplotypes were susceptibility haplotypes in the US population. They were haplotypes G/T (SNPs G4/U2, p=0.0446), A/A (SNPs G4/V+2, p=0.0433), ANA (SNPs G1/Q−1, p=0.003), A/T (SNPs G1/U2, p=0.0054), A/C (SNPs Q−1/V−1, p=0.0156), G/C (SNPs U1/Z1, p=0.0246), T/C (SNPs U2/V−1, p=0.0123) and T/G SNPs U2/V+2, p=0.0478). Four haplotypes were protective in the US population. They were haplotypes T/G (SNPs E3/G1, p=0.0118), T/C (SNPs Q−1/V−1, p=0.0123), G/T (SNPs Ul/Z1, p=0.0246) and C/C (SNPs U2/V−1, p=0.0225).

TABLE 36

| | | | | | |
|---|---|---|---|---|---|
| | SNP | | | | |
| | COMBI- | HAPLO- | FREQUENCIES | | P- |
| GENE | NATION | TYPE | CNTL | CASE | VALUE |

Specific IgE Combined
US and UK

| | | | | | |
|---|---|---|---|---|---|
| 962 | G4/G1 | GA | 0.208523 | 0.311853 | 0.0175 |
| 962 | G4/G1 | AG | 0.071601 | 0.009531 | 0.0211 |
| 962 | G4/S−1 | GG | 0.772436 | 0.886176 | 0.0066 |
| 962 | G1/M+2 | AG | 0.231629 | 0.346667 | 0.0107 |
| 962 | G1/P−2 | AA | 0.057605 | 0.15373 | 0.0054 |
| 962 | G1/Q−1 | AA | 0.049187 | 0.157002 | 0.0016 |
| 962 | G1/S−1 | AG | 0.23279 | 0.357599 | 0.0052 |
| 962 | G1/U2 | AT | 0.052762 | 0.147695 | 0.004 |

Specific IgE
UK Population

| | | | | | |
|---|---|---|---|---|---|
| 962 | G4/G1 | GA | 0.175073 | 0.282626 | 0.0258 |
| 962 | G1/M+2 | AG | 0.217702 | 0.329111 | 0.0289 |
| 962 | G1/M+2 | GC | 0.089986 | 0.010015 | 0.0171 |
| 962 | G1/S−1 | GG | 0.221485 | 0.339623 | 0.02103 |
| 962 | G1/S−1 | GC | 0.079171 | 0 | 0.01065 |
| 962 | G6/S−1 | CG | 0.681786 | 0.85033 | 0.0014 |
| 962 | G6/S−1 | CC | 0.101659 | 0 | 0.00239 |
| 962 | G6/V−1 | CC | 0.498551 | 0.660434 | 0.0084 |
| 962 | G6/V−1 | TC | 0.157531 | 0.042045 | 0.0085 |

Specific IgE
US Population

| | | | | | |
|---|---|---|---|---|---|
| 845 | R1/R−1 | GC | 0.504855 | 0.71875 | 0.0308 |
| 845 | R1/R−1 | AC | 0.17047 | 0 | 0.0237 |
| 845 | R1/K−2 | GG | 0.504855 | 0.721154 | 0.0392 |
| 845 | R1/K−2 | AG | 0.17047 | 0 | 0.0268 |
| 847 | J+1/D−1 | CA | 0.738791 | 0.938697 | 0.0113 |
| 847 | J+1/D−1 | CC | 0.176558 | 0.033525 | 0.0409 |
| 847 | D−1/A1 | AG | 0.817323 | 0.966667 | 0.0399 |
| 847 | D−1/A1 | CG | 0.175709 | 0.033333 | 0.0378 |
| 962 | E3/G1 | TG | 0.383347 | 0.110692 | 0.0118 |
| 962 | G4/U2 | GT | 0.191643 | 0.34375 | 0.0446 |
| 962 | G4/V+2 | AA | 0 | 0.03125 | 0.0433 |
| 962 | G1/Q−1 | AA | 0.042798 | 0.289956 | 0.003 |
| 962 | G1/U2 | AT | 0.039558 | 0.279877 | 0.0054 |
| 962 | Q−1/V−1 | AC | 0.217105 | 0.417425 | 0.0156 |
| 962 | Q−1/V−1 | TC | 0.433253 | 0.197514 | 0.0123 |
| 962 | U1/Z1 | GT | 0.3357 | 0.125 | 0.0164 |
| 962 | U1/Z1 | GC | 0.64507 | 0.84375 | 0.0246 |
| 962 | U2/V−1 | TC | 0.215811 | 0.40625 | 0.0123 |
| 962 | U2/V−1 | CC | 0.431687 | 0.209559 | 0.0225 |
| 962 | U2/V+2 | TG | 0.217236 | 0.375 | 0.0478 |

### Example 8

Genes Role in Asthma and Other Disorders

ADAM family proteins are known to interact with other cellular proteins. For example, the substrate of ADAM 19, NRG1, belongs to a group of growth factors (neuregulins) that are members of the epidermal growth factor family. The neuregulins participate in an array of biological effects that are mediated by the epidermal growth factor family of tyrosine kinase receptors. Data suggest that the proteolytically cleaved isoform of NRG1, NRG-β1, may induce the tyrosine phosphorylation of EGFR2 and EGFR3 in differentiated muscle cells (Shirakabe et. al., 2001, J. Biol. Chem. 276(12):9352-8).

Epidermal growth factor receptor (EGFR1) plays a pivotal role in the maintenance and repair of epithelial tissue. Following injury in bronchial epithelium, EGFR1 is upregulated in response to ligands acting on it or through transactivation of the EGFR1 receptor. This results in the increased proliferation of cells and airway remodeling at the point of insult, leading to the repair of the bronchial epithelium (Polosa et. al., 1999, Am. J. Respir. Cell Mol. Biol. 20:914-923; Holgate et. al., 1999, Clin. Exp. Allergy Suppl 2:90-95).

In asthma, the bronchial epithelium is highly abnormal, with structural changes involving separation of columnar cells from their basal attachments and functional changes that include increased expression and release of proinflammatory cytokines, growth factors, and mediator-generating enzymes. Beneath this damaged structure are the subepithelial myofibroblasts that have been activated to proliferate. This, in turn, causes excessive matrix deposition leading to abnormal thickening and increased density of the subepithelial basement membrane.

Immunocytochemical studies have shown that both TGF-β and EGFR1 are highly expressed at the area of injury and that parallel pathways could be operating in the repairing epithelial cells (Puddicombe et. al., 2000, FASEB J. 14:1362-1374). EGFR1 stimulates epithelial repair and TGF-β regulates the production of profibrogenic growth factors and proinflammatory cytokines leading to extracellular matrix synthesis. As EGFR1 is involved in regulating a number of different stages of epithelial repair (survival, migration, proliferation and differentiation), any inhibitory effects that act on the receptor may cause the epithelium to be held in a “state of repair” (Holgate et. al., 1999, Clin. Exp. Allergy Suppl 2:90-95).

It is possible that variant ADAM family proteins induce the epithelium into a continuous “state of repair” by functioning improperly and failing to release their substrates (members of the neuregulin family) that serve as the ligand for EGFR1. This, in turn, may cause the observed increase in EGFR1 expression. Under these circumstances, the TGF-β pathway remains active, producing a continuous source of proinflammatory products as well as growth factors that drive airway wall remodeling causing bronchial hyperresponsiveness, a phenotype of asthma.

Gene 845—ADAM 19

Human ADAM19 (meltrin-β) is a member of the disintegrin and metalloprotease family and maps to chromosome 5q32-q33. The transcript is ˜7.0 Kb in size and is found to be expressed in many tissues including lung (Wei P, et. al., Biochem Biophys Res Comm 280: 744-755 (2001)). Studies of ADAM19 expressed in muscle and bone suggest that it plays a role in osteogenesis and myogenesis (Kurisaki T, et. al., Mech Dev 73:211-215 (1998)). Further, it is purported to heterodimerize with ADAM12 and may be involved in aggregation and fusion of cells with different surface phenotypes (Yamamato S, et. al., Immunology Today 20:278-284 (1999)). In the lung, ADAM19 can be involved in the sequestering of cells, such as myofibroblasts, to areas of inflammation. ADAM 19 is most closely related to the asthma-associated gene Gene 216 (U.S. patent application Ser. No. 09/834,597). Mutations in ADAM19 could modulate the function of the gene. Four single nucleotide polymorphisms (SNPs) have been identified within the open reading frame (ORF) of ADAM19 that cause amino acid changes. One of those SNPs, Gene845_J_{—}1 is strongly associated with the disease. This amino acid change, serine to glycine, resides within the catalytic domain between two conserved residues, leucine and tryptophan. It is possible that this amino acid change in ADAM19 may alter the functional capacity of the catalytic domain in the protein leading to the onset of asthma and other respiratory disorders.

Gene 847—Neuregulin 2

Human Neuregulin 2 (NRG2) is a member of the neuregulin family of growth and differentiation factors and maps to chromosome 5q23-q33. The transcript size is ˜3.0 Kb in size and there are six alternatively transcribed species which encode six protein isoforms. NRG2 is expressed in a limited number of tissues, which includes lung. The NRG2 isoforms interact with the Erbb family of receptors, inducing the growth and differentiation of epithelial, neuronal, glial and other types of cells (Ring H et. al., Human Genetics 104:326-334 (1999)). In the lung, NRG2 may be involved in the differentiation of cell types, such as lung fibroblasts to myofibroblasts, which are recruited to the site of inflammation and partake in airway remodeling. Two SNPs have been identified within the ORF of NRG2 that cause amino acid changes. These amino acid changes in NRG2 can alter the functional capacity of the protein leading to the onset of asthma and other respiratory disorders.

Gene 891—Neuregulin 1

Human Neuregulin 1 (NRG1) is a member of the neuregulin family of growth and differentiation factors and maps to chromosome 8p21-p12. The transcript size is ˜2.0 Kb and there are nine alternatively transcribed species that encode nine protein isoforms. All NRG1 isoforms interact with the Erbb family of tyrosine kinase transmembrane receptors. The interaction of NRG1 isoforms with Erbb receptors 2/3 induces the growth and differentiation of epithelial, neuronal, glial, and other types of cells. NRG1 is the substrate of ADAM19, which is proteolytically cleaved allowing NRG1 to interact with Erbb2/3. In the lung, NRG1 maybe involved in the differentiation of cell types, such as lung fibroblasts to myofibroblasts, which are recruited to the site of inflammation and partake in airway remodeling. NRG1 has also been shown to activate the JAK-STAT pathway and regulate lung epithelial cell proliferation (Liu and Kern, Am. J. Respir. Mol. Biol. 27:306-13), thus implicating this gene in maintenance of epithelial integrity. It is possible that amino acid changes in NRG1 may alter the functional capacity of the protein leading to the onset of asthma and other respiratory disorders.

Gene 892—Endophilin1 (SH3GL2)

Human Endophilin 1 is a member of a family of proteins, which are adaptors that coordinate endocytosis, actin function and signaling cascades at the synapse and in non-neuronal cells. Endophilin 1 maps to 9p22. The transcript size is ˜2.7 Kb. ADAM9 and 15 have been shown to interact with Endophilin 1 by binding to the cytoplasmic domain of these proteins (Howard L, et. al., J Biol Chem 274:31693-31699 (1999)). Endophilin 1 may also interact with Gene216 at the cytoplasmic domain. The functional role of Endophilin 1 in non-neuronal cells is in membrane trafficking through clathrin-mediated endocytosis (Ringstad N, et. al., J Biol Chem [epub ahead of print] (2001)). This procedure is an important step in the process of modifying proteins en route to the membrane. It is possible that amino acid changes in Endophilin 1 may alter the functional capacity of the protein leading to the onset of asthma and other respiratory disorders.

Gene 893—Endophilin2 (SH3GL1)

Human Endophilin 2 is a member of a family of proteins, which are adaptors that coordinate endocytosis, actin function and signaling cascades at the synapse and in non-neuronal cells. Endophilin 2 maps to 19p13. The transcript size is ˜2.7 Kb. ADAM9 and 15 have been shown to interact with Endophilin 1 by binding to the cytoplasmic domain of these proteins (Howard L, et. al., J Biol Chem 274:31693-31699 (1999)). Endophilin 1 and 2 may interact with Gene216 at the cytoplasmic domain. The functional role of Endophilin 2 in non-neuronal cells, like Endophilin 1, is in membrane trafficking through clathrin-mediated endocytosis (Ringstad N, et. al., J Biol. Chem. 276(44): 40424-30 (2001)). This procedure is an important step in the process of modifying proteins en route to the membrane. It is possible that amino acid changes in Endophilin2 may alter the functional capacity of the protein leading to the onset of asthma and other respiratory disorders.

Gene 894—ADAM 3A

Human ADAM3a (cyritestin 1) is a member of the disintegrin and metalloprotease family and maps to chromosome 8p21-p12. The transcript is ˜2.6 Kb in size and is found to be expressed in testis (Adham I, et. al. DNA Cell Biol. 17: 161-168 (1998)). ADAM3a is involved in male fertility in mouse, however, in humans it appears to be non-functional (Grzmil P, et. al. Biochem J 357:551-556 (2001)). Based on the linkage analysis, ADAM3A and variants thereof can be involved in the onset of asthma and other respiratory disorders.

Gene 895—ADAM28

Human ADAM28 is a member of the disintegrin and metalloprotease family and maps to chromosome 8p21-p12. The transcript is 3.5 KB in size and highly expressed in epididymis and lymphocytes, and at lower levels in lung (Howard L, et. al., Biochem J 348:21-27 (2000)). Recently, ADAM28 has been shown to be a ligand for the leukocyte integrin alpha4beta1, implicating this gene in the interaction of lymphocytes with alpha4beta1-expressing leukocytes. Based on the linkage analysis, ADAM28 and variants thereof can be involved in the onset of asthma and other respiratory disorders.

Gene 896—ADAM7

Human ADAM7 is a member of the disintegrin and metalloprotease family and maps to chromosome 8p21-p12. There are two transcripts 4.0 and 3.0 Kb in size, which are expressed in the caput region of the epididymis and in the anterior pituitary gonadotropes. No expression was detected in the twenty-six other tissues examined including lung (Cornwall G A, Hsia N, Endocrinology 138:4262-4272 (1997) and Lin Y C, et. al. Biol Reprod 65:944-95 (2001)). Based on the linkage analysis, ADAM7 and variants thereof can be involved in the onset of asthma and other respiratory disorders.

Gene 897—ADAM9

Human ADAM9 is a member of the disintegrin and metalloprotease family and maps to chromosome 8q. The size of the transcript is ˜4.0 Kb and is expressed in many tissues including lung (Weskamp G, et. al., J Cell Biol 132:717-726 (1996)). ADAM9 has been shown to bind and proteolytically cleave the substrate heparin-binding EGF-like growth factor (HB-EGF). The secreted HB-EGF is a potent mitogen for a number of cell types and ADAM9 may act as a negative regulator (Izumi Y, et. al., EMBO 17:7260-7272 (1998)). Further, the cytoplasmic domain of ADAM9 has been shown to bind to Endophilin 1, which may modify the protein en route to its final destination at the cell surface. It is possible that amino acid changes in ADAM9 may alter the functional capacity of the protein leading to the onset of asthma and other respiratory disorders.

Gene 898—ADAM2

Human ADAM2 (Fertilin beta) is a member of the disintegrin and metalloprotease family and maps to chromosome 8p11.2. The size of the transcript is ˜2.8 Kb and is expressed in testis and prostate. ADAM2 is a cell adhesion molecule on the surface of mammalian sperm that participates in sperm-egg membrane binding (Evans J P, Bioessays 23:628-639 (2001)). Based on the linkage analysis, ADAM2 and variants thereof can be involved in the onset of asthma and other respiratory disorders.

Gene 899—ADAM18

Human ADAM18 is a member of the disintegrin and metalloprotease family and maps to chromosome 8p11.2. Based on the linkage analysis, ADAM18 and variants thereof can be involved in the onset of asthma and other respiratory disorders.

Gene 901—ADAMTS3

Human ADAMTS3 is an ADAM-related protein that possesses a disintegrin and metalloprotease domain as well as multiple copies of the thrombospondin motif. The gene maps to 4q13-q22 and the size of the transcript is ˜6.0 Kb. Like ADAMTS2, this gene has a limited expression profile: only expressed in adrenal gland, brain, breast, cervix, central nervous system, placenta, testis, and whole embryo. The enzyme encoded by this gene is similar in function to ADAMTS2; it excises the N-propeptide of type I, type II and type III procollagens (Tang B L, Int J Biochem Cell Biol 33:33-44 (2001)). Based on the linkage analysis, ADAMTS3 and variants thereof can be involved in the onset of asthma and other respiratory disorders.

Gene 902—ADAMTS9

Human ADAMTS9 is an ADAM-related protein that possesses a disintegrin and metalloprotease domain as well as multiple copies of the thrombospondin motif. The gene maps to 3p14.2-p14.3 and the size of the transcript is ˜4.0 Kb. It is expressed at low levels in adult tissues; however, RT/PCR analysis indicated that it was expressed in ovary, heart, kidney, lung, placenta and in many fetal tissues (Clark M E, et. al. Genomics 67:343-350 (2000)). Based on the linkage analysis, ADAMTS9 and variants thereof can be involved in the onset of asthma and other respiratory disorders.

Gene 903—Decysin

Human Decysin is a soluble ADAM-like protein that maps to chromosome 8p21-p12 between ADAM7 and 28. The transcript is ˜2.4 Kb in size and is expressed in limited number tissues that includes lung. Decysin is expressed in tissues where that demonstrate chronic antigen stimulation (Mueller C, et. al. J Exp Med 186:655-663 (1997)). The gene is expressed highly in mature dendritic cells that are localized to germinal centers. A continuous and high antigenic load in these sites may induce chronic interactions with dendritic and T-cells. Decysin maybe a key molecule in regulating the interaction of these cell types. Based on the linkage analysis, Decysin and variants thereof can be involved in the onset of asthma and other respiratory disorders.

Gene 962—ADAMTS2

Human ADAMTS2 is an ADAM-related protein that possesses a disintegrin and metalloprotease domain as well as multiple copies of the thrombospondin motif. The gene maps to chromosome 5q35 and the size of the transcript is ˜4.0 Kb. It has a limited expression profile: only found in breast, heart, kidney and uterus and skin. The enzyme encoded by this gene excises the N-propeptide of type I, type II and type V procollagens. Inactivating mutations in this gene cause Ehlers-Danlos syndrome type VIIC, a recessively inherited connective-tissue disorder (Colige A, et. al. Am J Hum Genet 65:308-317 (1999) and Shi-Wu L, et. al. Biochem J 355:271-278 (2001)). Based on the linkage analysis, ADAMTS2 and variants thereof can be involved in the onset of asthma and other respiratory disorders.

What is claimed is:

1. An isolated nucleic acid variant comprising a nucleotide sequence selected from the group shown in Table 2.

2. An isolated nucleic acid variant comprising a nucleotide sequence which contains at least one single nucleotide polymorphism as set forth in Tables 2-5 and 7, SEQ ID NOs. 1-9, and FIGS. 1-12.

3. An isolated nucleic acid variant comprising at least 15 contiguous nucleotides of a nucleotide sequence which contains at least one single nucleotide polymorphism as set forth in Tables 2-5 and 7, SEQ ID NOs. 1-9, and FIGS. 1-12.

4. An isolated nucleic acid comprising a nucleotide sequence that is complementary to the nucleotide sequence of the nucleic acid according to claim 2.

5. An isolated alternate splice variant comprising a nucleotide sequence shown in Tables 2-5 and 7, SEQ ID NOs. 1-9, and FIGS. 1-12.

6. An isolated alternate splice variant comprising at least 15 contiguous nucleotides of a nucleotide sequence selected from the sequences shown in Tables 2-5 and 7, SEQ ID NOs. 1-9, and FIGS. 1-12.

7. An isolated nucleic acid comprising a nucleotide sequence that is complementary to the nucleotide sequence according to claim 5.

8. A vector comprising the nucleic acid variant according to claim 2.

9. A vector comprising the nucleic acid variant according to claim 5.

10. A vector comprising the nucleic acid variant according to claim 3.

11. A vector comprising the nucleic acid variant according to claim 6.

12. A host cell comprising the vector according to claim 8, wherein the host cell is selected from the group consisting of bacterial, yeast, insect, mammalian, and plant cells.

13. A host cell comprising the vector according to claim 9, wherein the host cell is selected from the group consisting of bacterial, yeast, insect, mammalian, and plant cells.

14. A host cell comprising the vector according to claim 10, wherein the host cell is selected from the group consisting of bacterial, yeast, insect, mammalian, and plant cells.

15. A host cell comprising the vector according to claim 11, wherein the host cell is selected from the group consisting of bacterial, yeast, insect, mammalian, and plant cells.

16. An isolated polypeptide encoded by the nucleic acid variant according to claim 2.

17. An isolated polypeptide encoded by the alternate splice variant according to claim 5.

18. An antibody or antibody fragment that binds to the polypeptide according to claim 16.

19. An antibody or antibody fragment that binds to the polypeptide according to claim 17.

20. The antibody or antibody fragment according to claim 18 which is monoclonal.

21. The antibody or antibody fragment according to claim 19 which is monoclonal.

22. A pharmaceutical composition comprising the nucleic acid according to claim 2, and a physiologically acceptable carrier, excipient, or diluent.

23. A pharmaceutical composition comprising the nucleic acid according to claim 5, and a physiologically acceptable carrier, excipient, or diluent.

24. A pharmaceutical composition comprising the vector according to claim 8, and a physiologically acceptable carrier, excipient, or diluent.

25. A pharmaceutical composition comprising the vector according to claim 9, and a physiologically acceptable carrier, excipient, or diluent.

26. A pharmaceutical composition comprising the polypeptide according to claim 16, and a physiologically acceptable carrier, excipient, or diluent.

27. A pharmaceutical composition comprising the polypeptide according to claim 17, and a physiologically acceptable carrier, excipient, or diluent.

28. A pharmaceutical composition comprising the antibody according to claim 20, and a physiologically acceptable carrier, excipient, or diluent.

29. A pharmaceutical composition comprising the antibody according to claim 21, and a physiologically acceptable carrier, excipient, or diluent.

30. A kit for detecting an ADAM gene nucleotide sequence comprising:
a) the isolated nucleic acid variant according to claims 2 or 5; and
b) at least one component to detect hybridization of the isolated nucleic acid to an ADAM gene nucleotide sequence.

31. A kit for detecting an Interactor gene nucleotide sequence comprising:
a) the isolated nucleic acid variant according to claims 2 or 5; and
b) at least one component to detect hybridization of the isolated nucleic acid to an Interactor nucleotide sequence.

32. A kit for detecting an ADAM gene amino acid sequence comprising:
a) the antibody or antibody fragment according to claims 20 or 21; and
b) at least one component to detect binding of the antibody to an ADAM gene amino acid sequence.

33. A kit for detecting an Interactor gene amino acid sequence comprising:
a) the antibody or antibody fragment according to claims 20 or 21; and
b) at least one component to detect binding of the antibody to an Interactor gene amino acid sequence.

34. A method of diagnosing an ADAM gene-associated disorder in a human subject, comprising:
a) contacting the nucleic acid according to claim 1 with a biological sample obtained from the subject;
b) incubating the nucleic acid and biological sample under high stringency conditions that allow the nucleic acid to hybridize to a nucleic acid in the sample, and thereby form a complex; and
c) detecting the hybridization complex of (b), wherein detection of the complex indicates diagnosis of an ADAM gene-associated disorder.

35. The method of claim 34, wherein the disorder is selected from the group consisting of asthma, atopy, obesity, and inflammatory bowel disease.

36. A method of diagnosing an ADAM gene-associated disorder in a human subject, comprising:
a) contacting the antibody or antibody fragment according to claims 20 or 21 with a biological sample obtained from the subject;
b) incubating the antibody or antibody fragment and biological sample under conditions to allow the antibody or antibody fragment to bind to an amino acid sequence in the sample, and thereby form a complex; and
c) detecting the complex of (b), wherein detection of the complex indicates diagnosis of an ADAM gene-associated disorder.

37. The method according to claim 36, wherein the disorder is selected from the group consisting of asthma, atopy, obesity, and inflammatory bowel disease.

38. A method of diagnosing an Interactor gene-associated disorder in a human subject, comprising:
a) contacting the nucleic acid according to claim 1 with a biological sample obtained from the subject;
b) incubating the nucleic acid and biological sample under high stringency conditions that allow the nucleic acid to hybridize to a nucleic acid in the sample, and thereby form a complex; and
c) detecting the hybridization complex of (b), wherein detection of the complex indicates diagnosis of an Interactor gene-associated disorder.

39. The method of claim 38, wherein the disorder is selected from the group consisting of asthma, atopy, obesity, and inflammatory bowel disease.

40. A method of diagnosing an Interactor gene-associated disorder in a human subject, comprising:
a) contacting the antibody or antibody fragment according to claims 20 or 21 with a biological sample obtained from the subject;
b) incubating the antibody or antibody fragment and biological sample under conditions to allow the antibody or antibody fragment to bind to an amino acid sequence in the sample, and thereby form a complex; and
c) detecting the complex of (b), wherein detection of the complex indicates diagnosis of an ADAM gene-associated disorder.

41. The method according to claim 40, wherein the disorder is selected from the group consisting of asthma, atopy, obesity, and inflammatory bowel disease.

42. A method of determining an ADAM gene pharmacogenetic profile for a human subject comprising:
a) contacting the nucleic acid variant according to claims 2 or 5 with a biological sample obtained from the subject;
b) incubating the nucleic acid and biological sample under high stringency conditions to allow the nucleic acid to hybridize to a nucleic acid in the sample, and thereby form a complex;
c) detecting the hybridization complex of (b), wherein detection of the complex determines the ADAM gene pharmacogenetic profile.

43. A method of determining a ADAM gene pharmacogenetic profile for a human subject comprising:
a) contacting the antibody or antibody fragment according to claims 20 or 21 with a biological sample obtained from the subject;
b) incubating the antibody or antibody fragment with the biological sample under conditions that allow the antibody to bind to an amino acid sequence in the sample, and thereby form a complex; and
c) detecting the complex of (b), wherein detection of the complex determines the ADAM gene pharmacogenetic profile.

44. A method of determining an Interactor gene pharmacogenetic profile for a human subject comprising:
a) contacting the nucleic acid variant according to claims 2 or 5 with a biological sample obtained from the subject;
b) incubating the nucleic acid and biological sample under high stringency conditions to allow the nucleic acid to hybridize to a nucleic acid in the sample, and thereby form a complex;
c) detecting the hybridization complex of (b), wherein detection of the complex determines the Interactor gene pharmacogenetic profile.

45. A method of determining an Interactor gene pharmacogenetic profile for a human subject comprising:
a) contacting the antibody or antibody fragment according to claims 20 or 21 with a biological sample obtained from the subject;
b) incubating the antibody or antibody fragment with the biological sample under conditions that allow the antibody to bind to an amino acid sequence in the sample, and thereby form a complex; and
c) detecting the complex of (b), wherein detection of the complex determines the Interactor gene pharmacogenetic profile.

46. A method of identifying an ortholog of a human ADAM gene, comprising:
a) contacting the isolated nucleic acid variant according to claims 2 or 5 with a biological sample obtained from a non-human animal;
b) incubating the nucleic under conditions that allow the nucleic acid to hybridize to a nucleic acid in the sample, and thereby form a complex; and
c) detecting the hybridization complex of (a), wherein detection of the complex indicates identification of an ortholog of a human ADAM gene.

47. A method of identifying an ortholog of a human Interactor gene, comprising:
a) contacting the isolated nucleic acid variant according to claims 2 or 5 with a biological sample obtained from a non-human animal;
b) incubating the nucleic under conditions that allow the nucleic acid to hybridize to a nucleic acid in the sample, and thereby form a complex; and
c) detecting the hybridization complex of (a), wherein detection of the complex indicates identification of an ortholog of a human Interactor gene.

48. A method of treating an ADAM gene-associated disorder in a human subject comprising administering to the subject a pharmaceutical composition which comprises the vector according to claims 8 or 9, and a physiologically acceptable carrier, excipient, or diluent, in an amount effective to treat the disorder.

49. The method according to claim 48, wherein the ADAM gene-associated disorder is selected from the group consisting of asthma, atopy, obesity, and inflammatory bowel disease.

50. A method of treating a ADAM gene-associated disorder in a human subject comprising administering to the subject a pharmaceutical composition which comprises the host cell according to claims 12 or 13, and a physiologically acceptable carrier, excipient, or diluent, in an amount effective to treat the disorder.

51. The method according to claim 50, wherein the ADAM gene-associated disorder is selected from the group consisting of asthma, atopy, obesity, and inflammatory bowel disease.

52. A method of treating a ADAM gene-associated disorder in a human subject comprising administering to the subject a pharmaceutical composition which comprises the isolated nucleic acid variant according to claims 2 or 5, and a physiologically acceptable carrier, excipient or diluent, in an amount effective to treat the disorder.

53. The method according to claim 52, wherein the ADAM gene-associated disorder is selected from the group consisting of asthma, atopy, obesity, and inflammatory bowel disease.

54. A method of treating a ADAM gene-associated disorder in a human subject comprising administering to the subject a pharmaceutical composition which comprises the isolated polypeptide according to claims 16 or 17, and a physiologically acceptable carrier, excipient or diluent, in an amount effective to treat the disorder.

55. The method according to claim 54, wherein the ADAM gene-associated disorder is selected from the group consisting of asthma, atopy, obesity, and inflammatory bowel disease.

56. A method of treating an ADAM gene-associated disorder in a human subject comprising administering to the subject a pharmaceutical composition which comprises the antibody or antibody fragment according to claims 20 or 21, and a physiologically acceptable carrier, excipient or diluent, in an amount effective to treat the disorder.

57. The method according to claim 56, wherein the ADAM gene-associated disorder is selected from the group consisting of asthma, atopy, obesity, and inflammatory bowel disease.

58. A method of treating an Interactor gene-associated disorder in a human subject comprising administering to the subject a pharmaceutical composition which comprises the vector according to claims 8 or 9, and a physiologically acceptable carrier, excipient, or diluent, in an amount effective to treat the disorder.

59. The method according to claim 58, wherein the Interactor gene-associated disorder is selected from the group consisting of asthma, atopy, obesity, and inflammatory bowel disease.

60. A method of treating a Interactor gene-associated disorder in a human subject comprising administering to the subject a pharmaceutical composition which comprises the host cell according to claims 12 or 13, and a physiologically acceptable carrier, excipient, or diluent, in an amount effective to treat the disorder.

61. The method according to claim 60, wherein the Interactor gene-associated disorder is selected from the group consisting of asthma, atopy, obesity, and inflammatory bowel disease.

62. A method of treating a Interactor gene-associated disorder in a human subject comprising administering to the subject a pharmaceutical composition which comprises the isolated nucleic acid variant according to claims 2 or 5, and a physiologically acceptable carrier, excipient or diluent, in an amount effective to treat the disorder.

63. The method according to claim 62, wherein the Interactor gene-associated disorder is selected from the group consisting of asthma, atopy, obesity, and inflammatory bowel disease.

64. A method of treating a Interactor gene-associated disorder in a human subject comprising administering to the subject a pharmaceutical composition which comprises the isolated polypeptide according to claims 16 or 17, and a physiologically acceptable carrier, excipient or diluent, in an amount effective to treat the disorder.

65. The method according to claim 64, wherein the Interactor gene-associated disorder is selected from the group consisting of asthma, atopy, obesity, and inflammatory bowel disease.

66. A method of treating an Interactor gene-associated disorder in a human subject comprising administering to the subject a pharmaceutical composition which comprises the antibody or antibody fragment according to claims 20 or 21, and a physiologically acceptable carrier, excipient or diluent, in an amount effective to treat the disorder.

67. The method according to claim 66, wherein the Interactor gene-associated disorder is selected from the group consisting of asthma, atopy, obesity, and inflammatory bowel disease.

68. A transgenic mouse whose genome comprises an introduced null mutation in an endogenous gene which is orthologous to a human ADAM gene comprising a nucleotide sequence according to claims 2 or 5.

69. The transgenic mouse according to claim 68, wherein both alleles of the endogenous gene have been disrupted.

70. The transgenic mouse according to claim 69, wherein the mouse genome further comprises a human ADAM gene variant nucleotide sequence selected from the group shown in Tables 2-5 and 7, SEQ ID NOs. 1-9, and FIGS. 1-12.

71. The transgenic mouse according to claim 69, wherein the mouse genome further comprises a human Interactor gene variant nucleotide sequence selected from the group shown in Tables 2-5 and 7, SEQ ID NOs. 1-9, and FIGS. 1-12.

72. A method of making a homozygous transgenic knockout mouse comprising:
a) disrupting an endogenous gene in mouse embryonic stem cells, wherein the endogenous gene is orthologous to a human ADAM gene variant comprising a nucleotide sequence selected from the group shown in Tables 2-5 and 7, SEQ ID NOs. 1-9, and FIGS. 1-12;
b) introducing said embryonic stem cells into a mouse blastocyst and transplanting said blastocyst into a pseudopregnant mouse;
c) allowing said blastocyst to develop into a chimeric mouse;
d) breeding said chimeric mouse to produce offspring; and
e) screening said offspring to identify a homozygous transgenic knockout mouse.

73. A method of making a homozygous transgenic knockout mouse comprising:
a) disrupting an endogenous gene in mouse embryonic stem cells, wherein the endogenous gene is orthologous to a human Interactor gene variant comprising a nucleotide sequence selected from the group shown in Tables 2-5 and 7, SEQ ID NOs. 1-9, and FIGS. 1-12;
b) introducing said embryonic stem cells into a mouse blastocyst and transplanting said blastocyst into a pseudopregnant mouse;
c) allowing said blastocyst to develop into a chimeric mouse;
d) breeding said chimeric mouse to produce offspring; and
e) screening said offspring to identify a homozygous transgenic knockout mouse.

74. A method of forming a crystal of the isolated polypeptide according to claim 16 comprising:
a) incubating the polypeptide with a solution under conditions to allow crystalization; and
b) detecting the crystalization in (a), whereby crystalization indicates formation of a crystal.

75. A method of forming a crystal of the isolated polypeptide according to claim 17 comprising:
a) incubating the polypeptide with a solution under conditions to allow crystalization; and
b) detecting the crystalization in (a), whereby crystalization indicates formation of a crystal.

76. A cell line comprising the isolated nucleic acid variant according to claim 2.

77. A cell line comprising the isolated nucleic acid variant according to claim 5.

78. A biochip comprising the isolated nucleic acid variant according to claim 2.

79. A biochip comprising the isolated nucleic acid variant according to claim 5.

80. An isolated nucleic acid probe comprising at least 8 contiguous nucleotides of a nucleotide sequence selected from Tables 2-5 and 7.

81. An isolated nucleic acid probe comprising at least 8 contiguous nucleotides of a nucleotide sequence selected from SEQ ID NOs: 1-9, and FIGS. 1-12.

82. An isolated nucleic acid primer comprising at least 8 contiguous nucleotides of a nucleotide sequence selected from Tables 2-5 and 7.

83. An isolated nucleic acid primer comprising at least 8 contiguous nucleotides of a nucleotide sequence selected from SEQ ID NOs: 1-9, and FIGS. 1-12.

84. An isolated antisense nucleic acid comprising the nucleotide sequence according to claim 2.

85. An isolated antisense nucleic acid comprising the nucleotide sequence according to claim 5.

86. A method of identifying an ADAM gene ligand, comprising:
a) contacting the isolated polypeptide according to claims 16 or 18 with a test agent;
b) incubating the isolated polypeptide and the test agent under conditions that allow the polypeptide to bind to the test agent, and thereby form a complex; and
c) detecting the complex of (b), wherein detection of the complex indicates identification of an ADAM gene ligand.

87. A method of identifying an ADAM gene ligand, comprising:
a) contacting a polypeptide comprising at least 7 contiguous amino acids of the isolated polypeptide according to claims 16 or 18 with a test agent;
b) incubating the polypeptide and the test agent under conditions that allow the polypeptide to bind to the test agent, and thereby form a complex; and
c) detecting the complex of (b), wherein detection of the complex indicates identification of an ADAM gene ligand.

88. A method of identifying an ADAM gene ligand, comprising:
a) contacting the isolated nucleic acid variant according to claims 2 or 5 with a test agent;
b) incubating the isolated nucleic acid and the test agent under conditions that allow the nucleic acid to bind to the test agent, and thereby form a complex; and
c) detecting the complex of (b), wherein detection of the complex indicates identification of an ADAM gene ligand.

89. The method according to claim 86, wherein the test agent comprises a small molecule.

90. The method according to claim 87, wherein the test agent comprises a small molecule.

91. The method according to claim 88, wherein the test agent comprises a small molecule.

92. A method of identifying an Interactor gene ligand, comprising:
a) contacting the isolated polypeptide according to claims 16 or 17 with a test agent;
b) incubating the isolated polypeptide and the test agent under conditions that allow the polypeptide to bind to the test agent, and thereby form a complex; and
c) detecting the complex of (b), wherein detection of the complex indicates identification of an Interactor gene ligand.

93. A method of identifying an Interactor gene ligand, comprising:
a) contacting a polypeptide comprising at least 7 contiguous amino acids of the isolated polypeptide according to claims 16 or 17 with a test agent;
b) incubating the polypeptide and the test agent under conditions that allow the polypeptide to bind to the test agent, and thereby form a complex; and
c) detecting the complex of (b), wherein detection of the complex indicates identification of an Interactor gene ligand.

94. A method of identifying an Interactor gene ligand, comprising:
a) contacting the isolated nucleic acid variant according to claims 2 or 5 with a test agent;
b) incubating the isolated nucleic acid and the test agent under conditions that allow the nucleic acid to bind to the test agent, and thereby form a complex; and
c) detecting the complex of (b), wherein detection of the complex indicates identification of an Interactor gene ligand.

95. The method according to claim 92, wherein the test agent comprises a small molecule.

96. The method according to claim 93, wherein the test agent comprises a small molecule.

97. The method according to claim 94, wherein the test agent comprises a small molecule.

98. A method of treating an ADAM gene-associated disorder in a human subject comprising administering to the subject a pharmaceutical composition which comprises the ligand isolated by the method according to claim 86, and a physiologically acceptable carrier, excipient or diluent, in an amount effective to treat the disorder.

99. A method of treating an ADAM gene-associated disorder in a human subject comprising administering to the subject a pharmaceutical composition which comprises the ligand isolated by the method according to claim 87, and a physiologically acceptable carrier, excipient or diluent, in an amount effective to treat the disorder.

100. A method of treating an ADAM gene-associated disorder in a human subject comprising administering to the subject a pharmaceutical composition which comprises the ligand isolated by the method according to claim 88, and a physiologically acceptable carrier, excipient or diluent, in an amount effective to treat the disorder.

101. A method of treating an Interactor gene-associated disorder in a human subject comprising administering to the subject a pharmaceutical composition which comprises the ligand isolated by the method according to claim 92, and a physiologically acceptable carrier, excipient or diluent, in an amount effective to treat the disorder.

102. A method of treating an Interactor gene-associated disorder in a human subject comprising administering to the subject a pharmaceutical composition which comprises the ligand isolated by the method according to claim 93, and a physiologically acceptable carrier, excipient or diluent, in an amount effective to treat the disorder.

103. A method of treating an Interactor gene-associated disorder in a human subject comprising administering to the subject a pharmaceutical composition which comprises the ligand isolated by the method according to claim 94, and a physiologically acceptable carrier, excipient or diluent, in an amount effective to treat the disorder.

104. A method of diagnosing a respiratory disorder in a human subject, comprising:
a) contacting a nucleic acid sequence of an ADAM gene variant with a biological sample obtained from the subject;
b) incubating the nucleic acid and biological sample under high stringency conditions that allow the nucleic acid to hybridize to a nucleic acid in the sample, and thereby form a complex; and
c) detecting the hybridization complex of (b), wherein detection of the complex indicates diagnosis of a respiratory disorder.

105. The method according to claim 104, wherein the respiratory disorder is asthma or atopy.

106. A method of diagnosing a respiratory disorder in a human subject, comprising:
a) contacting a nucleic acid sequence of an Interactor gene variant with a biological sample obtained from the subject;
b) incubating the nucleic acid and biological sample under high stringency conditions that allow the nucleic acid to hybridize to a nucleic acid in the sample, and thereby form a complex; and
c) detecting the hybridization complex of (b), wherein detection of the complex indicates diagnosis of a respiratory disorder.

107. The method according to claim 106, wherein the respiratory disorder is asthma or atopy.

108. A method of diagnosing a respiratory disorder in a human subject, comprising:
a) contacting an ADAM gene polypeptide antibody or antibody fragment with a biological sample obtained from the subject;
b) incubating the antibody or antibody fragment and biological sample under conditions that allow the antibody or antibody fragment to bind to an amino acid sequence in the sample, and thereby form a complex; and
c) detecting the complex of (b), wherein detection of the complex indicates diagnosis of a respiratory disorder.

109. The method according to claim 108, wherein the respiratory disorder is asthma or atopy.

110. A method of diagnosing a respiratory disorder in a human subject, comprising:
a) contacting an Interactor gene polypeptide antibody or antibody fragment with a biological sample obtained from the subject;
b) incubating the antibody or antibody fragment and biological sample under conditions that allow the antibody or antibody fragment to bind to an amino sequence in the sample, and thereby form a complex; and
c) detecting the complex of (b), wherein detection of the complex indicates diagnosis of a respiratory disorder.

111. The method according to claim 110, wherein the respiratory disorder is asthma or atopy.

112. A method of treating a respiratory disorder in a human subject comprising administering to the subject a pharmaceutical composition which comprises an isolated nucleic acid sequence of an ADAM gene, and a physiologically acceptable carrier, excipient, or diluent, in an amount effective to treat the respiratory disorder.

113. The method according to claim 112, wherein the respiratory disorder is asthma or atopy.

114. A method of treating a respiratory disorder in a human subject comprising administering to the subject a pharmaceutical composition which comprises an isolated nucleic acid sequence of an Interactor gene, and a physiologically acceptable carrier, excipient, or diluent, in an amount effective to treat the respiratory disorder.

115. The method according to claim 114, wherein the respiratory disorder is asthma or atopy.

116. A method of treating a respiratory disorder in a human subject comprising administering to the subject a pharmaceutical composition which comprises a vector comprising an ADAM gene nucleotide sequence, and a physiologically acceptable carrier, excipient, or diluent, in an amount effective to treat the respiratory disorder.

117. The method according to claim 116, wherein the respiratory disorder is asthma or atopy.

118. A method of treating a respiratory disorder in a human subject comprising administering to the subject a pharmaceutical composition which comprises a vector comprising an Interactor gene nucleotide sequence, and a physiologically acceptable carrier, excipient, or diluent, in an amount effective to treat the respiratory disorder.

119. The method according to claim 118, wherein the respiratory disorder is asthma or atopy.

120. A method of treating a respiratory disorder in a human subject comprising administering to the subject a pharmaceutical composition which comprises a host cell comprising an ADAM gene nucleotide sequence, and a physiologically acceptable carrier, excipient, or diluent, in an amount effective to treat the respiratory disorder.

121. The method according to claim 120, wherein the respiratory disorder is asthma or atopy.

122. A method of treating a respiratory disorder in a human subject comprising administering to the subject a pharmaceutical composition which comprises a host cell comprising an Interactor gene nucleotide sequence, and a physiologically acceptable carrier, excipient, or diluent, in an amount effective to treat the respiratory disorder.

123. The method of claim 122, wherein the respiratory disorder is asthma or atopy.

124. A method of treating a respiratory disorder in a human subject comprising administering to the subject a pharmaceutical composition which comprises an isolated polypeptide encoded by an ADAM gene nucleotide sequence, and a physiologically acceptable carrier, excipient, or diluent, in an amount effective to treat the respiratory disorder.

125. The method of claim 124, wherein the respiratory disorder is asthma or atopy.

126. A method of treating a respiratory disorder in a human subject comprising administering to the subject a pharmaceutical composition which comprises an isolated polypeptide encoded by an Interactor gene nucleotide sequence, and a physiologically acceptable carrier, excipient, or diluent, in an amount effective to treat the respiratory disorder.

127. The method of claim 126, wherein the respiratory disorder is asthma or atopy.

128. A method of treating a respiratory disorder in a human subject comprising administering to the subject a pharmaceutical composition which comprises an ADAM protein antibody or antibody fragment, and a physiologically acceptable carrier, excipient, or diluent, in an amount effective to treat the respiratory disorder.

129. The method according to claim 128, wherein the respiratory disorder is asthma or atopy.

130. A method of treating a respiratory disorder in a human subject comprising administering to the subject a pharmaceutical composition which comprises Interactor protein antibody or antibody fragment, and a physiologically acceptable carrier, excipient, or diluent, in an amount effective to treat the respiratory disorder.

131. The method according to claim 130, wherein the respiratory disorder is asthma or atopy.

132. A method of diagnosing an ADAM or Interactor gene-associated disorder in a human subject, comprising:
a) contacting a nucleic acid shown in Table 7 with a biological sample obtained from the subject;
b) incubating the nucleic acid and biological sample under high stringency conditions that allow the nucleic acid to hybridize to a nucleic acid in the sample, and thereby form a complex; and
c) detecting the hybridization complex of (b), wherein detection of the complex indicates diagnosis of an ADAM or Interactor gene-associated disorder.

133. The method of claim 132, wherein the disorder is selected from the group consisting of asthma, atopy, obesity, and inflammatory bowel disease.

134. An isolated nucleic acid variant of Gene 803 which contains at least allele A at single nucleotide polymorphism K 2.

135. An isolated nucleic acid variant of Gene 845 which contains at least one allele selected from the group consisting of:
a. allele T at single nucleotide polymorphism P+1;
b. allele G at single nucleotide polymorphism J 1; and
c. allele T at single nucleotide polymorphism D−1.

136. An isolated nucleic acid variant of Gene 847 which contains at least allele G at single nucleotide polymorphism C+1.

137. An isolated nucleic acid variant of Gene 962 which contains at least one allele selected from the group consisting of:
a. allele C at single nucleotide polymorphism M+2;
b. allele A at single nucleotide polymorphism P−2;
c. allele A at single nucleotide polymorphism Q−1;
d. allele T at single nucleotide polymorphism U2;
e. allele C at single nucleotide polymorphism V−1;
f. allele G at single nucleotide polymorphism G4;
g. allele A at single nucleotide polymorphism G1;
h. allele C at single nucleotide polymorphism G6;
i. allele G at single nucleotide polymorphism M+2;
j. allele G at single nucleotide polymorphism S−1; and
k. allele C at single nucleotide polymorphism Z1.

138. An isolated nucleic acid variant of Gene 803 which contains at least one haplotype selected from the group consisting of:
a. haplotype C/A at single polymorphisms K3/K2;
b. haplotype A/G at single polymorphisms K2/I−1;
C. haplotype A/C at single polymorphisms K2/I1;
d. haplotype A/A at single polymorphisms K2/E+2;
e. haplotype G/C at single polymorphisms K2/I1; and
f. haplotype G/C at single polymorphisms K2/E+2.

139. An isolated nucleic acid variant of Gene 845 which contains at least one haplotype selected from the group consisting of:
a. haplotype G/G at single polymorphisms R1/K−2;
b. haplotype C/T at single polymorphisms K1/D−1;
C. haplotype G/C at single polymorphisms K−2/H+1;
d. haplotype T/G at single polymorphisms R−1/J1;
e. haplotype G/T at single polymorphisms K−2/D1;
f. haplotype G/C at single polymorphisms J1/D1;
g. haplotype A/T at single polymorphisms J1/D1;
h. haplotype C/T at single polymorphisms H−1/D1;
i. haplotype C/T at single polymorphisms D1/D−1;
j. haplotype T/T at single polymorphisms D1/D−1;
k. haplotype T/C at single polymorphisms P+1/H+1;
l. haplotype C/C at single polymorphisms K1/H+1;
m. haplotype G/C at single polymorphisms R1/R−1;
n. haplotype A/G at single polymorphisms R1/K−2;
o. haplotype C/C at single polymorphisms K1/D−1;
p. haplotype A/C at single polymorphisms J1/D1;
q. haplotype C/C at single polymorphisms D1/D−1;
r. haplotype C/G at single polymorphisms K1/H+1; and
s. haplotype A/C at single polymorphisms R1/R−1.

140. An isolated nucleic acid variant of Gene 847 which contains at least one haplotype selected from the group consisting of:
a. haplotype G/C at single polymorphisms K1/D−1;
b. haplotype G/A at single polymorphisms K1/C+1;
c. haplotype C/T at single polymorphisms J+1/E+1;
d. haplotype C/A at single polymorphisms J+1/D−1;
e. haplotype C/A at single polymorphisms J+1/C+1;
f. haplotype A/C at single polymorphisms D−1/A2;
g. haplotype A/C at single polymorphisms C+1/A2;
h. haplotype A/G at single polymorphisms D−1/A1;
i. haplotype G/G at single polymorphisms K1/C+1;
j. haplotype C/C at single polymorphisms J+1/D−1;
k. haplotype C/G at single polymorphisms J+1/C+1; and
l. haplotype C/G at single polymorphisms D−1/A1.

141. An isolated nucleic acid variant of Gene 962 which contains at least one haplotype selected from the group consisting of:
a. haplotype A/A at single polymorphisms G1/Q−1;
b. haplotype A/C at single polymorphisms G1/V−1;
c. haplotype G/A at single polymorphisms G4/G1;
d. haplotype G/A at single polymorphisms G4/Q−1;
e. haplotype G/T at single polymorphisms G4/U2;
f. haplotype A/A at single polymorphisms G4/V+2;
g. haplotype A/T at single polymorphisms G1/U2;
h. haplotype T/G at single polymorphisms U2/V+2;
i. haplotype A/C at single polymorphisms G2/S−1;
j. haplotype T/A at single polymorphisms E+2/G1;
k. haplotype A/A at single polymorphisms G1/P−2;
l. haplotype G/C at single polymorphisms G4/G6;
m. haplotype C/G at single polymorphisms G6/S−1;
n. haplotype G/A at single polymorphisms G4/Q−1;
o. haplotype G/A at single polymorphisms M+2/P−2;
p. haplotype G/G at single polymorphisms G4/S−1;
q. haplotype A/G at single polymorphisms G1/M+2;
r. haplotype A/G at single polymorphisms G1/S−1;
s. haplotype G/G at single polymorphisms G1/S−1;
t. haplotype C/C at single polymorphisms G6/V−1;
u. haplotype A/C at single polymorphisms Q−1/V−1;
v. haplotype G/C at single polymorphisms U1/Z1;
w. haplotype T/C at single polymorphisms U2/V−1;
x. haplotype T/G at single polymorphisms E3/G1;
y. haplotype T/C at single polymorphisms Q−1/V−1;
z. haplotype G/T at single polymorphisms U1/Z1;
aa. haplotype C/C at single polymorphisms U2/V−1;
bb. haplotype A/C at single polymorphisms H+2/S−1;
cc. haplotype C/T at single polymorphisms E+2/V−1;
dd. haplotype G/T at single polymorphisms G1/G6;
ee. haplotype C/C at single polymorphisms G6/S−1;
ff. haplotype G/C at single polymorphisms G1/M+2;
gg. haplotype G/C at single polymorphisms G1/S−1;
hh. haplotype C/C at single polymorphisms G6/S−1;
ii. haplotype T/C at single polymorphisms G6/V−1;
jj. haplotype A/G at single polymorphisms G4/G1;
kk. haplotype C/T at single polymorphisms E+2/V−1;
ll. haplotype G/A at single polymorphisms G1/Q−1;
mm. haplotype T/G at single polymorphisms G6/S−1; and
nn. haplotype G/G at single polymorphisms M+2/P−2.

142. An isolated nucleic acid which is complementary to the nucleic acid of any one of claims 134-141.

143. A probe comprising the isolated nucleic acid of any one of claims 134-141.

144. A primer comprising the isolated nucleic acid of any one of claims 131-141.

145. An isolated amino acid sequence encoded by the isolated nucleic acid of any one of claims 134-141.

146. An antibody which binds to the isolated amino acid sequence of claim 166, wherein the antibody is polyclonal or monoclonal.

147. A vector comprising the isolated nucleic acid of any one of claims 134-141.

148. A pharmaceutical composition comprising the isolated nucleic acid of any one of claims 134-141.
